(19) United States
(12) Patent Application Publication
(10) Pub. No.: US 2024/0292746 A1
(43) Pub. Date: Aug. 29, 2024

(54) ORGANIC ELECTROLUMINESCENT COMPOUND, ORGANIC ELECTROLUMINESCENT MATERIAL, AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Hyo-Jung LEE, Gyeonggi-do (KR); Jeong-Eun YANG, Gyeonggi-do (KR); So-Young JUNG, Gyeonggi-do (KR); Sang-Hee CHO, Gyeonggi-do (KR); Du-Yong PARK, Gyeonggi-do (KR)

(21) Appl. No.: 18/414,731

(22) Filed: Jan. 17, 2024

(30) Foreign Application Priority Data
Feb. 1, 2023 (KR) 10-2023-0013830
Dec. 4, 2023 (KR) 10-2023-0173630

(51) Int. Cl. H10K 85/60; C07D 498/04; C09K 11/06; H10K 50/11

(52) U.S. Cl. CPC H10K 85/657; C07D 498/04; C09K 11/06; H10K 85/636; H10K 85/654; C09K 2211/1018; H10K 50/11

(57) ABSTRACT

The present disclosure relates to an organic electroluminescent compound represented by Formula 1, an organic electroluminescent material, and an organic electroluminescent device comprising the same. By comprising the organic electroluminescent compound or the organic electroluminescent material of the present disclosure, an organic electroluminescent device having improved driving voltage, luminous efficiency and/or lifetime characteristics can be provided.

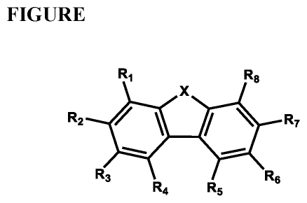

### TECHNICAL FIELD

The present disclosure relates to an organic electroluminescent compound, an organic electroluminescent material, and organic electroluminescent device comprising the same.

### BACKGROUND ART

In 1987, Tang et al. of Eastman Kodak first developed a small molecular green organic electroluminescent device (OLED) by using TPD/Alq3 bi-layer consisting of a light-emitting layer and a charge transport layer. Thereafter, the development of OLEDs was rapidly effected and OLEDs having been commercialized. Currently, OLEDs mainly use phosphorescent materials having excellent luminous efficiency in panel implementation. OLEDs having high luminous efficiency and/or long lifetime are required for long-term use and high resolution of a display.

Korean Patent Application Laying-open No. 10-2015-0093181 A and No. 10-2021-0038785 A disclose an organic electroluminescent device comprising compounds having a structure, in which a 5-membered heteroaryl is fused to a moiety such as dibenzofuran or dibenzothiophene. However, the aforementioned references do not disclose the compounds of the present disclosure, and there is still a need to develop organic electroluminescent materials to improve the performance of OLEDs.

### DISCLOSURE OF INVENTION

### Technical Problem

The objective of the present disclosure is firstly, to provide an organic electroluminescent compound effective for producing an organic electroluminescent device having improved driving voltage, luminous efficiency, and/or lifetime property, and secondly, to provide an organic electroluminescent material and an organic electroluminescent device comprising the organic electroluminescent compound.

### Solution to Problem

As a result of intensive research to solve the above technical problems, the present inventors have found that the above objective can be achieved by an organic electroluminescent compound represented by Formula 1 and completed the present disclosure.

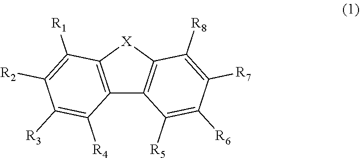

In Formula 1,
X represents O, S or Se;
R₁ to R₈ each independently represent hydrogen, deuterium, a halogen, a cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, a substituted or unsubstituted (C1-C30)alkoxy, a substituted or unsubstituted amino, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30) arylamino, a substituted or unsubstituted (C2-C30)alkenyl(3- to 30-membered)heteroarylamino, or a substituted or unsubstituted (C6-C30)aryl(3- to 30-membered)heteroarylamino;
R₁ and R₂, R₂ and R₃, or R₃ and R₄ fuse with Formula 1-1 to form a ring(s), and R₅ and R₆, R₆ and R₇, or R₇ and R₈ fuse with formulas 1-2 or 1-3 to form a ring(s);

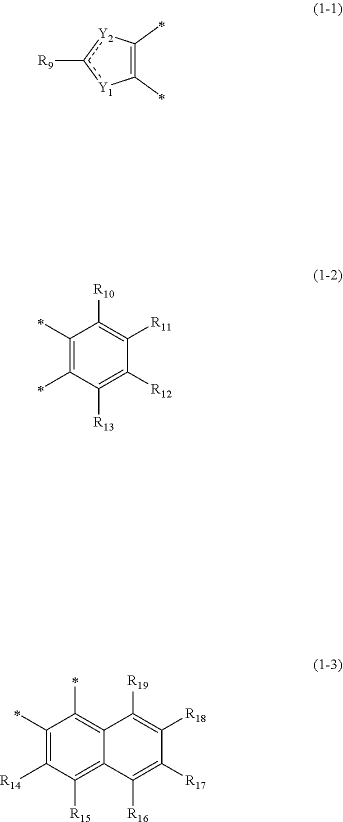

in formulas 1-1 to 1-3,
Y₁ and Y₂ each independently represent O, S, CR₂₀R₂₁, or NR₂₂;
R₉ to R₁₉ are each independently the same as the definition of R₁; and
* represents a site fused with Formula 1.

### Advantageous Effects of Invention

By using the organic electroluminescent compound and the organic electroluminescent material of the present disclosure, an organic electroluminescent device having improved driving voltage, luminous efficiency and/or lifetime properties can be provided.

### BRIEF DESCRIPTION OF THE FIGURE

The FIGURE illustrates a representative formula of the organic electroluminescent compound of the present disclosure.

### MODE FOR THE INVENTION

Hereinafter, the present disclosure will be described in detail. However, the following description is intended to explain the present disclosure, and is not meant to restrict the scope of the present disclosure.

The term “organic electroluminescent compound” in the present disclosure means a compound that may be used in an organic electroluminescent device, and may be comprised in any layer constituting an organic electroluminescent device, as necessary.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc. The hole transfer zone material may consist of one or more selected from the group comprised of a hole transport material, a hole injection material, an electron blocking material, a hole auxiliary material, and a light-emitting auxiliary material.

The organic electroluminescent material of the present disclosure may comprise at least one compound represented by Formula 1. The compound of Formula 1 may be included in at least one of the layers constituting organic electroluminescent device, and may be included in light-emitting layer and/or at least one of the layers constituting the hole transfer zone, but is not limited thereto. When the compound of Formula 1 is included in a hole transport layer, a hole auxiliary layer, a light-emitting layer, or light-emitting auxiliary layer, it may be included as a hole transport material, a hole auxiliary material, a host material, or a light-emitting auxiliary material.

Herein, the term “(C1-C30)alkyl(ene)” is meant to be a linear or branched alkyl(ene) having 1 to 30 carbon atoms constituting the chain, in which the number of carbon atoms is preferably 1 to 20, and more preferably 1 to 10. The above alkyl may include methyl, ethyl, n-propyl, iso-propyl, n-butyl, isobutyl, tert-butyl, etc. The term “(C2-C30)alkenyl” is meant to be a linear or branched alkenyl having 2 to 30 carbon atoms constituting the chain, in which the number of carbon atoms is preferably 2 to 20, and more preferably 2 to 10. The above alkenyl may include vinyl, 1-propenyl, 2-propenyl, 1-butenyl, 2-butenyl, 3-butenyl, 2-methylbut-2-enyl, etc. The term “(C2-C30)alkynyl” is meant to be a linear or branched alkynyl having 2 to 30 carbon atoms constituting the chain, in which the number of carbon atoms is preferably 2 to 20, and more preferably 2 to 10. The above alkynyl may include ethynyl, 1-propynyl, 2-propynyl, 1-butynyl, 2-butynyl, 3-butynyl, 1-methylpent-2-ynyl, etc. The term “(C3-C30)cycloalkyl(ene)” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, etc. The term “(3- to 7-membered)heterocycloalkyl” is meant to be a cycloalkyl having 3 to 7, preferably 5 to 7, ring backbone atoms, and including at least one heteroatom selected from the group consisting of B, N, O, S, Si, and P, and preferably the group consisting of O, S, and N. The above heterocycloalkyl may include tetrahydrofuran, pyrrolidine, thiolan, tetrahydropyran, etc. The term “(C6-C30)aryl(ene)” is meant to be a monocyclic or fused ring radical derived from an aromatic hydrocarbon having 6 to 30 ring backbone carbon atoms, and the above aryl may be partially saturated. The number of the ring backbone carbon atoms is preferably 6 to 25, more preferably 6 to 18. The above aryl may comprise a spiro structure. The above aryl may include phenyl, biphenyl, terphenyl, naphthyl, binaphthyl, phenylnaphthyl, naphthylphenyl, phenylterphenyl, fluorenyl, phenylfluorenyl, diphenylfluorenyl, dimethylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, etc. More specifically, the aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenyl, 4′-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b] fluorenyl, 11,11-dimethyl-5-benzo[b]fluorenyl, 11,11-dimethyl-6-benzo[b]fluorenyl, 11,11-dimethyl-7-benzo[b] fluorenyl, 11,11-dimethyl-8-benzo[b]fluorenyl, 11,11-dimethyl-9-benzo[b]fluorenyl, 11,11-dimethyl-10-benzo[b] fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

Herein, the term “(3- to 30-membered)heteroaryl(ene)” is an aryl(ene) group having 3 to 30 ring backbone atoms, and including at least one heteroatoms selected from the group consisting of B, N, O, S, Si, and P. The number of heteroatoms is preferably 1 to 4 and the above heteroaryl may be a monocyclic ring, or a fused ring condensed with at least one benzene ring, and may be partially saturated. In addition, the above heteroaryl(ene) may comprise one formed by linking at least one heteroaryl or aryl group to a heteroaryl(ene) group via a single bond, and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, benzonaphthofuranyl, benzophenanthrofuranyl, dibenzothiophenyl, benzonaphthothiophenyl, benzimidazolyl, benzothiazolyl, benzoisothiazolyl, benzophenanthrothiophenyl, benzoisoxazolyl, benzoxazolyl, phenanthroxazolyl, phenanthrothiazolyl, isoindolyl, indolyl, benzoindolyl, indazolyl, benzothiadiazolyl, quinolyl, isoquinolyl, cinnolinyl, quinazolinyl, benzoquinazolinyl, quinoxalinyl, benzoquinoxalinyl, naphthyridinyl, carbazolyl, benzocarbazolyl, dibenzocarbazolyl, phenoxazinyl, phenothiazinyl, phenanthridinyl, benzodioxolyl, dihydroacridinyl, etc. More specifically, the heteroaryl may include 1-pyrrolyl, 2-pyrrolyl, 3-pyrrolyl, pyrazinyl, 2-pyridyl, 2-pyrimidinyl, 4-pyrimidinyl, 5-pytimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridinyl, 3-imidazopyridinyl, 5-imidazopyridinyl, 6-imidazopyridinyl, 7-imidazopyridinyl, 8-imidazopyridinyl, 3-pyridinyl, 4-pyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d] pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. In the present disclosure, the term “halogen” includes F, Cl, Br, and I.

In addition, “ortho (O-),” “meta (m-),” and “para (p-)” are prefixes, which represent the relative positions of substituents, respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

In addition, “substituted” in the expression “substituted or unsubstituted” means that a hydrogen atom in a certain functional group is replaced with another atom or another functional group (i.e., a substituent). Herein, the substituent(s) of the substituted alkyl, the substituted cycloalkyl, the substituted aryl, the substituted heteroaryl, the substituted alkoxy and the substituted fused ring group of an aliphatic ring(s) and an aromatic ring(s), each independently, are at least one selected from the group consisting of deuterium; a halogen; a cyano; a carboxyl; a nitro; a hydroxyl; a phosphine oxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl, a (C3-C30)cycloalkenyl; a (3- to 7-membered)heterocycloalkyl; a (C6-C30)aryloxy; a (C6-C30)arylthio; a (C6-C30)aryl unsubstituted or substituted with at least one of a (C1-C30) alkyl(s) and a di(C6-C30)arylamino(s); a (3- to 30-membered)heteroaryl unsubstituted or substituted with at least one of a (C6-C30)aryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; a fused ring group of a (C3-C30)aliphatic ring(s) and a (C6-C30)aromatic ring(s); an amino; a mono- or di-(C1-C30)alkylamino; a mono- or di-(C2-C30)alkenylamino; a mono- or di-(C6-C30)arylamino unsubstituted or substituted with a (C1-C30)alkyl(s); a mono- or di-(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a di(C6-C30)arylboronyl; a di(C1-C30) alkylboronyl; a (C1-C30)alkyl(C6-C30)arylboronyl; a (C6-C30)aryl(C1-C30)alkyl; and a (C1-C30)alkyl(C6-C30)aryl. According to one embodiment of the present disclosure, the substituent(s), each independently, are at least one selected from the group consisting of a (C1-C30)alkyl; (C6-C30)aryl unsubstituted or substituted with at least one of a (C1-C30)alkyl(s) and di(C6-C30)arylamino(s); a (3- to 30-membered)heteroaryl unsubstituted or substituted with a (C6-C30)aryl(s); and a substituted or unsubstituted di(C6-C30)arylamino. According to another embodiment of the present disclosure, the substituent(s), each independently, are at least one selected from the group consisting of a (C1-C6)alkyl; a (C6-C20)aryl unsubstituted or substituted with at least one of a (C1-C6)alkyl(s) and a di(C6-C12)arylamino(s); a (5- to 15-membered)heteroaryl unsubstituted or substituted with a (C6-C12)aryl(s); and a substituted or unsubstituted di(C6-C18)arylamino. For example, the substituent(s), each independently, may be methyl; a phenyl unsubstituted or substituted with a naphthyl(s) or diphenylamono(s); a naphthyl; a biphenyl; a phenanthrenyl; a terphenyl; a dimethylfluorenyl; a pyrimidinyl substituted with a phenyl(s); a triazinyl substituted with at least one of a phenyl(s), naphthyl(s) and biphenyl(s); a dibenzofuranyl; a dibenzothiophenyl; a carbazolyl substituted with a phenyl(s); an amino substituted with at least one of a phenyl(s), a naphthyl(s), a biphenyl(s) and a phenanthrenyl(s).

In Formula 1, X represents O, S, or Se.

In Formula 1, R₁ to R₁₉ each independently represent hydrogen, deuterium, a halogen, a cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, a substituted or unsubstituted (C1-C30)alkoxy, a substituted or unsubstituted amino, a substituted or unsubstituted mono- or di-(C1-C30)alkylamino, a substituted or unsubstituted mono or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino. According to one embodiment of the present disclosure, R₁ to R₁₉ may each independently represent hydrogen, a substituted or unsubstituted (C6-C18)aryl, a substituted or unsubstituted (5- to 20-membered)heteroaryl, a substituted or unsubstituted di-(C6-C20)arylamino, or a substituted or unsubstituted (C6-C18)aryl(5- to 15-membered)heteroarylamino. According to another embodiment of the present disclosure, R₁ to R₁₉ may each independently represent hydrogen; a (C6-C18)aryl unsubstituted or substituted with at least one of a (5- to 15-membered)heteroaryl(s) and a di-(C6-C18)arylamino(s); a (5- to 20-membered)heteroaryl unsubstituted or substituted with at least one of a (C1-C6)alkyl(s), a (C6-C18)aryl(s) and a (5- to 15-membered)heteroaryl(s); a di(C6-C20)arylamino unsubstituted or substituted with at least one of a (C1-C6)alkyl(s) and di(C6-C12)arylamino(s); or a (C6-C18)aryl(5- to 15-membered)heteroarylamino unsubstituted or substituted with a (C6-C12)aryl(s). According to another embodiment of the present disclosure, R₁ to R₁₉ may each independently represent hydrogen, a substituted or unsubstituted phenyl, a substituted or unsubstituted naphthyl, a substituted or unsubstituted pyridyl, a substituted or unsubstituted pyrimidinyl, a substituted or unsubstituted triazinyl, a substituted or unsubstituted quinazolinyl, a substituted or unsubstituted quinoxalinyl, a substituted or unsubstituted (13-membered)heteroaryl, a substituted or unsubstituted benzoquinazolinyl, a substituted or unsubstituted benzoquinoxalinyl, a substituted or unsubstituted diphenylamino, a substituted or unsubstituted phenylnaphthylamino, a substituted or unsubstituted phenylbiphenylamino, a substituted or unsubstituted phenylterphenylamino, a substituted or unsubstituted phenylfluorenylamino, a substituted or unsubstituted phenyldibenzofuranylamino, a substituted or unsubstituted phenylcarbazolylamino, a substituted or unsubstituted naphthylbiphenylamino, a substituted or unsubstituted dibiphenylamino, a substituted or unsubstituted biphenyldibenzofuranylamino, or a substituted or unsubstituted biphenyldibenzothiophenylamino. For example, R₁ to R₁₉ may each independently represent hydrogen; a phenyl unsubstituted or substituted with a diphenyltriazinyl(s), a phenylbiphenyltriazinyl(s), a dinaphthyltriazinyl(s), a diphenylamino(s), a phenylnaphthylamino(s), a phenylbiphenylamino(s), a phenylphenanthrenylamino(s) or a dibiphenylamino(s); a naphthyl unsubstituted or substituted with a diphenyltriazinyl(s); a biphenyl; a pyridyl unsubstituted or substituted with at least one of a phenyl(s) and a pyrimidinyl(s); a pyrimidinyl substituted with a phenyl(s); a triazinyl substituted with at least one of a phenyl(s), a naphthyl(s), a biphenyl(s) and a phenylcarbazolyl(s); a quinazolinyl substituted with a phenyl(s) or a naphthyl(s); a quinoxalinyl substituted with a phenyl(s); a (13-membered)heteroaryl unsubstituted or substituted with at least one of a methyl(s) and a phenyl(s); a benzoquinazolinyl substituted with a phenyl(s); a benzoquinoxalinyl substituted with a phenyl(s); an amino substituted with at least one of a phenyl(s), a phenyl(s) substituted with a diphenylamino(s), a naphthyl(s), a biphenyl(s), a naphthylphenyl(s), a terphenyl(s), a dibenzofluorenyl(s), a dibenzofuranyl(s), a dibenzothiophenyl(s) and a phenylcarbazolyl(s).

According to one embodiment of the present disclosure, at least one of R₁ to R₁₉ comprises a substituted or unsubstituted nitrogen-containing heteroaryl or a substituted or unsubstituted amino. The nitrogen-containing heteroaryl may be pyridyl, pyrimidinyl, triazinyl, quinazolinyl, quinoxalinyl, benzoquinazolinyl, benzoquinoxalinyl, (13-membered)heteroaryl comprising one or more of nitrogen, (13-membered)heteroaryl comprising oxygen or sulfur, and one or more of nitrogen, etc.

In Formula 1-1, Y₁ and Y₂ each independently represent O, S, CR₂₀R₂₁, or NR₂₂. According to one embodiment of the present disclosure, one of Y₁ and Y₂ is NR₂₂ and the other is O, S, or CR₂₀R₂₁. According to another embodiment of the present disclosure, one of Y₁ and Y₂ is NR₂₂ and the other is O or S.

In formulas 1-1 to 1-3, * is a site fused with Formula 1.

According to one embodiment of the present disclosure, Formula 1 may be represented by one of the following formulas 2-1 to 2-12.

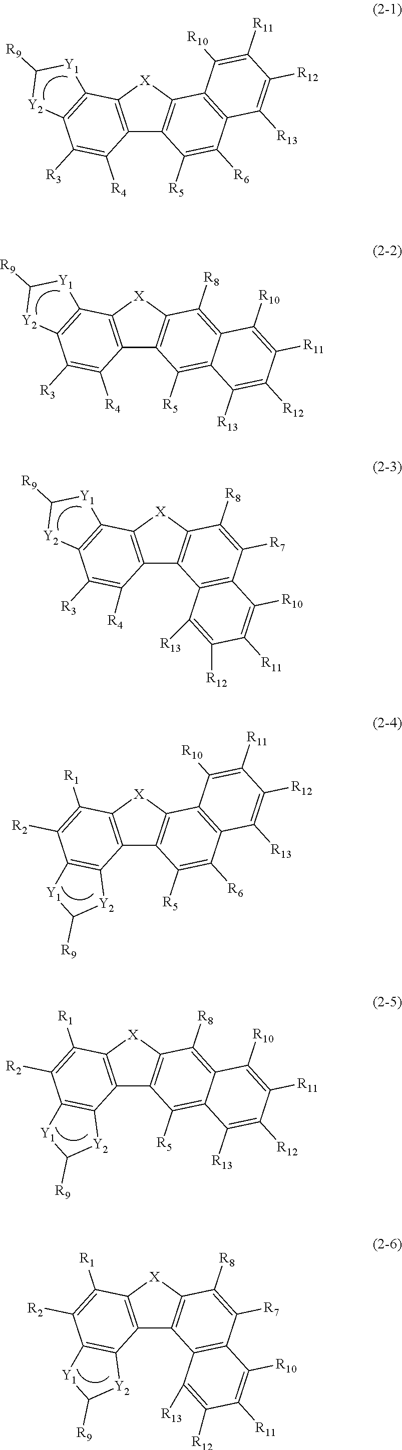

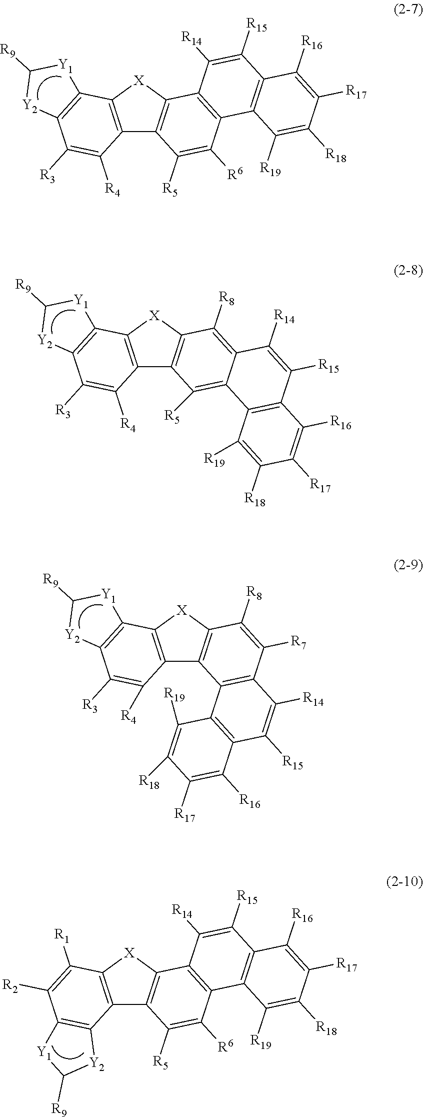

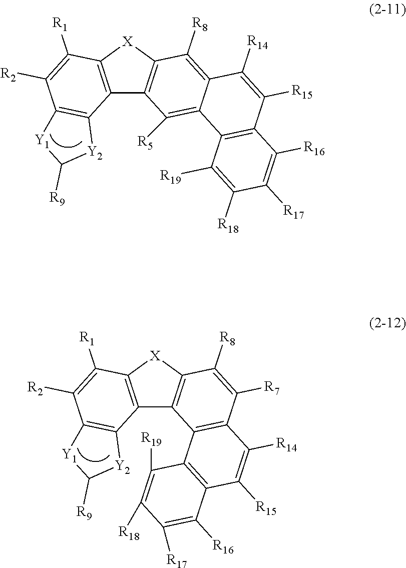

In formulas 2-1 to 2-11, X, R₁ to R₁₉, Y₁ and Y₂ are as defined in Formula 1.

The compound represented by Formula 1 may be selected from the following compounds, but is not limited thereto.

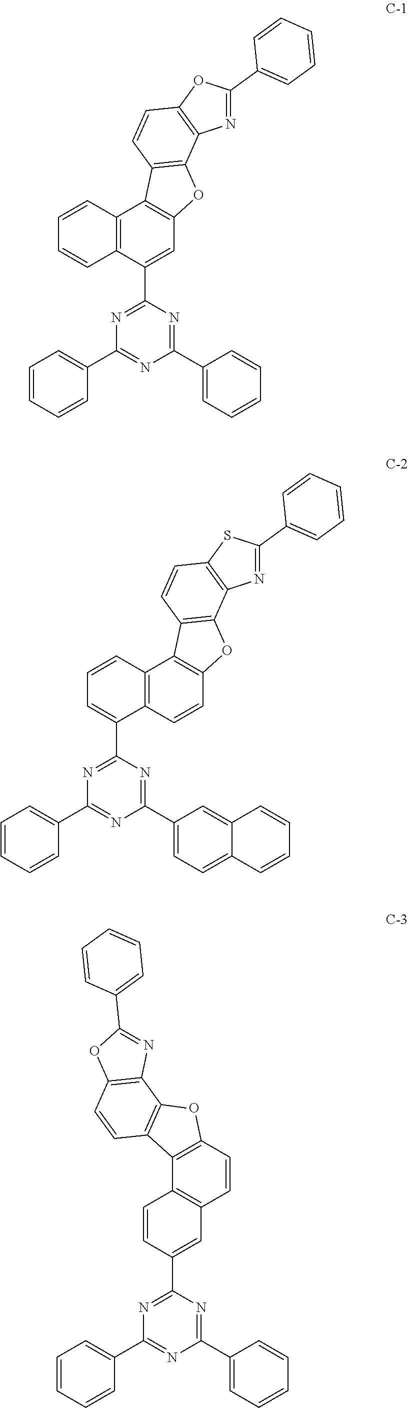 [SMILES: c1ccc(-c2nc(-c3ccccc3)nc(-c3cc4oc5c(ccc6oc(-c7ccccc7)nc65)c4c4ccccc34)n2)cc1 ; c1ccc(-c2nc(-c3ccc4ccccc4c3)nc(-c3cccc4c3ccc3oc5c(ccc6sc(-c7ccccc7)nc65)c34)n2)cc1 ; c1ccc(-c2nc(-c3ccccc3)nc(-c3ccc4c(ccc5oc6c(ccc7oc(-c8ccccc8)nc76)c54)c3)n2)cc1]

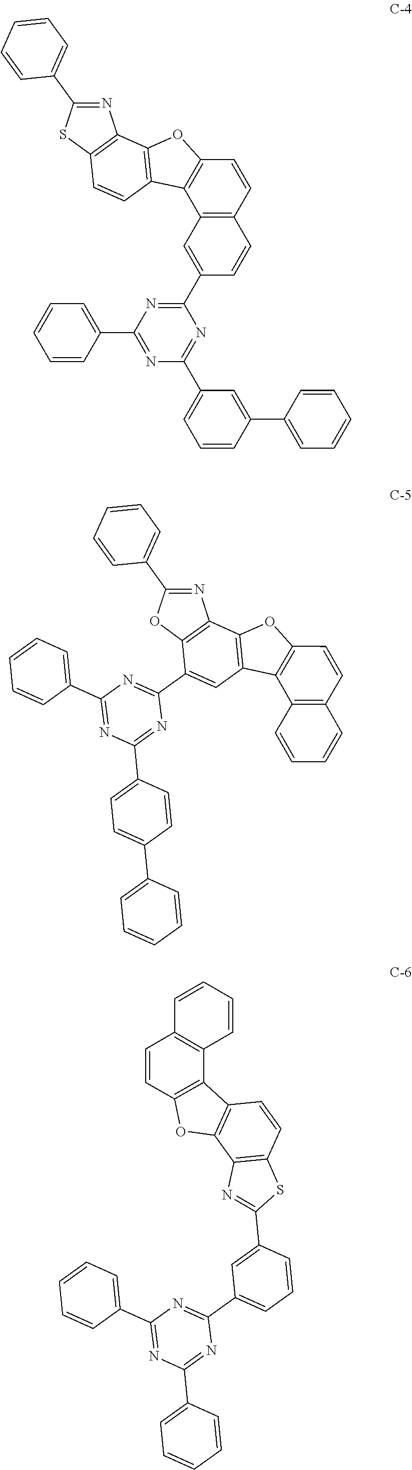 [SMILES: c1ccc(-c2cccc(-c3nc(-c4ccccc4)nc(-c4ccc5ccc6oc7c(ccc8sc(-c9ccccc9)nc87)c6c5c4)n3)c2)cc1 ; c1ccc(-c2ccc(-c3nc(-c4ccccc4)nc(-c4cc5c(oc6ccc7ccccc7c65)c5nc(-c6ccccc6)oc45)n3)cc2)cc1 ; c1ccc(-c2nc(-c3ccccc3)nc(-c3cccc(-c4nc5c(ccc6c5oc5ccc7ccccc7c56)s4)c3)n2)cc1]

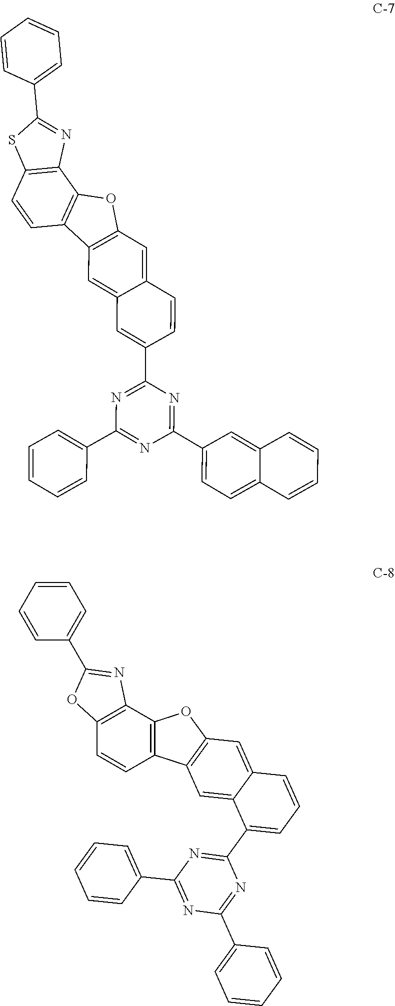 [SMILES: c1ccc(-c2nc(-c3ccc4ccccc4c3)nc(-c3ccc4cc5oc6c(ccc7sc(-c8ccccc8)nc76)c5cc4c3)n2)cc1 ; c1ccc(-c2nc(-c3ccccc3)nc(-c3cccc4cc5oc6c(ccc7oc(-c8ccccc8)nc76)c5cc34)n2)cc1]

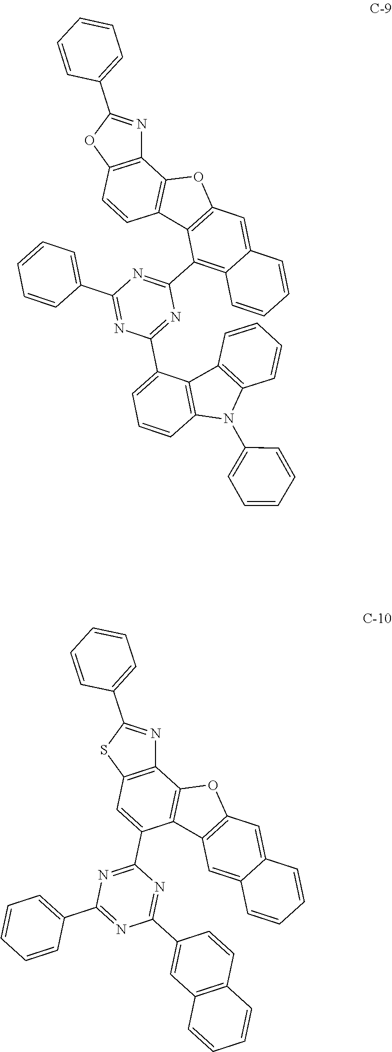 [SMILES: c1ccc(-c2nc(-c3c4ccccc4cc4oc5c(ccc6oc(-c7ccccc7)nc65)c34)nc(-c3cccc4c3c3ccccc3n4-c3ccccc3)n2)cc1 ; c1ccc(-c2nc(-c3ccc4ccccc4c3)nc(-c3cc4sc(-c5ccccc5)nc4c4oc5cc6ccccc6cc5c34)n2)cc1]

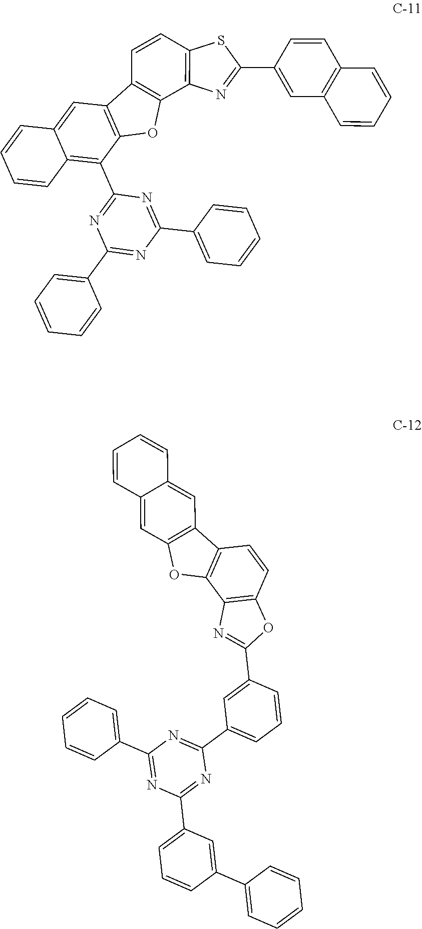 [SMILES: c1ccc(-c2nc(-c3ccccc3)nc(-c3c4ccccc4cc4c3oc3c4ccc4sc(-c5ccc6ccccc6c5)nc43)n2)cc1 ; c1ccc(-c2cccc(-c3nc(-c4ccccc4)nc(-c4cccc(-c5nc6c(ccc7c8cc9ccccc9cc8oc76)o5)c4)n3)c2)cc1]

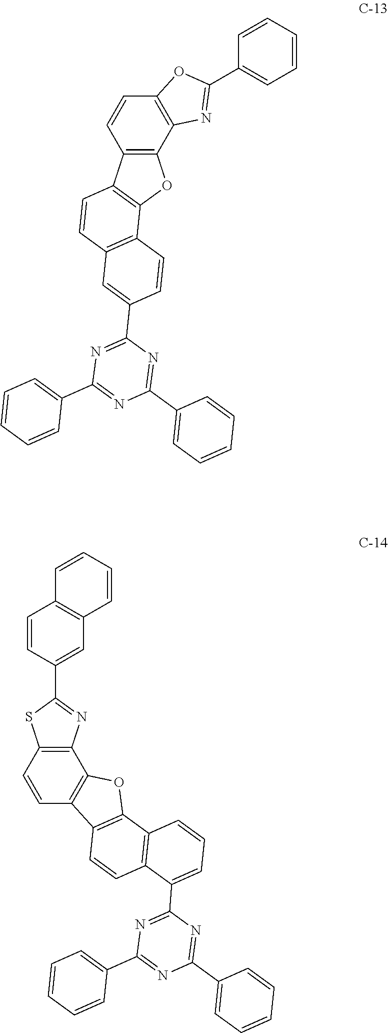 [SMILES: c1ccc(-c2nc(-c3ccccc3)nc(-c3ccc4c(ccc5c6ccc7oc(-c8ccccc8)nc7c6oc45)c3)n2)cc1 ; c1ccc(-c2nc(-c3ccccc3)nc(-c3cccc4c3ccc3c5ccc6sc(-c7ccc8ccccc8c7)nc6c5oc43)n2)cc1]

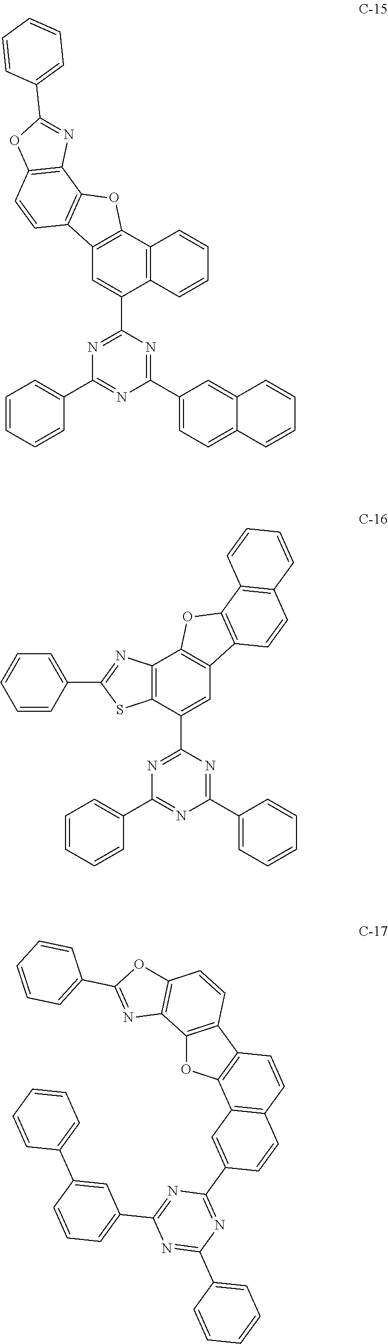 [SMILES: c1ccc(-c2nc(-c3ccc4ccccc4c3)nc(-c3cc4c5ccc6oc(-c7ccccc7)nc6c5oc4c4ccccc34)n2)cc1 ; c1ccc(-c2nc(-c3ccccc3)nc(-c3cc4c5ccc6ccccc6c5oc4c4nc(-c5ccccc5)sc34)n2)cc1 ; c1ccc(-c2cccc(-c3nc(-c4ccccc4)nc(-c4ccc5ccc6c7ccc8oc(-c9ccccc9)nc8c7oc6c5c4)n3)c2)cc1]

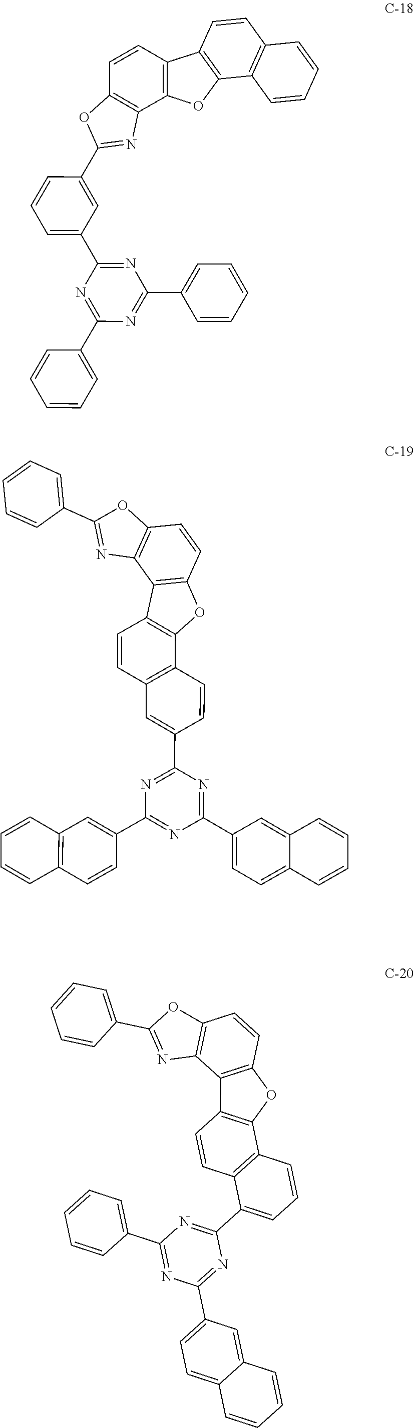 [SMILES: c1ccc(-c2nc(-c3ccccc3)nc(-c3cccc(-c4nc5c(ccc6c7ccc8ccccc8c7oc65)o4)c3)n2)cc1 ; c1ccc(-c2nc3c(ccc4oc5c6ccc(-c7nc(-c8ccc9ccccc9c8)nc(-c8ccc9ccccc9c8)n7)cc6ccc5c43)o2)cc1 ; c1ccc(-c2nc(-c3ccc4ccccc4c3)nc(-c3cccc4c3ccc3c4oc4ccc5oc(-c6ccccc6)nc5c43)n2)cc1]

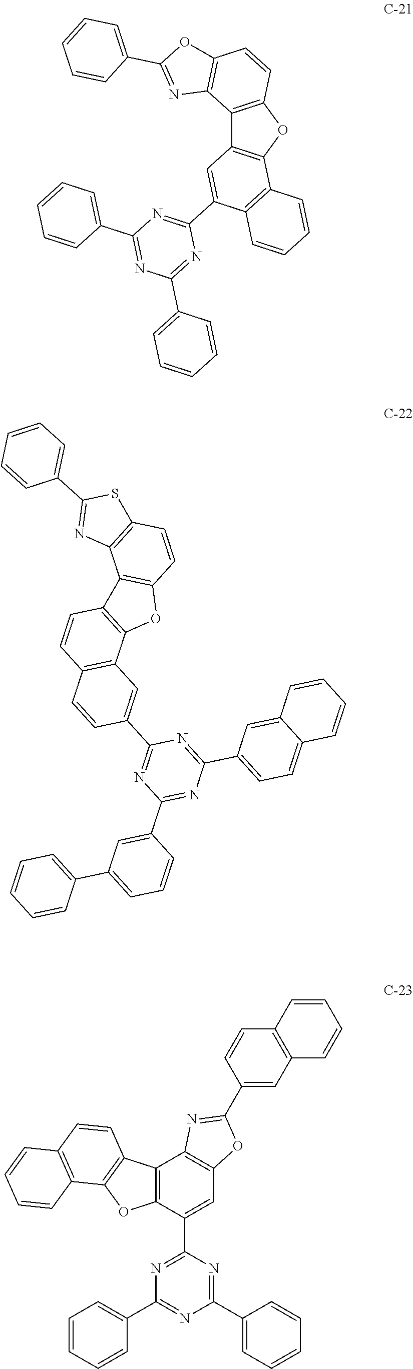 [SMILES: c1ccc(-c2nc(-c3ccccc3)nc(-c3cc4c(oc5ccc6oc(-c7ccccc7)nc6c54)c4ccccc34)n2)cc1 ; c1ccc(-c2cccc(-c3nc(-c4ccc5ccccc5c4)nc(-c4ccc5ccc6c(oc7ccc8sc(-c9ccccc9)nc8c76)c5c4)n3)c2)cc1 ; c1ccc(-c2nc(-c3ccccc3)nc(-c3cc4oc(-c5ccc6ccccc6c5)nc4c4c3oc3c5ccccc5ccc34)n2)cc1]

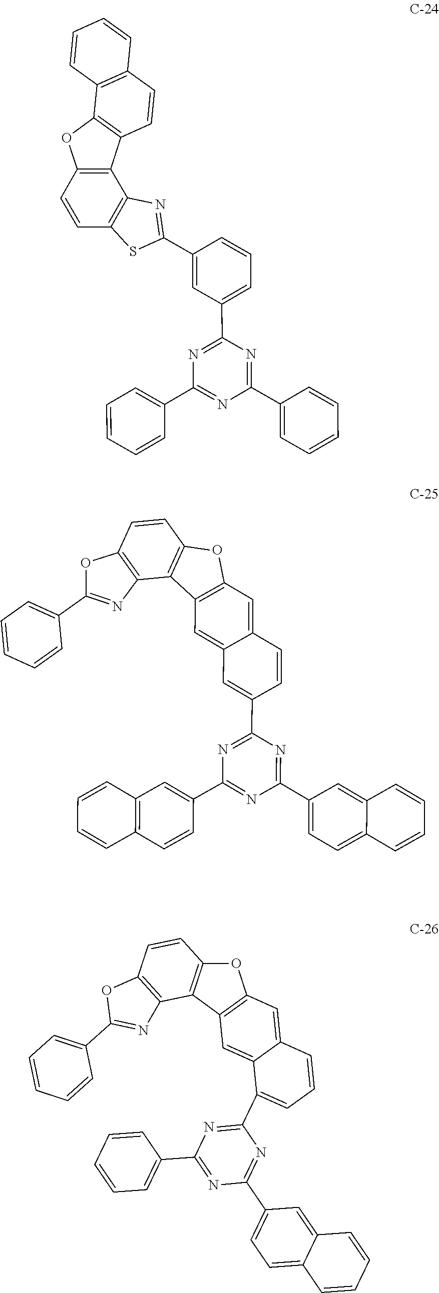 [SMILES: c1ccc(-c2nc(-c3ccccc3)nc(-c3cccc(-c4nc5c(ccc6oc7c8ccccc8ccc7c65)s4)c3)n2)cc1 ; c1ccc(-c2nc3c(ccc4oc5cc6ccc(-c7nc(-c8ccc9ccccc9c8)nc(-c8ccc9ccccc9c8)n7)cc6cc5c43)o2)cc1 ; c1ccc(-c2nc(-c3ccc4ccccc4c3)nc(-c3cccc4cc5oc6ccc7oc(-c8ccccc8)nc7c6c5cc34)n2)cc1]

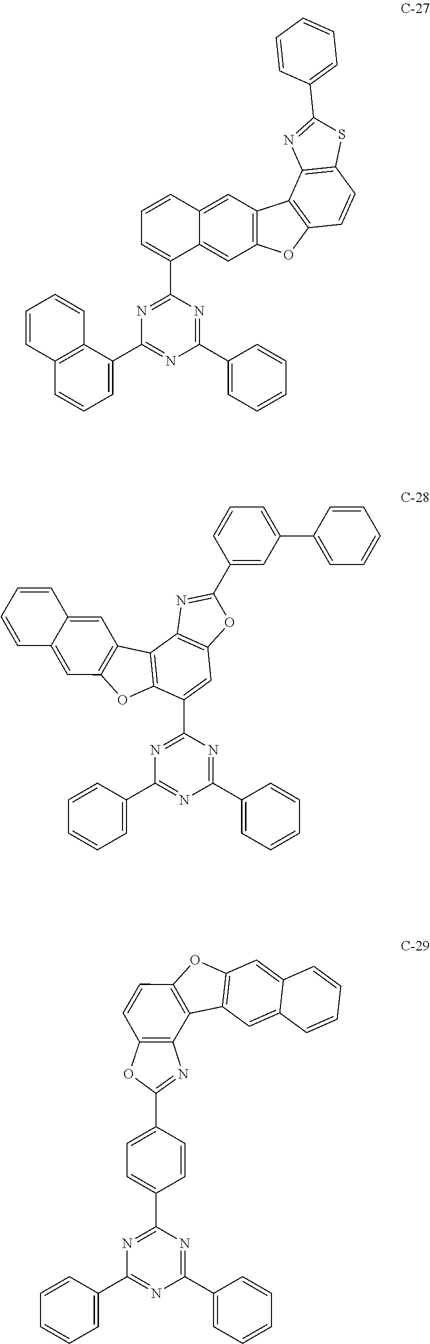 [SMILES: c1ccc(-c2nc(-c3cccc4ccccc34)nc(-c3cccc4cc5c(cc34)oc3ccc4sc(-c6ccccc6)nc4c35)n2)cc1 ; c1ccc(-c2cccc(-c3nc4c(cc(-c5nc(-c6ccccc6)nc(-c6ccccc6)n5)c5oc6cc7ccccc7cc6c54)o3)c2)cc1 ; c1ccc(-c2nc(-c3ccccc3)nc(-c3ccc(-c4nc5c(ccc6oc7cc8ccccc8cc7c65)o4)cc3)n2)cc1]

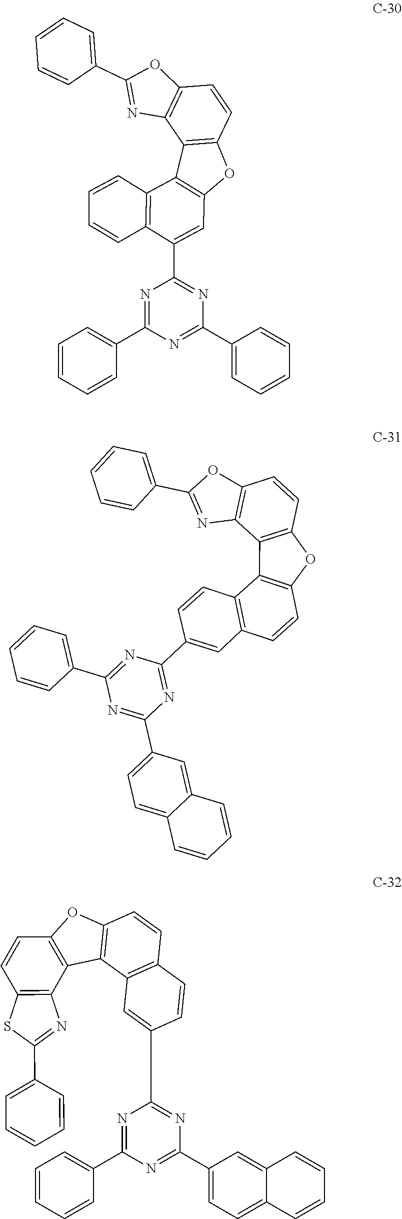 [SMILES: c1ccc(-c2nc(-c3ccccc3)nc(-c3cc4oc5ccc6oc(-c7ccccc7)nc6c5c4c4ccccc34)n2)cc1 ; c1ccc(-c2nc(-c3ccc4ccccc4c3)nc(-c3ccc4c(ccc5oc6ccc7oc(-c8ccccc8)nc7c6c54)c3)n2)cc1 ; c1ccc(-c2nc(-c3ccc4ccccc4c3)nc(-c3ccc4ccc5oc6ccc7sc(-c8ccccc8)nc7c6c5c4c3)n2)cc1]

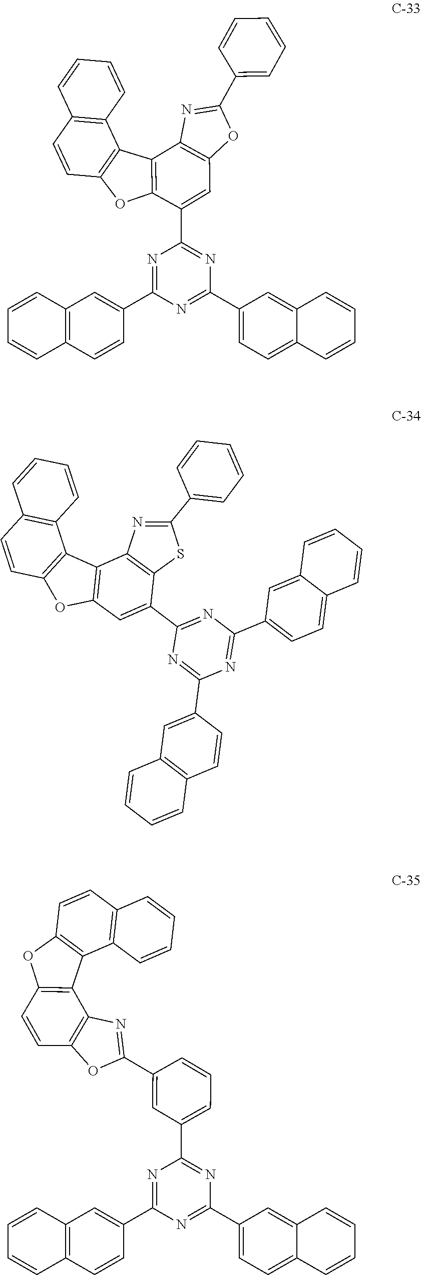 [SMILES: c1ccc(-c2nc3c(cc(-c4nc(-c5ccc6ccccc6c5)nc(-c5ccc6ccccc6c5)n4)c4oc5ccc6ccccc6c5c43)o2)cc1 ; c1ccc(-c2nc3c(s2)c(-c2nc(-c4ccc5ccccc5c4)nc(-c4ccc5ccccc5c4)n2)cc2oc4ccc5ccccc5c4c23)cc1 ; c1cc(-c2nc(-c3ccc4ccccc4c3)nc(-c3ccc4ccccc4c3)n2)cc(-c2nc3c(ccc4oc5ccc6ccccc6c5c43)o2)c1]

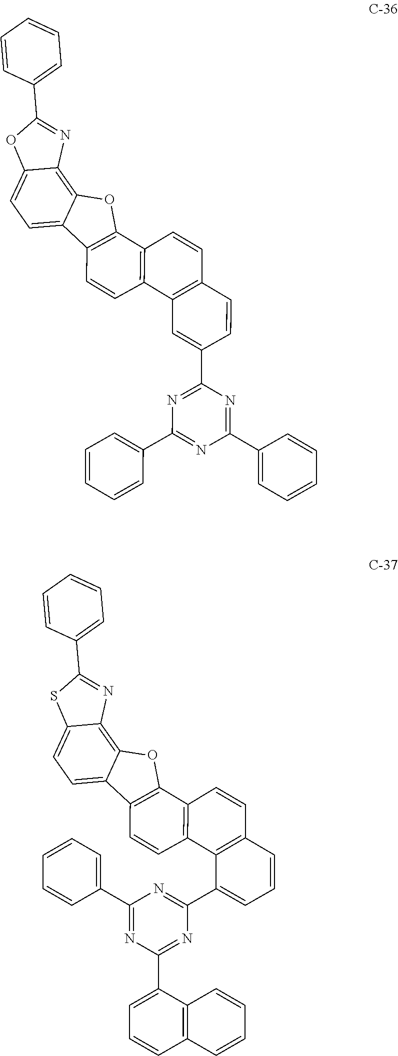 [SMILES: c1ccc(-c2nc(-c3ccccc3)nc(-c3ccc4ccc5c(ccc6c7ccc8oc(-c9ccccc9)nc8c7oc56)c4c3)n2)cc1 ; c1ccc(-c2nc(-c3cccc4ccccc34)nc(-c3cccc4ccc5c(ccc6c7ccc8sc(-c9ccccc9)nc8c7oc65)c34)n2)cc1]

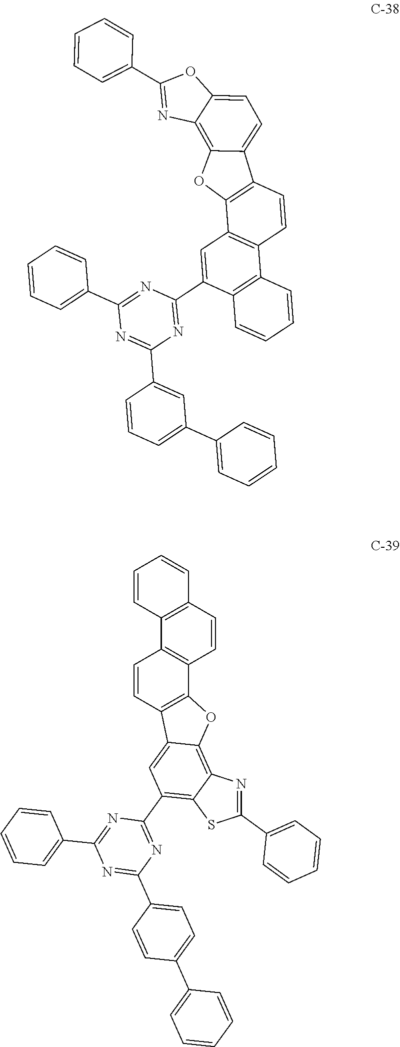 [SMILES: c1ccc(-c2cccc(-c3nc(-c4ccccc4)nc(-c4cc5c(ccc6c7ccc8oc(-c9ccccc9)nc8c7oc56)c5ccccc45)n3)c2)cc1 ; c1ccc(-c2ccc(-c3nc(-c4ccccc4)nc(-c4cc5c6ccc7c8ccccc8ccc7c6oc5c5nc(-c6ccccc6)sc45)n3)cc2)cc1]

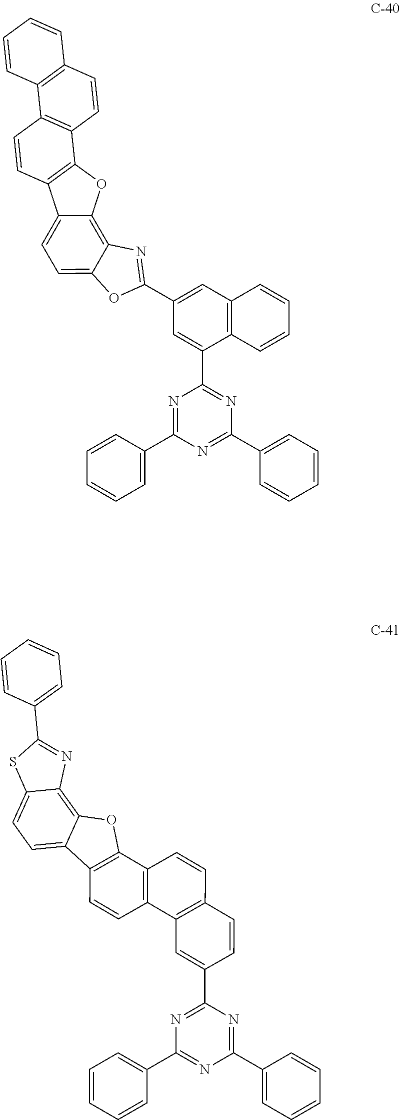 [SMILES: c1ccc(-c2nc(-c3ccccc3)nc(-c3cc(-c4nc5c(ccc6c7ccc8c9ccccc9ccc8c7oc65)o4)cc4ccccc34)n2)cc1 ; c1ccc(-c2nc(-c3ccccc3)nc(-c3ccc4ccc5c(ccc6c7ccc8sc(-c9ccccc9)nc8c7oc56)c4c3)n2)cc1]

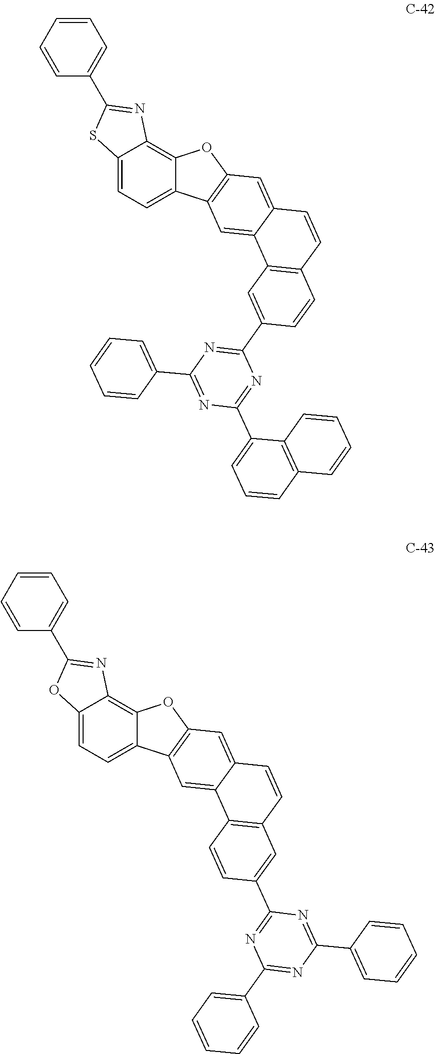 [SMILES: c1ccc(-c2nc(-c3ccc4ccc5cc6oc7c(ccc8sc(-c9ccccc9)nc87)c6cc5c4c3)nc(-c3cccc4ccccc34)n2)cc1 ; c1ccc(-c2nc(-c3ccccc3)nc(-c3ccc4c(ccc5cc6oc7c(ccc8oc(-c9ccccc9)nc87)c6cc54)c3)n2)cc1]

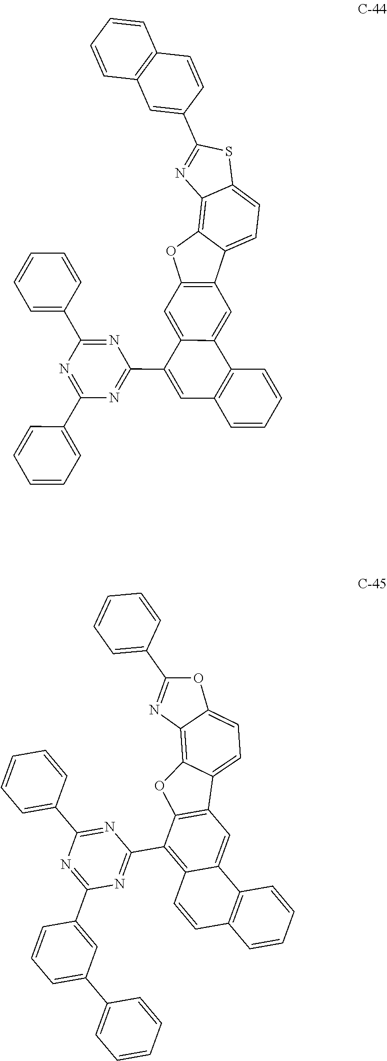 [SMILES: c1ccc(-c2nc(-c3ccccc3)nc(-c3cc4ccccc4c4cc5c(cc34)oc3c5ccc4sc(-c5ccc6ccccc6c5)nc43)n2)cc1 ; c1ccc(-c2cccc(-c3nc(-c4ccccc4)nc(-c4c5ccc6ccccc6c5cc5c4oc4c5ccc5oc(-c6ccccc6)nc54)n3)c2)cc1]

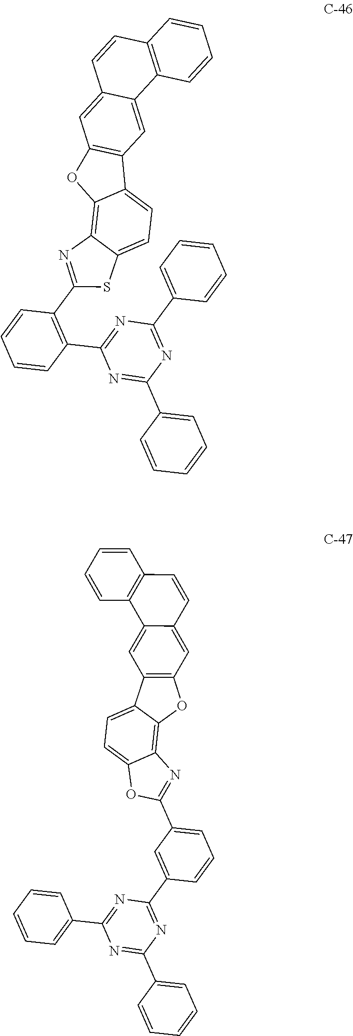 [SMILES: c1ccc(-c2nc(-c3ccccc3)nc(-c3ccccc3-c3nc4c(ccc5c6cc7c(ccc8ccccc87)cc6oc54)s3)n2)cc1 ; c1ccc(-c2nc(-c3ccccc3)nc(-c3cccc(-c4nc5c(ccc6c7cc8c(ccc9ccccc98)cc7oc65)o4)c3)n2)cc1]

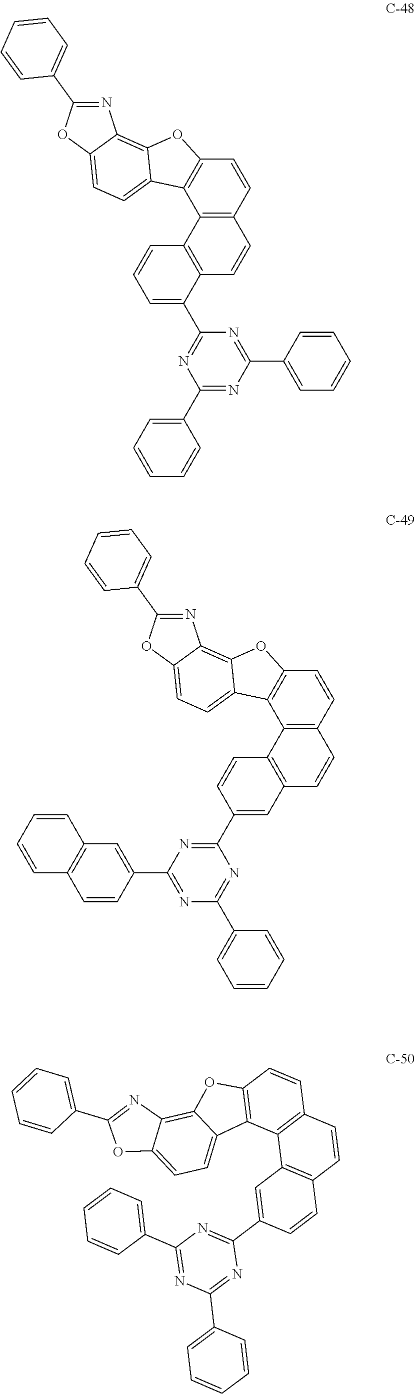 [SMILES: c1ccc(-c2nc(-c3ccccc3)nc(-c3cccc4c3ccc3ccc5oc6c(ccc7oc(-c8ccccc8)nc76)c5c34)n2)cc1 ; c1ccc(-c2nc(-c3ccc4ccccc4c3)nc(-c3ccc4c(ccc5ccc6oc7c(ccc8oc(-c9ccccc9)nc87)c6c54)c3)n2)cc1 ; c1ccc(-c2nc(-c3ccccc3)nc(-c3ccc4ccc5ccc6oc7c(ccc8oc(-c9ccccc9)nc87)c6c5c4c3)n2)cc1]

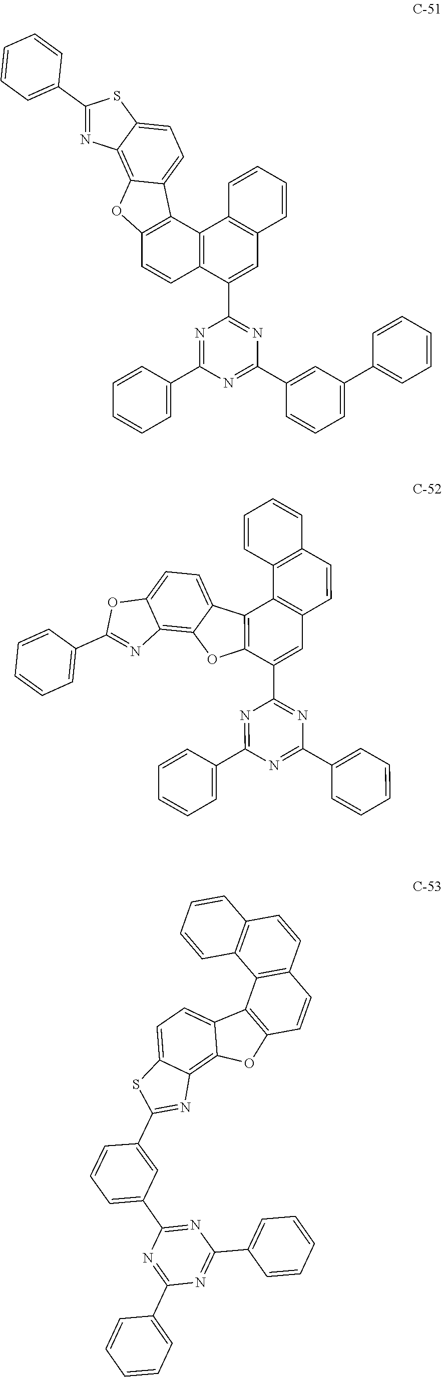 [SMILES: c1ccc(-c2cccc(-c3nc(-c4ccccc4)nc(-c4cc5ccccc5c5c4ccc4oc6c(ccc7sc(-c8ccccc8)nc76)c45)n3)c2)cc1 ; c1ccc(-c2nc(-c3ccccc3)nc(-c3cc4ccc5ccccc5c4c4c3oc3c4ccc4oc(-c5ccccc5)nc43)n2)cc1 ; c1ccc(-c2nc(-c3ccccc3)nc(-c3cccc(-c4nc5c(ccc6c5oc5ccc7ccc8ccccc8c7c56)s4)c3)n2)cc1]

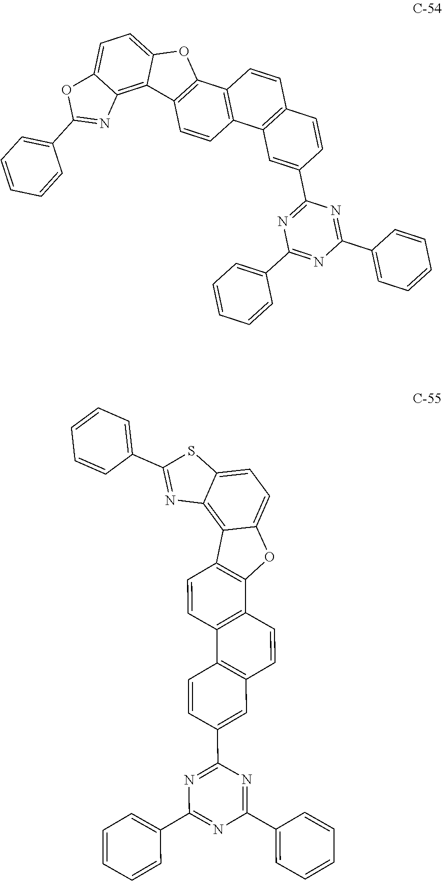 [SMILES: c1ccc(-c2nc(-c3ccccc3)nc(-c3ccc4ccc5c(ccc6c5oc5ccc7oc(-c8ccccc8)nc7c56)c4c3)n2)cc1 ; c1ccc(-c2nc(-c3ccccc3)nc(-c3ccc4c(ccc5c4ccc4c5oc5ccc6sc(-c7ccccc7)nc6c54)c3)n2)cc1]

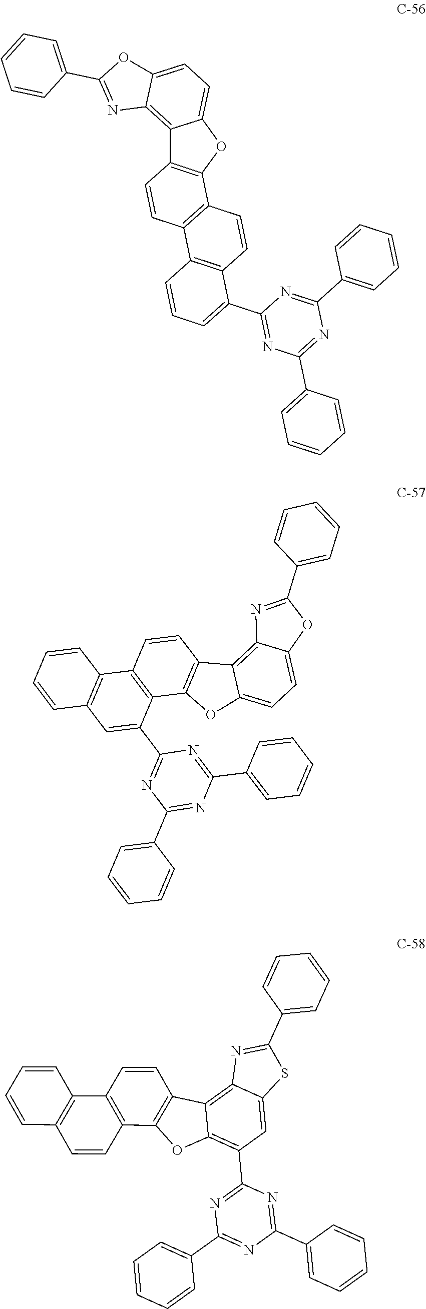 [SMILES: c1ccc(-c2nc(-c3ccccc3)nc(-c3cccc4c3ccc3c4ccc4c3oc3ccc5oc(-c6ccccc6)nc5c34)n2)cc1 ; c1ccc(-c2nc(-c3ccccc3)nc(-c3cc4ccccc4c4ccc5c(oc6ccc7oc(-c8ccccc8)nc7c65)c34)n2)cc1 ; c1ccc(-c2nc(-c3ccccc3)nc(-c3cc4sc(-c5ccccc5)nc4c4c3oc3c5ccc6ccccc6c5ccc34)n2)cc1]

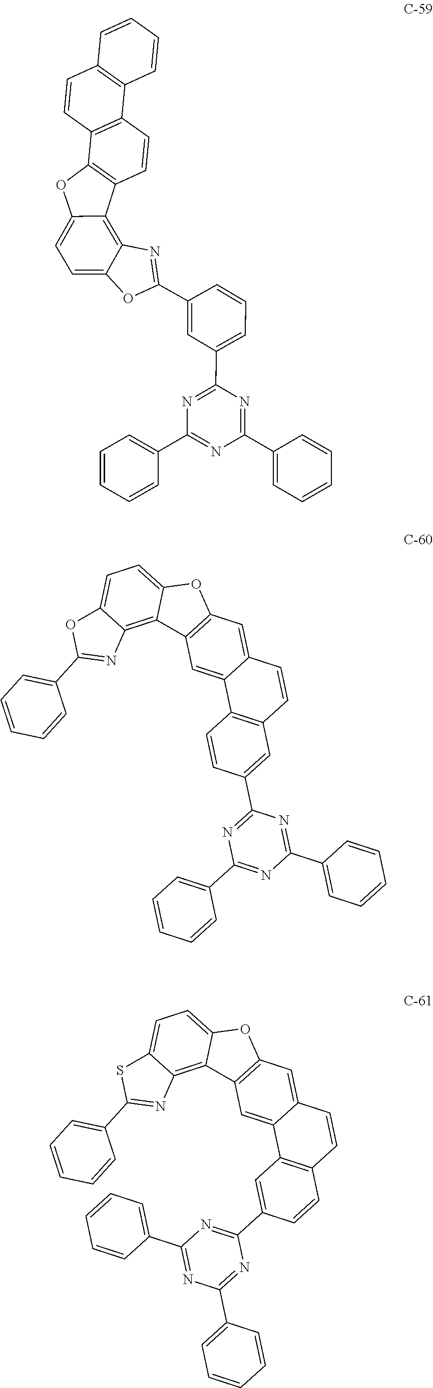 [SMILES: c1ccc(-c2nc(-c3ccccc3)nc(-c3cccc(-c4nc5c(ccc6oc7c8ccc9ccccc9c8ccc7c65)o4)c3)n2)cc1 ; c1ccc(-c2nc(-c3ccccc3)nc(-c3ccc4c(ccc5cc6oc7ccc8oc(-c9ccccc9)nc8c7c6cc54)c3)n2)cc1 ; c1ccc(-c2nc(-c3ccccc3)nc(-c3ccc4ccc5cc6oc7ccc8sc(-c9ccccc9)nc8c7c6cc5c4c3)n2)cc1]

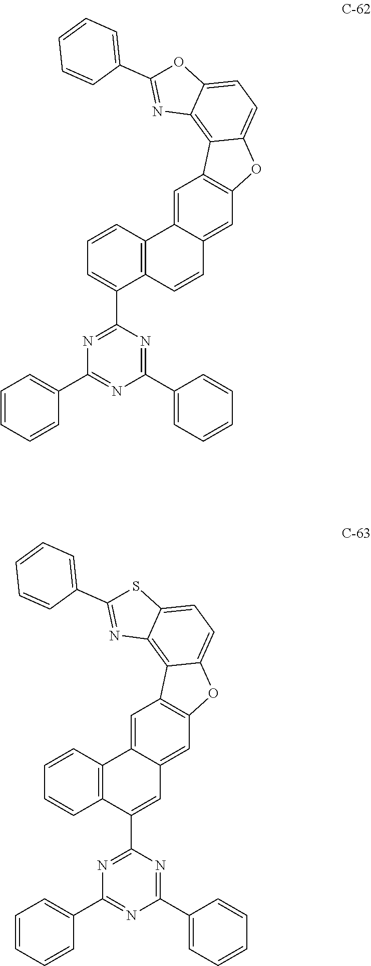 [SMILES: c1ccc(-c2nc(-c3ccccc3)nc(-c3cccc4c3ccc3cc5oc6ccc7oc(-c8ccccc8)nc7c6c5cc34)n2)cc1 ; c1ccc(-c2nc(-c3ccccc3)nc(-c3cc4cc5oc6ccc7sc(-c8ccccc8)nc7c6c5cc4c4ccccc34)n2)cc1]

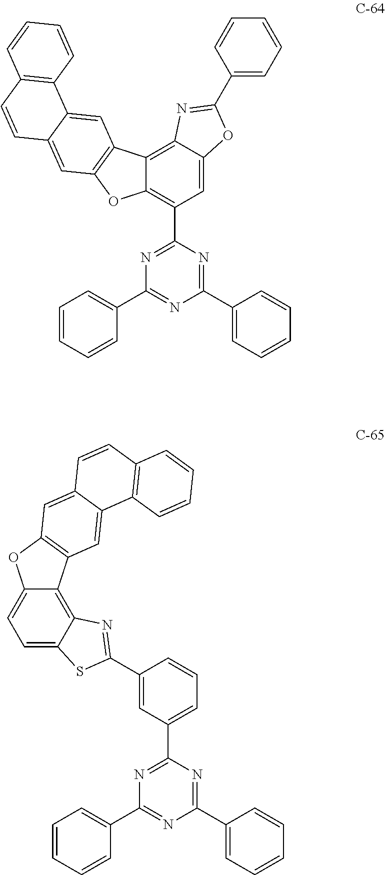 [SMILES: c1ccc(-c2nc(-c3ccccc3)nc(-c3cc4oc(-c5ccccc5)nc4c4c3oc3cc5ccc6ccccc6c5cc34)n2)cc1 ; c1ccc(-c2nc(-c3ccccc3)nc(-c3cccc(-c4nc5c(ccc6oc7cc8ccc9ccccc9c8cc7c65)s4)c3)n2)cc1]

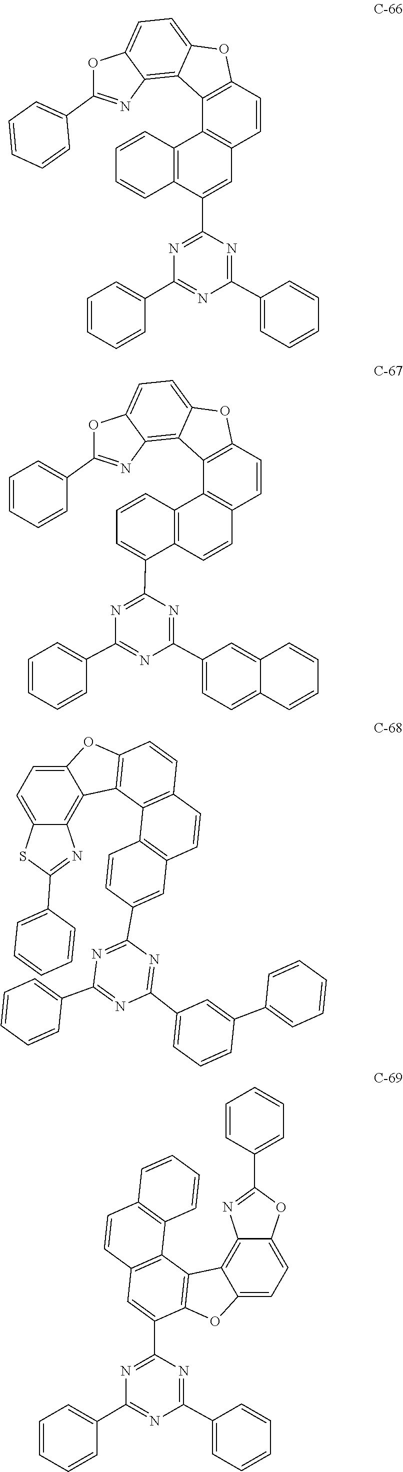

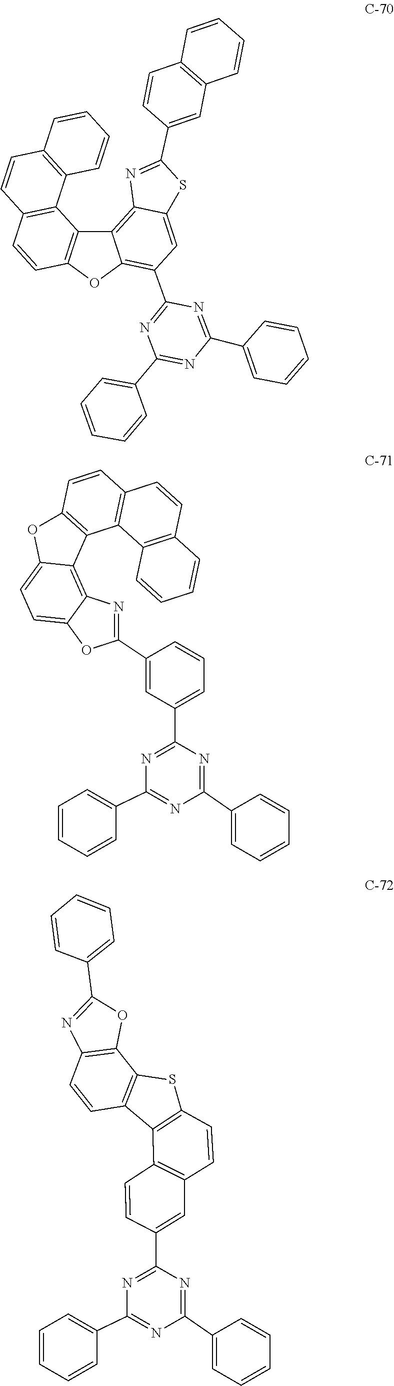

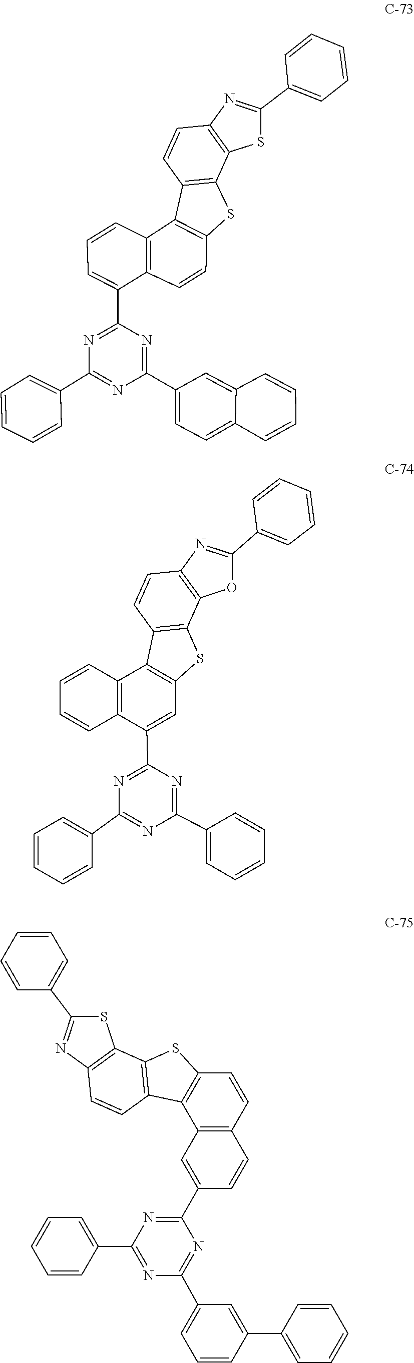

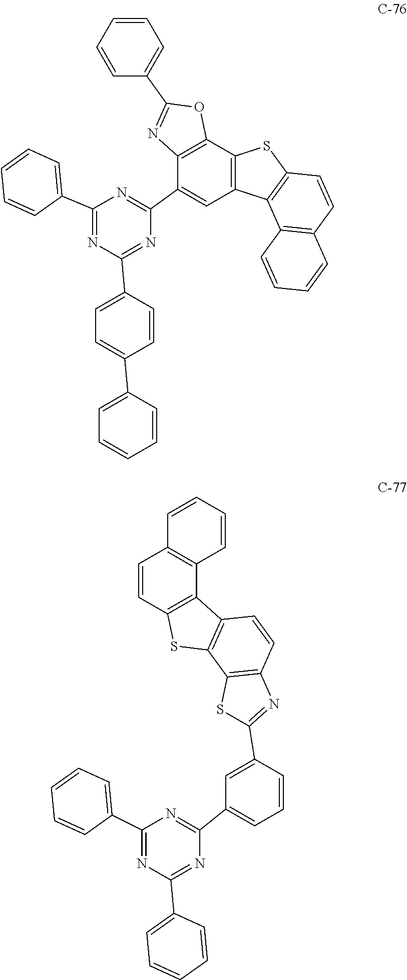 [SMILES: c1ccc(-c2ccc(-c3nc(-c4ccccc4)nc(-c4cc5c(sc6ccc7ccccc7c65)c5oc(-c6ccccc6)nc45)n3)cc2)cc1 ; c1ccc(-c2nc(-c3ccccc3)nc(-c3cccc(-c4nc5ccc6c(sc7ccc8ccccc8c76)c5s4)c3)n2)cc1]

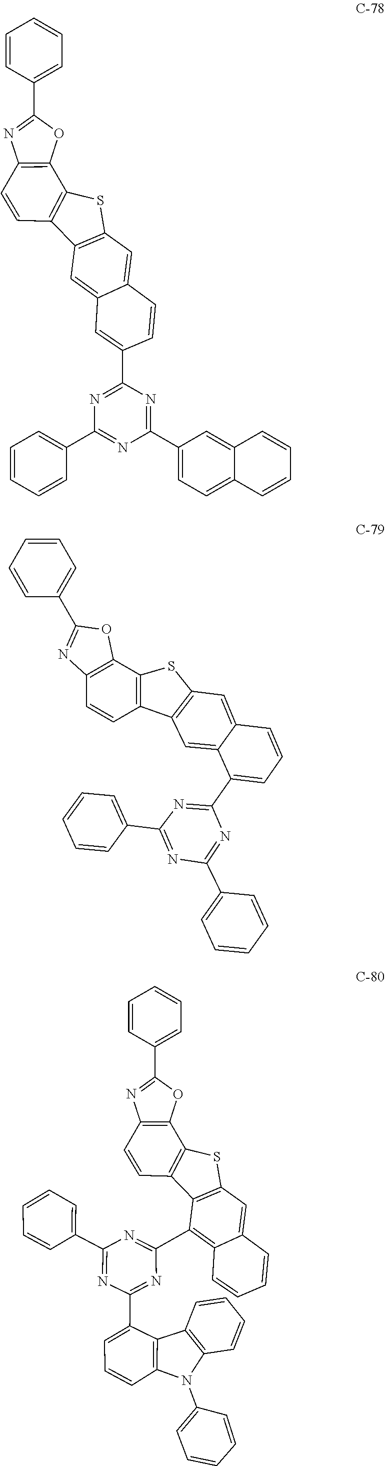 [SMILES: c1ccc(-c2nc(-c3ccc4ccccc4c3)nc(-c3ccc4cc5sc6c(ccc7nc(-c8ccccc8)oc76)c5cc4c3)n2)cc1 ; c1ccc(-c2nc(-c3ccccc3)nc(-c3cccc4cc5sc6c(ccc7nc(-c8ccccc8)oc76)c5cc34)n2)cc1 ; c1ccc(-c2nc(-c3c4ccccc4cc4sc5c(ccc6nc(-c7ccccc7)oc65)c34)nc(-c3cccc4c3c3ccccc3n4-c3ccccc3)n2)cc1]

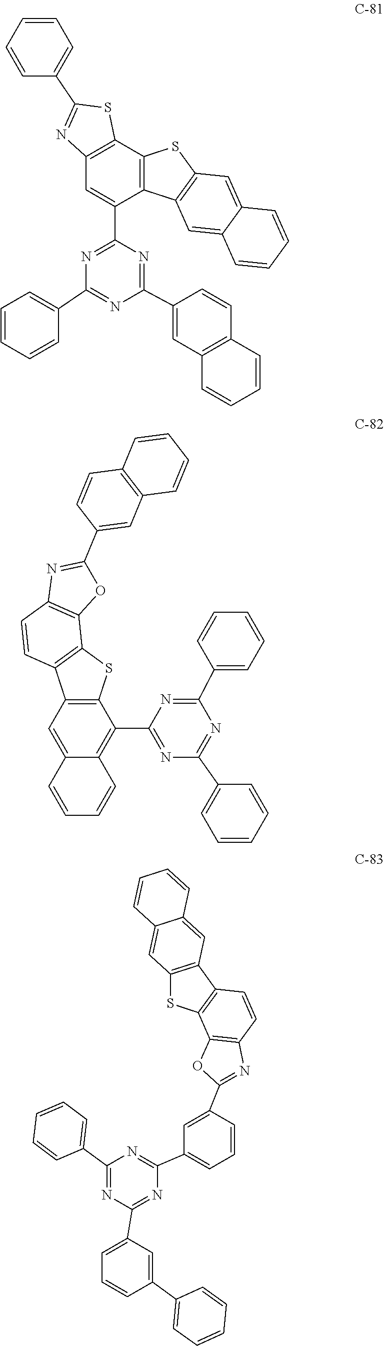 [SMILES: c1ccc(-c2nc(-c3ccc4ccccc4c3)nc(-c3cc4nc(-c5ccccc5)sc4c4sc5cc6ccccc6cc5c34)n2)cc1 ; c1ccc(-c2nc(-c3ccccc3)nc(-c3c4ccccc4cc4c3sc3c4ccc4nc(-c5ccc6ccccc6c5)oc43)n2)cc1 ; c1ccc(-c2cccc(-c3nc(-c4ccccc4)nc(-c4cccc(-c5nc6ccc7c8cc9ccccc9cc8sc7c6o5)c4)n3)c2)cc1]

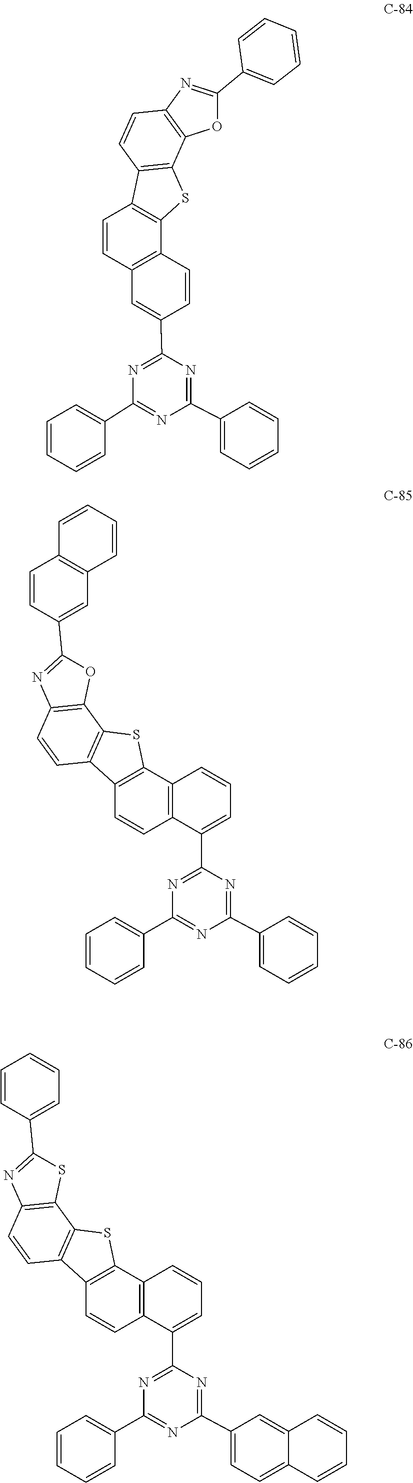

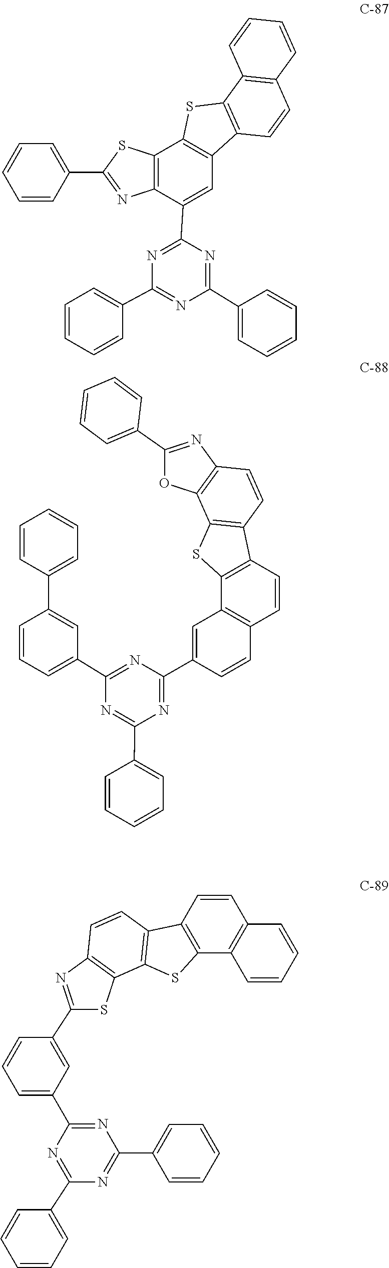

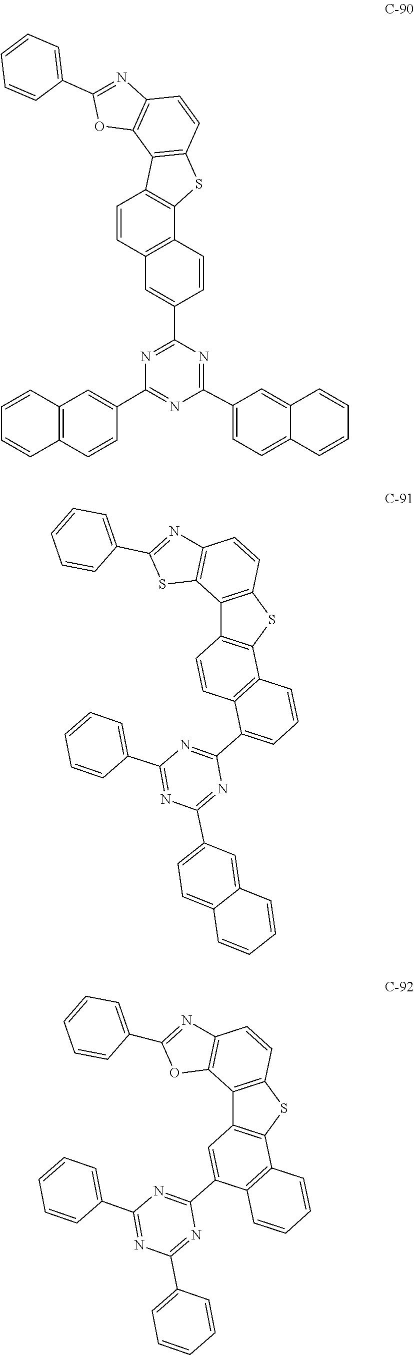 [SMILES: c1ccc(-c2nc3ccc4sc5c6ccc(-c7nc(-c8ccc9ccccc9c8)nc(-c8ccc9ccccc9c8)n7)cc6ccc5c4c3o2)cc1 ; c1ccc(-c2nc(-c3ccc4ccccc4c3)nc(-c3cccc4c3ccc3c4sc4ccc5nc(-c6ccccc6)sc5c43)n2)cc1 ; c1ccc(-c2nc(-c3ccccc3)nc(-c3cc4c(sc5ccc6nc(-c7ccccc7)oc6c54)c4ccccc34)n2)cc1]

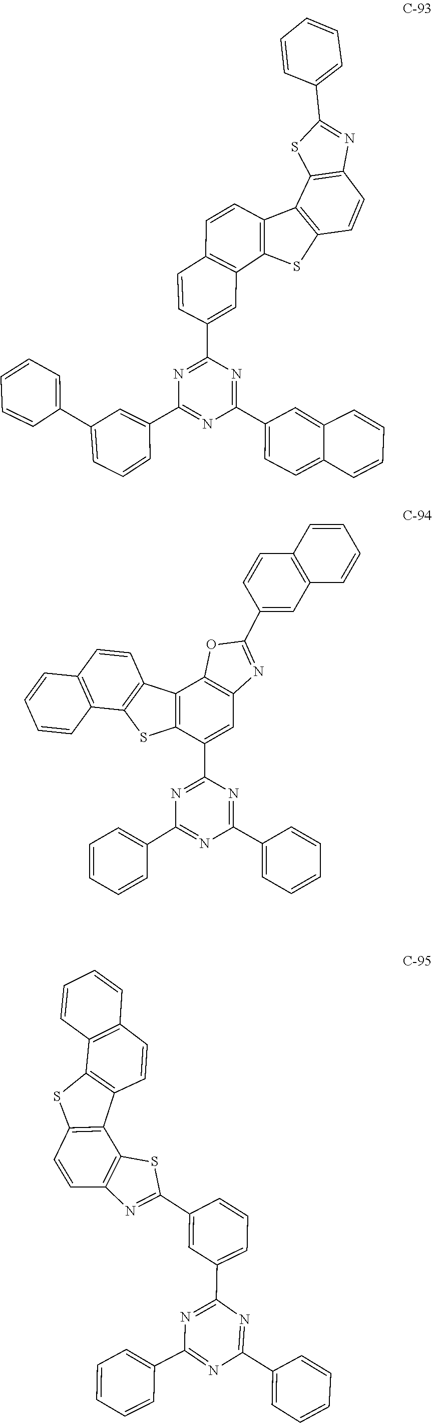 [SMILES: c1ccc(-c2cccc(-c3nc(-c4ccc5ccccc5c4)nc(-c4ccc5ccc6c(sc7ccc8nc(-c9ccccc9)sc8c76)c5c4)n3)c2)cc1 ; c1ccc(-c2nc(-c3ccccc3)nc(-c3cc4nc(-c5ccc6ccccc6c5)oc4c4c3sc3c5ccccc5ccc34)n2)cc1 ; c1ccc(-c2nc(-c3ccccc3)nc(-c3cccc(-c4nc5ccc6sc7c8ccccc8ccc7c6c5s4)c3)n2)cc1]

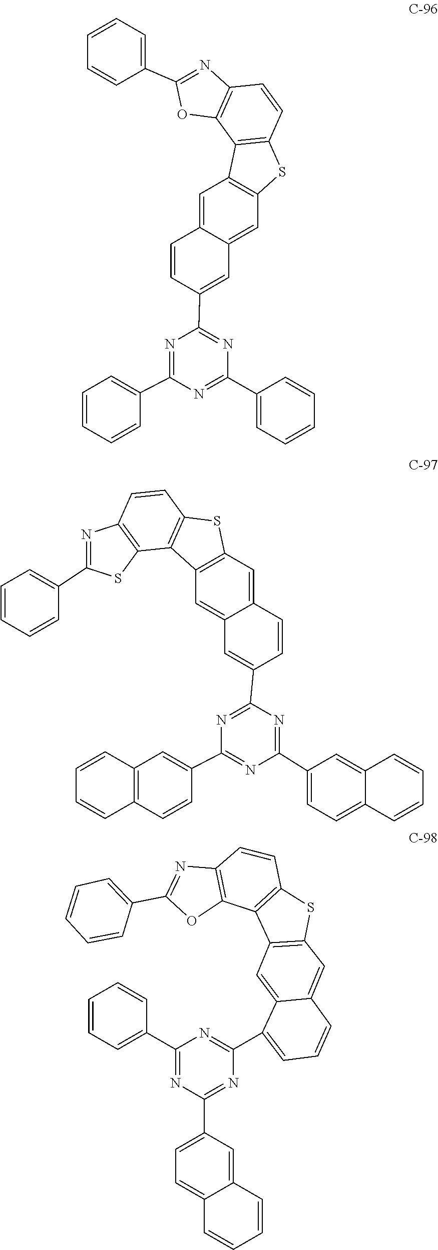 [SMILES: c1ccc(-c2nc(-c3ccccc3)nc(-c3ccc4cc5c(cc4c3)sc3ccc4nc(-c6ccccc6)oc4c35)n2)cc1 ; c1ccc(-c2nc3ccc4sc5cc6ccc(-c7nc(-c8ccc9ccccc9c8)nc(-c8ccc9ccccc9c8)n7)cc6cc5c4c3s2)cc1 ; c1ccc(-c2nc(-c3ccc4ccccc4c3)nc(-c3cccc4cc5sc6ccc7nc(-c8ccccc8)oc7c6c5cc34)n2)cc1]

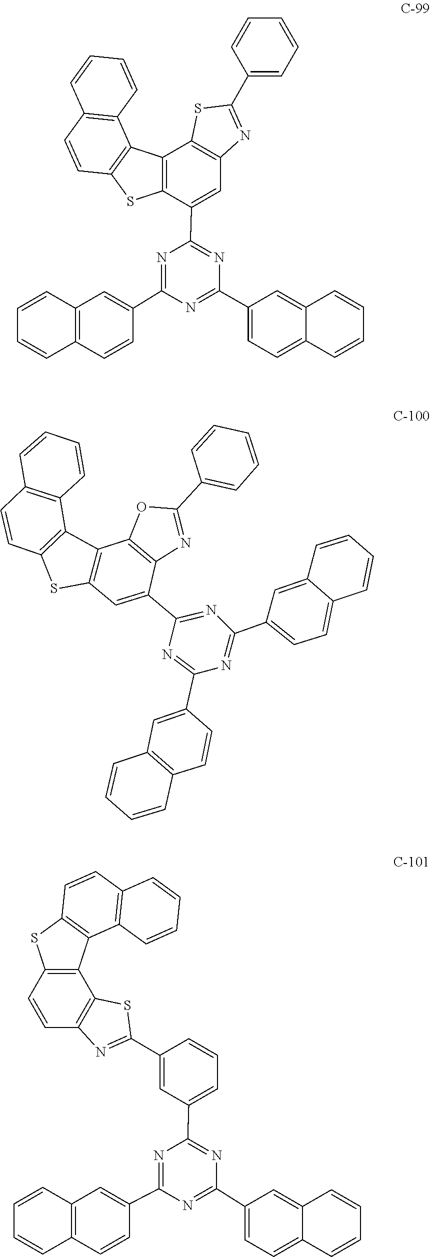 [SMILES: c1ccc(-c2nc3cc(-c4nc(-c5ccc6ccccc6c5)nc(-c5ccc6ccccc6c5)n4)c4sc5ccc6ccccc6c5c4c3s2)cc1 ; c1ccc(-c2nc3c(-c4nc(-c5ccc6ccccc6c5)nc(-c5ccc6ccccc6c5)n4)cc4sc5ccc6ccccc6c5c4c3o2)cc1 ; c1cc(-c2nc(-c3ccc4ccccc4c3)nc(-c3ccc4ccccc4c3)n2)cc(-c2nc3ccc4sc5ccc6ccccc6c5c4c3s2)c1]

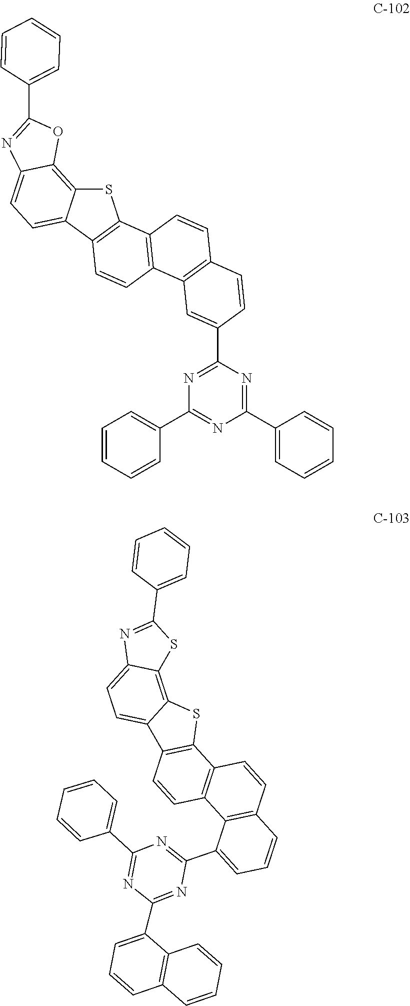 [SMILES: c1ccc(-c2nc(-c3ccccc3)nc(-c3ccc4ccc5c(ccc6c7ccc8nc(-c9ccccc9)oc8c7sc56)c4c3)n2)cc1 ; c1ccc(-c2nc(-c3cccc4ccccc34)nc(-c3cccc4ccc5c(ccc6c7ccc8nc(-c9ccccc9)sc8c7sc65)c34)n2)cc1]

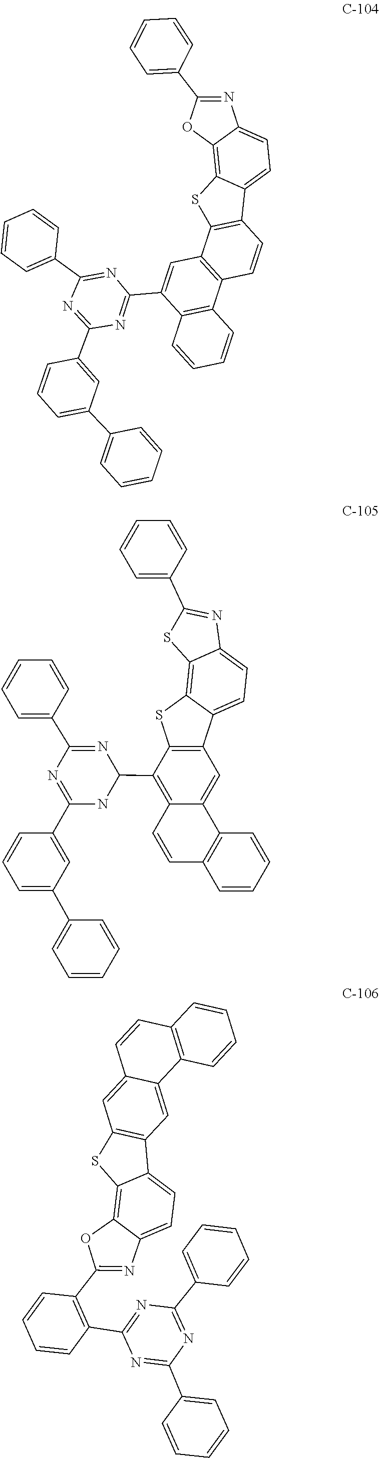

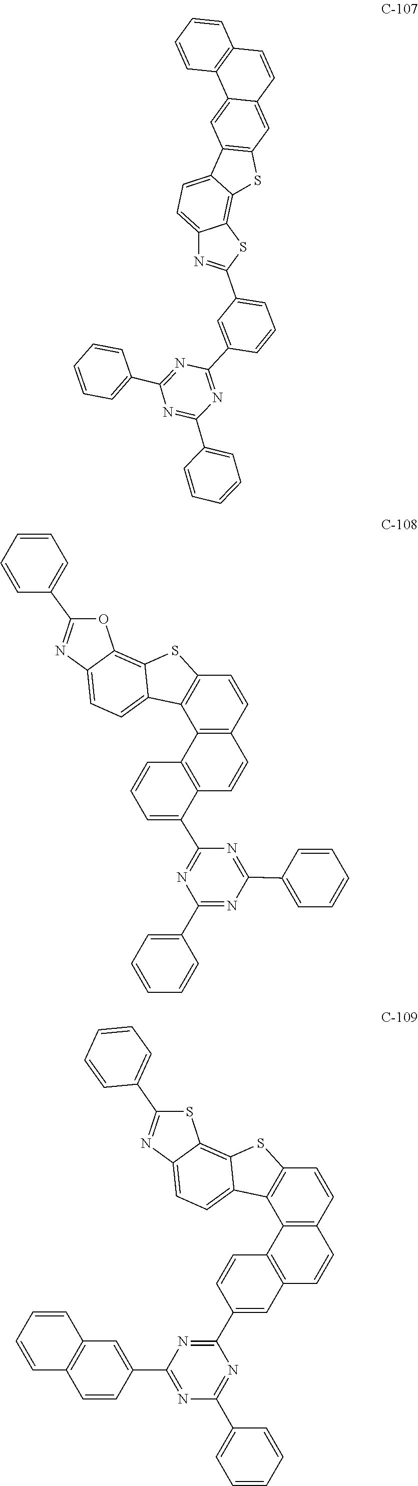

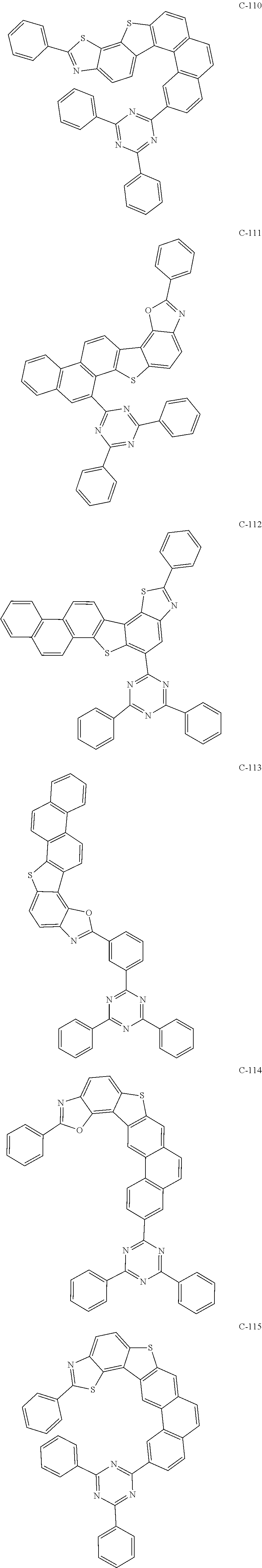

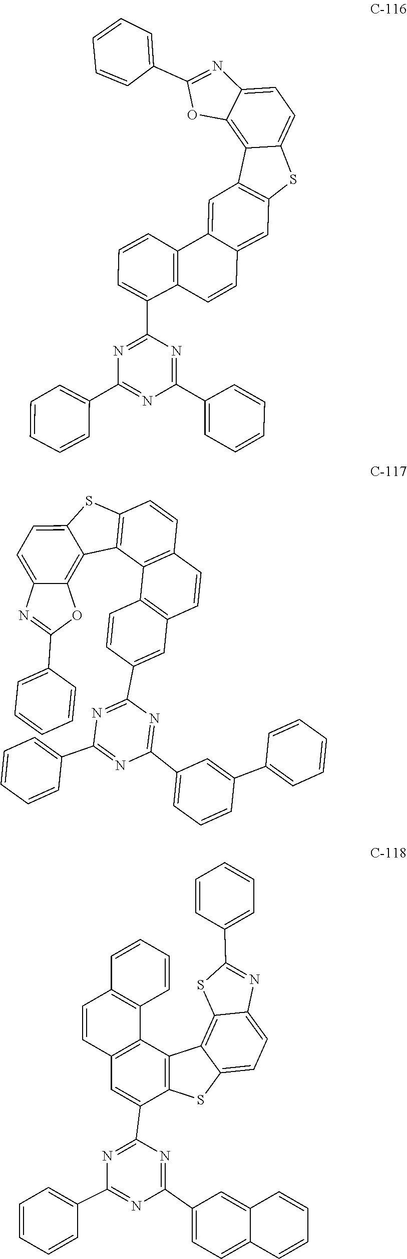

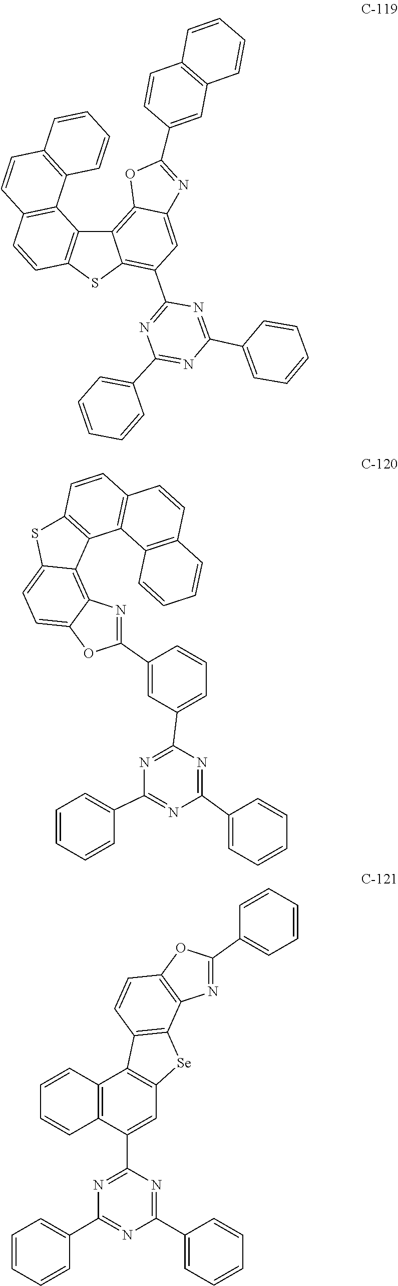

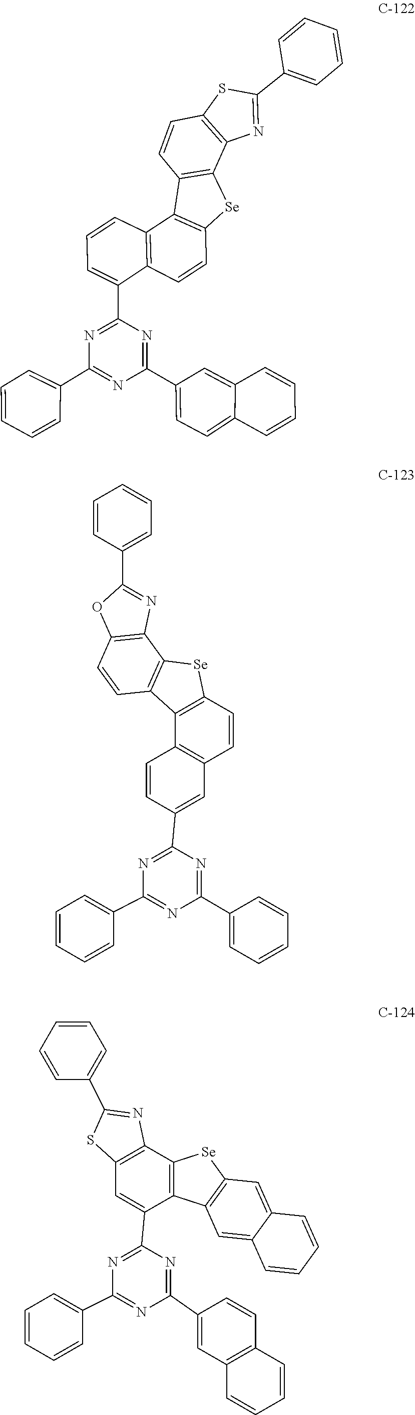 [SMILES: c1ccc(-c2nc(-c3ccc4ccccc4c3)nc(-c3cccc4c3ccc3[se]c5c(ccc6sc(-c7ccccc7)nc65)c34)n2)cc1 ; c1ccc(-c2nc(-c3ccccc3)nc(-c3ccc4c(ccc5[se]c6c(ccc7oc(-c8ccccc8)nc76)c54)c3)n2)cc1 ; c1ccc(-c2nc(-c3ccc4ccccc4c3)nc(-c3cc4sc(-c5ccccc5)nc4c4[se]c5cc6ccccc6cc5c34)n2)cc1]

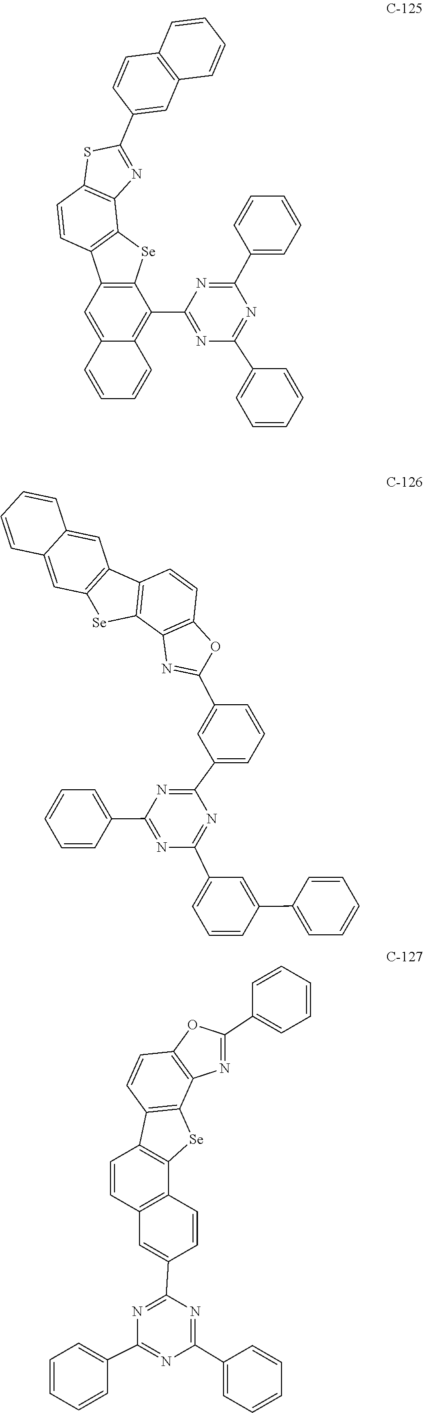

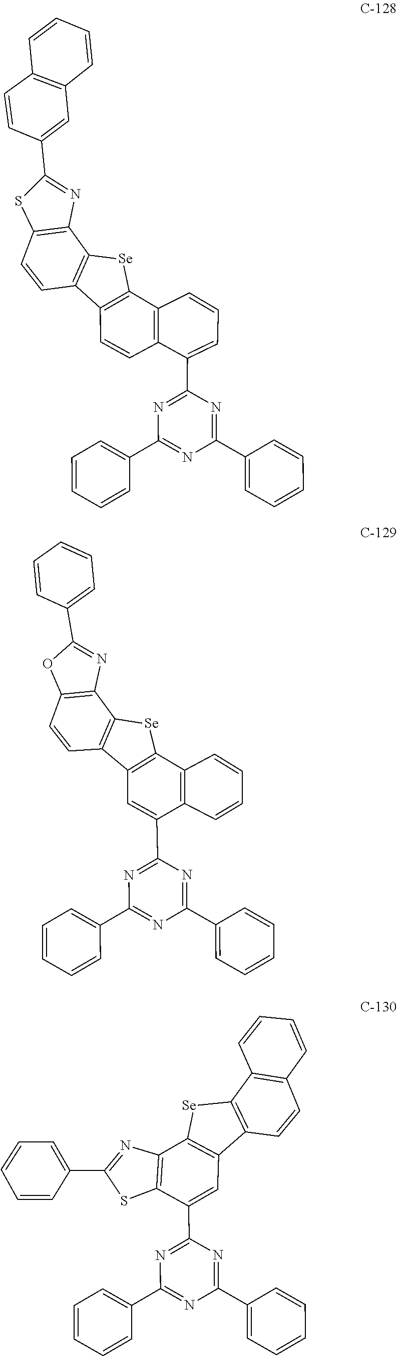

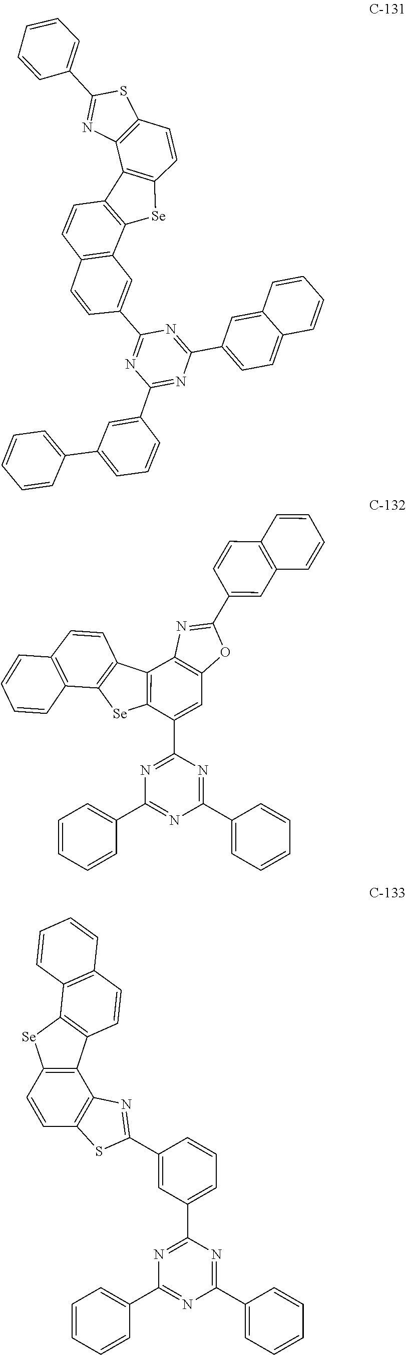 [SMILES: c1ccc(-c2cccc(-c3nc(-c4ccc5ccccc5c4)nc(-c4ccc5ccc6c([se]c7ccc8sc(-c9ccccc9)nc8c76)c5c4)n3)c2)cc1 ; c1ccc(-c2nc(-c3ccccc3)nc(-c3cc4oc(-c5ccc6ccccc6c5)nc4c4c3[se]c3c5ccccc5ccc34)n2)cc1 ; c1ccc(-c2nc(-c3ccccc3)nc(-c3cccc(-c4nc5c(ccc6[se]c7c8ccccc8ccc7c65)s4)c3)n2)cc1]

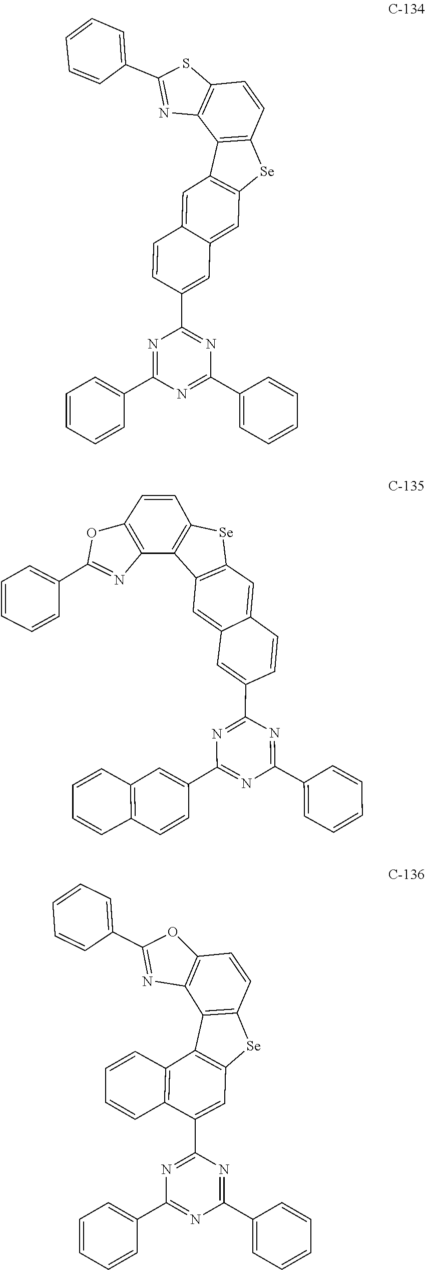 [SMILES: c1ccc(-c2nc(-c3ccccc3)nc(-c3ccc4cc5c(cc4c3)[se]c3ccc4sc(-c6ccccc6)nc4c35)n2)cc1 ; c1ccc(-c2nc(-c3ccc4ccccc4c3)nc(-c3ccc4cc5[se]c6ccc7oc(-c8ccccc8)nc7c6c5cc4c3)n2)cc1 ; c1ccc(-c2nc(-c3ccccc3)nc(-c3cc4[se]c5ccc6oc(-c7ccccc7)nc6c5c4c4ccccc34)n2)cc1]

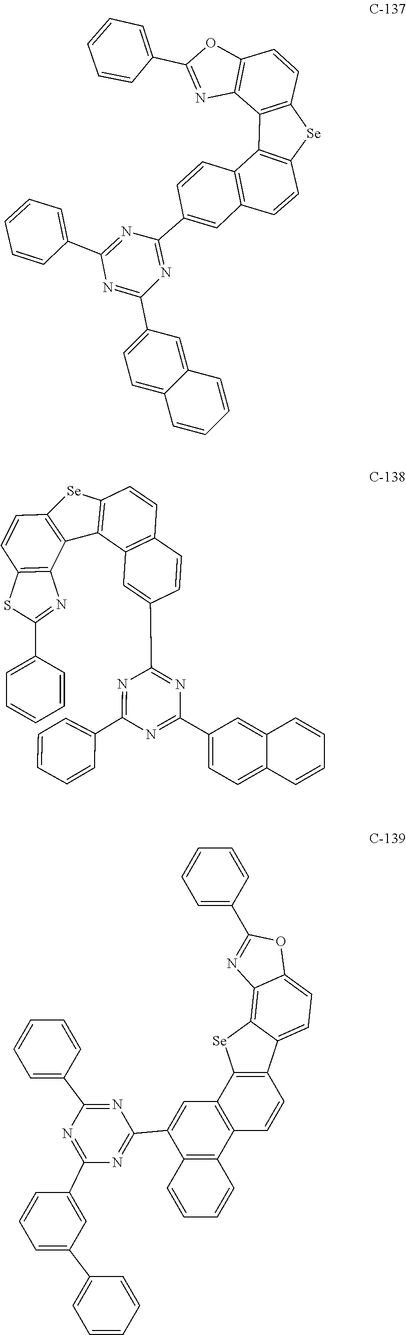 [SMILES: c1ccc(-c2nc(-c3ccc4ccccc4c3)nc(-c3ccc4c(ccc5[se]c6ccc7oc(-c8ccccc8)nc7c6c54)c3)n2)cc1 ; c1ccc(-c2nc(-c3ccc4ccccc4c3)nc(-c3ccc4ccc5[se]c6ccc7sc(-c8ccccc8)nc7c6c5c4c3)n2)cc1 ; c1ccc(-c2cccc(-c3nc(-c4ccccc4)nc(-c4cc5c(ccc6c7ccc8oc(-c9ccccc9)nc8c7[se]c56)c5ccccc45)n3)c2)cc1]

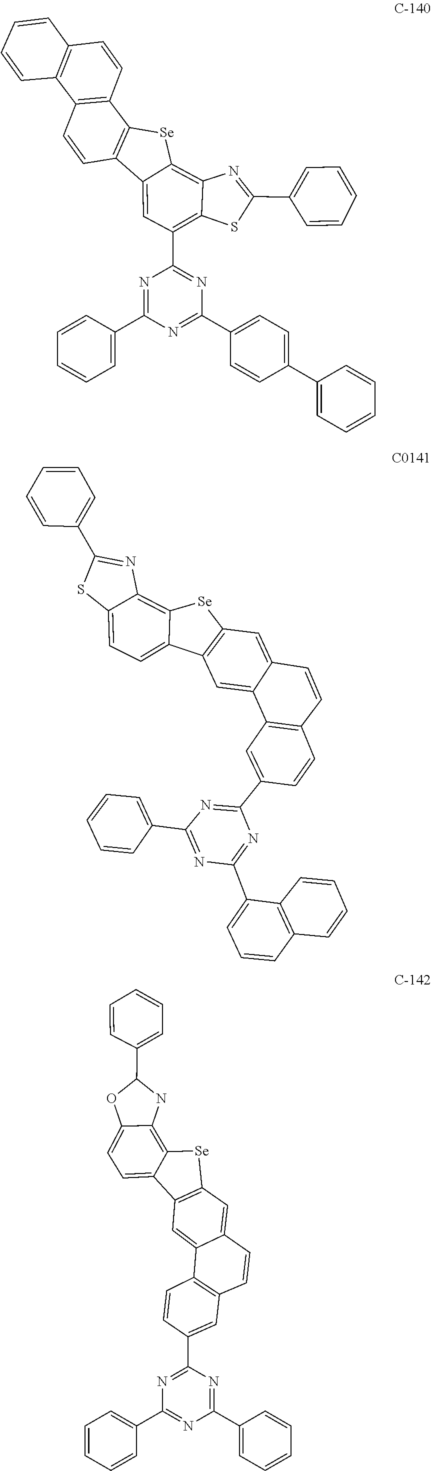

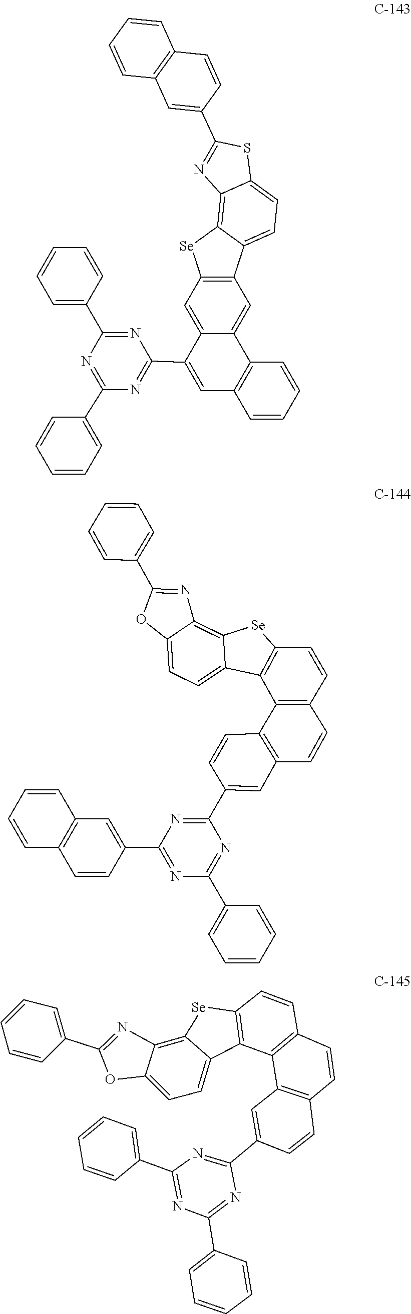

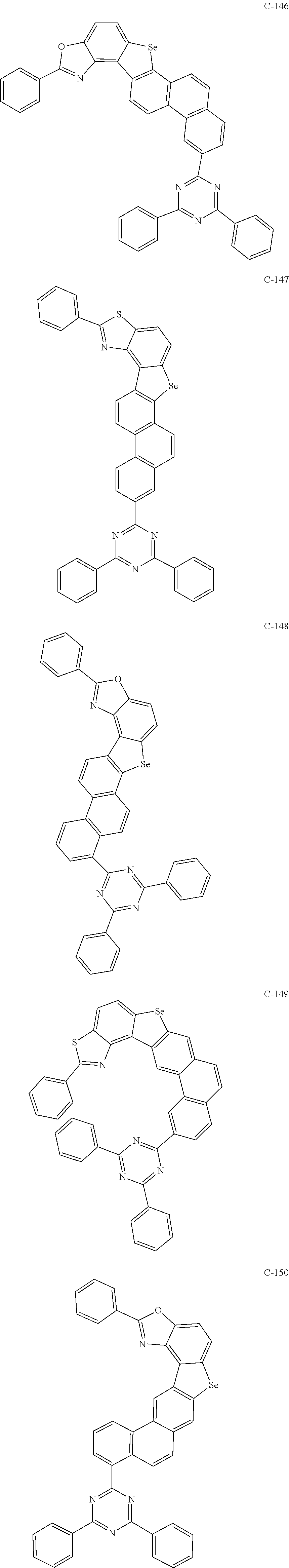

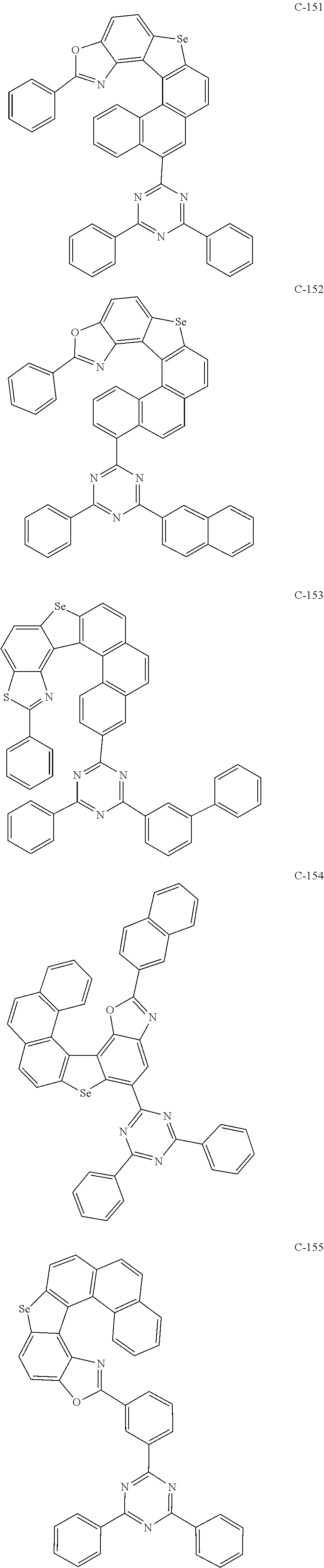

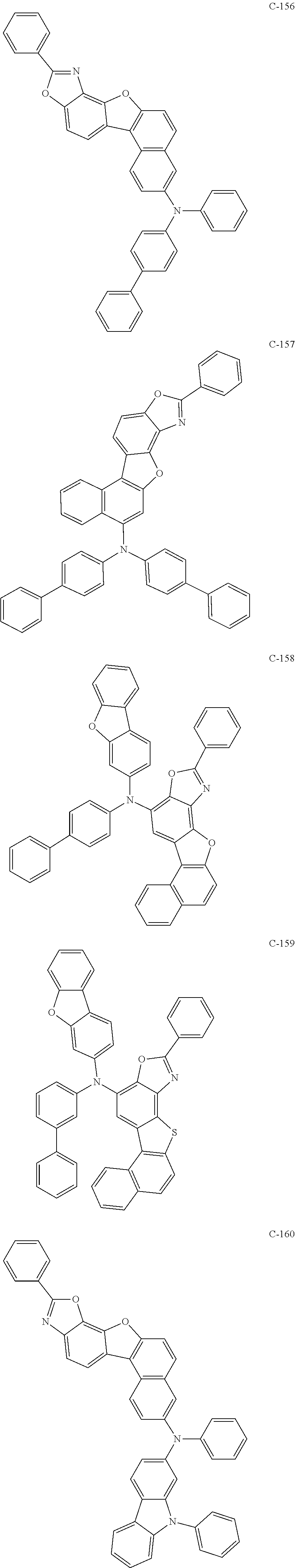 [SMILES: c1ccc(-c2ccc(N(c3ccccc3)c3ccc4c(ccc5oc6c(ccc7oc(-c8ccccc8)nc76)c54)c3)cc2)cc1 ; c1ccc(-c2ccc(N(c3ccc(-c4ccccc4)cc3)c3cc4oc5c(ccc6oc(-c7ccccc7)nc65)c4c4ccccc34)cc2)cc1 ; c1ccc(-c2ccc(N(c3ccc4c(c3)oc3ccccc34)c3cc4c(oc5ccc6ccccc6c54)c4nc(-c5ccccc5)oc34)cc2)cc1 ; c1ccc(-c2cccc(N(c3ccc4c(c3)oc3ccccc34)c3cc4c(sc5ccc6ccccc6c54)c4nc(-c5ccccc5)oc34)c2)cc1 ; c1ccc(-c2nc3ccc4c(oc5ccc6cc(N(c7ccccc7)c7ccc8c9ccccc9n(-c9ccccc9)c8c7)ccc6c54)c3o2)cc1]

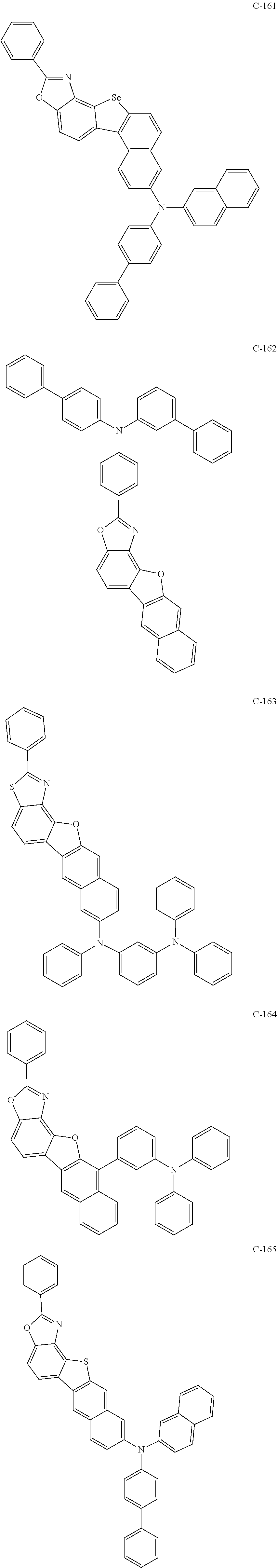 [SMILES: c1ccc(-c2ccc(N(c3ccc4ccccc4c3)c3ccc4c(ccc5[se]c6c(ccc7oc(-c8ccccc8)nc76)c54)c3)cc2)cc1 ; c1ccc(-c2ccc(N(c3ccc(-c4nc5c(ccc6c7cc8ccccc8cc7oc65)o4)cc3)c3cccc(-c4ccccc4)c3)cc2)cc1 ; c1ccc(-c2nc3c(ccc4c5cc6cc(N(c7ccccc7)c7cccc(N(c8ccccc8)c8ccccc8)c7)ccc6cc5oc43)s2)cc1 ; c1ccc(-c2nc3c(ccc4c5cc6ccccc6c(-c6cccc(N(c7ccccc7)c7ccccc7)c6)c5oc43)o2)cc1 ; c1ccc(-c2ccc(N(c3ccc4ccccc4c3)c3ccc4cc5c(cc4c3)sc3c5ccc4oc(-c5ccccc5)nc43)cc2)cc1]

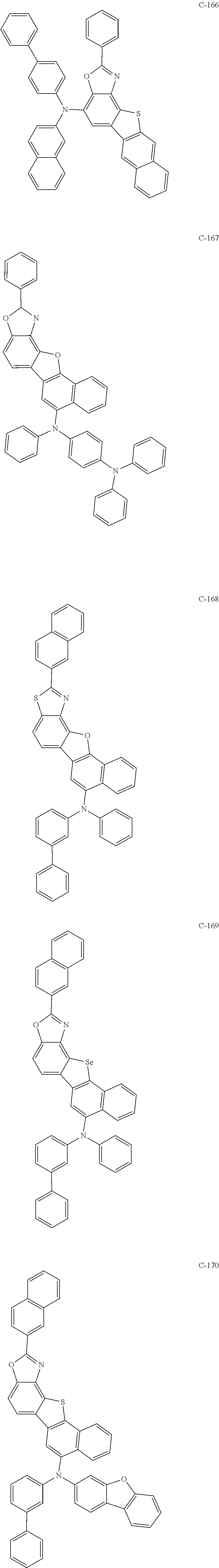

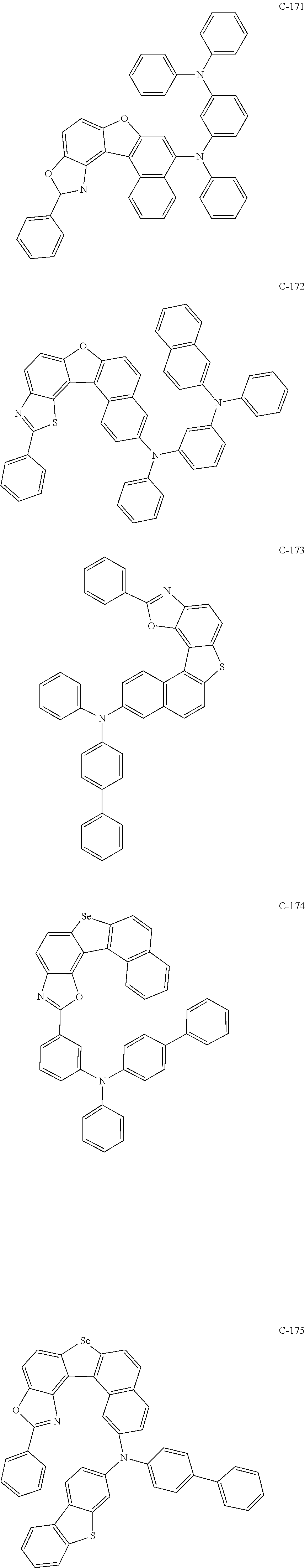

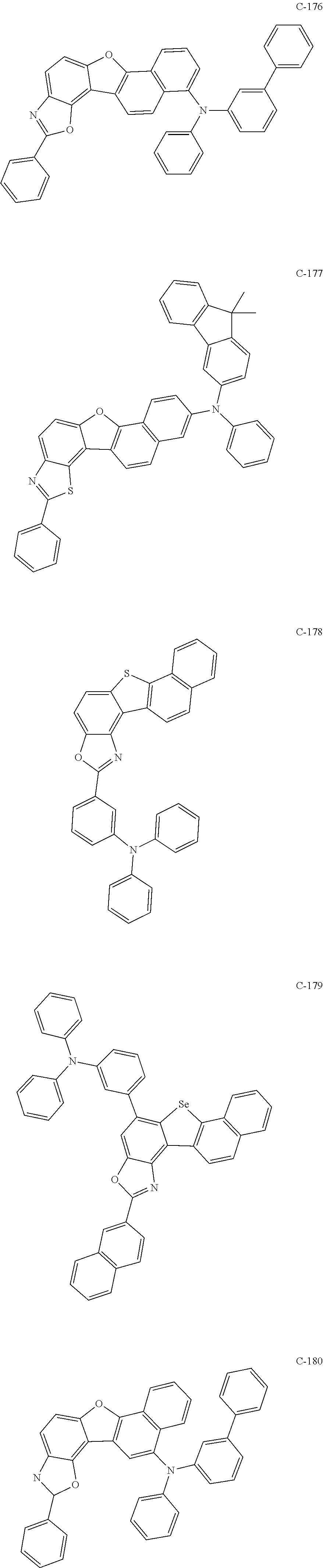

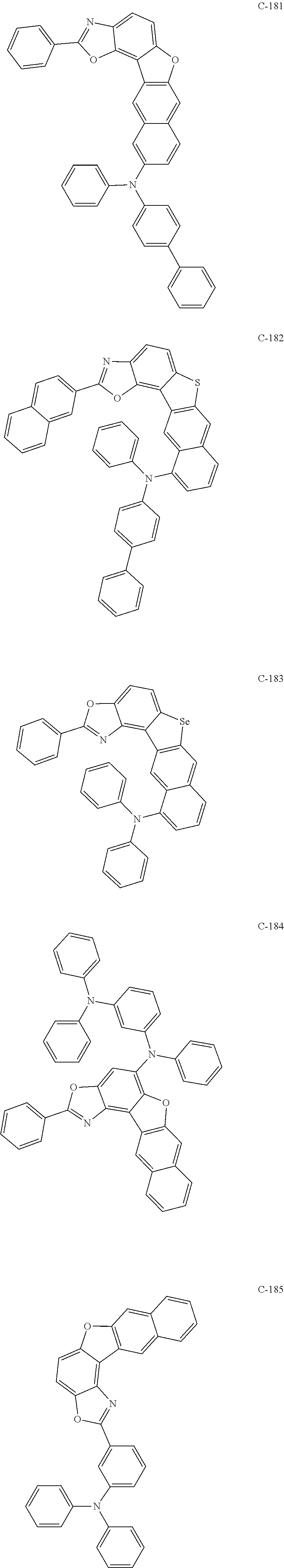 [SMILES: c1ccc(-c2ccc(N(c3ccccc3)c3ccc4cc5oc6ccc7nc(-c8ccccc8)oc7c6c5cc4c3)cc2)cc1 ; c1ccc(-c2ccc(N(c3ccccc3)c3cccc4cc5sc6ccc7nc(-c8ccc9ccccc9c8)oc7c6c5cc34)cc2)cc1 ; c1ccc(-c2nc3c(ccc4[se]c5cc6cccc(N(c7ccccc7)c7ccccc7)c6cc5c43)o2)cc1 ; c1ccc(-c2nc3c(cc(N(c4ccccc4)c4cccc(N(c5ccccc5)c5ccccc5)c4)c4oc5cc6ccccc6cc5c43)o2)cc1 ; c1ccc(N(c2ccccc2)c2cccc(-c3nc4c(ccc5oc6cc7ccccc7cc6c54)o3)c2)cc1]

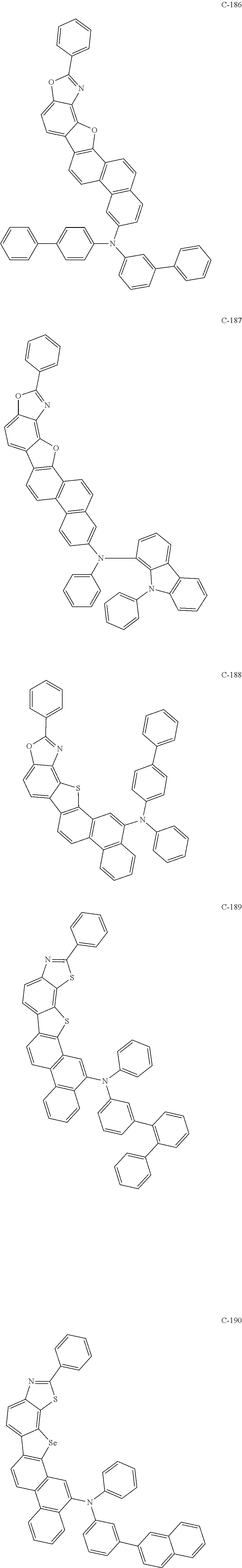 [SMILES: c1ccc(-c2ccc(N(c3cccc(-c4ccccc4)c3)c3ccc4ccc5c(ccc6c7ccc8oc(-c9ccccc9)nc8c7oc56)c4c3)cc2)cc1 ; c1ccc(-c2nc3c(ccc4c5ccc6c7ccc(N(c8ccccc8)c8cccc9c%10ccccc%10n(-c%10ccccc%10)c89)cc7ccc6c5oc43)o2)cc1 ; c1ccc(-c2ccc(N(c3ccccc3)c3cc4c(ccc5c6ccc7oc(-c8ccccc8)nc7c6sc45)c4ccccc34)cc2)cc1 ; c1ccc(-c2nc3ccc4c5ccc6c7ccccc7c(N(c7ccccc7)c7cccc(-c8ccccc8-c8ccccc8)c7)cc6c5sc4c3s2)cc1 ; c1ccc(-c2nc3ccc4c5ccc6c7ccccc7c(N(c7ccccc7)c7cccc(-c8ccc9ccccc9c8)c7)cc6c5[se]c4c3s2)cc1]

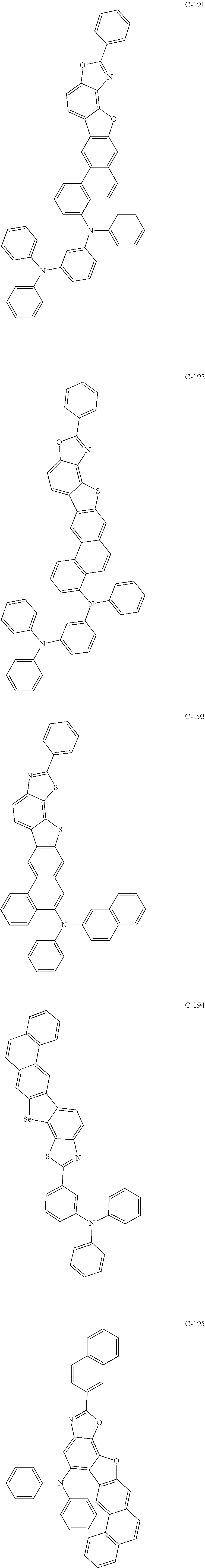 [SMILES: c1ccc(-c2nc3c(ccc4c5cc6c(ccc7c(N(c8ccccc8)c8cccc(N(c9ccccc9)c9ccccc9)c8)cccc76)cc5oc43)o2)cc1 ; c1ccc(-c2nc3c(ccc4c5cc6c(ccc7c(N(c8ccccc8)c8cccc(N(c9ccccc9)c9ccccc9)c8)cccc76)cc5sc43)o2)cc1 ; c1ccc(-c2nc3ccc4c5cc6c(cc(N(c7ccccc7)c7ccc8ccccc8c7)c7ccccc76)cc5sc4c3s2)cc1 ; c1ccc(N(c2ccccc2)c2cccc(-c3nc4ccc5c6cc7c(ccc8ccccc87)cc6[se]c5c4s3)c2)cc1 ; c1ccc(N(c2ccccc2)c2cc3nc(-c4ccc5ccccc5c4)oc3c3oc4cc5ccc6ccccc6c5cc4c23)cc1]

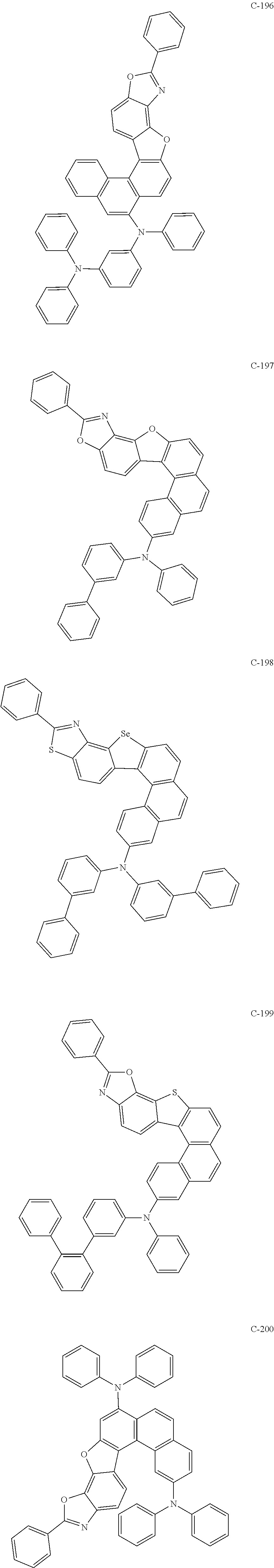 [SMILES: c1ccc(-c2nc3c(ccc4c3oc3ccc5c(N(c6ccccc6)c6cccc(N(c7ccccc7)c7ccccc7)c6)cc6ccccc6c5c34)o2)cc1 ; c1ccc(-c2cccc(N(c3ccccc3)c3ccc4c(ccc5ccc6oc7c(ccc8oc(-c9ccccc9)nc87)c6c54)c3)c2)cc1 ; c1ccc(-c2cccc(N(c3cccc(-c4ccccc4)c3)c3ccc4c(ccc5ccc6[se]c7c(ccc8sc(-c9ccccc9)nc87)c6c54)c3)c2)cc1 ; c1ccc(-c2nc3ccc4c(sc5ccc6ccc7cc(N(c8ccccc8)c8cccc(-c9ccccc9-c9ccccc9)c8)ccc7c6c54)c3o2)cc1 ; c1ccc(-c2nc3ccc4c(oc5cc(N(c6ccccc6)c6ccccc6)c6ccc7ccc(N(c8ccccc8)c8ccccc8)cc7c6c54)c3o2)cc1]

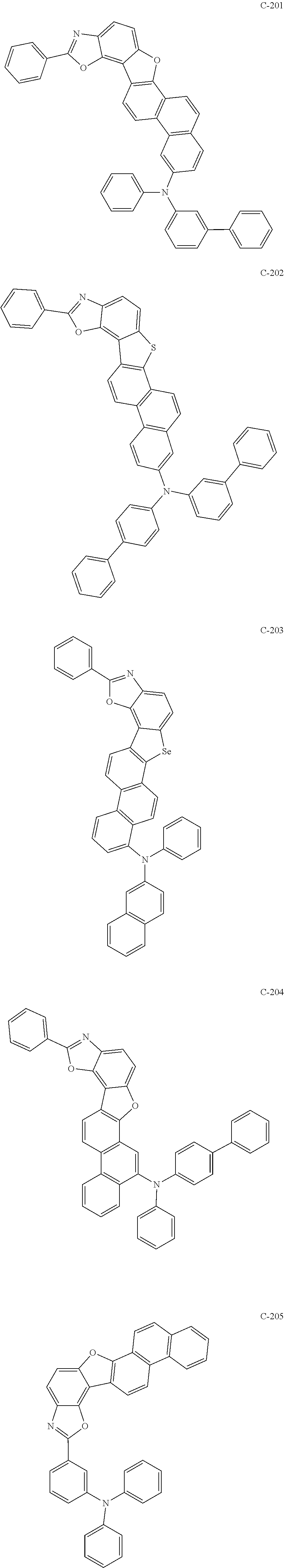 [SMILES: c1ccc(-c2cccc(N(c3ccccc3)c3ccc4ccc5c(ccc6c5oc5ccc7nc(-c8ccccc8)oc7c56)c4c3)c2)cc1 ; c1ccc(-c2ccc(N(c3cccc(-c4ccccc4)c3)c3ccc4c(ccc5c4ccc4c5sc5ccc6nc(-c7ccccc7)oc6c54)c3)cc2)cc1 ; c1ccc(-c2nc3ccc4[se]c5c6ccc7c(N(c8ccccc8)c8ccc9ccccc9c8)cccc7c6ccc5c4c3o2)cc1 ; c1ccc(-c2ccc(N(c3ccccc3)c3cc4c(ccc5c4oc4ccc6nc(-c7ccccc7)oc6c45)c4ccccc34)cc2)cc1 ; c1ccc(N(c2ccccc2)c2cccc(-c3nc4ccc5oc6c7ccc8ccccc8c7ccc6c5c4o3)c2)cc1]

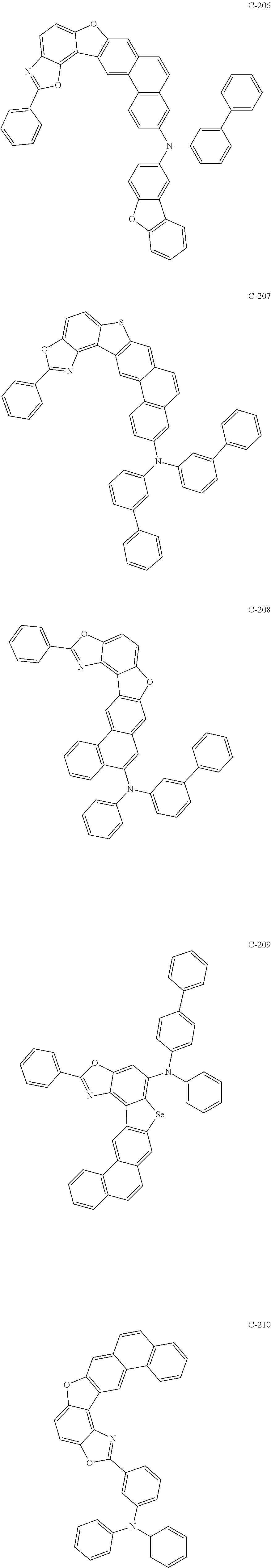 [SMILES: c1ccc(-c2cccc(N(c3ccc4c(ccc5cc6oc7ccc8nc(-c9ccccc9)oc8c7c6cc54)c3)c3ccc4oc5ccccc5c4c3)c2)cc1 ; c1ccc(-c2cccc(N(c3cccc(-c4ccccc4)c3)c3ccc4c(ccc5cc6sc7ccc8oc(-c9ccccc9)nc8c7c6cc54)c3)c2)cc1 ; c1ccc(-c2cccc(N(c3ccccc3)c3cc4cc5oc6ccc7oc(-c8ccccc8)nc7c6c5cc4c4ccccc34)c2)cc1 ; c1ccc(-c2ccc(N(c3ccccc3)c3cc4oc(-c5ccccc5)nc4c4c3[se]c3cc5ccc6ccccc6c5cc34)cc2)cc1 ; c1ccc(N(c2ccccc2)c2cccc(-c3nc4c(ccc5oc6cc7ccc8ccccc8c7cc6c54)o3)c2)cc1]

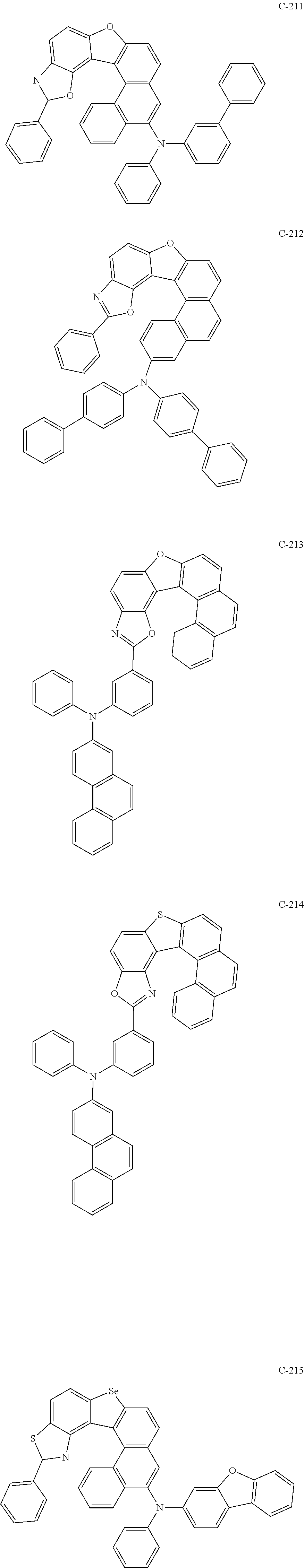

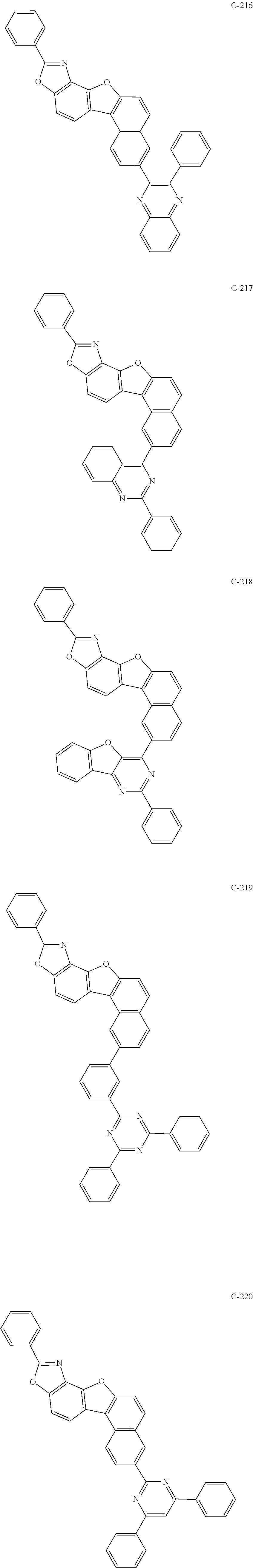 [SMILES: c1ccc(-c2nc3c(ccc4c3oc3ccc5cc(-c6nc7ccccc7nc6-c6ccccc6)ccc5c34)o2)cc1 ; c1ccc(-c2nc(-c3ccc4ccc5oc6c(ccc7oc(-c8ccccc8)nc76)c5c4c3)c3ccccc3n2)cc1 ; c1ccc(-c2nc(-c3ccc4ccc5oc6c(ccc7oc(-c8ccccc8)nc76)c5c4c3)c3oc4ccccc4c3n2)cc1 ; c1ccc(-c2nc(-c3ccccc3)nc(-c3cccc(-c4ccc5ccc6oc7c(ccc8oc(-c9ccccc9)nc87)c6c5c4)c3)n2)cc1 ; c1ccc(-c2cc(-c3ccccc3)nc(-c3ccc4c(ccc5oc6c(ccc7oc(-c8ccccc8)nc76)c54)c3)n2)cc1]

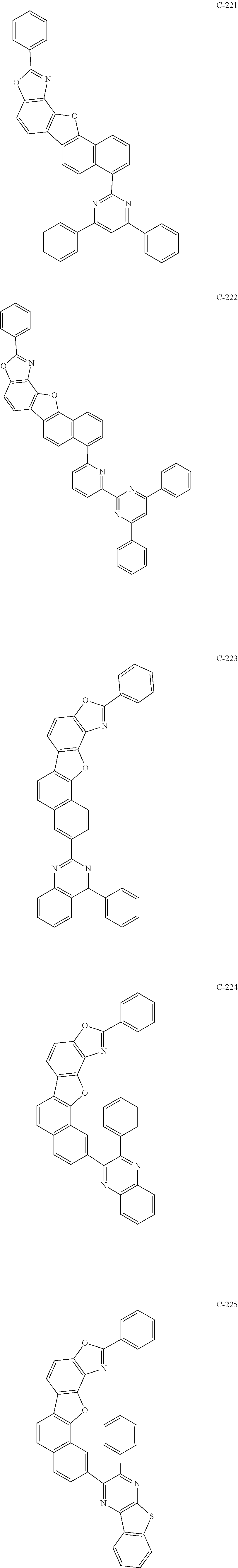 [SMILES: c1ccc(-c2cc(-c3ccccc3)nc(-c3cccc4c3ccc3c5ccc6oc(-c7ccccc7)nc6c5oc43)n2)cc1 ; c1ccc(-c2cc(-c3ccccc3)nc(-c3cccc(-c4cccc5c4ccc4c6ccc7oc(-c8ccccc8)nc7c6oc54)n3)n2)cc1 ; c1ccc(-c2nc3c(ccc4c5ccc6cc(-c7nc(-c8ccccc8)c8ccccc8n7)ccc6c5oc43)o2)cc1 ; c1ccc(-c2nc3c(ccc4c5ccc6ccc(-c7nc8ccccc8nc7-c7ccccc7)cc6c5oc43)o2)cc1 ; c1ccc(-c2nc3c(ccc4c5ccc6ccc(-c7nc8c(nc7-c7ccccc7)sc7ccccc78)cc6c5oc43)o2)cc1]

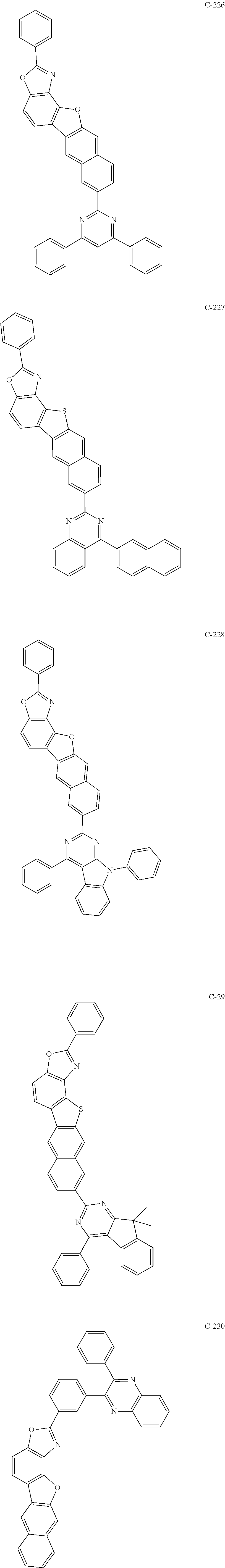

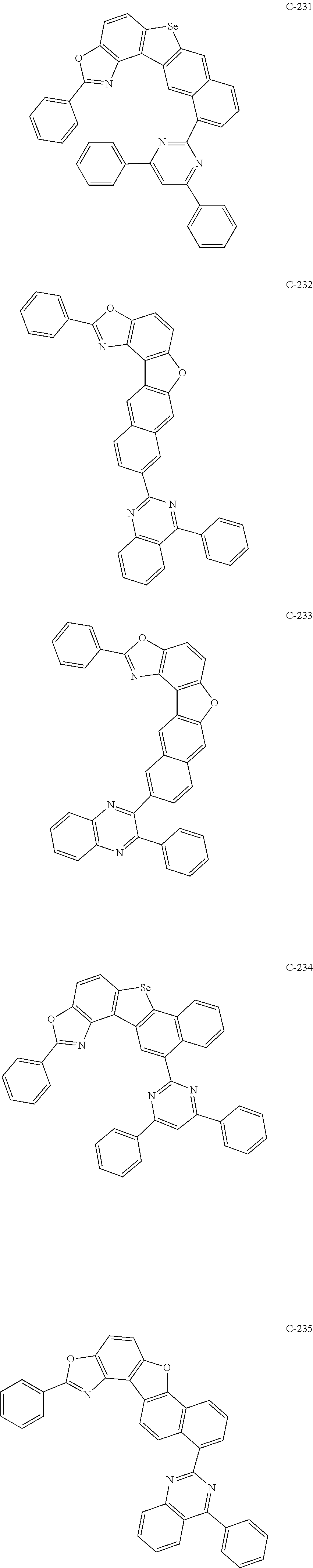 [SMILES: c1ccc(-c2cc(-c3ccccc3)nc(-c3cccc4cc5[se]c6ccc7oc(-c8ccccc8)nc7c6c5cc34)n2)cc1 ; c1ccc(-c2nc3c(ccc4oc5cc6cc(-c7nc(-c8ccccc8)c8ccccc8n7)ccc6cc5c43)o2)cc1 ; c1ccc(-c2nc3c(ccc4oc5cc6ccc(-c7nc8ccccc8nc7-c7ccccc7)cc6cc5c43)o2)cc1 ; c1ccc(-c2cc(-c3ccccc3)nc(-c3cc4c([se]c5ccc6oc(-c7ccccc7)nc6c54)c4ccccc34)n2)cc1 ; c1ccc(-c2nc3c(ccc4oc5c6cccc(-c7nc(-c8ccccc8)c8ccccc8n7)c6ccc5c43)o2)cc1]

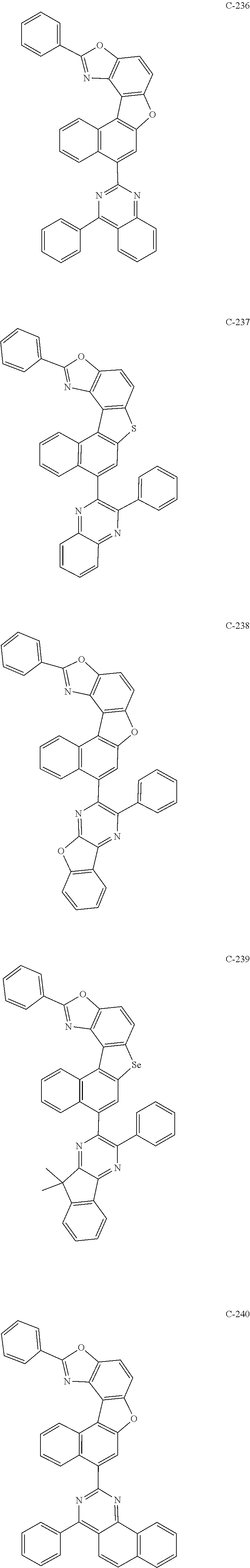 [SMILES: c1ccc(-c2nc3c(ccc4oc5cc(-c6nc(-c7ccccc7)c7ccccc7n6)c6ccccc6c5c43)o2)cc1 ; c1ccc(-c2nc3c(ccc4sc5cc(-c6nc7ccccc7nc6-c6ccccc6)c6ccccc6c5c43)o2)cc1 ; c1ccc(-c2nc3c(ccc4oc5cc(-c6nc7oc8ccccc8c7nc6-c6ccccc6)c6ccccc6c5c43)o2)cc1 ; CC1(C)c2ccccc2-c2nc(-c3ccccc3)c(-c3cc4[se]c5ccc6oc(-c7ccccc7)nc6c5c4c4ccccc34)nc21 ; c1ccc(-c2nc3c(ccc4oc5cc(-c6nc(-c7ccccc7)c7ccc8ccccc8c7n6)c6ccccc6c5c43)o2)cc1]

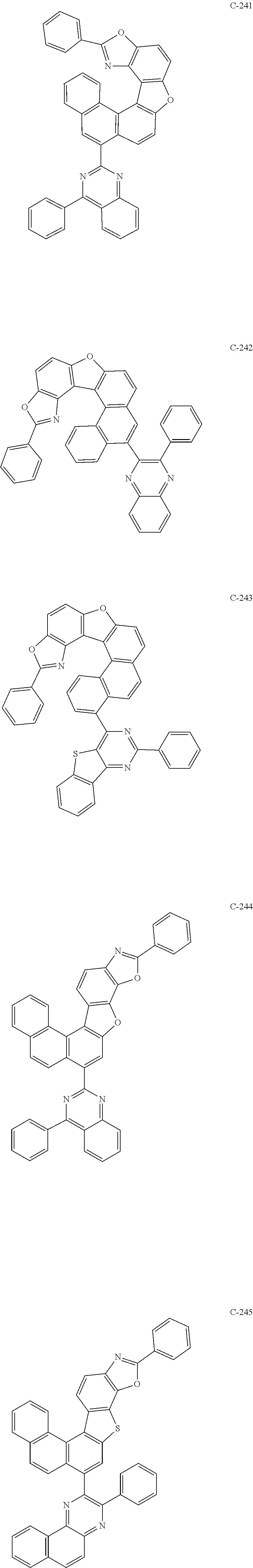

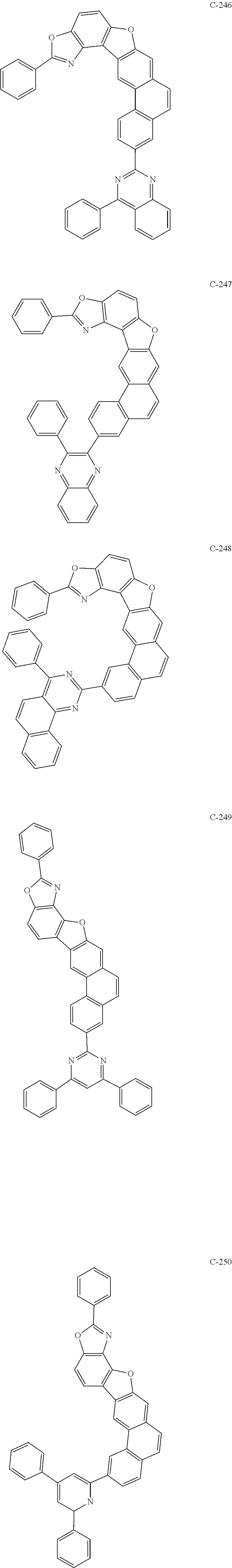

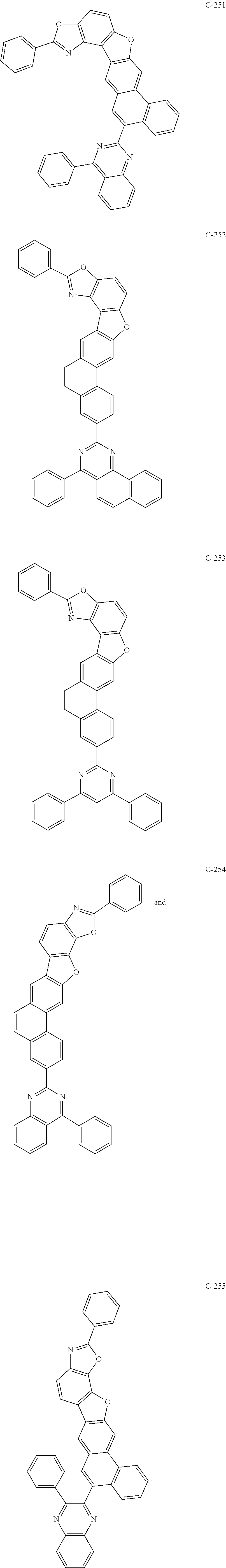 [SMILES: c1ccc(-c2nc3c(ccc4oc5cc6c(cc(-c7nc(-c8ccccc8)c8ccccc8n7)c7ccccc76)cc5c43)o2)cc1 ; c1ccc(-c2nc3c(ccc4oc5cc6c(ccc7cc(-c8nc(-c9ccccc9)c9ccc%10ccccc%10c9n8)ccc76)cc5c43)o2)cc1 ; c1ccc(-c2cc(-c3ccccc3)nc(-c3ccc4c(ccc5cc6c(cc54)oc4ccc5oc(-c7ccccc7)nc5c46)c3)n2)cc1 ; c1ccc(-c2nc3ccc4c5cc6ccc7cc(-c8nc(-c9ccccc9)c9ccccc9n8)ccc7c6cc5oc4c3o2)cc1 ; c1ccc(-c2nc3ccc4c5cc6cc(-c7nc8ccccc8nc7-c7ccccc7)c7ccccc7c6cc5oc4c3o2)cc1]

The compound represented by Formula 1 according to the present disclosure may be produced by referring to the following Reaction Schemes, but is not limited thereto.

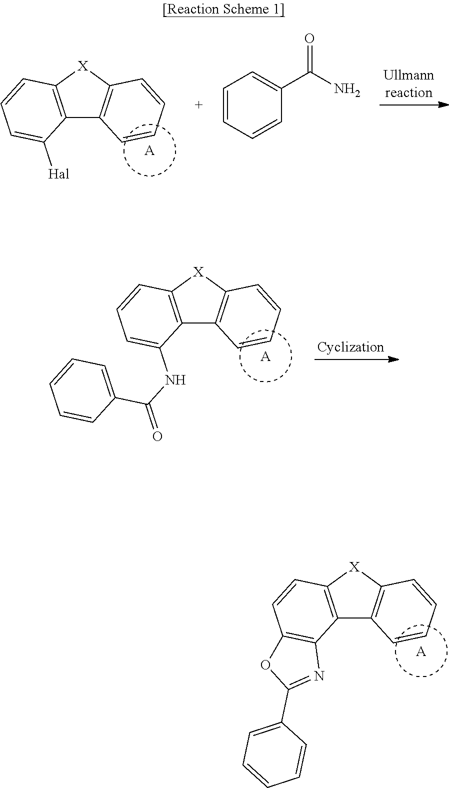

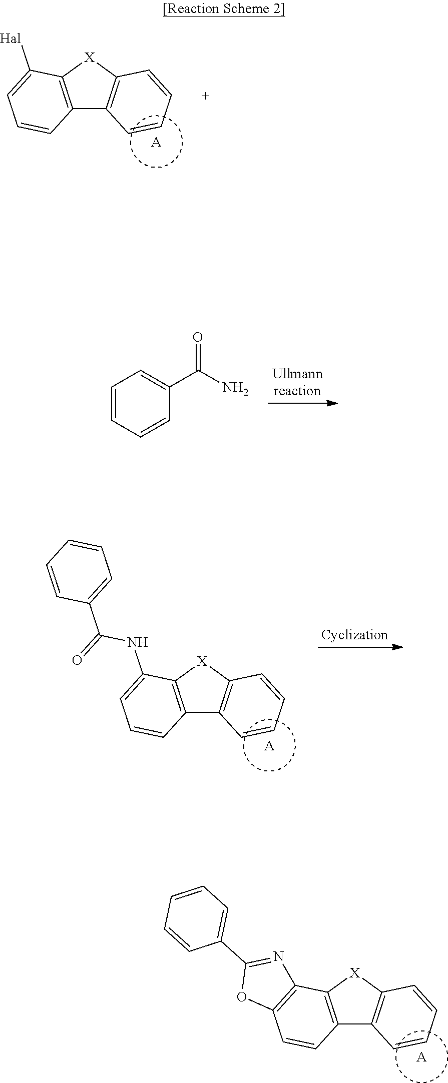

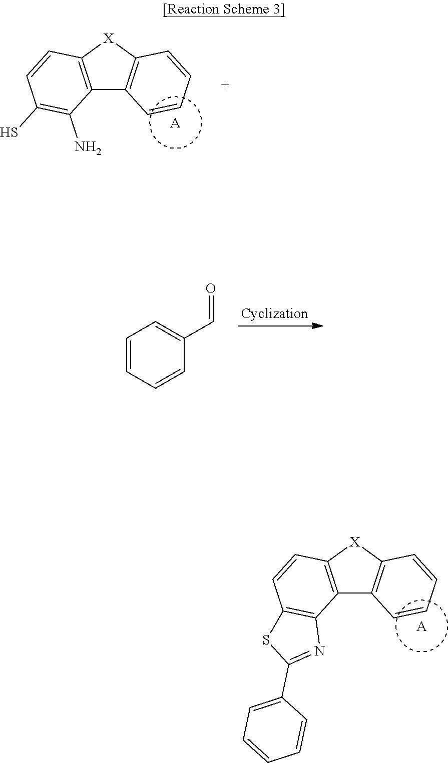

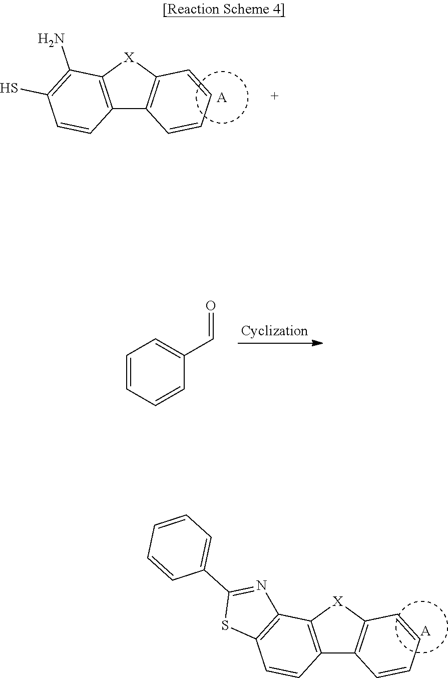

In reaction schemes 1 to 4, ring A is a fused ring of benzene or naphthalene, X is as defined in Formula 1, and Hal represents halogen.

As a host compound that can be used in combination with the organic electroluminescent compound of the present disclosure, a compound represented by any one the following formulas 11 to 14 may be exemplified, but it is not limited thereto.

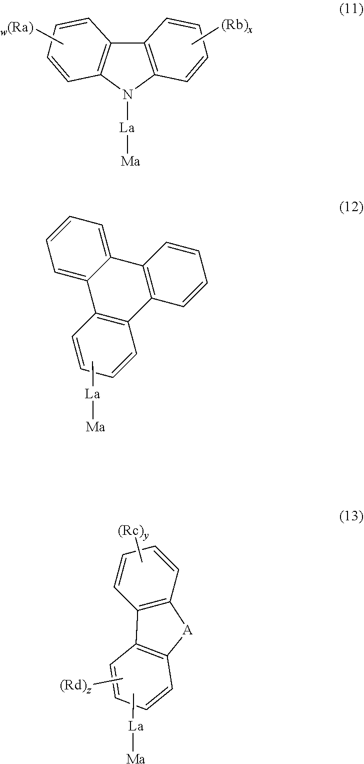

In formulas 11 to 13,
Ma represents a substituted or unsubstituted (C6-C30)aryl, a substituted or unsubstituted mono- or di-(C6-C30)arylamino, a substituted or unsubstituted (C6-C30)aryl(3- to 30-membered)heteroarylamino, or a substituted or unsubstituted (3- to 30-membered)heteroaryl;
La represents a single bond, a substituted or unsubstituted (C6-C30)arylene, or a substituted or unsubstituted (3- to 30-membered)heteroarylene;
A represents S, O, N(Re) or C(Rf)(Rg);
Ra to Rd, each independently, represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C60)aryl, a substituted or unsubstituted (3- to 30-membered)heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted (C1-C30)alkyl(C6-C30)arylamino, or a substituted or unsubstituted mono- or di-(C6-C30)arylamino; or may be linked to an adjacent substituent(s) to form a substituted or unsubstituted mono- or polycyclic (3- to 30-membered) alicyclic or aromatic ring, or the combination thereof, and the formed alicyclic or aromatic ring, or the combination thereof may contain at least one heteroatom selected from N, O, and S;
Re to Rg, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or Rf and Rg may be linked to each other to form a substituted or unsubstituted, mono- or polycyclic, (3- to 30-membered) alicyclic or aromatic ring, or the combination thereof, and the formed alicyclic or aromatic ring, or the combination thereof may contain at least one heteroatom selected from N, O, and S;
w to y, each independently, represent an integer of 1 to 4, and z represents an integer of 1 to 3; where if w to z are each an integer of 2 or more, each of Ra to each of Rd may be the same as or different from each other; and
the heteroaryl(ene) contains at least one heteroatom selected from B, N, O, S, Si, and P.

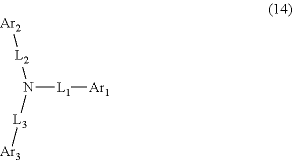

In Formula 14,
L₁ to L₃, each independently, represent a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;
Ar₁ to Ar₃, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or -L₄-N(Arₐ)(Ar_{b});
L₄ represents a single bond, a substituted or unsubstituted (C6-C30)arylene, or a substituted or unsubstituted (3- to 30-membered)heteroarylene;
Arₐ and Ar_{b}, each independently, represent hydrogen, a substituted or unsubstituted (C1-C30)alkyl, a substituted or unsubstituted (C2-C30)alkenyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl; and

With a proviso, the case where all of L₁ to L₃ are single bonds and all of Ar₁ to Ar₃ are hydrogen is excluded. According to one embodiment of the present disclosure, in Formula 14, L₁ to L₃, each independently, may be a single bond, a substituted or unsubstituted (C6-C30)arylene, or a substituted or unsubstituted (3- to 30-membered)heteroarylene. According to another embodiment of the present disclosure, in Formula 14, L₁ to L₃, each independently, may be a single bond, a substituted or unsubstituted (C6-C20)arylene, or a substituted or unsubstituted (3- to 20-membered)heteroarylene. For example, L₁ to L₃, each independently, may be a single bond, a substituted or unsubstituted phenylene, naphthylene, biphenylene, phenanthrenylene, o-terphenylene, dibenzofuranylene, carbazolylene, and pyridinylene, etc.

In Formula 14, Ar₁ to Ar₃, each independently, may be hydrogen, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted fused ring group of a (C3-C30) aliphatic ring(s) and a (C6-C30) aromatic ring(s), or -L₄-N(Arₐ)(Ar_{b}). According to one embodiment of the present disclosure, in Formula 14, Ar₁ to Ar₃, each independently, may be hydrogen, a substituted or unsubstituted (C6-C25)aryl, a substituted or unsubstituted (3- to 25-membered)heteroaryl, a substituted or unsubstituted (C3-C25)cycloalkyl, a substituted or unsubstituted fused ring group of a (C3-C25) aliphatic ring(s) and a (C6-C25) aromatic ring(s), or -L₄-N(Arₐ)(Ar_{b}).

In addition, L₄ may be a single bond or a substituted or unsubstituted (C6-C30)arylene.

Arₐ and Ar_{b}, each independently, may be a substituted or unsubstituted (C6-C30)aryl, or a substituted or unsubstituted (3- to 30-membered)heteroaryl. According to one embodiment of the present disclosure, Arₐ and Ar_{b}, each independently, may be a substituted or unsubstituted (C6-C20)aryl, or a substituted or unsubstituted (3- to 20-membered)heteroaryl.

According to one embodiment of the present disclosure, at least one of Ar₁ to Ar₃ in Formula 14 may be represented by any one of the following formulas 15-1 to 15-5.

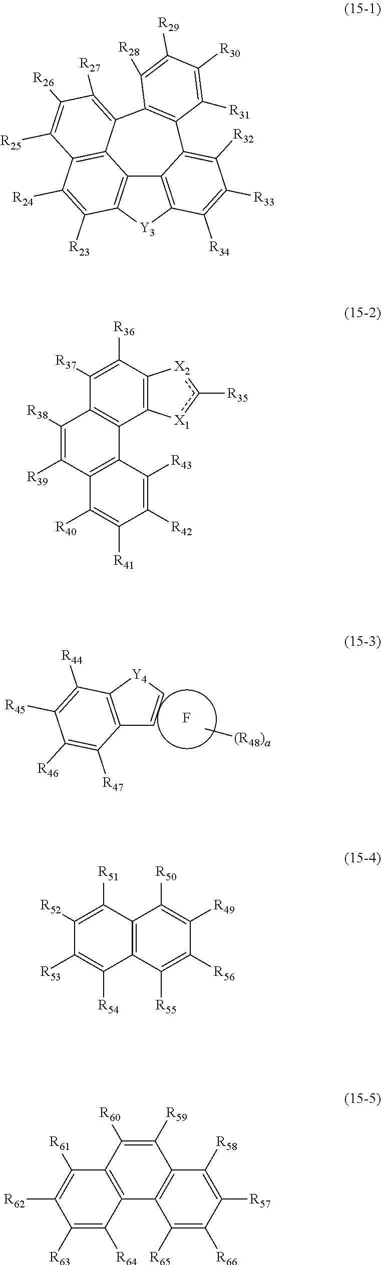

In Formula 15-1,
Y₃ represents O, S, N(R₆₇), or C(R₆₈)(R₆₉);
R₆₇ represents a site linked to any one of L₁ to L₃; or a substituted or unsubstituted (C6-C30)aryl, or a substituted or unsubstituted (3- to 30-membered)heteroaryl;
R₆₈ and R₆₉, each independently, represent a site linked to any one of L₁ to L₃; or a substituted or unsubstituted (C1-C30)alkyl, a substituted or unsubstituted (C6-C30)aryl, or a substituted or unsubstituted (3- to 30-membered)heteroaryl; or R₆₈ and R₆₉ may be linked to each other to form a ring(s); and
R₂₃ to R₃₄, each independently, represent a site linked to any one of L₁ to L₃; hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring group of a (C3-C30) aliphatic ring(s) and a (C6-C30) aromatic ring(s); or may be linked to adjacent substituent(s) to form a ring(s).

In Formula 15-2, R₃₅ represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring group of a (C3-C30) aliphatic ring(s) and a (C6-C30) aromatic ring(s);
R₃₆ to R₄₃, each independently, represent a site linked to any one of L₁ to L₃; hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring group of a (C3-C30) aliphatic ring(s) and a (C6-C30) aromatic ring(s); or may be linked to adjacent substituent(s) to form a ring(s); and
X₁ and X₂, each independently, represent —O—, —S—, —Se— or —N═, and any one of X₁ and X₂ is —N═.

In Formula 15-3, Y₄ represents O, S, N(R₇₀), or C(R₇₁)(R₇₂);
R₇₀ represents a site linked to any one of L₁ to L₃; or a substituted or unsubstituted (C6-C30)aryl;
R₇₁ and R₇₂, each independently, represent a site linked to any one of L₁ to L₃; or a substituted or unsubstituted (C1-C30)alkyl, a substituted or unsubstituted (C6-C30)aryl, or a substituted or unsubstituted (3- to 30-membered)heteroaryl; or R₇₁ and R₇₂ may be linked to each other to form a ring(s);
ring F is benzene, naphthalene or phenanthrene;
a represents an integer of 3 to 10; and
R₄₄ to R₄₈, each independently, represent a site linked to any one of L₁ to L₃; hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring group of a (C3-C30) aliphatic ring(s) and a (C6-C30) aromatic ring(s); or may be linked to adjacent substituent(s) to form a ring(s).

In Formula 15-4, R₄₉ to R₅₆, each independently, represent a site linked to any one of L₁ to L₃; hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring group of a (C3-C30) aliphatic ring(s) and a (C6-C30) aromatic ring(s); or may be linked to adjacent substituent(s) to form a ring(s).

In Formula 15-5, R₅₇ to R₆₆, each independently, represent a site linked to any one of L₁ to L₃; hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring group of a (C3-C30) aliphatic ring(s) and a (C6-C30) aromatic ring(s); or may be linked to adjacent substituent(s) to form a ring(s).

According to one embodiment of the present disclosure, in Formula 15-1, Y₃ represents N(R₆₇);
R₆₇ represents a site linked to any one of L₁ to L₃;
R₂₃ to R₃₄, each independently, represent a site linked to any one of L₁ to L₃; or hydrogen.

According to one embodiment of the present disclosure, in Formula 15-2, R₃₅ represents a substituted or unsubstituted (C6-C30)aryl, or a substituted or unsubstituted (3- to 30-membered)heteroaryl;
R₃₆ to R₄₃, each independently, represent a site linked to any one of L₁ to L₃; hydrogen, or a substituted or unsubstituted (C6-C30)aryl;
X₁ and X₂, each independently, represent —O—, —S— or —N═, and any one of X₁ and X₂ is —N═.

According to one embodiment of the present disclosure, in Formula 15-3, Y₄ represents 0, S, N(R₇₀), or C(R₇₁)(R₇₂);
R₇₀ represents a substituted or unsubstituted (C6-C30)aryl;
R₇₁ and R₇₂, each independently, represent a site linked to any one of L₁ to L₃, or a substituted or unsubstituted (C1-C30)alkyl, or a substituted or unsubstituted (C6-C30)aryl;
ring F is benzene, naphthalene or phenanthrene;
a represents an integer of 3 to 10; and
R₄₄ to R₄₈, each independently, represent a site linked to any one of L₁ to L₃; hydrogen, or a substituted or unsubstituted (C6-C30)aryl.

According to one embodiment of the present disclosure, in Formula 15-4, R₄₉ to R₅₆, each independently, represent a site linked to any one of L₁ to L₃; hydrogen, or a substituted or unsubstituted (C6-C30)aryl.

According to one embodiment of the present disclosure, in Formula 15-5, R₅₇ to R₆₆, each independently, represent a site linked to any one of L₁ to L₃; or hydrogen.

Specifically, Ar₁ to Ar₃, each independently, represent a substituted or unsubstituted phenanthrooxazolinyl, a substituted or unsubstituted phenanthrothiazolinyl, a substituted or unsubstituted carbazolyl, a substituted or unsubstituted phenyl, a substituted or unsubstituted o-terphenyl, a substituted or unsubstituted p-terphenyl, a substituted or unsubstituted naphthyl, a substituted or unsubstituted biphenyl, a substituted or unsubstituted benzophenanthrofuranyl, a substituted or unsubstituted dibenzofuranyl, a substituted or unsubstituted dibenzothiophenyl, a substituted or unsubstituted benzonaphthofuranyl, a substituted or unsubstituted fluorenyl, a substituted or unsubstituted benzofluorenyl, a substituted or unsubstituted spirobifluorenyl, a substituted or unsubstituted pyridinyl, a substituted or unsubstituted benzimiadazolyl, a substituted or unsubstituted benzonaphthofuranyl, a substituted or unsubstituted benzonaphthothiophenyl, a substituted or unsubstituted cyclohexyl, a substituted or unsubstituted phenanthrenyl, a substituted or unsubstituted anthracenyl, a substituted or unsubstituted phenoxazinyl, a substituted or unsubstituted fluoranthenyl, a substituted or unsubstituted cumyl or -L₄-N(Arₐ)(Ar_{b}). For example, Ar₁ to Ar₃, each independently, may be phenanthrooxazolinyl substituted or unsubstituted with a phenyl(s), a biphenyl(s), a pyridinyl(s) or a phenanthrenyl(s); a phenanthrothiazolinyl substituted or unsubstituted with a phenyl(s) or a biphenyl(s); a carbazolyl substituted or unsubstituted with a phenyl(s); a phenyl substituted or unsubstituted with deuterium or an anthracenyl; an o-terphenyl; a p-terphenyl; a naphthyl substituted or unsubstituted with a phenyl(s); a biphenyl substituted or unsubstituted with a phenanthrooxazolinyl(s); a substituted or unsubstituted benzophenanthrofuranyl; a dibenzofuranyl substituted or unsubstituted with a phenyl(s); a dibenzothiophenyl substituted or unsubstituted with a phenyl(s); a benzonaphthofuranyl(s); a benzonaphthothiophenyl(s); a dimethylfluorenyl(s); a phenylfluorenyl(s); a diphenylfluorenyl(s); a dimethylbenzofluorenyl(s); a spirobifluorenyl(s); a pyridinyl substituted or unsubstituted with a phenyl(s); a benzimidazolyl substituted or unsubstituted with a phenyl(s); a cyclohexyl(s); a phenanthrenyl(s); a anthracenyl(s); a phenoxazinyl(s); a fluoranthenyl(s); a cumyl(s); and -L₄-N(Arₐ)(Ar_{b}), etc.

In addition, L₄ represents a single bond or a substituted or unsubstituted (C6-C20)arylene. According to one embodiment of the present disclosure, L₄ represents a substituted or unsubstituted (C6-C12)arylene. For example, L₄ may be phenylene, etc.

Arₐ and Ar_{b}, each independently, represent a substituted or unsubstituted (C6-C20)aryl, or a substituted or unsubstituted (3- to 20-membered)heteroaryl. For example, Arₐ and Ar_{b}, each independently, may be phenyl, biphenyl, naphthyl, and dibenzofuranyl, etc.

The compound represented by Formula 14 may be selected from the following compounds, but is not limited thereto.

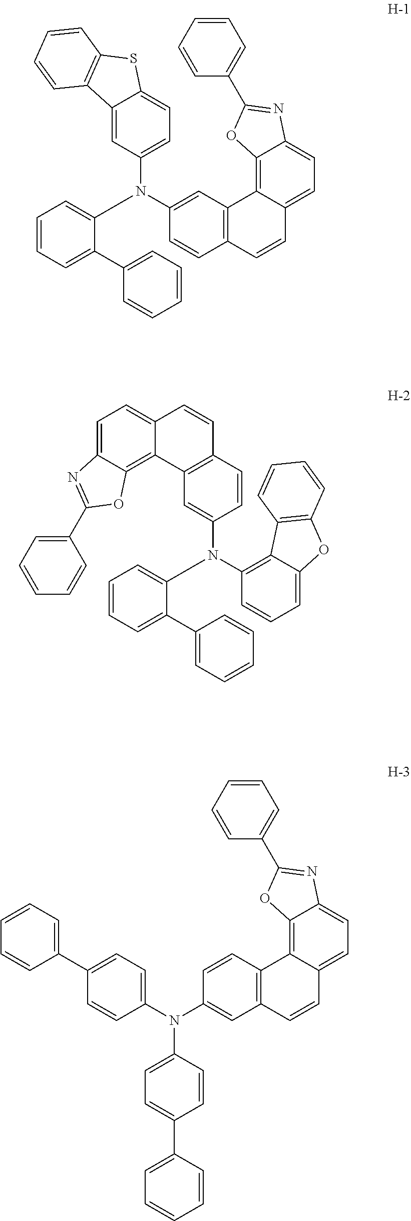 [SMILES: c1ccc(-c2nc3ccc4ccc5ccc(N(c6ccc7sc8ccccc8c7c6)c6ccccc6-c6ccccc6)cc5c4c3o2)cc1 ; c1ccc(-c2nc3ccc4ccc5ccc(N(c6ccccc6-c6ccccc6)c6cccc7oc8ccccc8c67)cc5c4c3o2)cc1 ; c1ccc(-c2ccc(N(c3ccc(-c4ccccc4)cc3)c3ccc4c(ccc5ccc6nc(-c7ccccc7)oc6c54)c3)cc2)cc1]

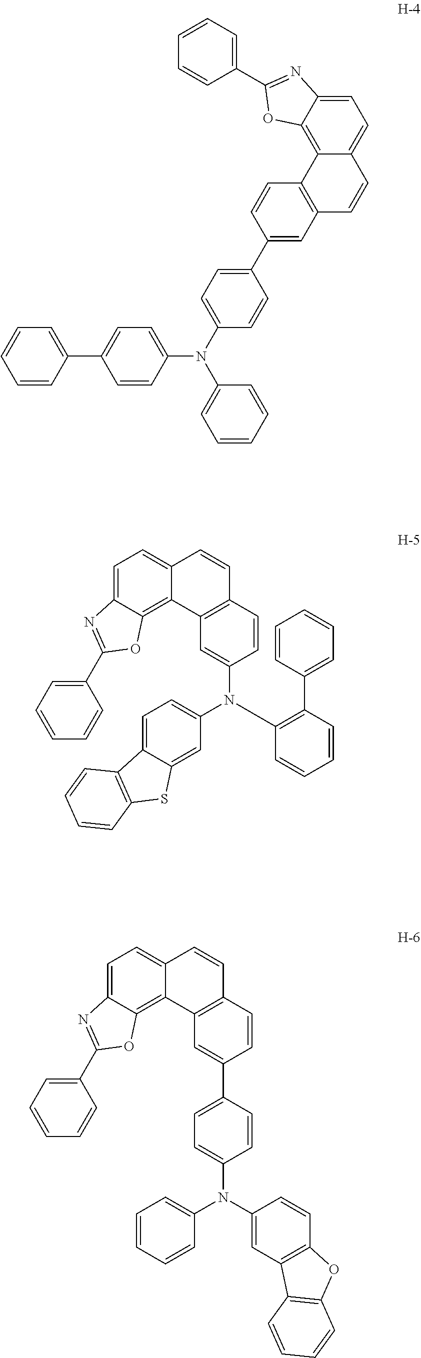 [SMILES: c1ccc(-c2ccc(N(c3ccccc3)c3ccc(-c4ccc5c(ccc6ccc7nc(-c8ccccc8)oc7c65)c4)cc3)cc2)cc1 ; c1ccc(-c2nc3ccc4ccc5ccc(N(c6ccc7c(c6)sc6ccccc67)c6ccccc6-c6ccccc6)cc5c4c3o2)cc1 ; c1ccc(-c2nc3ccc4ccc5ccc(-c6ccc(N(c7ccccc7)c7ccc8oc9ccccc9c8c7)cc6)cc5c4c3o2)cc1]

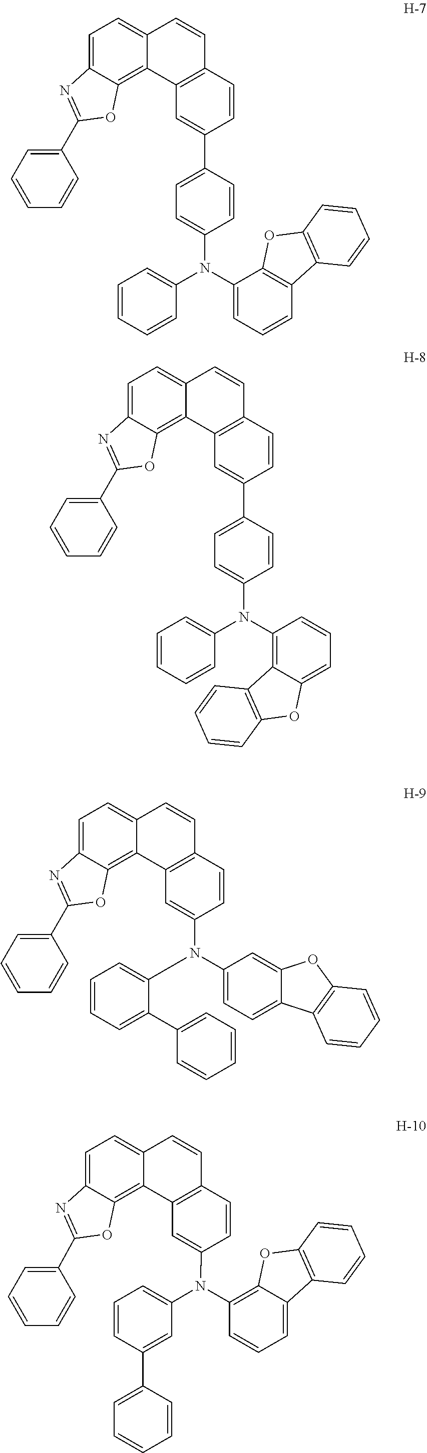

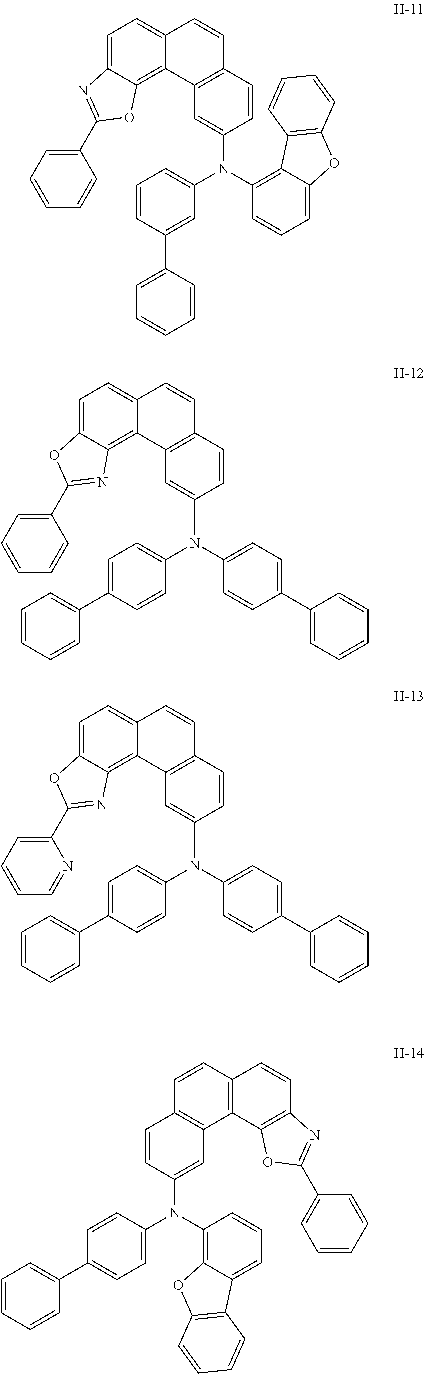 [SMILES: c1ccc(-c2cccc(N(c3ccc4ccc5ccc6nc(-c7ccccc7)oc6c5c4c3)c3cccc4oc5ccccc5c34)c2)cc1 ; c1ccc(-c2ccc(N(c3ccc(-c4ccccc4)cc3)c3ccc4ccc5ccc6oc(-c7ccccc7)nc6c5c4c3)cc2)cc1 ; c1ccc(-c2ccc(N(c3ccc(-c4ccccc4)cc3)c3ccc4ccc5ccc6oc(-c7ccccn7)nc6c5c4c3)cc2)cc1 ; c1ccc(-c2ccc(N(c3ccc4ccc5ccc6nc(-c7ccccc7)oc6c5c4c3)c3cccc4c3oc3ccccc34)cc2)cc1]

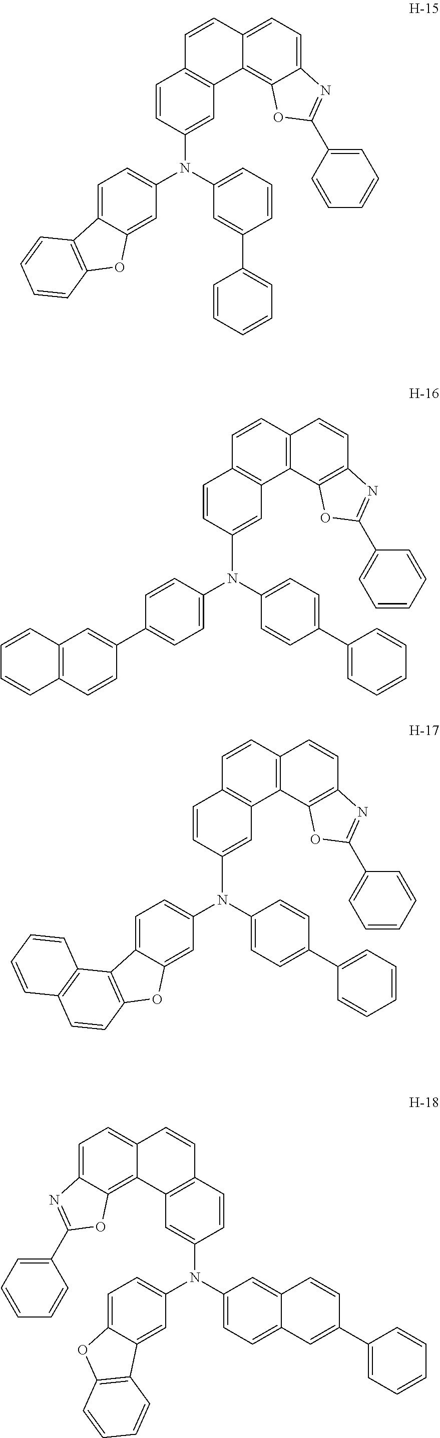 [SMILES: c1ccc(-c2cccc(N(c3ccc4c(c3)oc3ccccc34)c3ccc4ccc5ccc6nc(-c7ccccc7)oc6c5c4c3)c2)cc1 ; c1ccc(-c2ccc(N(c3ccc(-c4ccc5ccccc5c4)cc3)c3ccc4ccc5ccc6nc(-c7ccccc7)oc6c5c4c3)cc2)cc1 ; c1ccc(-c2ccc(N(c3ccc4c(c3)oc3ccc5ccccc5c34)c3ccc4ccc5ccc6nc(-c7ccccc7)oc6c5c4c3)cc2)cc1 ; c1ccc(-c2ccc3cc(N(c4ccc5oc6ccccc6c5c4)c4ccc5ccc6ccc7nc(-c8ccccc8)oc7c6c5c4)ccc3c2)cc1]

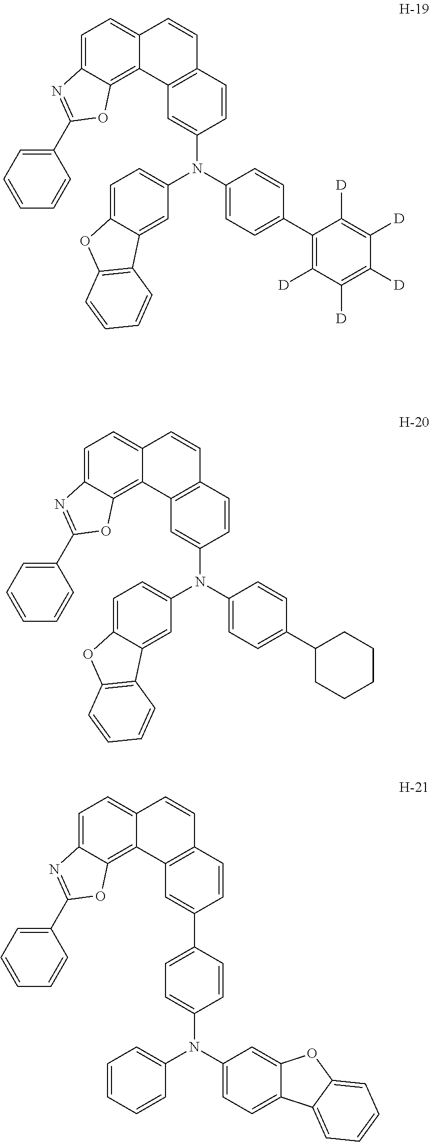 [SMILES: [2H]c1c([2H])c([2H])c(-c2ccc(N(c3ccc4oc5ccccc5c4c3)c3ccc4ccc5ccc6nc(-c7ccccc7)oc6c5c4c3)cc2)c([2H])c1[2H] ; c1ccc(-c2nc3ccc4ccc5ccc(N(c6ccc(C7CCCCC7)cc6)c6ccc7oc8ccccc8c7c6)cc5c4c3o2)cc1 ; c1ccc(-c2nc3ccc4ccc5ccc(-c6ccc(N(c7ccccc7)c7ccc8c(c7)oc7ccccc78)cc6)cc5c4c3o2)cc1]

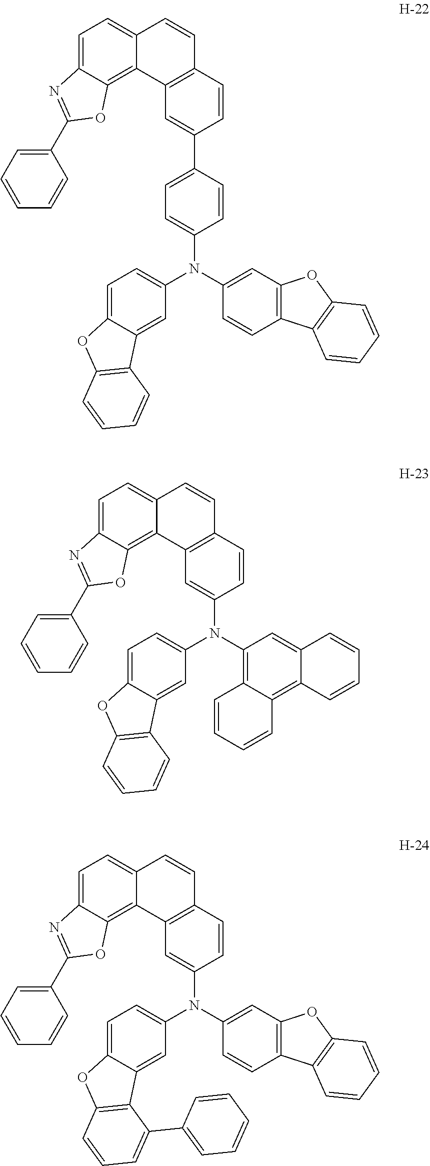 [SMILES: c1ccc(-c2nc3ccc4ccc5ccc(-c6ccc(N(c7ccc8c(c7)oc7ccccc78)c7ccc8oc9ccccc9c8c7)cc6)cc5c4c3o2)cc1 ; c1ccc(-c2nc3ccc4ccc5ccc(N(c6ccc7oc8ccccc8c7c6)c6cc7ccccc7c7ccccc67)cc5c4c3o2)cc1 ; c1ccc(-c2nc3ccc4ccc5ccc(N(c6ccc7c(c6)oc6ccccc67)c6ccc7oc8cccc(-c9ccccc9)c8c7c6)cc5c4c3o2)cc1]

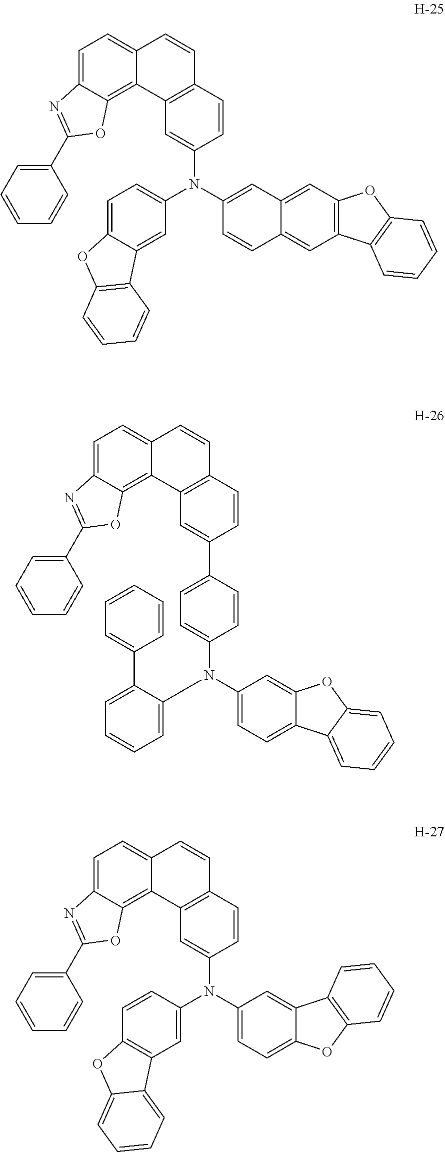 [SMILES: c1ccc(-c2nc3ccc4ccc5ccc(N(c6ccc7cc8c(cc7c6)oc6ccccc68)c6ccc7oc8ccccc8c7c6)cc5c4c3o2)cc1 ; c1ccc(-c2nc3ccc4ccc5ccc(-c6ccc(N(c7ccc8c(c7)oc7ccccc78)c7ccccc7-c7ccccc7)cc6)cc5c4c3o2)cc1 ; c1ccc(-c2nc3ccc4ccc5ccc(N(c6ccc7oc8ccccc8c7c6)c6ccc7oc8ccccc8c7c6)cc5c4c3o2)cc1]

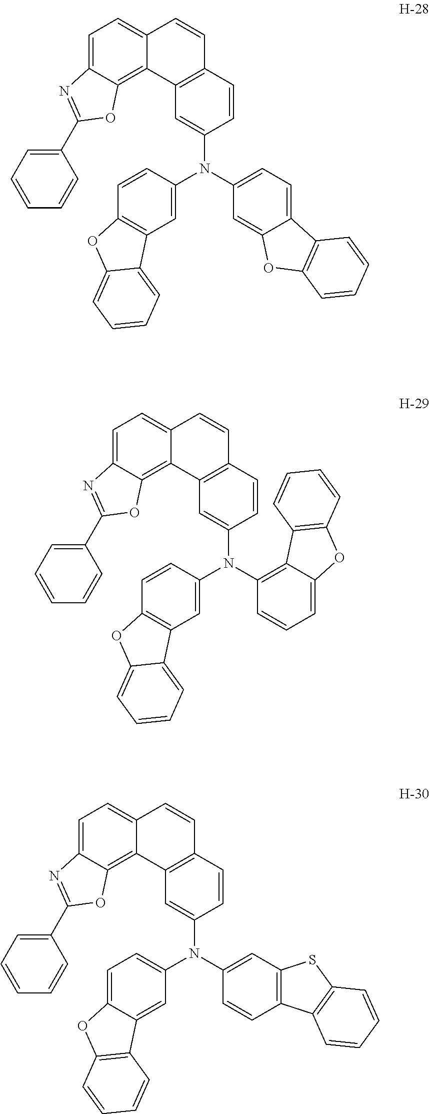

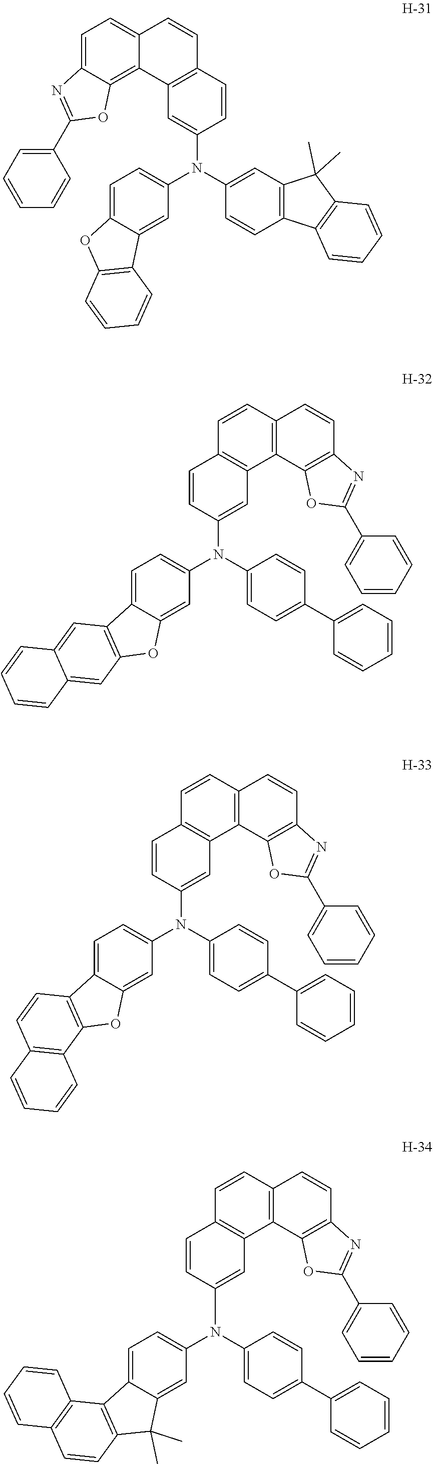 [SMILES: CC1(C)c2ccccc2-c2ccc(N(c3ccc4oc5ccccc5c4c3)c3ccc4ccc5ccc6nc(-c7ccccc7)oc6c5c4c3)cc21 ; c1ccc(-c2ccc(N(c3ccc4c(c3)oc3cc5ccccc5cc34)c3ccc4ccc5ccc6nc(-c7ccccc7)oc6c5c4c3)cc2)cc1 ; c1ccc(-c2ccc(N(c3ccc4c(c3)oc3c5ccccc5ccc43)c3ccc4ccc5ccc6nc(-c7ccccc7)oc6c5c4c3)cc2)cc1 ; CC1(C)c2cc(N(c3ccc(-c4ccccc4)cc3)c3ccc4ccc5ccc6nc(-c7ccccc7)oc6c5c4c3)ccc2-c2c1ccc1ccccc21]

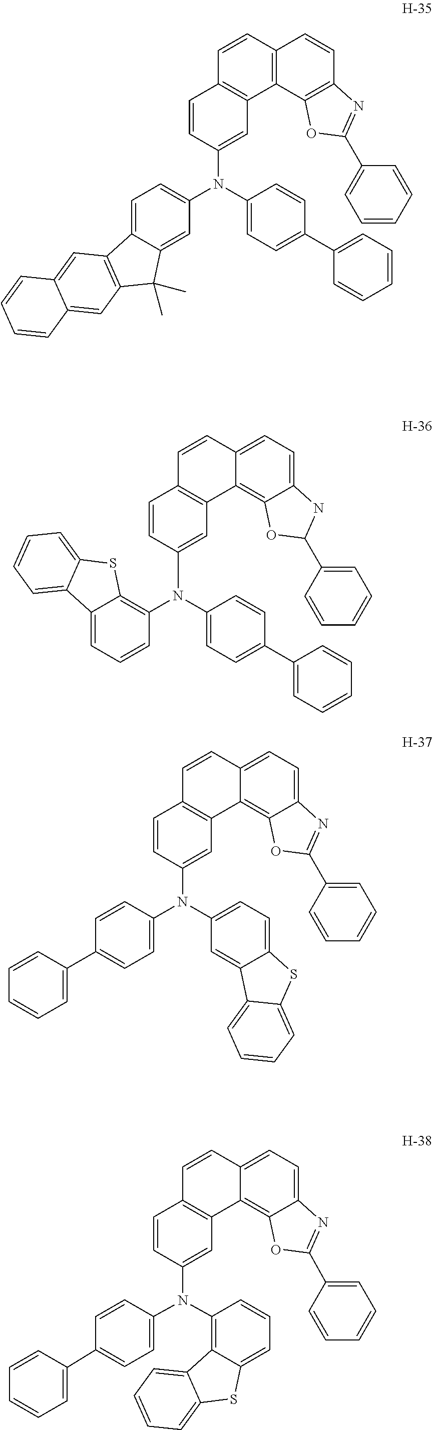 [SMILES: CC1(C)c2cc(N(c3ccc(-c4ccccc4)cc3)c3ccc4ccc5ccc6nc(-c7ccccc7)oc6c5c4c3)ccc2-c2cc3ccccc3cc21 ; c1ccc(-c2ccc(N(c3ccc4ccc5ccc6c(c5c4c3)OC(c3ccccc3)N6)c3cccc4c3sc3ccccc34)cc2)cc1 ; c1ccc(-c2ccc(N(c3ccc4sc5ccccc5c4c3)c3ccc4ccc5ccc6nc(-c7ccccc7)oc6c5c4c3)cc2)cc1 ; c1ccc(-c2ccc(N(c3ccc4ccc5ccc6nc(-c7ccccc7)oc6c5c4c3)c3cccc4sc5ccccc5c34)cc2)cc1]

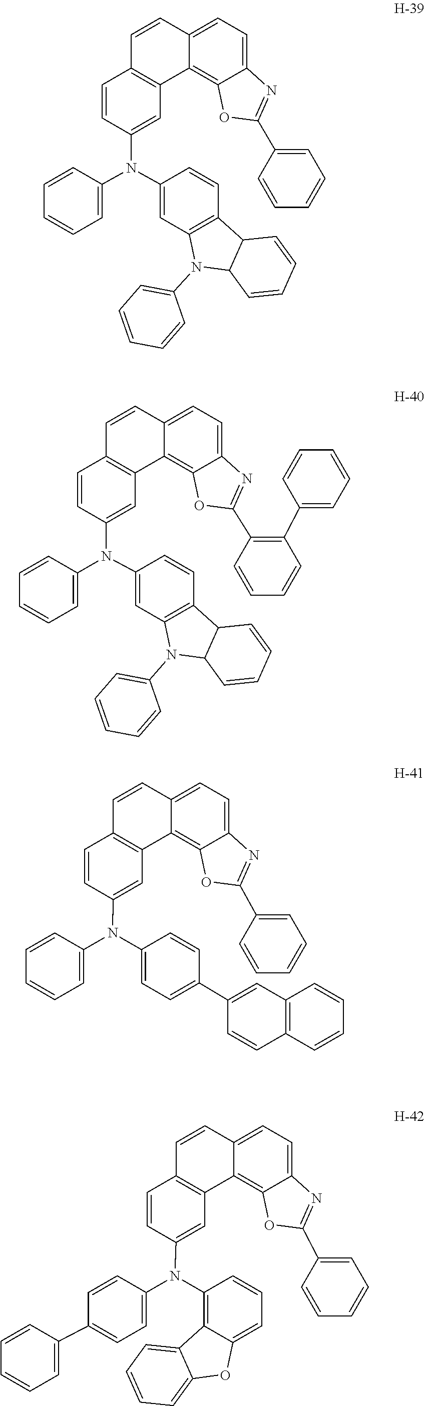

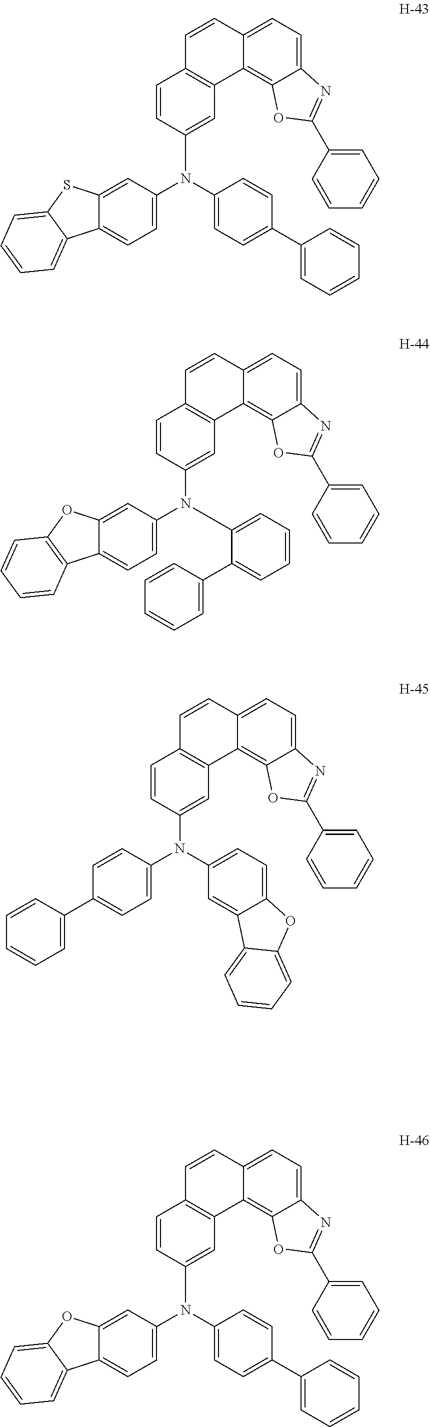 [SMILES: c1ccc(-c2ccc(N(c3ccc4c(c3)sc3ccccc34)c3ccc4ccc5ccc6nc(-c7ccccc7)oc6c5c4c3)cc2)cc1 ; c1ccc(-c2nc3ccc4ccc5ccc(N(c6ccc7c(c6)oc6ccccc67)c6ccccc6-c6ccccc6)cc5c4c3o2)cc1 ; c1ccc(-c2ccc(N(c3ccc4oc5ccccc5c4c3)c3ccc4ccc5ccc6nc(-c7ccccc7)oc6c5c4c3)cc2)cc1 ; c1ccc(-c2ccc(N(c3ccc4c(c3)oc3ccccc34)c3ccc4ccc5ccc6nc(-c7ccccc7)oc6c5c4c3)cc2)cc1]

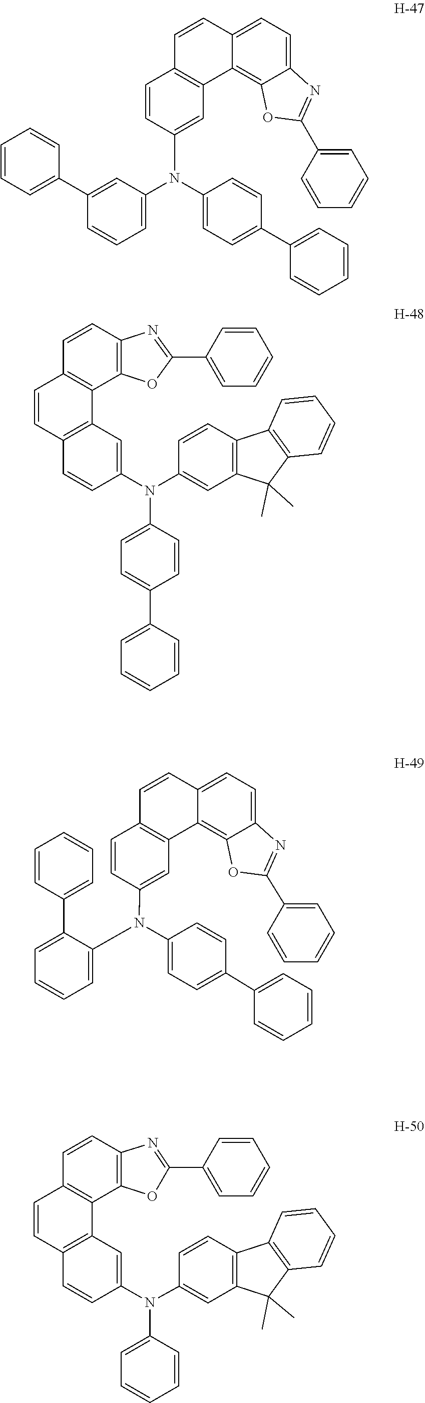

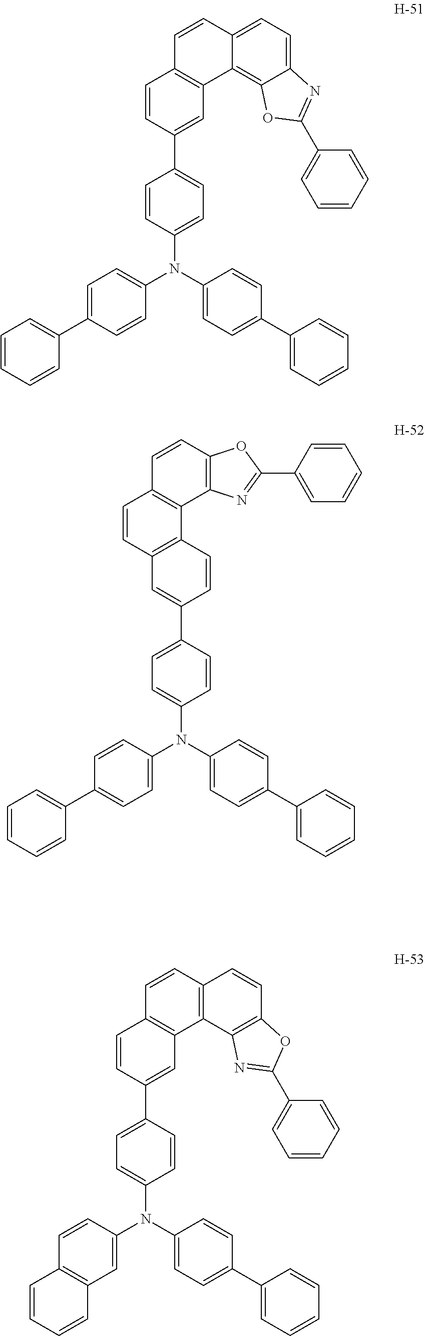 [SMILES: c1ccc(-c2ccc(N(c3ccc(-c4ccccc4)cc3)c3ccc(-c4ccc5ccc6ccc7nc(-c8ccccc8)oc7c6c5c4)cc3)cc2)cc1 ; c1ccc(-c2ccc(N(c3ccc(-c4ccccc4)cc3)c3ccc(-c4ccc5c(ccc6ccc7oc(-c8ccccc8)nc7c65)c4)cc3)cc2)cc1 ; c1ccc(-c2ccc(N(c3ccc(-c4ccc5ccc6ccc7oc(-c8ccccc8)nc7c6c5c4)cc3)c3ccc4ccccc4c3)cc2)cc1]

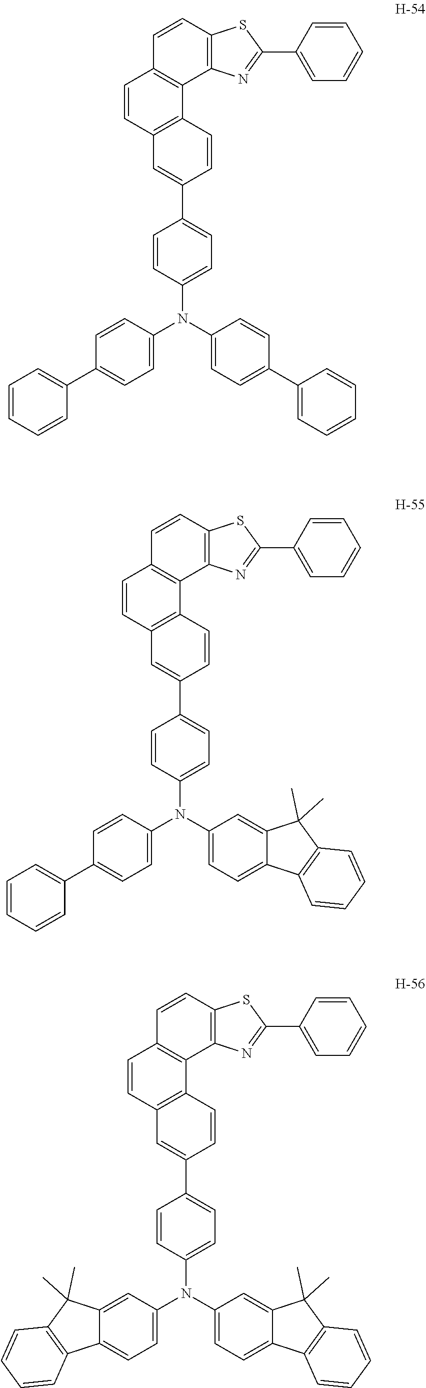 [SMILES: c1ccc(-c2ccc(N(c3ccc(-c4ccccc4)cc3)c3ccc(-c4ccc5c(ccc6ccc7sc(-c8ccccc8)nc7c65)c4)cc3)cc2)cc1 ; CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21 ; CC1(C)c2ccccc2-c2ccc(N(c3ccc(-c4ccc5c(ccc6ccc7sc(-c8ccccc8)nc7c65)c4)cc3)c3ccc4c(c3)C(C)(C)c3ccccc3-4)cc21]

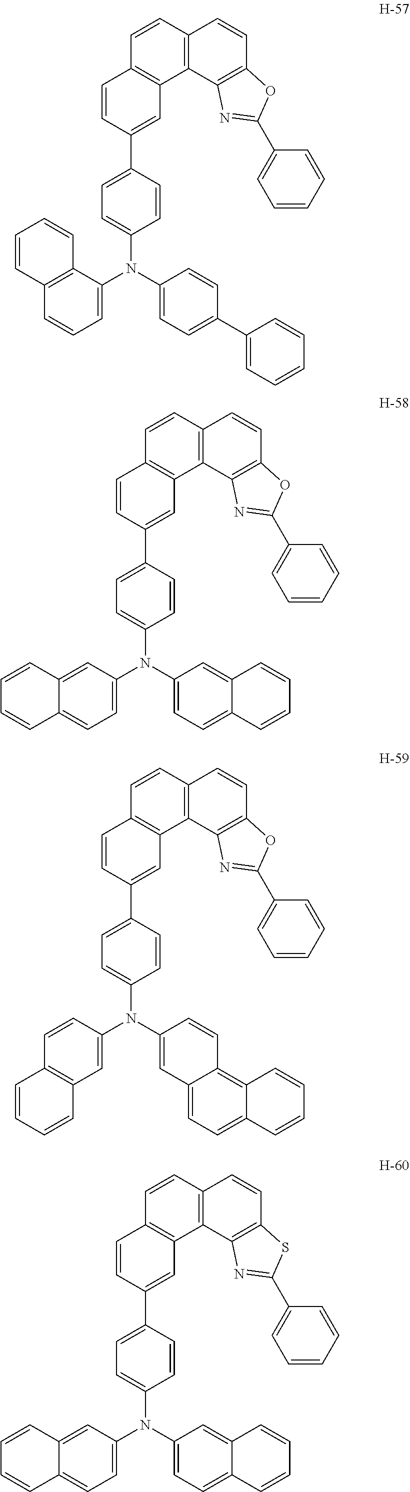

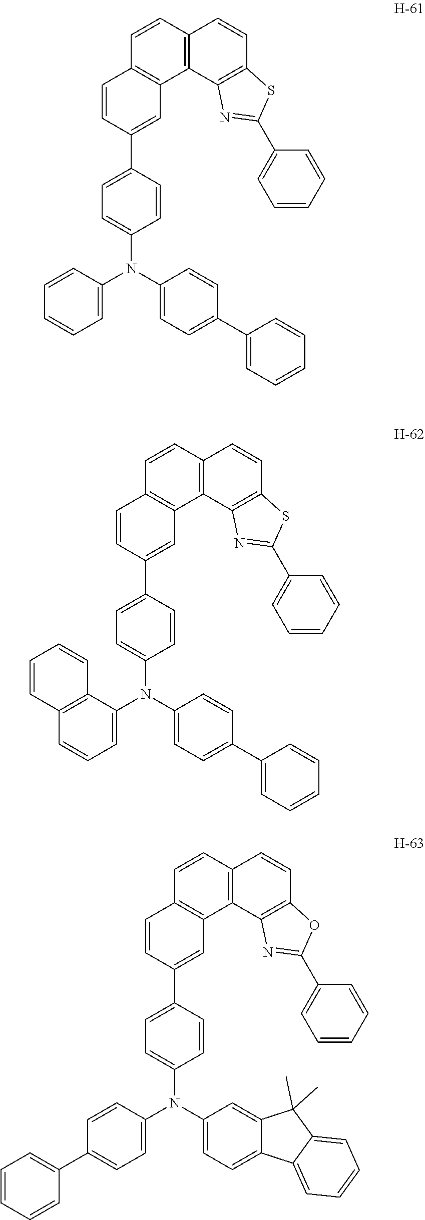 [SMILES: c1ccc(-c2ccc(N(c3ccccc3)c3ccc(-c4ccc5ccc6ccc7sc(-c8ccccc8)nc7c6c5c4)cc3)cc2)cc1 ; c1ccc(-c2ccc(N(c3ccc(-c4ccc5ccc6ccc7sc(-c8ccccc8)nc7c6c5c4)cc3)c3cccc4ccccc34)cc2)cc1 ; CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21]

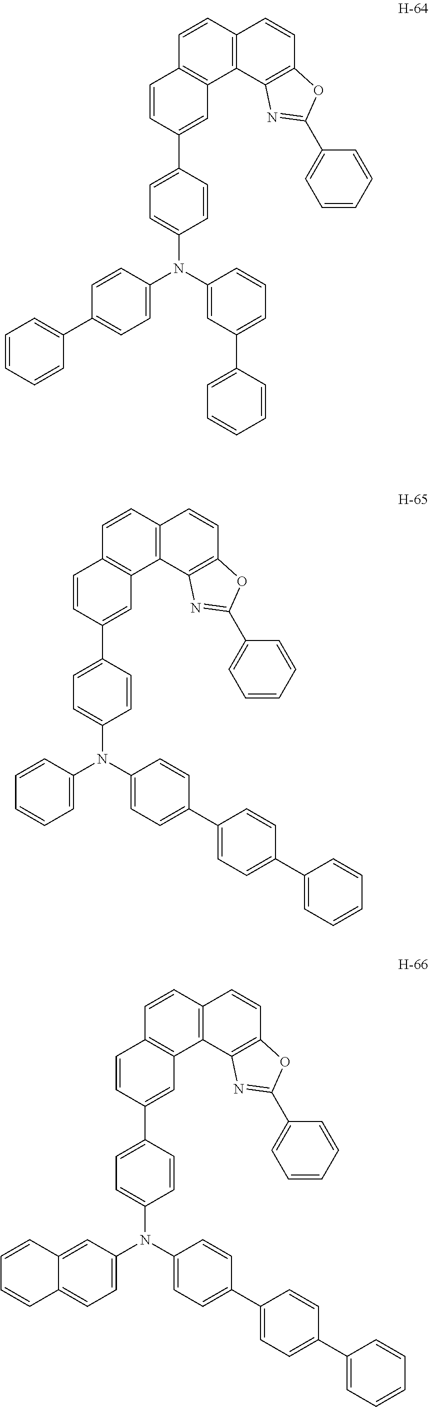 [SMILES: c1ccc(-c2ccc(N(c3ccc(-c4ccc5ccc6ccc7oc(-c8ccccc8)nc7c6c5c4)cc3)c3cccc(-c4ccccc4)c3)cc2)cc1 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1]

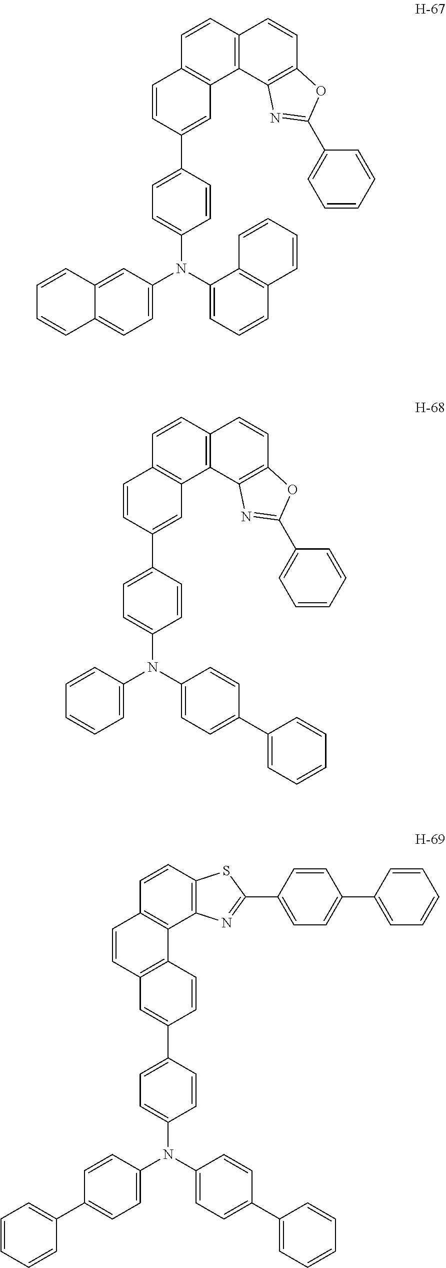 [SMILES: c1ccc(-c2nc3c(ccc4ccc5ccc(-c6ccc(N(c7ccc8ccccc8c7)c7cccc8ccccc78)cc6)cc5c43)o2)cc1 ; c1ccc(-c2ccc(N(c3ccccc3)c3ccc(-c4ccc5ccc6ccc7oc(-c8ccccc8)nc7c6c5c4)cc3)cc2)cc1 ; c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1]

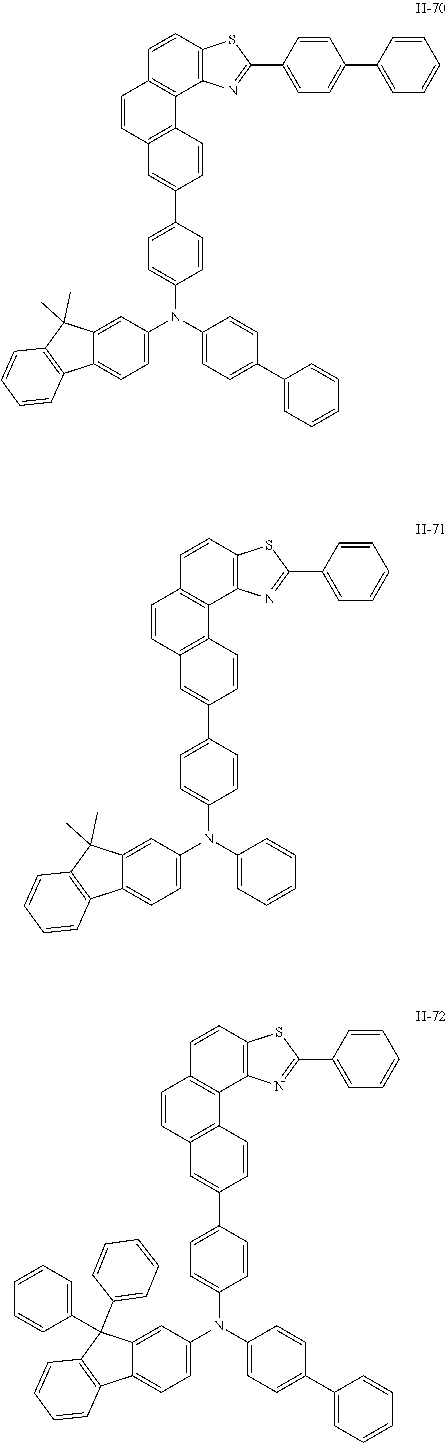 [SMILES: CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21 ; CC1(C)c2ccccc2-c2ccc(N(c3ccccc3)c3ccc(-c4ccc5c(ccc6ccc7sc(-c8ccccc8)nc7c65)c4)cc3)cc21 ; c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1]

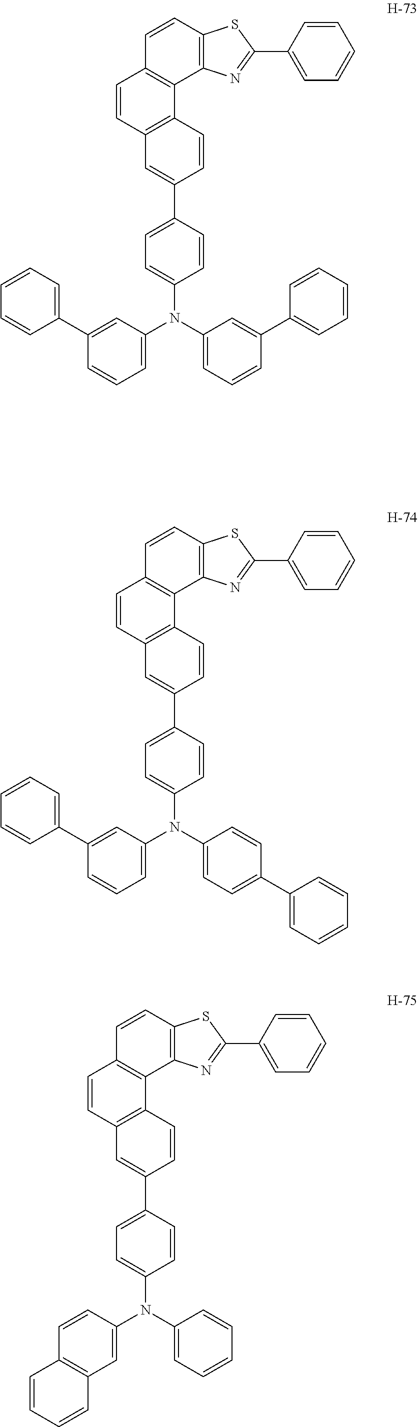 [SMILES: c1ccc(-c2cccc(N(c3ccc(-c4ccc5c(ccc6ccc7sc(-c8ccccc8)nc7c65)c4)cc3)c3cccc(-c4ccccc4)c3)c2)cc1 ; c1ccc(-c2ccc(N(c3ccc(-c4ccc5c(ccc6ccc7sc(-c8ccccc8)nc7c65)c4)cc3)c3cccc(-c4ccccc4)c3)cc2)cc1 ; c1ccc(-c2nc3c(ccc4ccc5cc(-c6ccc(N(c7ccccc7)c7ccc8ccccc8c7)cc6)ccc5c43)s2)cc1]

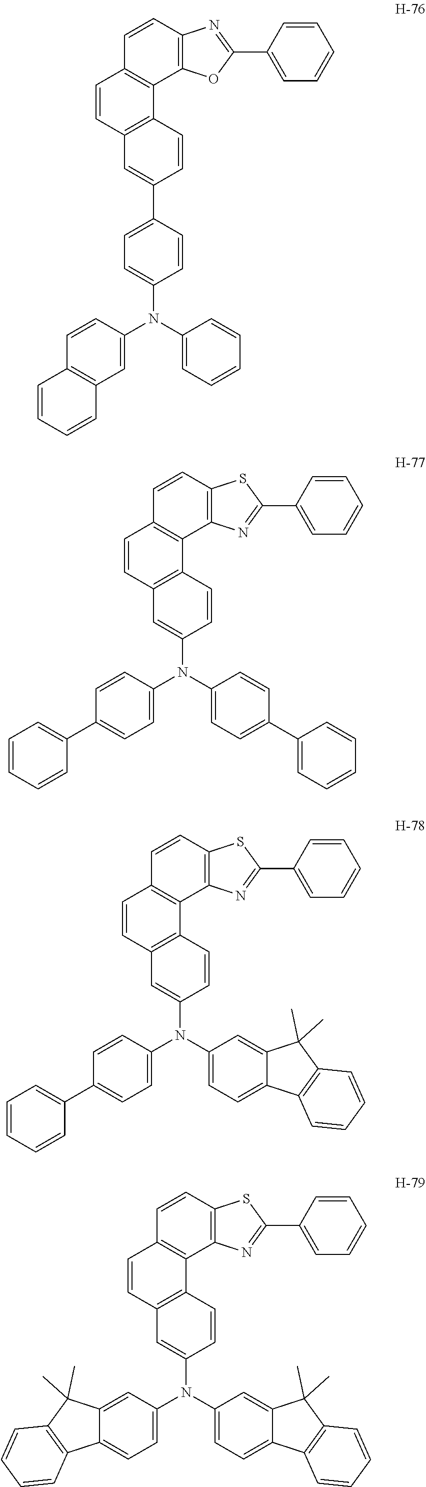 [SMILES: c1ccc(-c2nc3ccc4ccc5cc(-c6ccc(N(c7ccccc7)c7ccc8ccccc8c7)cc6)ccc5c4c3o2)cc1 ; c1ccc(-c2ccc(N(c3ccc(-c4ccccc4)cc3)c3ccc4c(ccc5ccc6sc(-c7ccccc7)nc6c54)c3)cc2)cc1 ; CC1(C)c2ccccc2-c2ccc(N(c3ccc(-c4ccccc4)cc3)c3ccc4c(ccc5ccc6sc(-c7ccccc7)nc6c54)c3)cc21 ; CC1(C)c2ccccc2-c2ccc(N(c3ccc4c(c3)C(C)(C)c3ccccc3-4)c3ccc4c(ccc5ccc6sc(-c7ccccc7)nc6c54)c3)cc21]

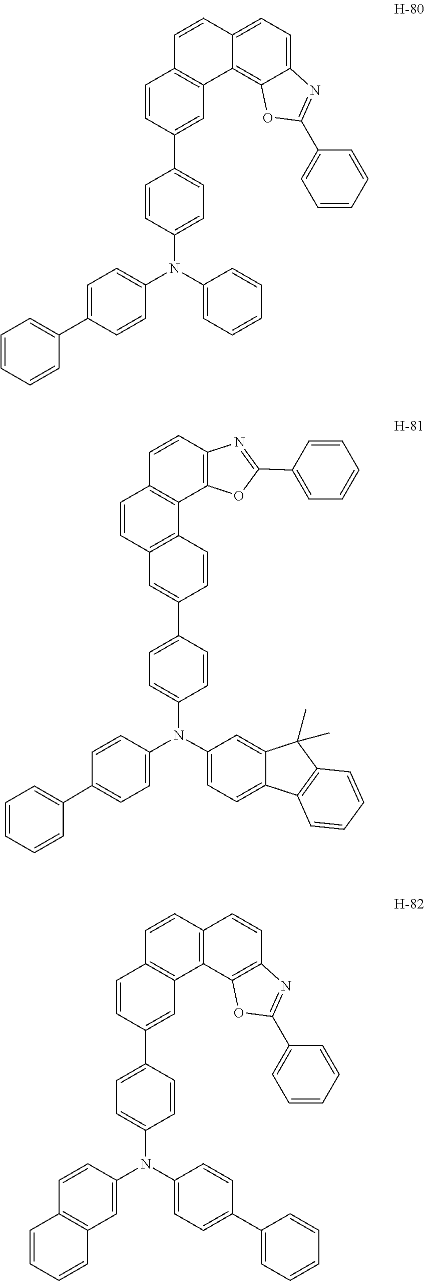

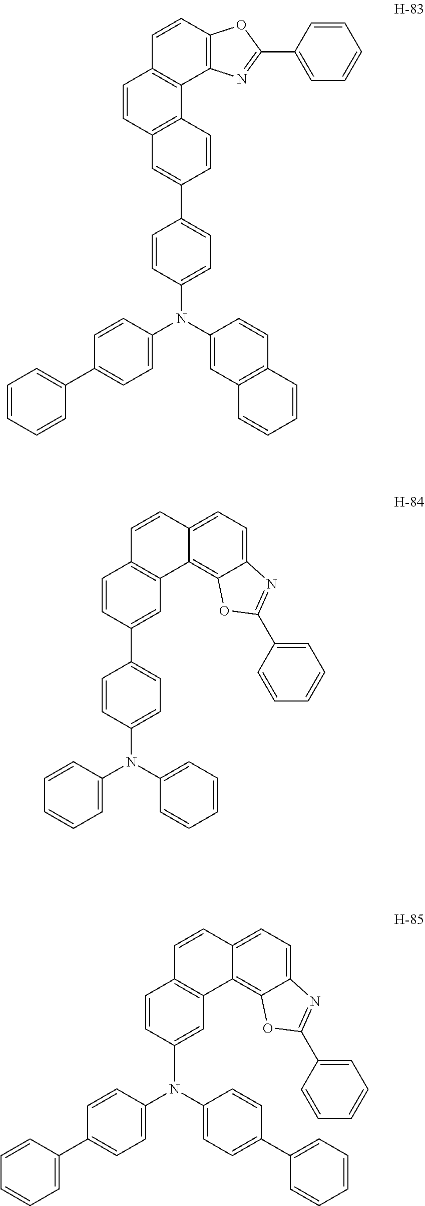 [SMILES: c1ccc(-c2ccc(N(c3ccc(-c4ccc5c(ccc6ccc7oc(-c8ccccc8)nc7c65)c4)cc3)c3ccc4ccccc4c3)cc2)cc1 ; c1ccc(-c2nc3ccc4ccc5ccc(-c6ccc(N(c7ccccc7)c7ccccc7)cc6)cc5c4c3o2)cc1 ; c1ccc(-c2ccc(N(c3ccc(-c4ccccc4)cc3)c3ccc4ccc5ccc6nc(-c7ccccc7)oc6c5c4c3)cc2)cc1]

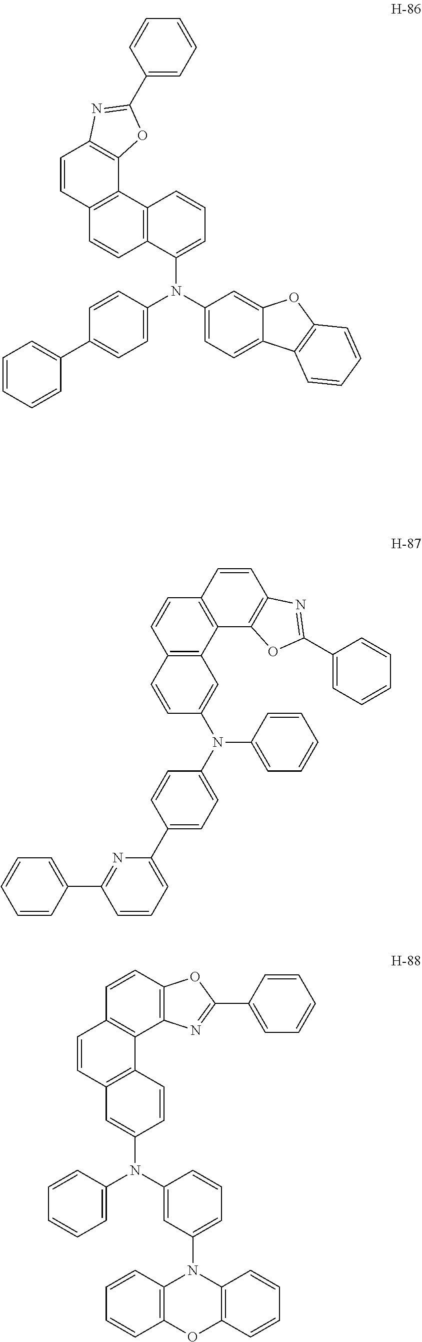 [SMILES: c1ccc(-c2ccc(N(c3ccc4c(c3)oc3ccccc34)c3cccc4c3ccc3ccc5nc(-c6ccccc6)oc5c34)cc2)cc1 ; c1ccc(-c2cccc(-c3ccc(N(c4ccccc4)c4ccc5ccc6ccc7nc(-c8ccccc8)oc7c6c5c4)cc3)n2)cc1 ; c1ccc(-c2nc3c(ccc4ccc5cc(N(c6ccccc6)c6cccc(N7c8ccccc8Oc8ccccc87)c6)ccc5c43)o2)cc1]

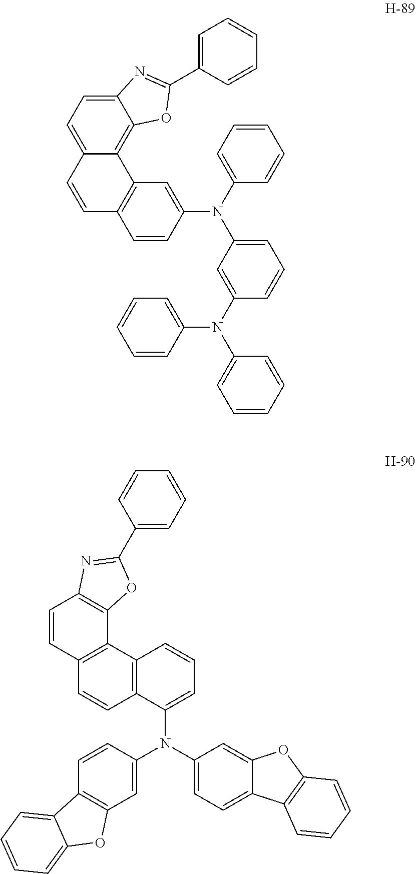 [SMILES: c1ccc(-c2nc3ccc4ccc5ccc(N(c6ccccc6)c6cccc(N(c7ccccc7)c7ccccc7)c6)cc5c4c3o2)cc1 ; c1ccc(-c2nc3ccc4ccc5c(N(c6ccc7c(c6)oc6ccccc67)c6ccc7c(c6)oc6ccccc67)cccc5c4c3o2)cc1]

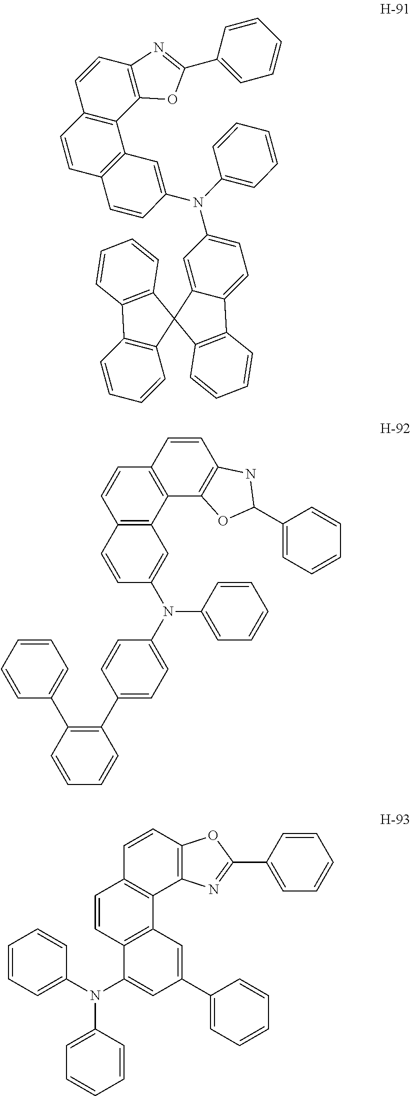

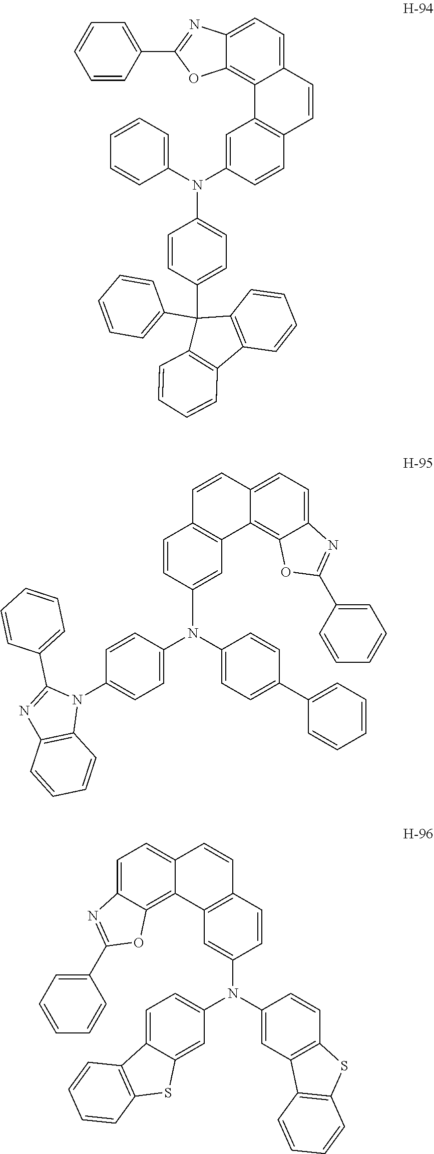 [SMILES: c1ccc(-c2nc3ccc4ccc5ccc(N(c6ccccc6)c6ccc(C7(c8ccccc8)c8ccccc8-c8ccccc87)cc6)cc5c4c3o2)cc1 ; c1ccc(-c2ccc(N(c3ccc(-n4c(-c5ccccc5)nc5ccccc54)cc3)c3ccc4ccc5ccc6nc(-c7ccccc7)oc6c5c4c3)cc2)cc1 ; c1ccc(-c2nc3ccc4ccc5ccc(N(c6ccc7c(c6)sc6ccccc67)c6ccc7sc8ccccc8c7c6)cc5c4c3o2)cc1]

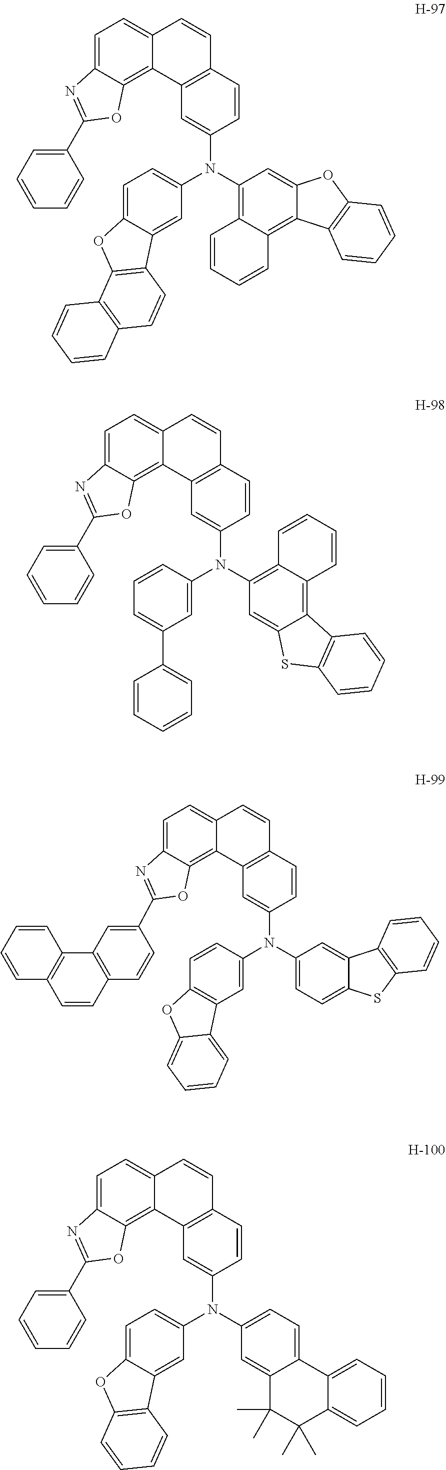 [SMILES: c1ccc(-c2nc3ccc4ccc5ccc(N(c6ccc7oc8c9ccccc9ccc8c7c6)c6cc7oc8ccccc8c7c7ccccc67)cc5c4c3o2)cc1 ; c1ccc(-c2cccc(N(c3ccc4ccc5ccc6nc(-c7ccccc7)oc6c5c4c3)c3cc4sc5ccccc5c4c4ccccc34)c2)cc1 ; c1ccc2c(c1)ccc1ccc(-c3nc4ccc5ccc6ccc(N(c7ccc8oc9ccccc9c8c7)c7ccc8sc9ccccc9c8c7)cc6c5c4o3)cc12 ; CC1(C)c2ccccc2-c2ccc(N(c3ccc4oc5ccccc5c4c3)c3ccc4ccc5ccc6nc(-c7ccccc7)oc6c5c4c3)cc2C1(C)C]

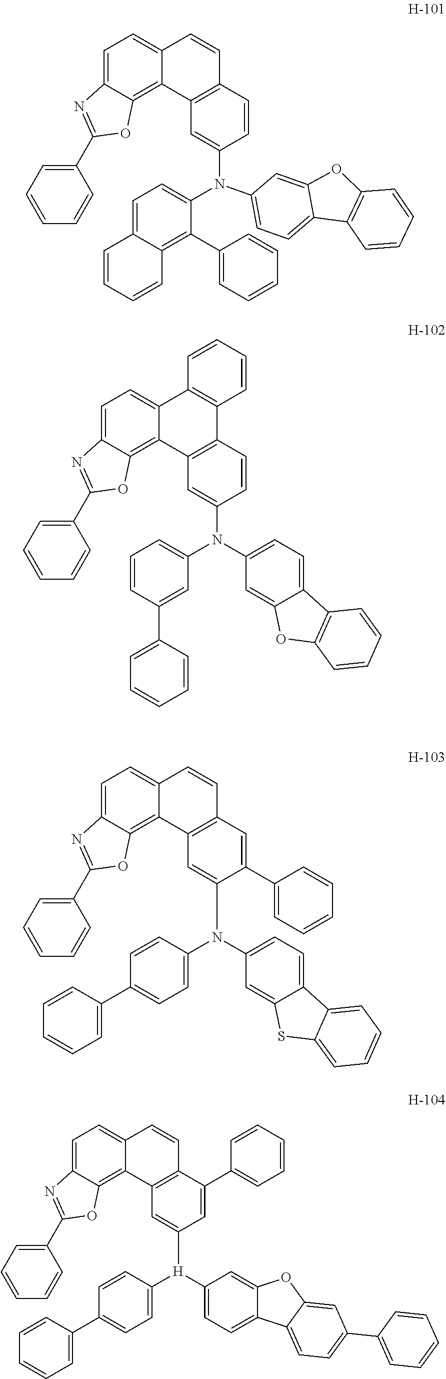

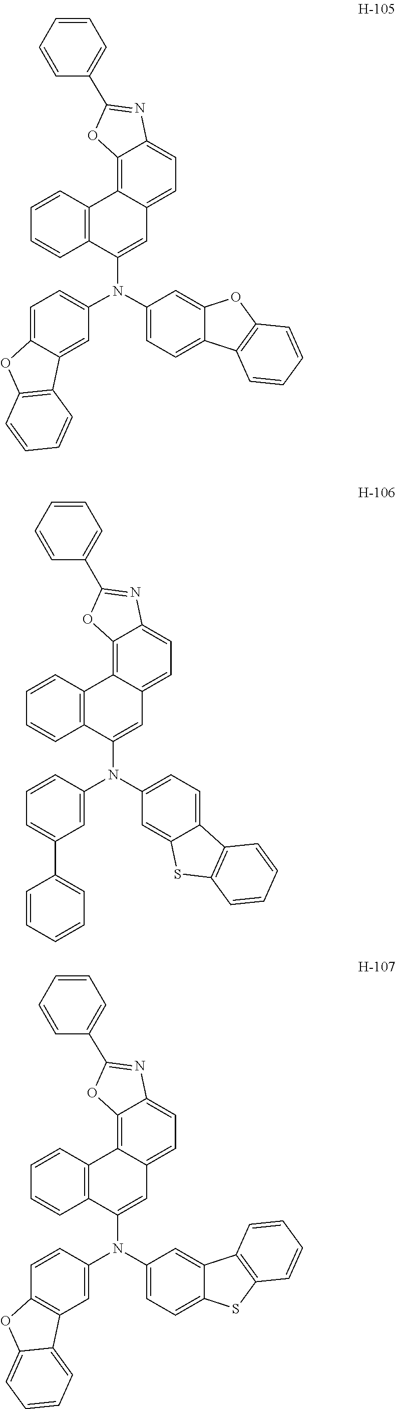

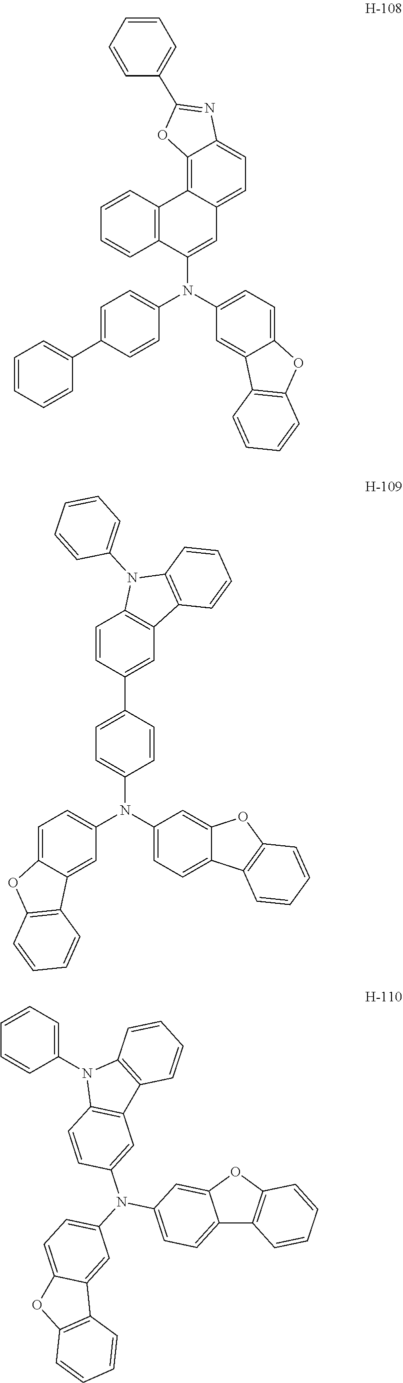

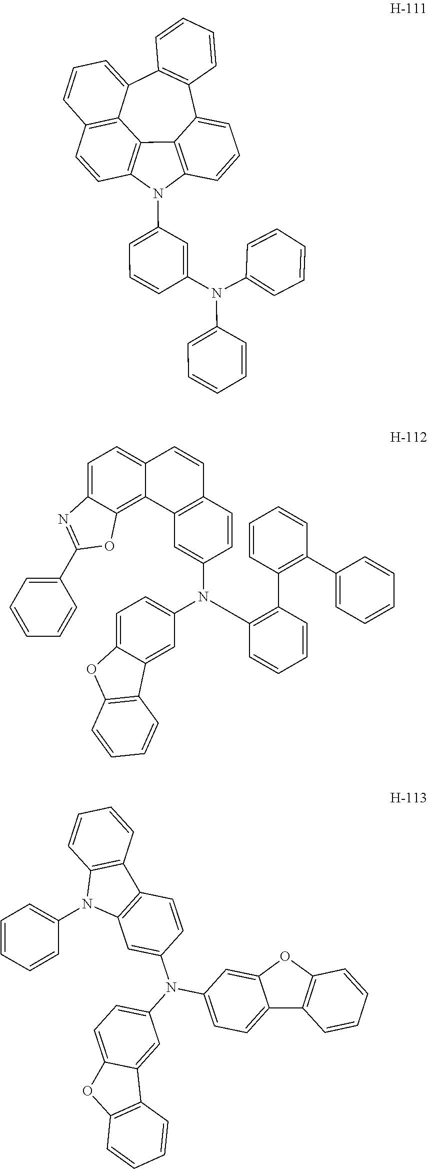 [SMILES: c1ccc(N(c2ccccc2)c2cccc(-n3c4cccc5c4c4c6c(cccc6ccc43)-c3ccccc3-5)c2)cc1 ; c1ccc(-c2nc3ccc4ccc5ccc(N(c6ccc7oc8ccccc8c7c6)c6ccccc6-c6ccccc6-c6ccccc6)cc5c4c3o2)cc1 ; c1ccc(-n2c3ccccc3c3ccc(N(c4ccc5c(c4)oc4ccccc45)c4ccc5oc6ccccc6c5c4)cc32)cc1]

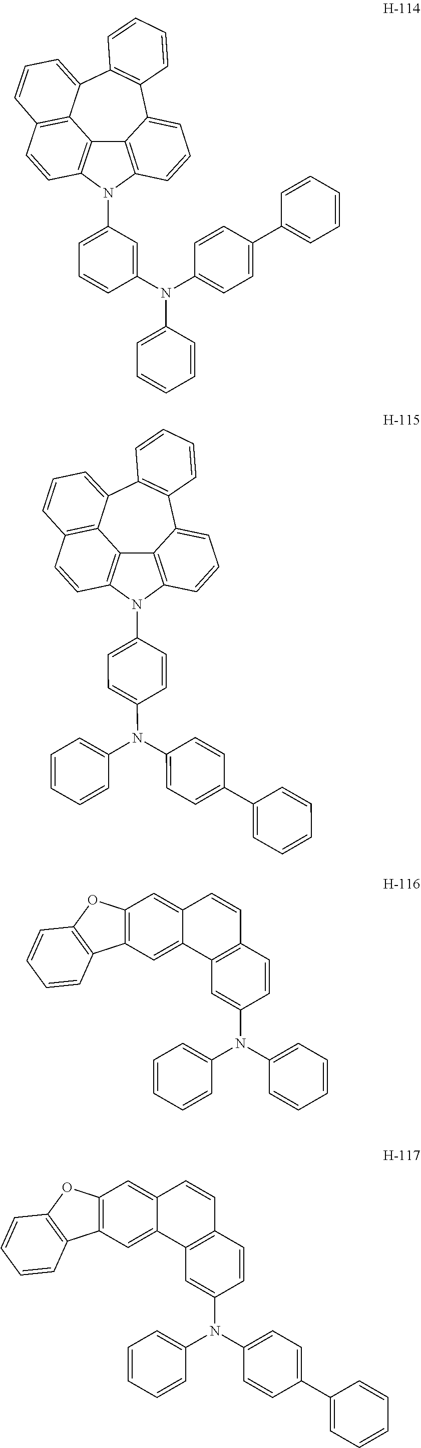

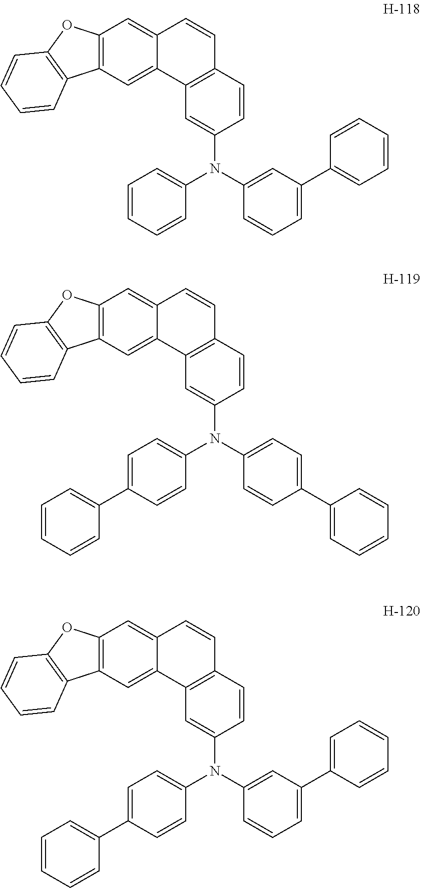 [SMILES: c1ccc(-c2cccc(N(c3ccccc3)c3ccc4ccc5cc6oc7ccccc7c6cc5c4c3)c2)cc1 ; c1ccc(-c2ccc(N(c3ccc(-c4ccccc4)cc3)c3ccc4ccc5cc6oc7ccccc7c6cc5c4c3)cc2)cc1 ; c1ccc(-c2ccc(N(c3cccc(-c4ccccc4)c3)c3ccc4ccc5cc6oc7ccccc7c6cc5c4c3)cc2)cc1]

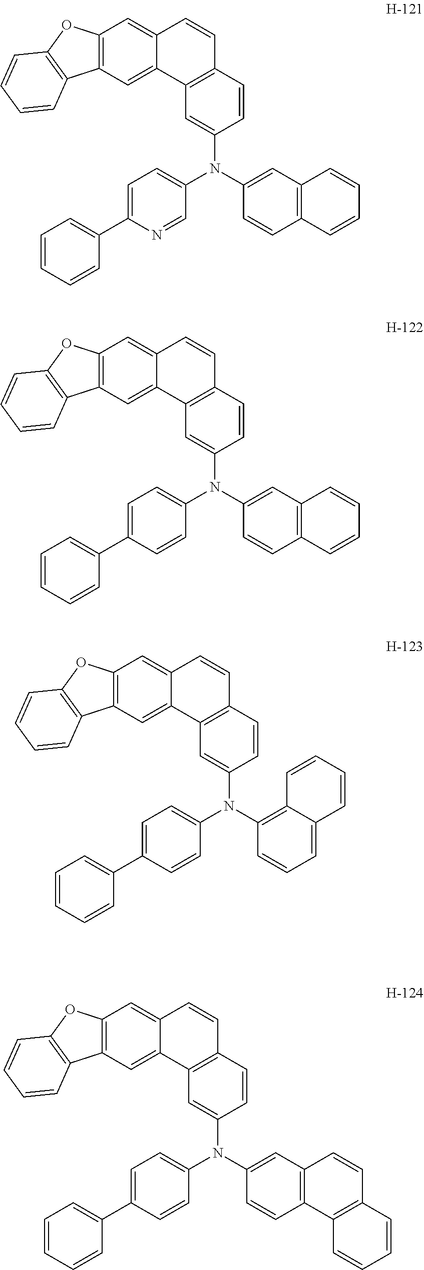 [SMILES: c1ccc(-c2ccc(N(c3ccc4ccccc4c3)c3ccc4ccc5cc6oc7ccccc7c6cc5c4c3)cn2)cc1 ; c1ccc(-c2ccc(N(c3ccc4ccccc4c3)c3ccc4ccc5cc6oc7ccccc7c6cc5c4c3)cc2)cc1 ; c1ccc(-c2ccc(N(c3ccc4ccc5cc6oc7ccccc7c6cc5c4c3)c3cccc4ccccc34)cc2)cc1 ; c1ccc(-c2ccc(N(c3ccc4c(ccc5ccccc54)c3)c3ccc4ccc5cc6oc7ccccc7c6cc5c4c3)cc2)cc1]

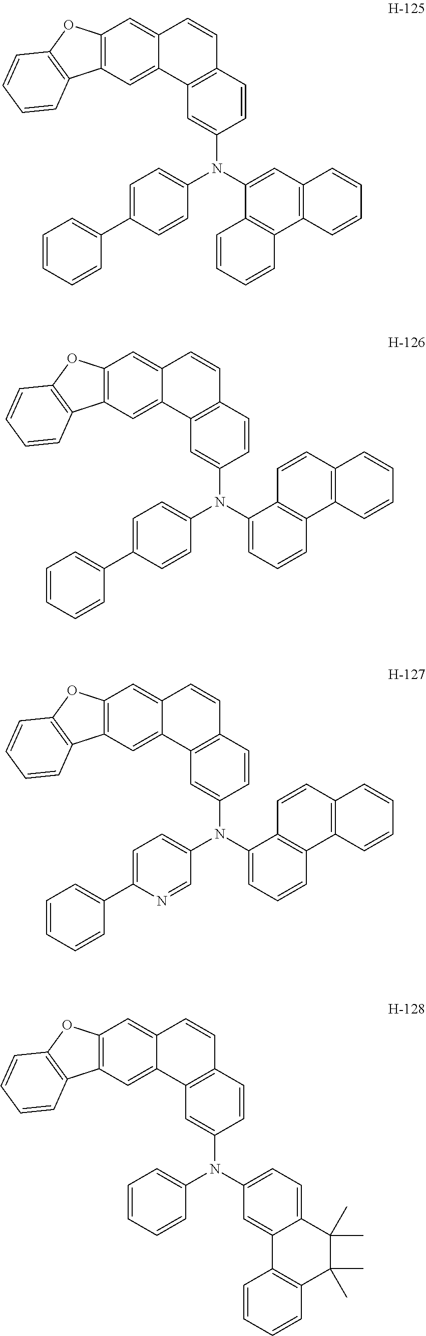 [SMILES: c1ccc(-c2ccc(N(c3ccc4ccc5cc6oc7ccccc7c6cc5c4c3)c3cc4ccccc4c4ccccc34)cc2)cc1 ; c1ccc(-c2ccc(N(c3ccc4ccc5cc6oc7ccccc7c6cc5c4c3)c3cccc4c3ccc3ccccc34)cc2)cc1 ; c1ccc(-c2ccc(N(c3ccc4ccc5cc6oc7ccccc7c6cc5c4c3)c3cccc4c3ccc3ccccc34)cn2)cc1 ; CC1(C)c2ccccc2-c2cc(N(c3ccccc3)c3ccc4ccc5cc6oc7ccccc7c6cc5c4c3)ccc2C1(C)C]

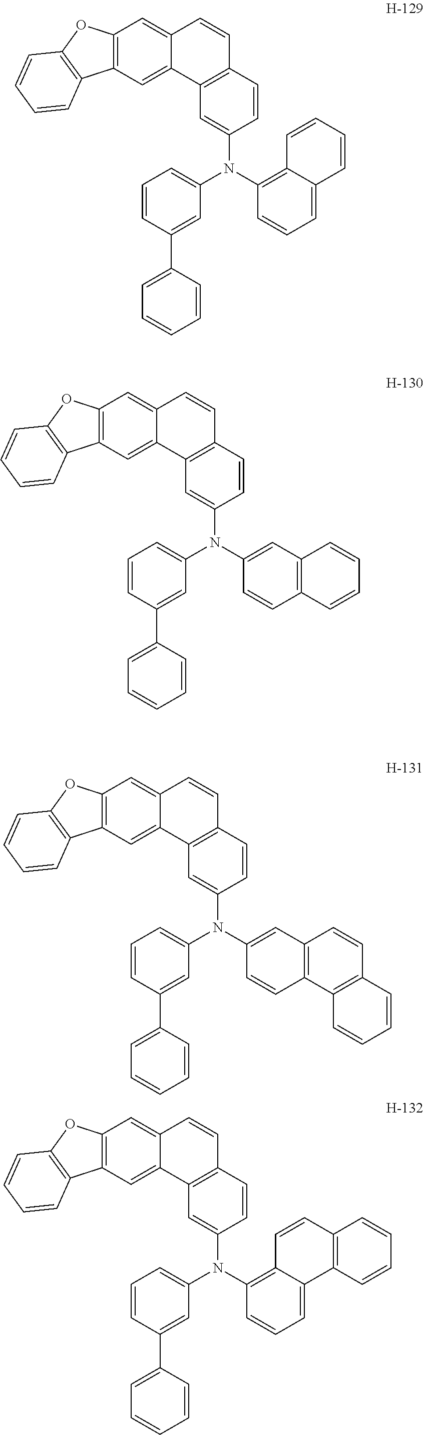

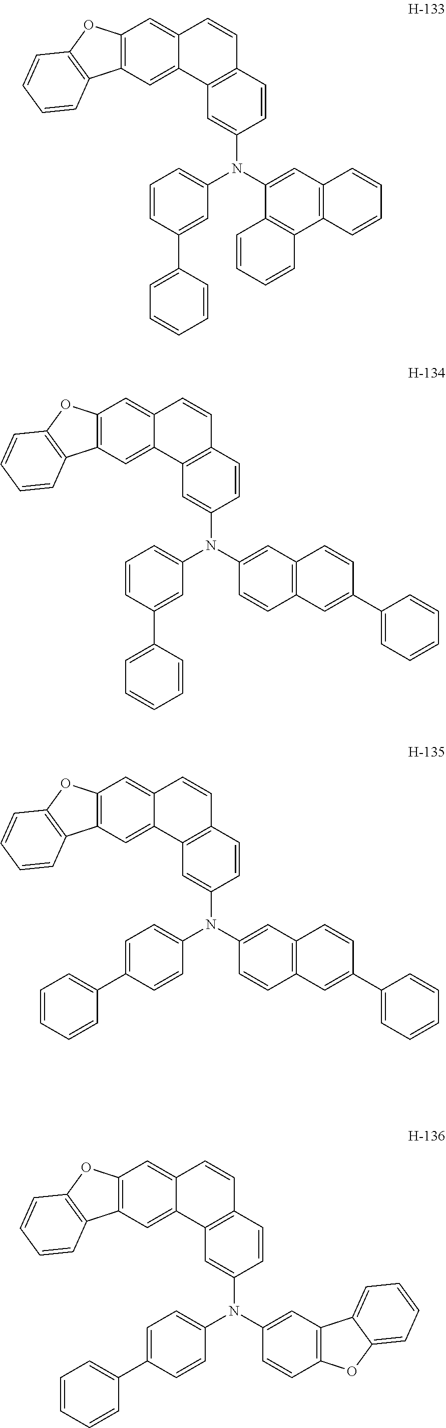 [SMILES: c1ccc(-c2cccc(N(c3ccc4ccc5cc6oc7ccccc7c6cc5c4c3)c3cc4ccccc4c4ccccc34)c2)cc1 ; c1ccc(-c2cccc(N(c3ccc4cc(-c5ccccc5)ccc4c3)c3ccc4ccc5cc6oc7ccccc7c6cc5c4c3)c2)cc1 ; c1ccc(-c2ccc(N(c3ccc4cc(-c5ccccc5)ccc4c3)c3ccc4ccc5cc6oc7ccccc7c6cc5c4c3)cc2)cc1 ; c1ccc(-c2ccc(N(c3ccc4ccc5cc6oc7ccccc7c6cc5c4c3)c3ccc4oc5ccccc5c4c3)cc2)cc1]

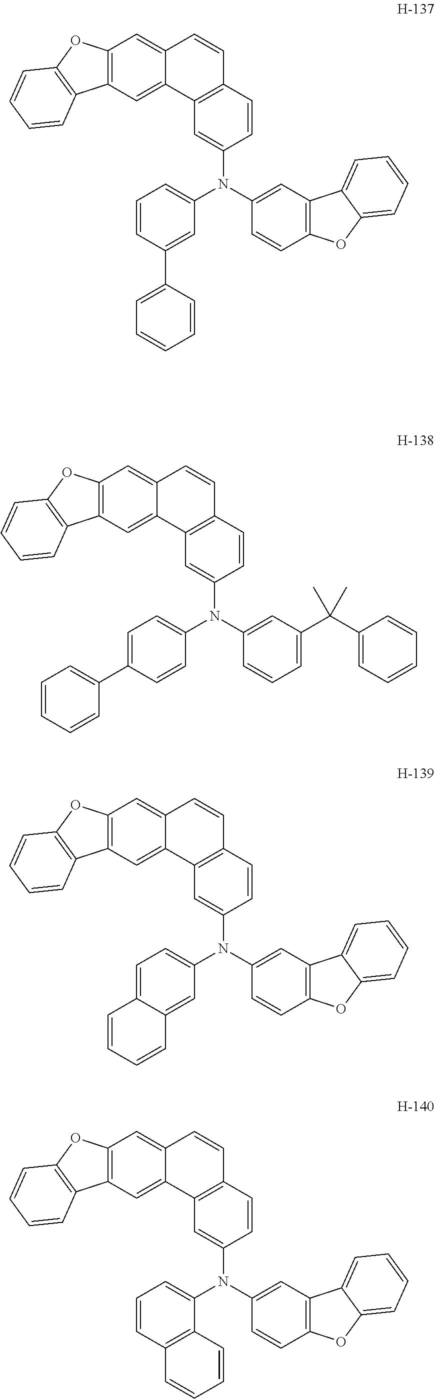 [SMILES: c1ccc(-c2cccc(N(c3ccc4ccc5cc6oc7ccccc7c6cc5c4c3)c3ccc4oc5ccccc5c4c3)c2)cc1 ; CC(C)(c1ccccc1)c1cccc(N(c2ccc(-c3ccccc3)cc2)c2ccc3ccc4cc5oc6ccccc6c5cc4c3c2)c1 ; c1ccc2cc(N(c3ccc4ccc5cc6oc7ccccc7c6cc5c4c3)c3ccc4oc5ccccc5c4c3)ccc2c1 ; c1ccc2c(N(c3ccc4ccc5cc6oc7ccccc7c6cc5c4c3)c3ccc4oc5ccccc5c4c3)cccc2c1]

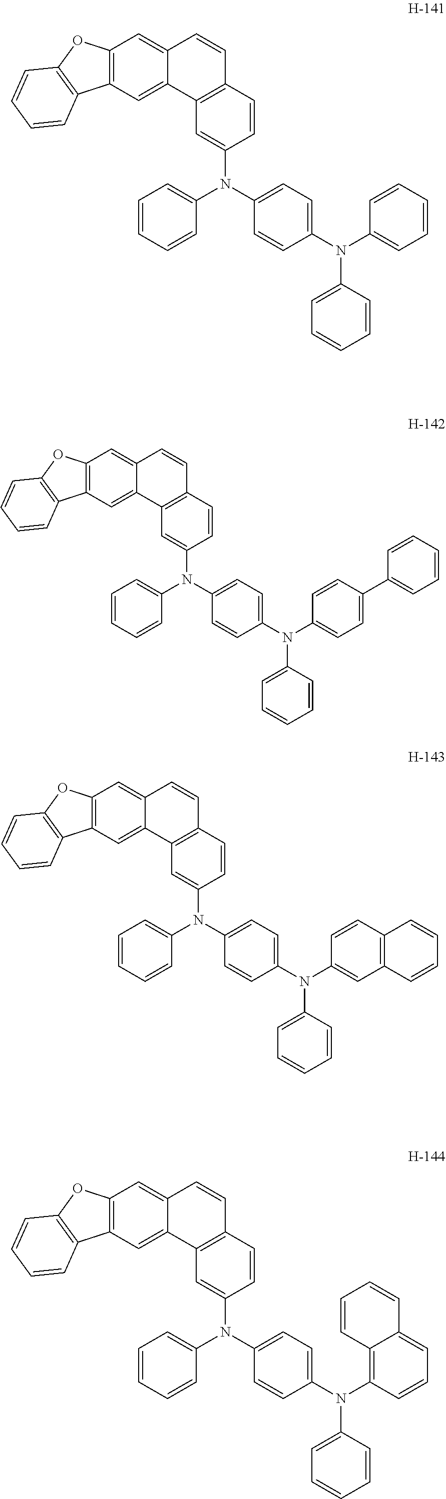 [SMILES: c1ccc(N(c2ccccc2)c2ccc(N(c3ccccc3)c3ccc4ccc5cc6oc7ccccc7c6cc5c4c3)cc2)cc1 ; c1ccc(-c2ccc(N(c3ccccc3)c3ccc(N(c4ccccc4)c4ccc5ccc6cc7oc8ccccc8c7cc6c5c4)cc3)cc2)cc1 ; c1ccc(N(c2ccc(N(c3ccccc3)c3ccc4ccc5cc6oc7ccccc7c6cc5c4c3)cc2)c2ccc3ccccc3c2)cc1 ; c1ccc(N(c2ccc(N(c3ccccc3)c3cccc4ccccc34)cc2)c2ccc3ccc4cc5oc6ccccc6c5cc4c3c2)cc1]

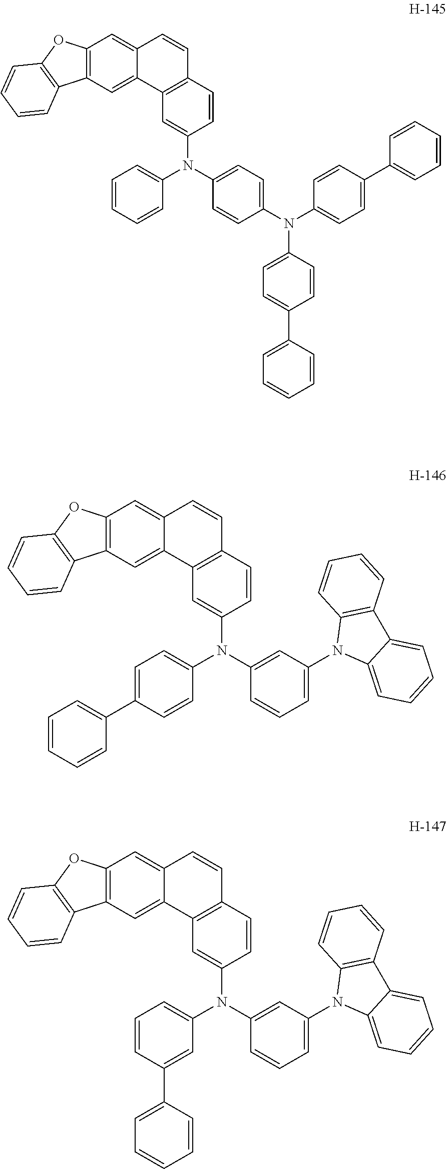 [SMILES: c1ccc(-c2ccc(N(c3ccc(-c4ccccc4)cc3)c3ccc(N(c4ccccc4)c4ccc5ccc6cc7oc8ccccc8c7cc6c5c4)cc3)cc2)cc1 ; c1ccc(-c2ccc(N(c3cccc(-n4c5ccccc5c5ccccc54)c3)c3ccc4ccc5cc6oc7ccccc7c6cc5c4c3)cc2)cc1 ; c1ccc(-c2cccc(N(c3cccc(-n4c5ccccc5c5ccccc54)c3)c3ccc4ccc5cc6oc7ccccc7c6cc5c4c3)c2)cc1]

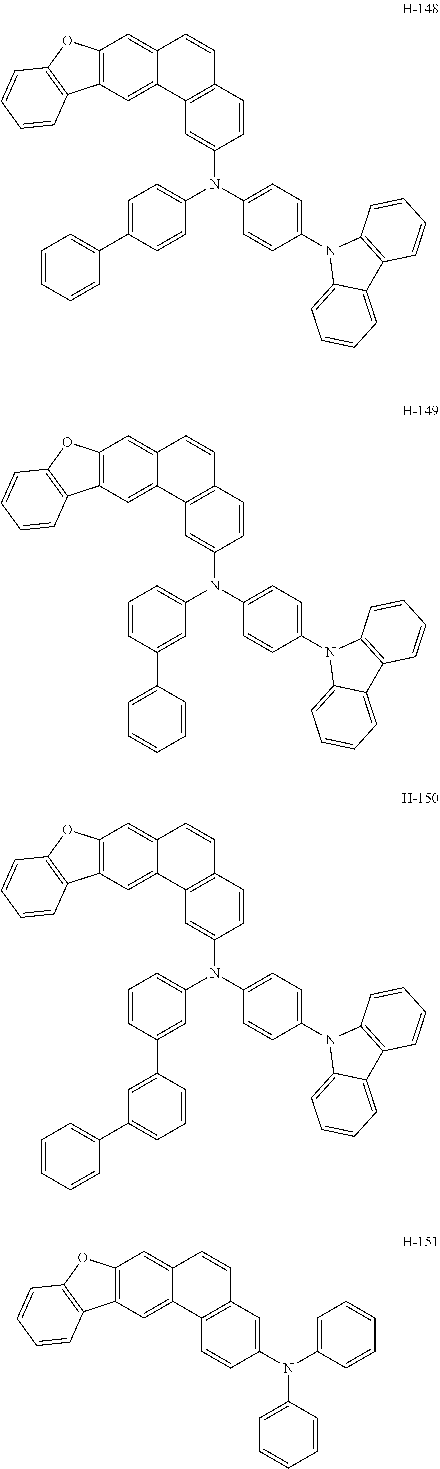 [SMILES: c1ccc(-c2ccc(N(c3ccc(-n4c5ccccc5c5ccccc54)cc3)c3ccc4ccc5cc6oc7ccccc7c6cc5c4c3)cc2)cc1 ; c1ccc(-c2cccc(N(c3ccc(-n4c5ccccc5c5ccccc54)cc3)c3ccc4ccc5cc6oc7ccccc7c6cc5c4c3)c2)cc1 ; c1ccc(-c2cccc(-c3cccc(N(c4ccc(-n5c6ccccc6c6ccccc65)cc4)c4ccc5ccc6cc7oc8ccccc8c7cc6c5c4)c3)c2)cc1 ; c1ccc(N(c2ccccc2)c2ccc3c(ccc4cc5oc6ccccc6c5cc43)c2)cc1]

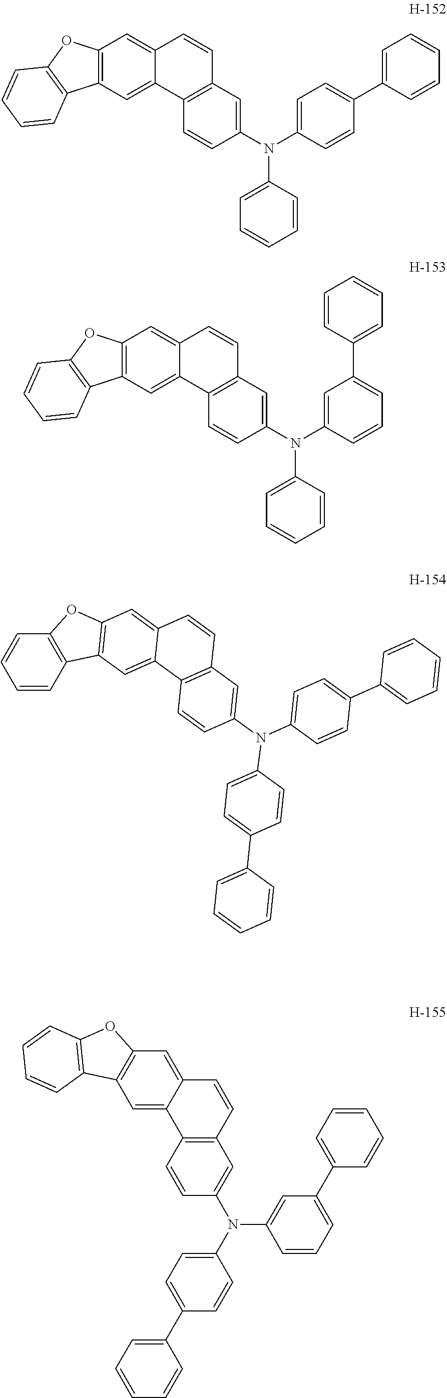 [SMILES: c1ccc(-c2ccc(N(c3ccccc3)c3ccc4c(ccc5cc6oc7ccccc7c6cc54)c3)cc2)cc1 ; c1ccc(-c2cccc(N(c3ccccc3)c3ccc4c(ccc5cc6oc7ccccc7c6cc54)c3)c2)cc1 ; c1ccc(-c2ccc(N(c3ccc(-c4ccccc4)cc3)c3ccc4c(ccc5cc6oc7ccccc7c6cc54)c3)cc2)cc1 ; c1ccc(-c2ccc(N(c3cccc(-c4ccccc4)c3)c3ccc4c(ccc5cc6oc7ccccc7c6cc54)c3)cc2)cc1]

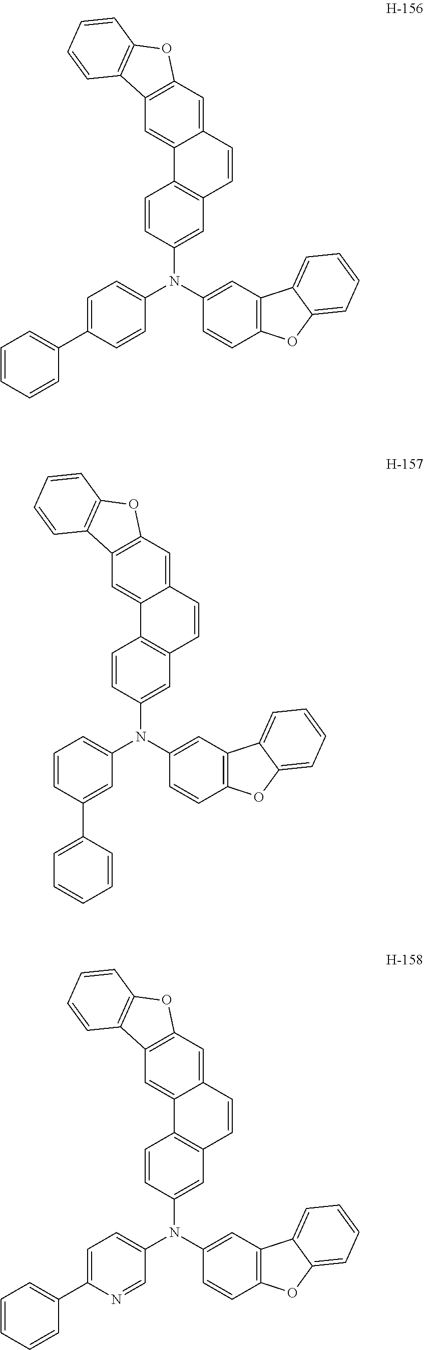 [SMILES: c1ccc(-c2ccc(N(c3ccc4c(ccc5cc6oc7ccccc7c6cc54)c3)c3ccc4oc5ccccc5c4c3)cc2)cc1 ; c1ccc(-c2cccc(N(c3ccc4c(ccc5cc6oc7ccccc7c6cc54)c3)c3ccc4oc5ccccc5c4c3)c2)cc1 ; c1ccc(-c2ccc(N(c3ccc4c(ccc5cc6oc7ccccc7c6cc54)c3)c3ccc4oc5ccccc5c4c3)cn2)cc1]

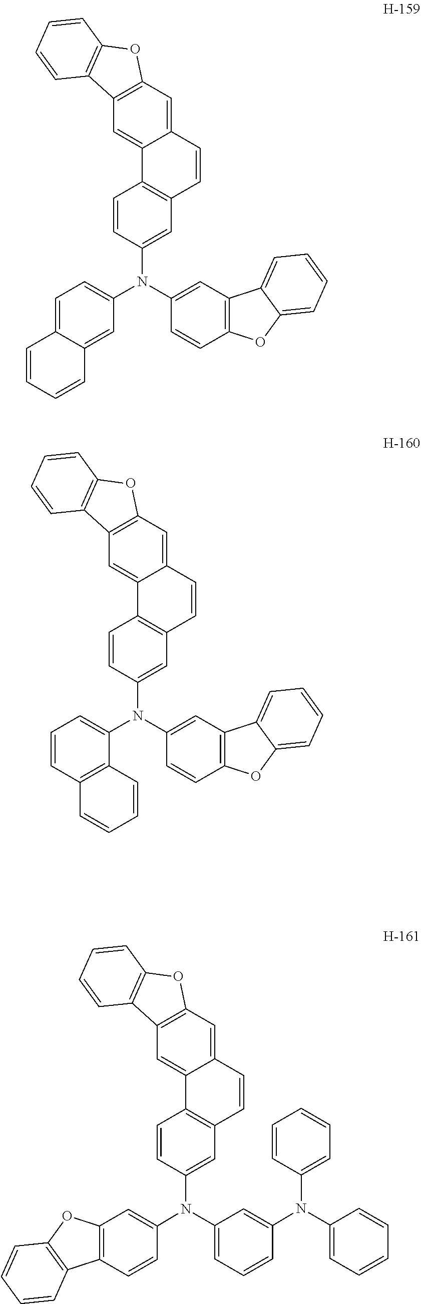 [SMILES: c1ccc2cc(N(c3ccc4c(ccc5cc6oc7ccccc7c6cc54)c3)c3ccc4oc5ccccc5c4c3)ccc2c1 ; c1ccc2c(N(c3ccc4c(ccc5cc6oc7ccccc7c6cc54)c3)c3ccc4oc5ccccc5c4c3)cccc2c1 ; c1ccc(N(c2ccccc2)c2cccc(N(c3ccc4c(ccc5cc6oc7ccccc7c6cc54)c3)c3ccc4c(c3)oc3ccccc34)c2)cc1]

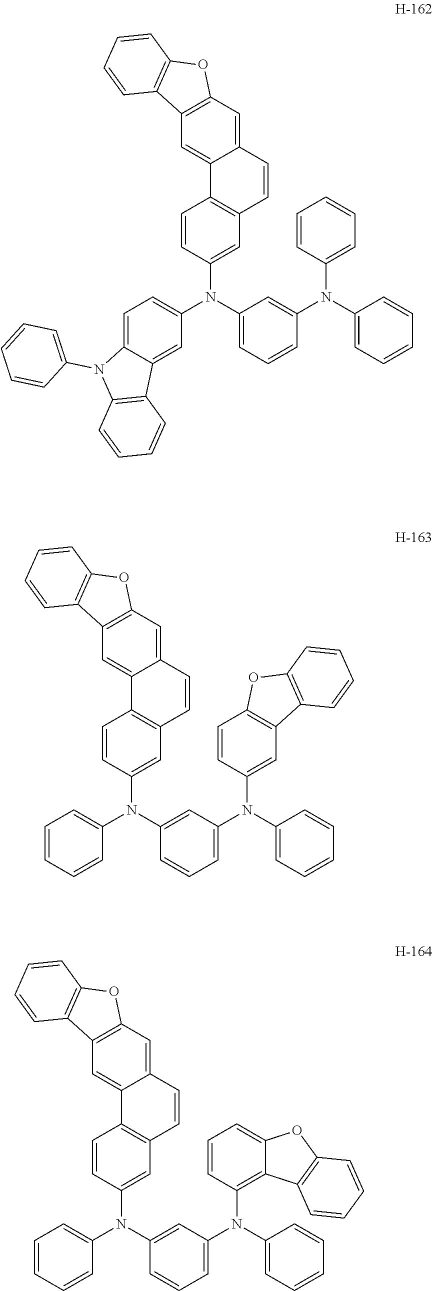 [SMILES: c1ccc(N(c2ccccc2)c2cccc(N(c3ccc4c(ccc5cc6oc7ccccc7c6cc54)c3)c3ccc4c(c3)c3ccccc3n4-c3ccccc3)c2)cc1 ; c1ccc(N(c2cccc(N(c3ccccc3)c3ccc4oc5ccccc5c4c3)c2)c2ccc3c(ccc4cc5oc6ccccc6c5cc43)c2)cc1 ; c1ccc(N(c2cccc(N(c3ccccc3)c3cccc4oc5ccccc5c34)c2)c2ccc3c(ccc4cc5oc6ccccc6c5cc43)c2)cc1]

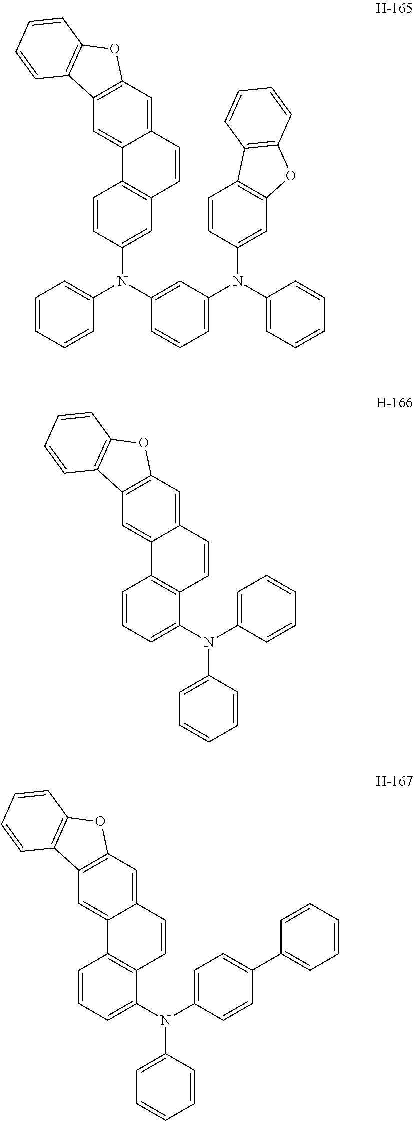 [SMILES: c1ccc(N(c2cccc(N(c3ccccc3)c3ccc4c(c3)oc3ccccc34)c2)c2ccc3c(ccc4cc5oc6ccccc6c5cc43)c2)cc1 ; c1ccc(N(c2ccccc2)c2cccc3c2ccc2cc4oc5ccccc5c4cc23)cc1 ; c1ccc(-c2ccc(N(c3ccccc3)c3cccc4c3ccc3cc5oc6ccccc6c5cc34)cc2)cc1]

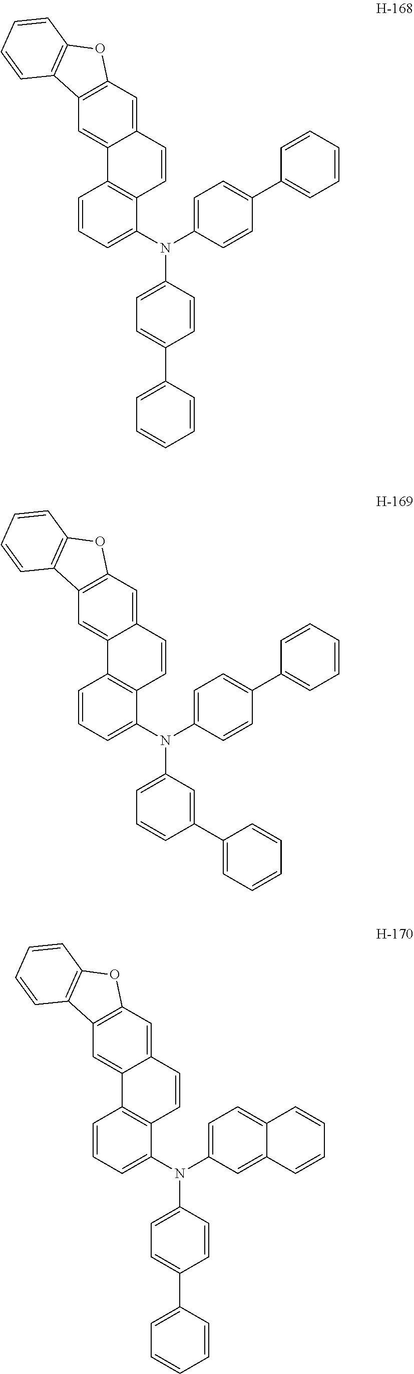 [SMILES: c1ccc(-c2ccc(N(c3ccc(-c4ccccc4)cc3)c3cccc4c3ccc3cc5oc6ccccc6c5cc34)cc2)cc1 ; c1ccc(-c2ccc(N(c3cccc(-c4ccccc4)c3)c3cccc4c3ccc3cc5oc6ccccc6c5cc34)cc2)cc1 ; c1ccc(-c2ccc(N(c3ccc4ccccc4c3)c3cccc4c3ccc3cc5oc6ccccc6c5cc34)cc2)cc1]

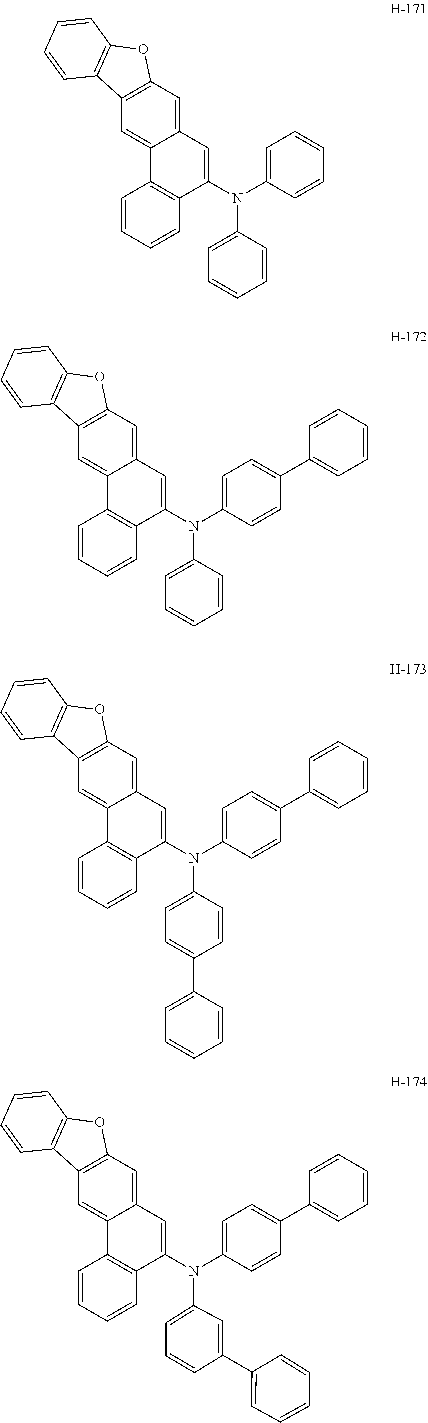

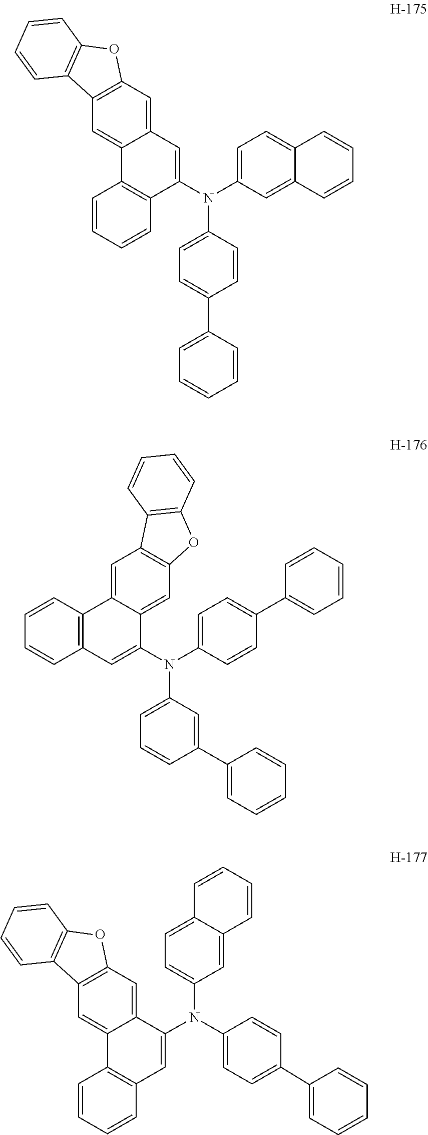 [SMILES: c1ccc(-c2ccc(N(c3ccc4ccccc4c3)c3cc4cc5oc6ccccc6c5cc4c4ccccc34)cc2)cc1 ; c1ccc(-c2ccc(N(c3cccc(-c4ccccc4)c3)c3cc4ccccc4c4cc5c(cc34)oc3ccccc35)cc2)cc1 ; c1ccc(-c2ccc(N(c3ccc4ccccc4c3)c3cc4ccccc4c4cc5c(cc34)oc3ccccc35)cc2)cc1]

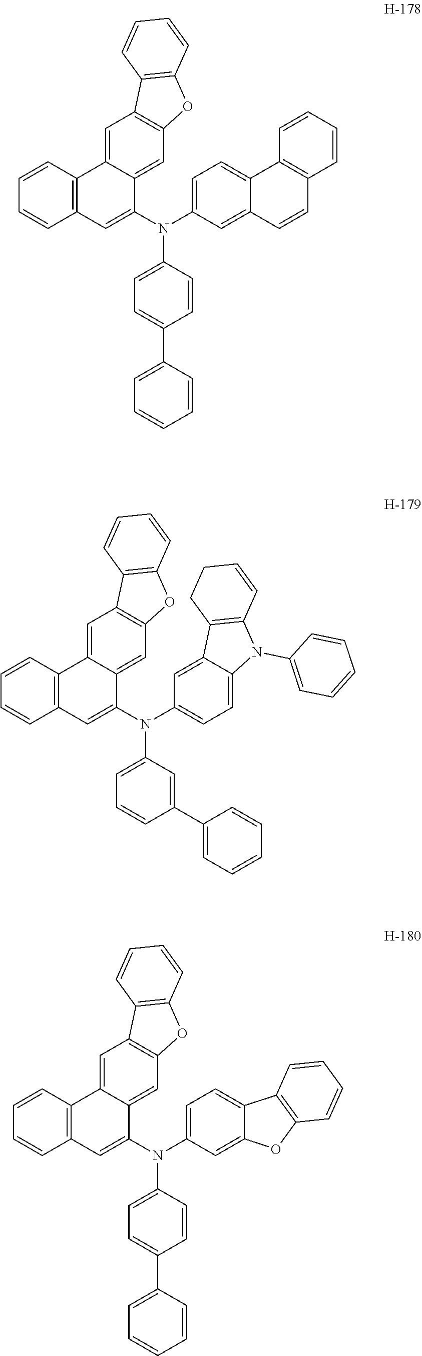 [SMILES: c1ccc(-c2ccc(N(c3ccc4c(ccc5ccccc54)c3)c3cc4ccccc4c4cc5c(cc34)oc3ccccc35)cc2)cc1 ; C1=Cc2c(c3cc(N(c4cccc(-c5ccccc5)c4)c4cc5ccccc5c5cc6c(cc45)oc4ccccc46)ccc3n2-c2ccccc2)CC1 ; c1ccc(-c2ccc(N(c3ccc4c(c3)oc3ccccc34)c3cc4ccccc4c4cc5c(cc34)oc3ccccc35)cc2)cc1]

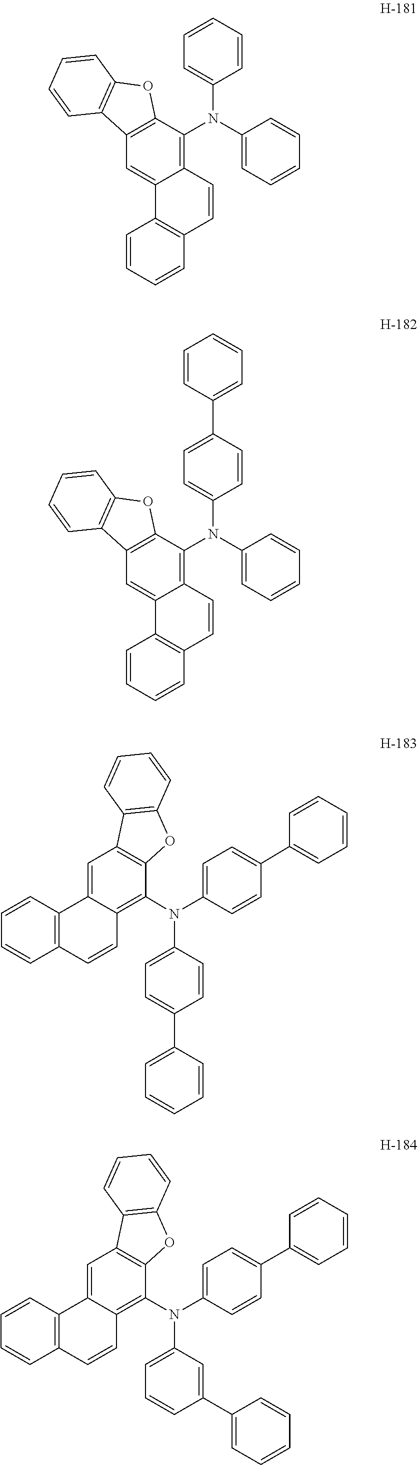 [SMILES: c1ccc(N(c2ccccc2)c2c3ccc4ccccc4c3cc3c2oc2ccccc23)cc1 ; c1ccc(-c2ccc(N(c3ccccc3)c3c4ccc5ccccc5c4cc4c3oc3ccccc34)cc2)cc1 ; c1ccc(-c2ccc(N(c3ccc(-c4ccccc4)cc3)c3c4ccc5ccccc5c4cc4c3oc3ccccc34)cc2)cc1 ; c1ccc(-c2ccc(N(c3cccc(-c4ccccc4)c3)c3c4ccc5ccccc5c4cc4c3oc3ccccc34)cc2)cc1]

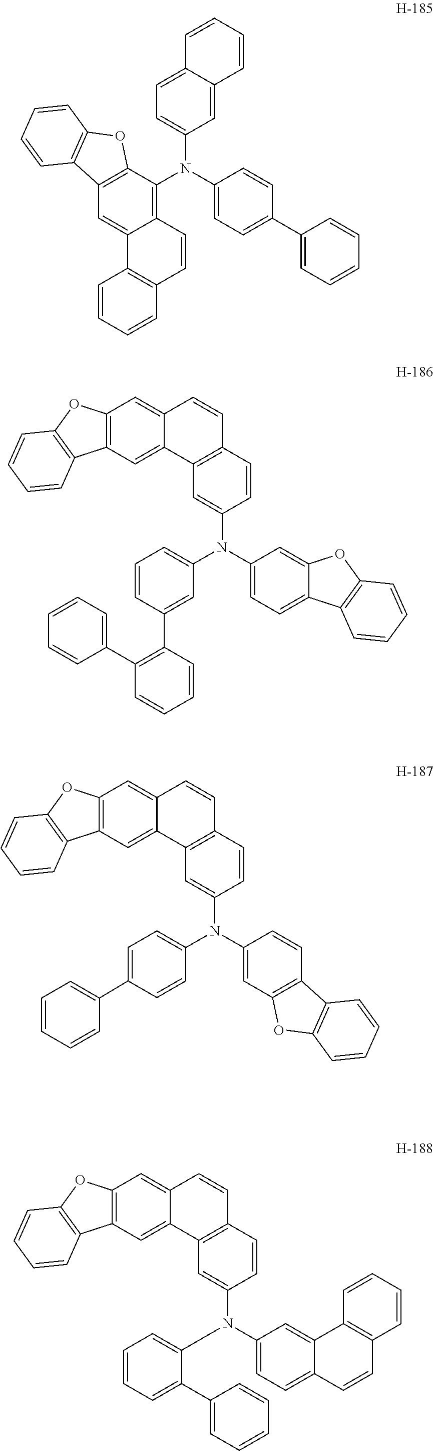 [SMILES: c1ccc(-c2ccc(N(c3ccc4ccccc4c3)c3c4ccc5ccccc5c4cc4c3oc3ccccc34)cc2)cc1 ; c1ccc(-c2ccccc2-c2cccc(N(c3ccc4c(c3)oc3ccccc34)c3ccc4ccc5cc6oc7ccccc7c6cc5c4c3)c2)cc1 ; c1ccc(-c2ccc(N(c3ccc4c(c3)oc3ccccc34)c3ccc4ccc5cc6oc7ccccc7c6cc5c4c3)cc2)cc1 ; c1ccc(-c2ccccc2N(c2ccc3ccc4ccccc4c3c2)c2ccc3ccc4cc5oc6ccccc6c5cc4c3c2)cc1]

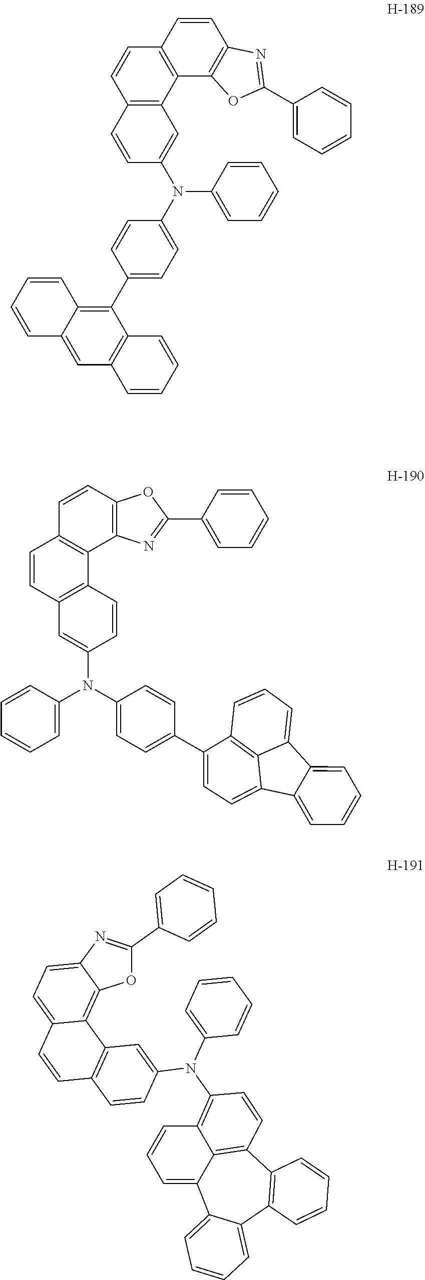

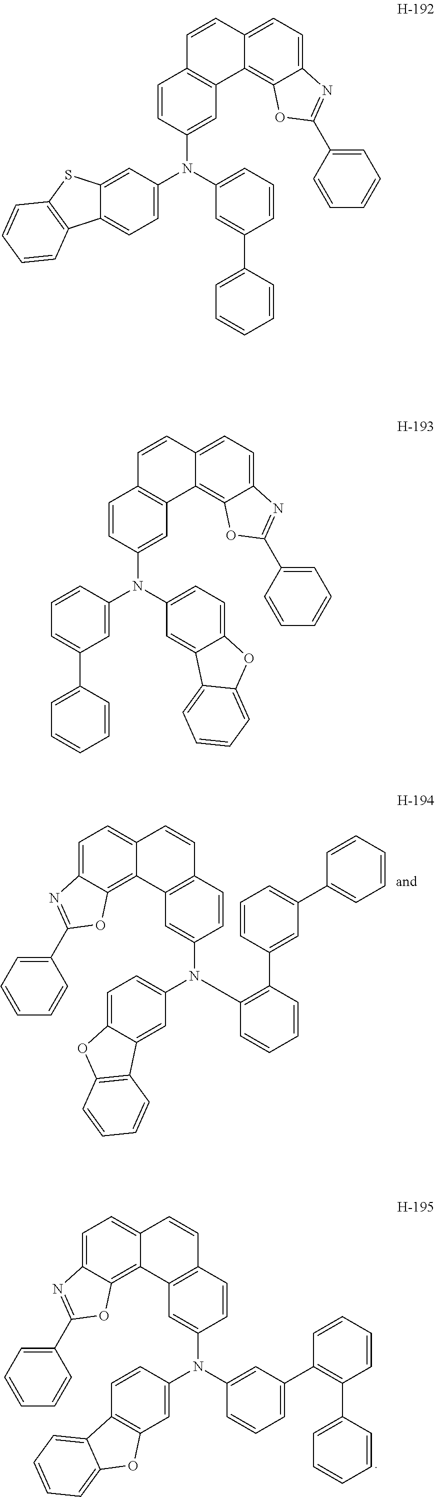

Any one of the compound of formulas 11 to 14 according to the present disclosure may be prepared by a synthetic method known to one skilled in the art, but is not limited thereto.

The present disclosure provides an organic electroluminescent material comprising the compound represented by Formula 1 and any one of the compounds represented by formulas 11 to 14. According to one embodiment of the present disclosure, the compound represented by formula 1 and one or more compound represented by formulas H-1 to H-195 may be used in an organic electroluminescent material. In addition, the present disclosure provides an organic electroluminescent device comprising the organic electroluminescent material.

The present disclosure provides an organic electroluminescent material comprising the organic electroluminescent compound of Formula 1 and an organic electroluminescent device comprising the organic electroluminescent material.

The organic electroluminescent material may be a light-emitting material, a hole transport material, a hole auxiliary material, or a light-emitting auxiliary material, and when there are two or more hole transport layers, the material may be a hole transport material (a hole auxiliary material) comprised in the hole transport layer adjacent to the light-emitting layer.

The material may consist of the organic electroluminescent compound of the present disclosure alone, or may further comprise conventional materials generally used in an organic electroluminescent material.

The hole transport zone of present disclosure may be composed of one or more layers selected from the group consisting of a hole transport layer, a hole injection layer, an electron blocking layer, and a hole auxiliary layer, and the each layers may be composed of one or more layers.

According to one embodiment of the present disclosure, the hole transport zone comprises a hole transport layer. In addition, the hole transport zone may comprise a hole transport layer and further comprise one or more layers of a hole injection layer, an electron blocking layer, and a hole auxiliary layer.

The organic electroluminescent device according to the present disclosure comprises a first electrode; a second electrode; and at least one organic layer between the first and second electrodes, and the organic layer may comprise at least one organic electroluminescent compound of Formula 1.

One of the first and second electrodes may be an anode, and the other may be a cathode. The organic layer may comprise a light-emitting layer, and may further comprise at least one layer selected from a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer.

The organic electroluminescent compound of Formula 1 of the present disclosure may be comprised in any one or more layers of the light-emitting layer, the hole injection layer, the hole transport layer, the hole auxiliary layer, the light-emitting auxiliary layer, the electron transport layer, the electron buffer layer, the an electron injection layer, the interlayer, the hole blocking layer, and the electron blocking layer. In some cases, the organic electroluminescent compound of Formula 1 may preferably be comprised in one or more of the hole transport layer, the hole auxiliary layer or the light-emitting auxiliary layer, or the light-emitting layer. When there are two or more hole transport layers, the organic electroluminescent compound may be used in at least one of the layers.

For example, when used in the hole transport layer, the organic electroluminescent compound of the present disclosure may be comprised as a hole transport material. In addition, when used in the light-emitting layer, the organic electroluminescent compound of the present disclosure may be comprised as a host material.

The light-emitting layer may comprise one or more hosts and one or more dopants. If necessary, the light-emitting layer may comprise a co-host material, i.e., a plurality of host materials of two or more. The organic electroluminescent compound of the present disclosure may be used as a co-host material.

The host used in the present disclosure may be a phosphorescent host compound or a fluorescent host compound, and the host compounds are not particularly limited.

The dopant comprised in the organic electroluminescent device according to the present disclosure may be at least one phosphorescent or fluorescent dopant, preferably at least one phosphorescent dopant. The phosphorescent dopant materials applied to the organic electroluminescent device according to the present disclosure are not particularly limited, but may be selected from metallated complex compounds of iridium (Ir), osmium (Os), copper (Cu), and platinum (Pt), may be preferably selected from ortho-metallated complex compounds of iridium (Ir), osmium (Os), copper (Cu), and platinum (Pt), and may be more preferably an ortho-metallated iridium complex compound.

The compound represented by following Formula 101 may be used as a phosphorescent dopant material comprised in the organic electroluminescent device of the present disclosure, but is not limited thereto.

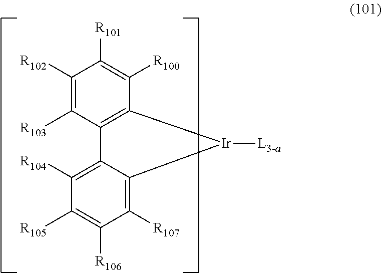

In Formula 101,
L is selected from the following structures 1 to 3;

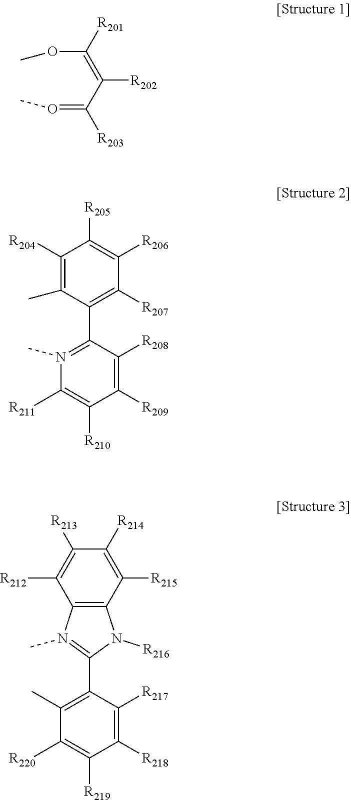

R₁₀₀ to R₁₀₃, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent substituent(s) to form a ring(s), e.g., a substituted or unsubstituted quinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline ring, together with pyridine;
R₁₀₄ to R₁₀₇, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent(s) to form a ring(s), e.g., a substituted or unsubstituted naphthyl, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine ring, together with benzene;
R₂₀₁ to R₂₂₀, each independently, represent hydrogen, deuterium, a halogen, a (C1-C30)alkyl unsubstituted or substituted with deuterium and/or a halogen(s), a substituted or unsubstituted (C3-C30)cycloalkyl, or a substituted or unsubstituted (C6-C30)aryl; or may be linked to an adjacent substituent(s) to form a a substituted or unsubstituted ring; and
s represents an integer of 1 to 3.

The specific examples of the phosphorescent dopant compound are as follows, but are not limited thereto.

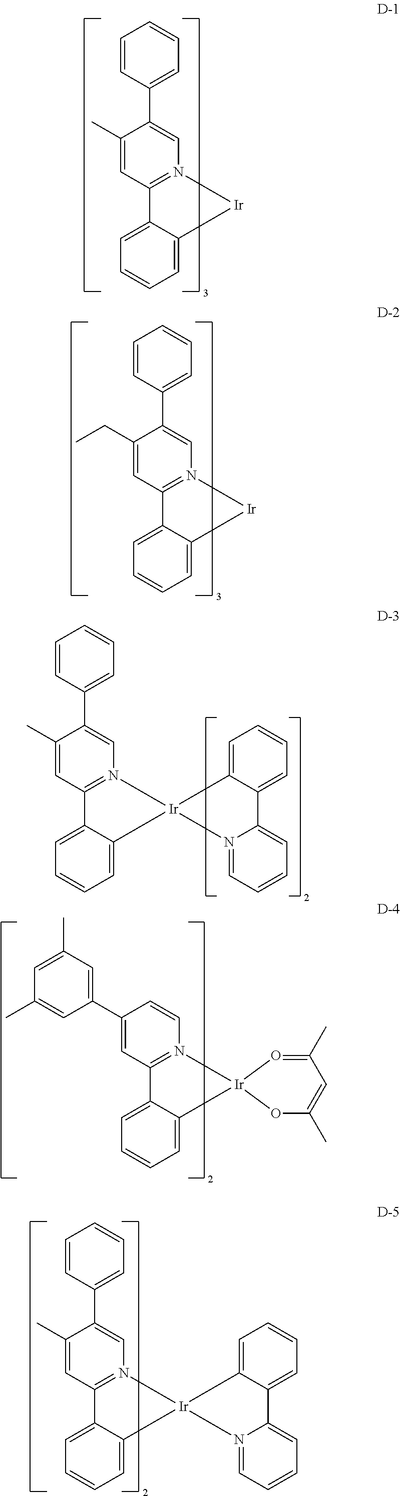

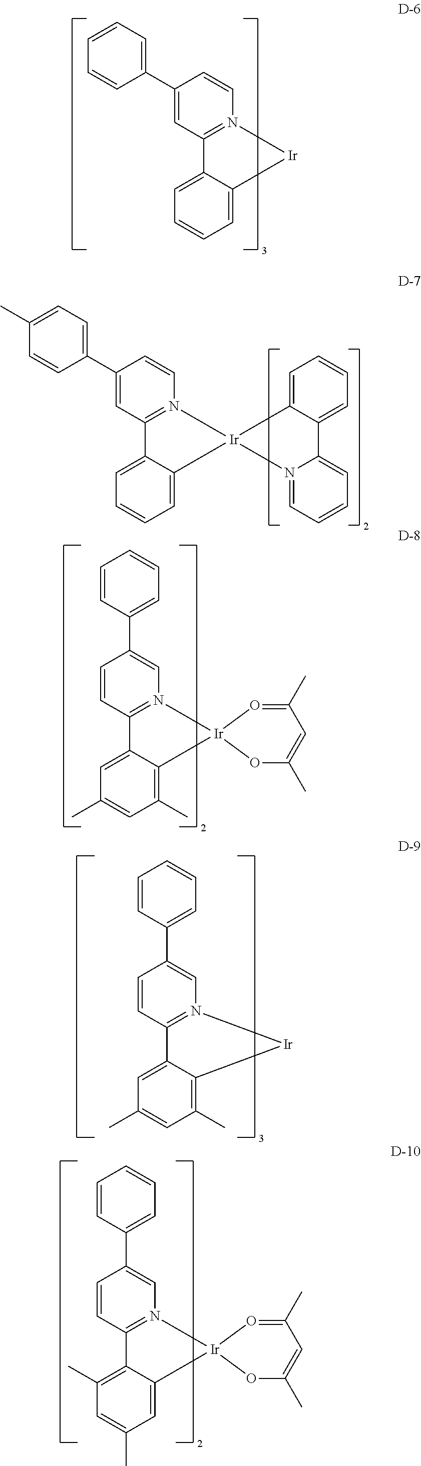

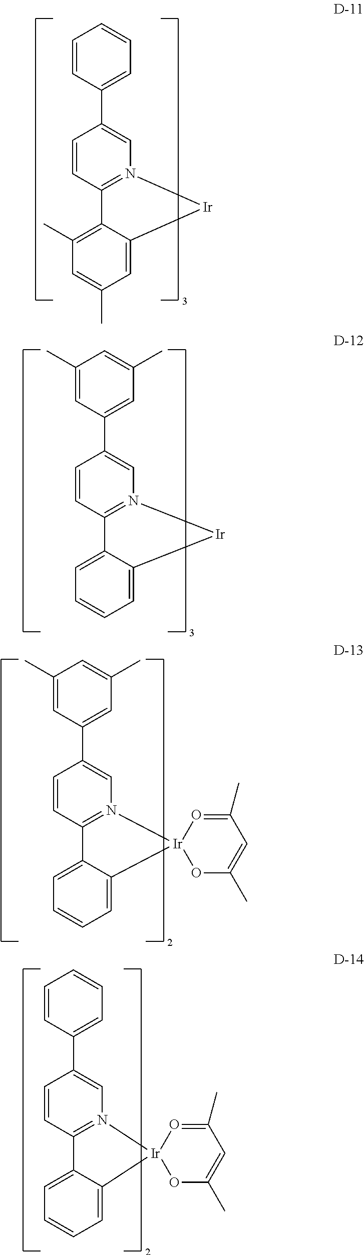

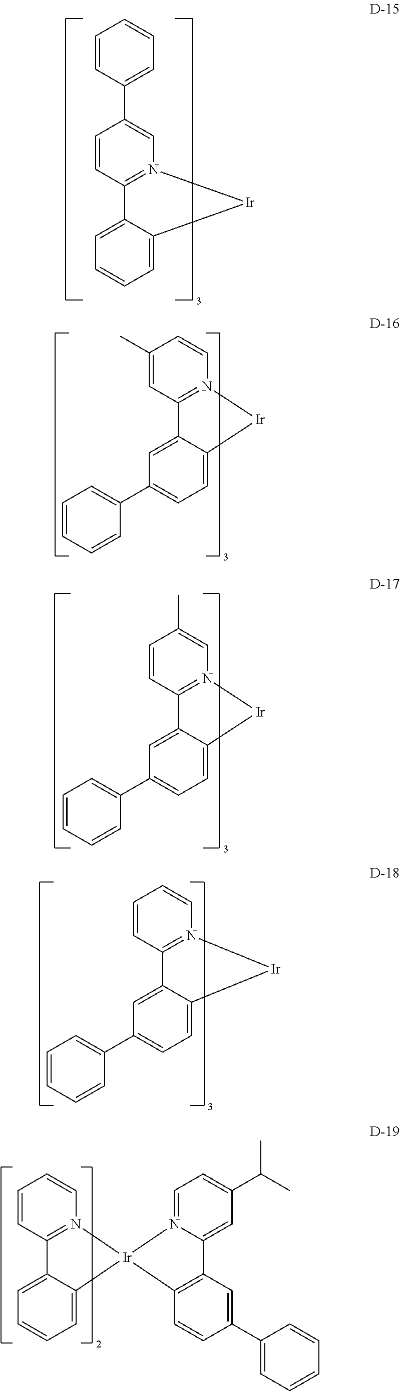

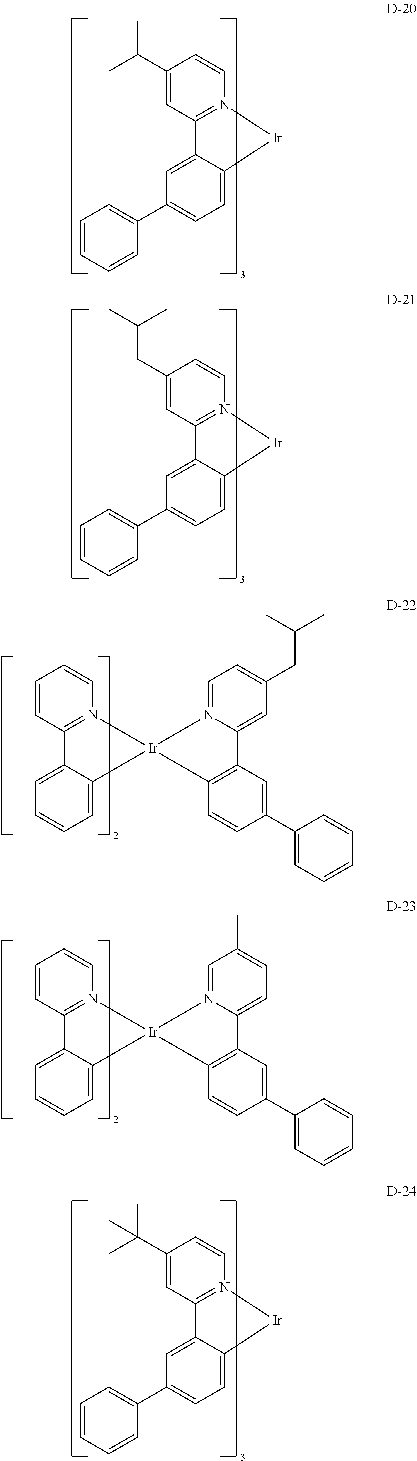

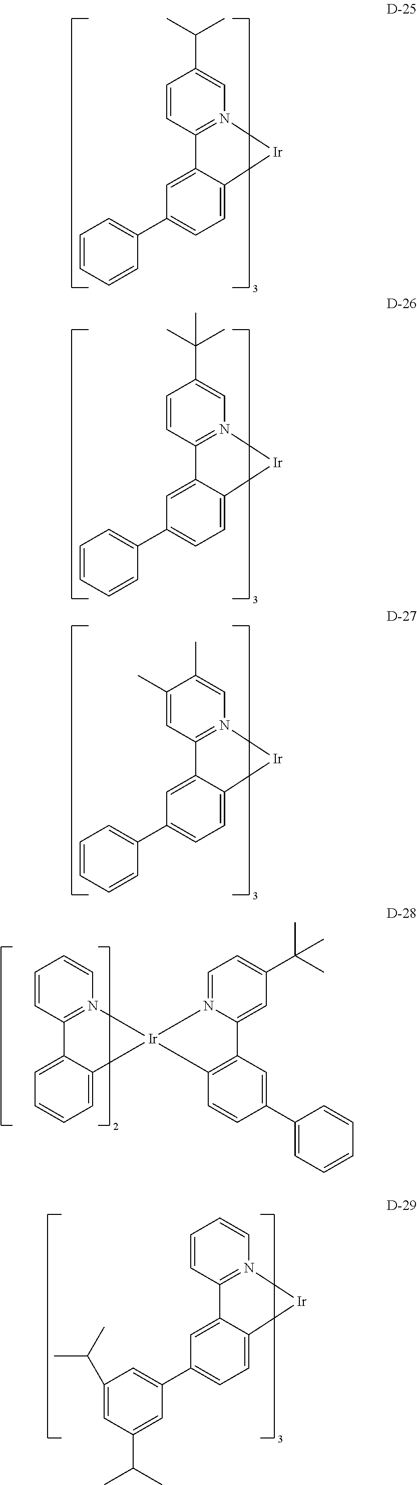

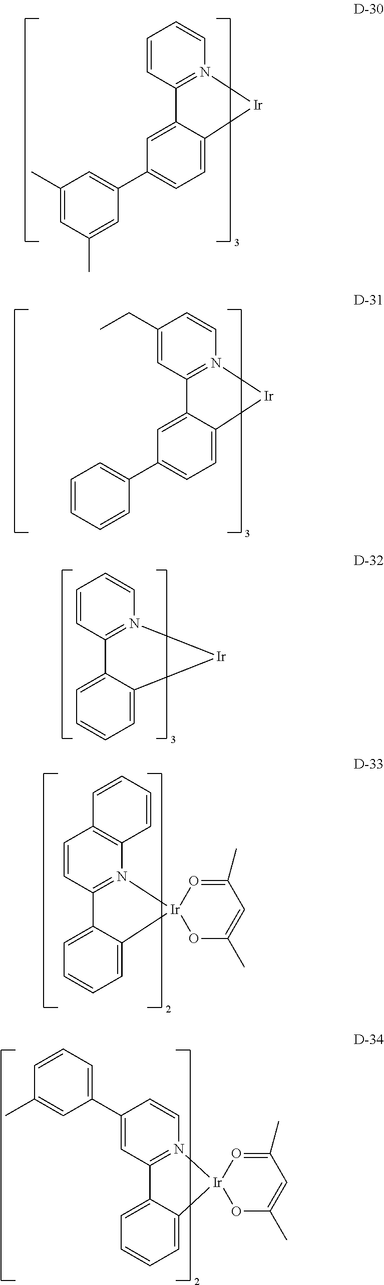

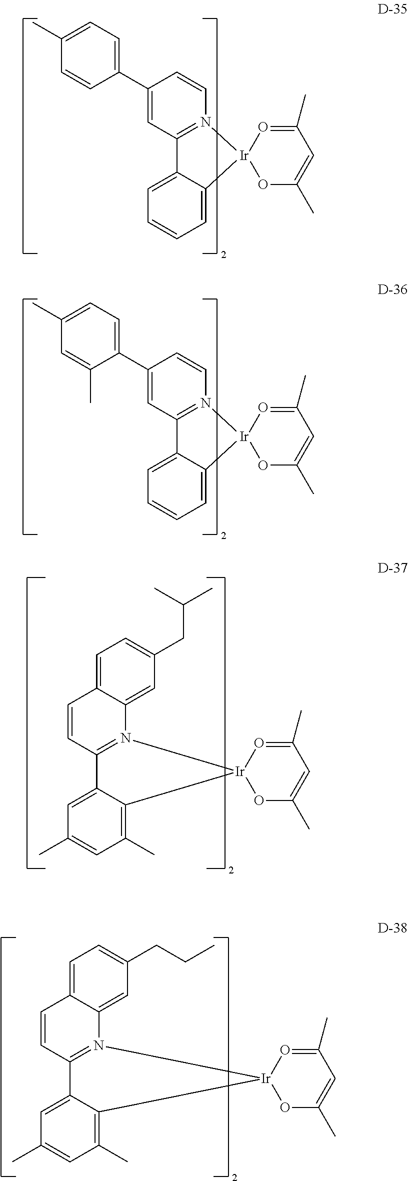

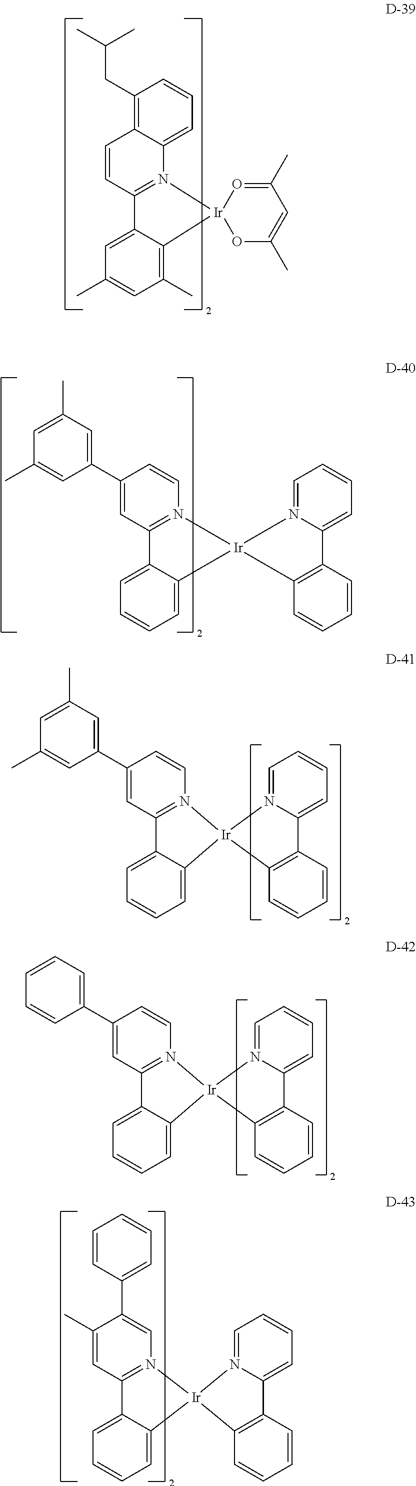

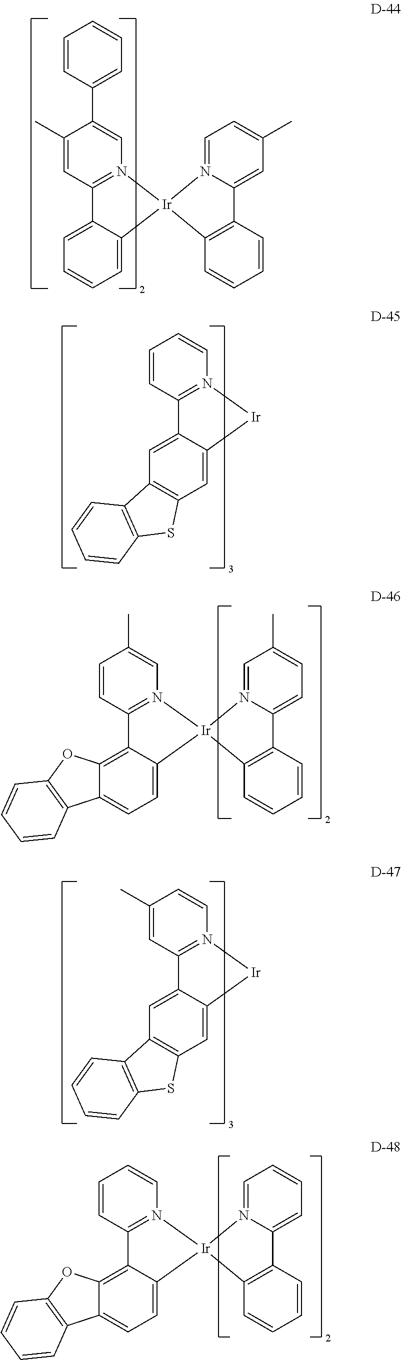

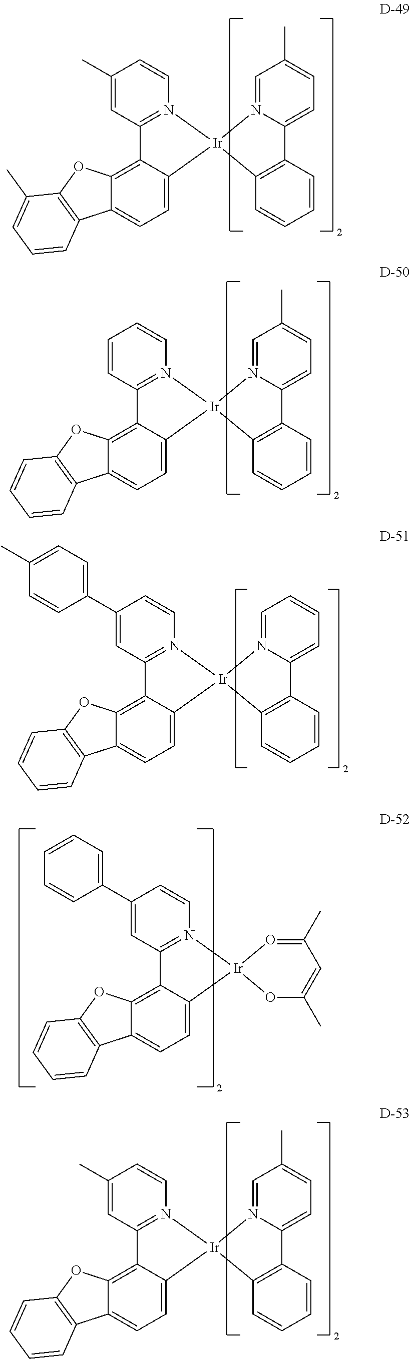

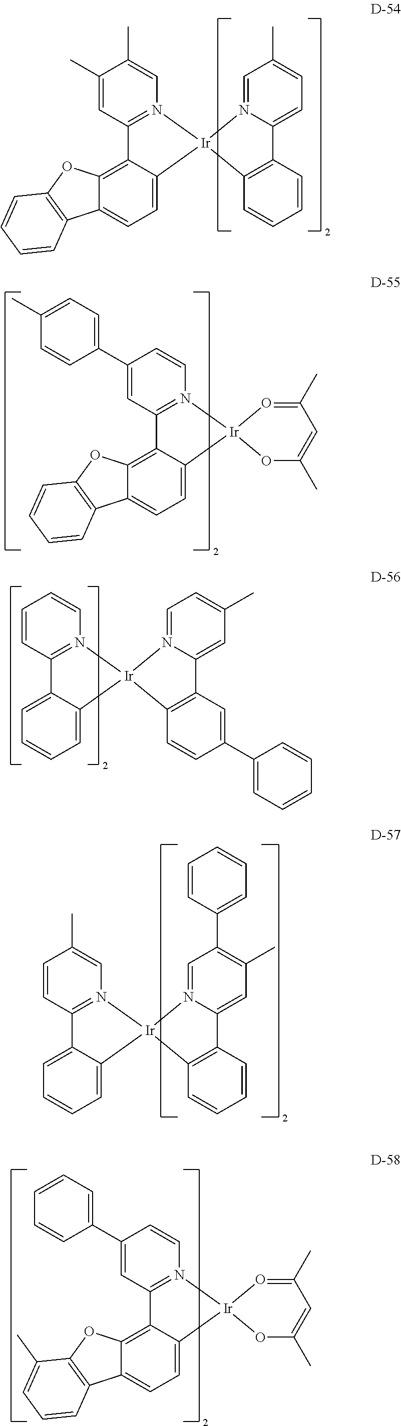

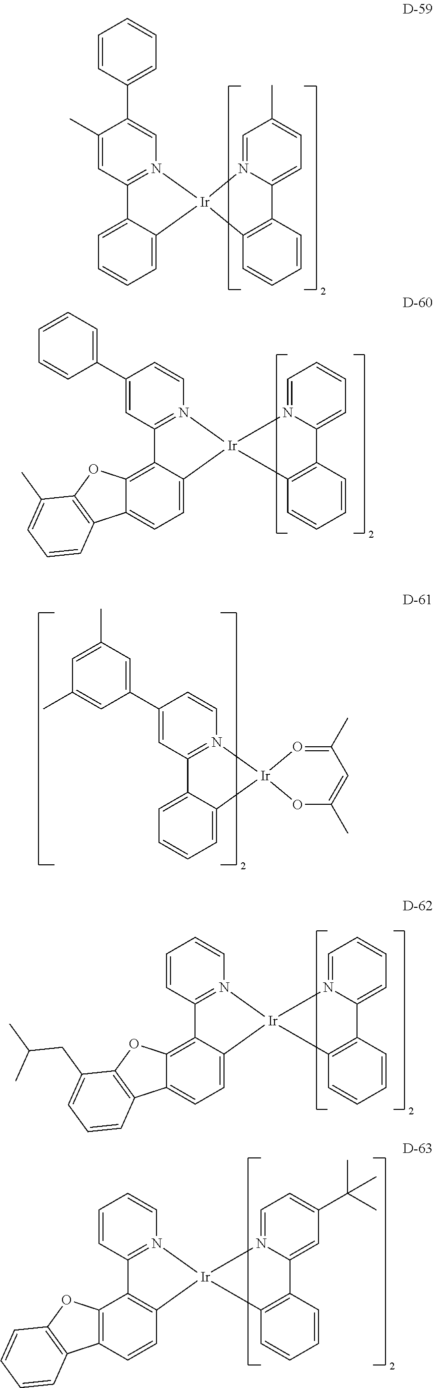

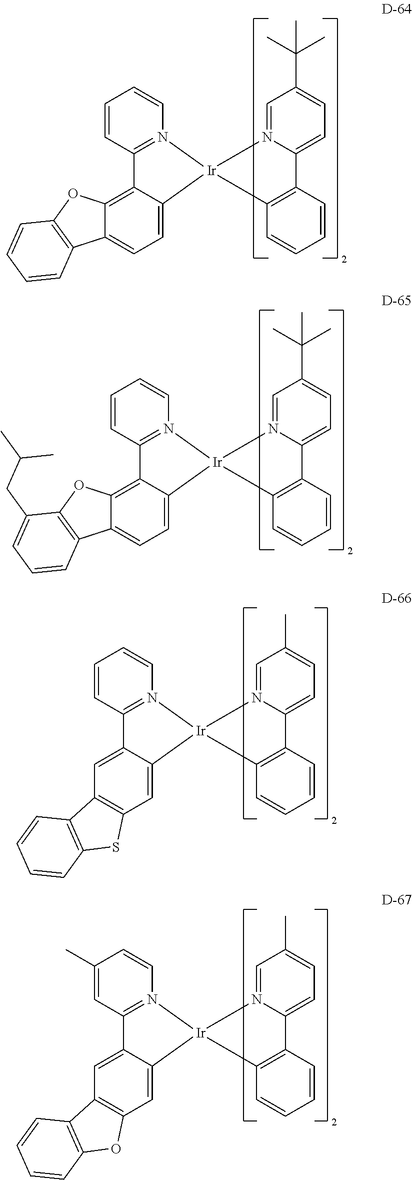

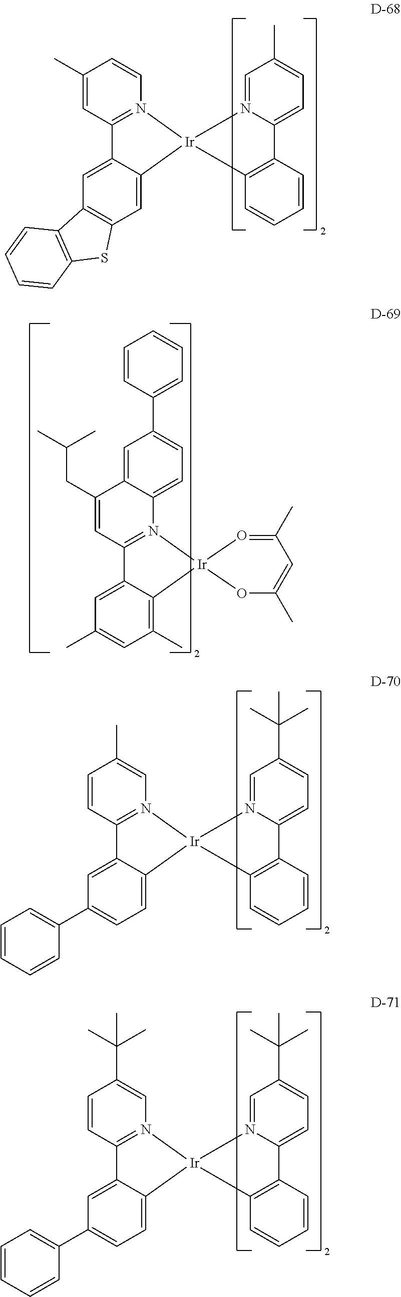

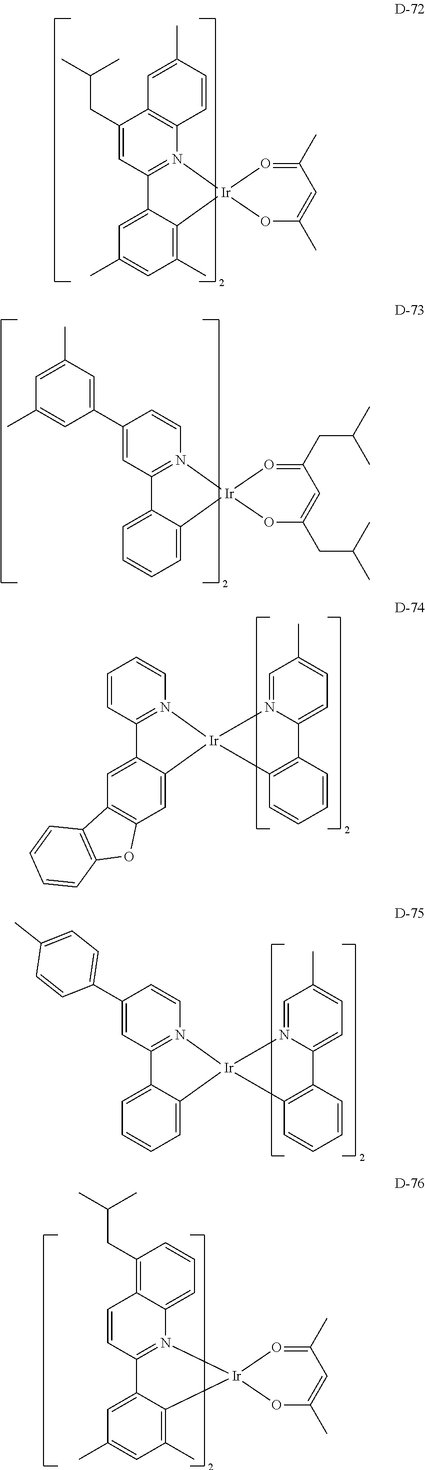

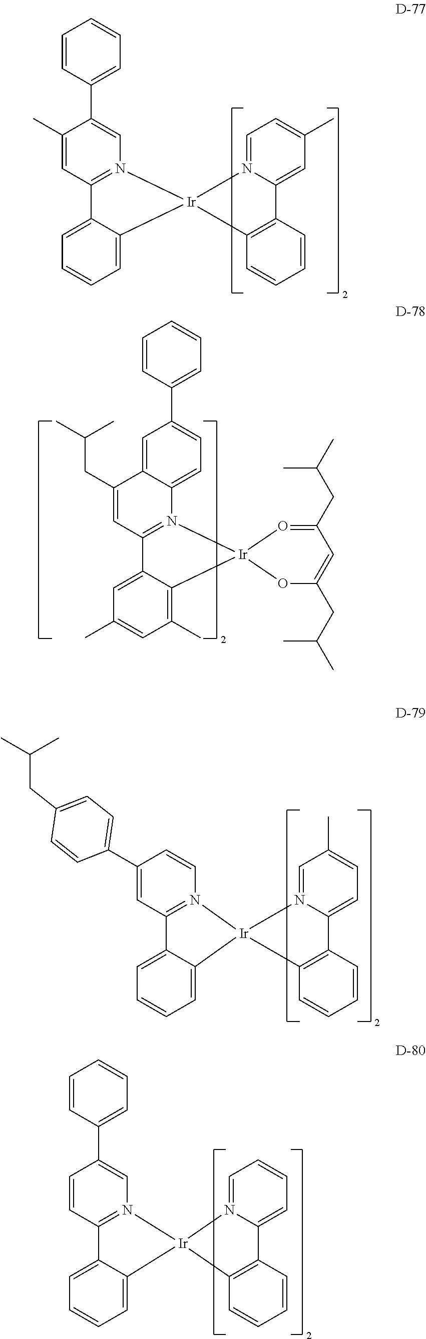

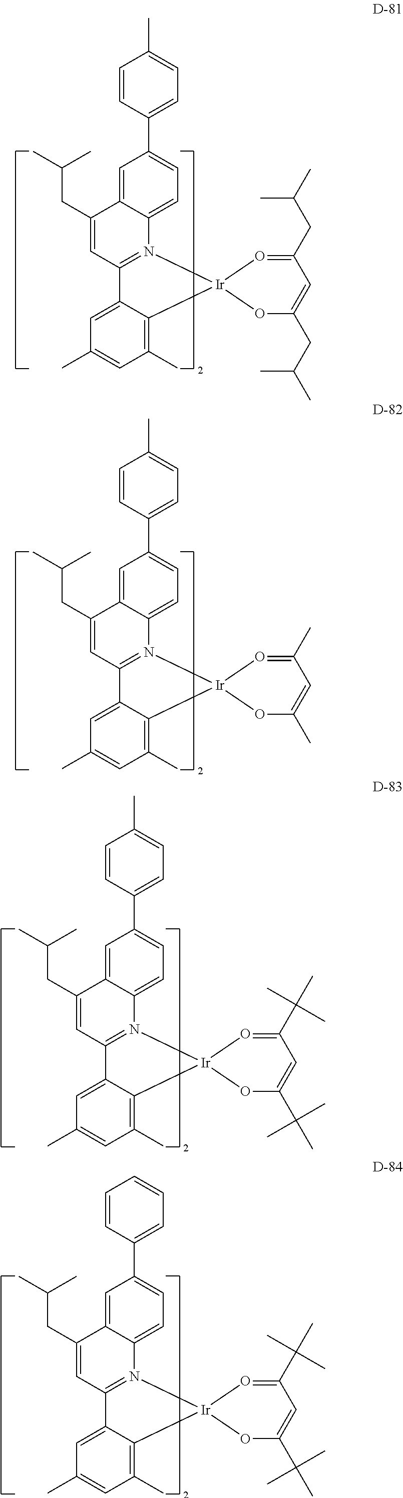

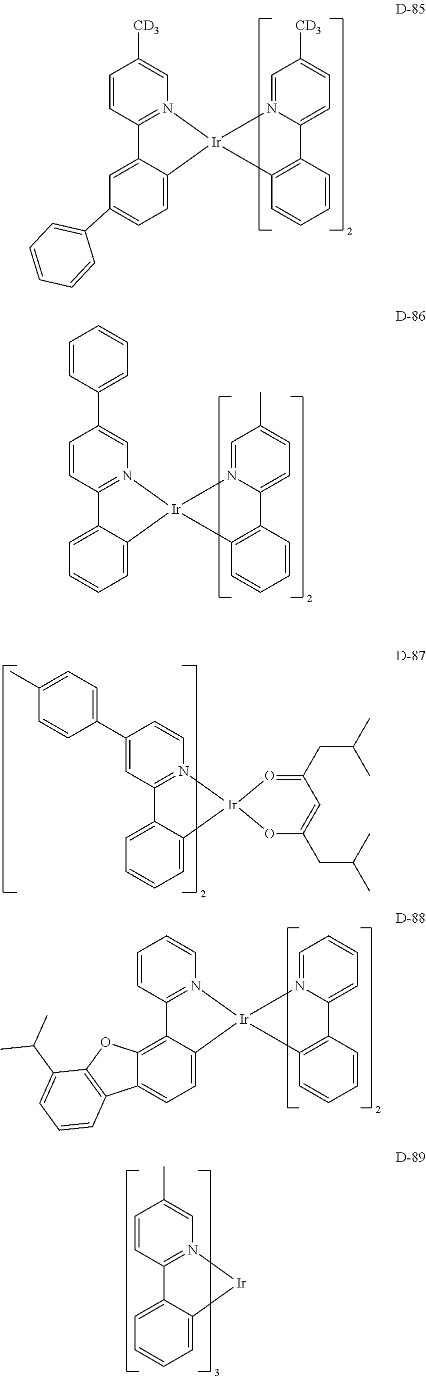

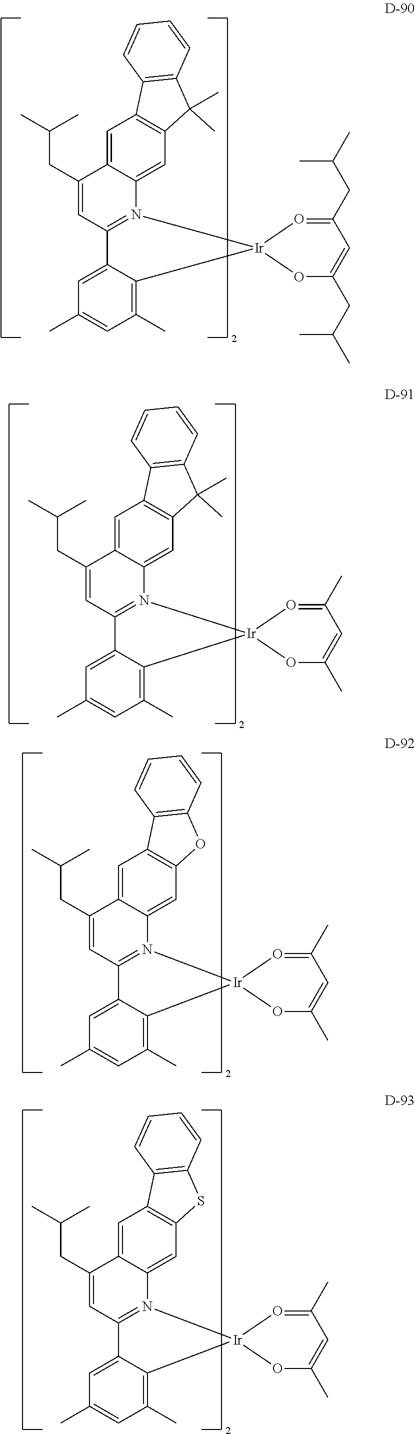

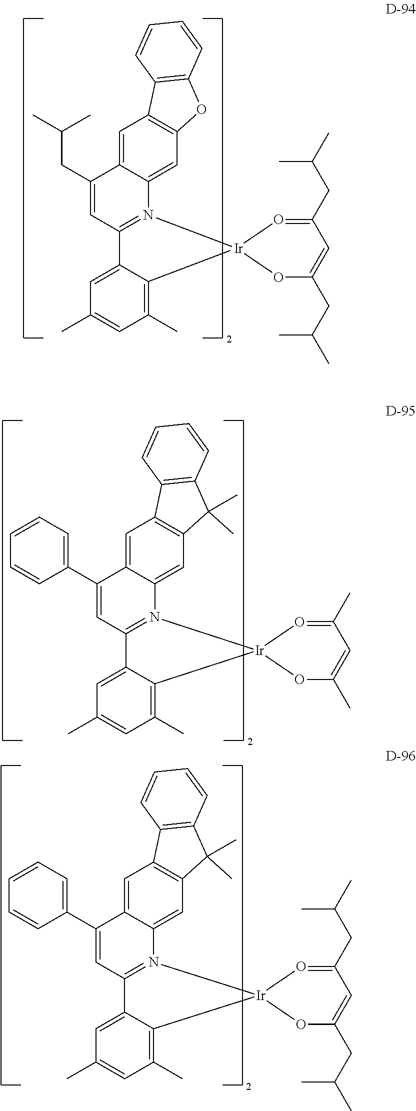

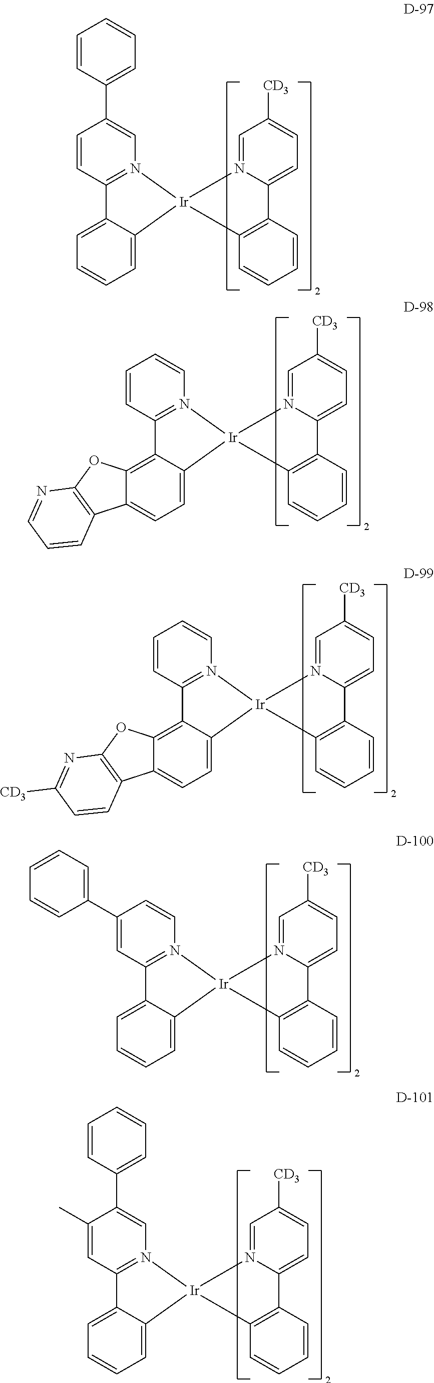

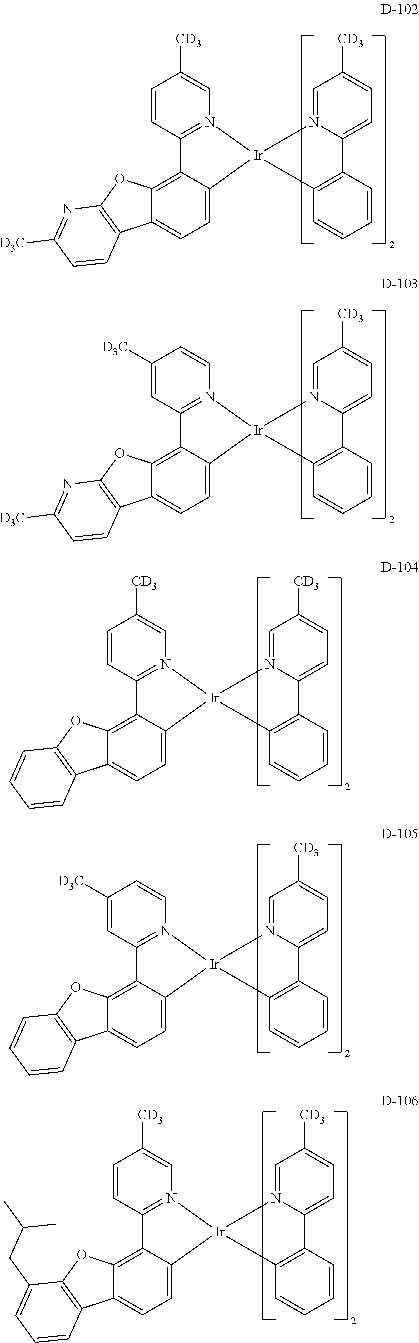

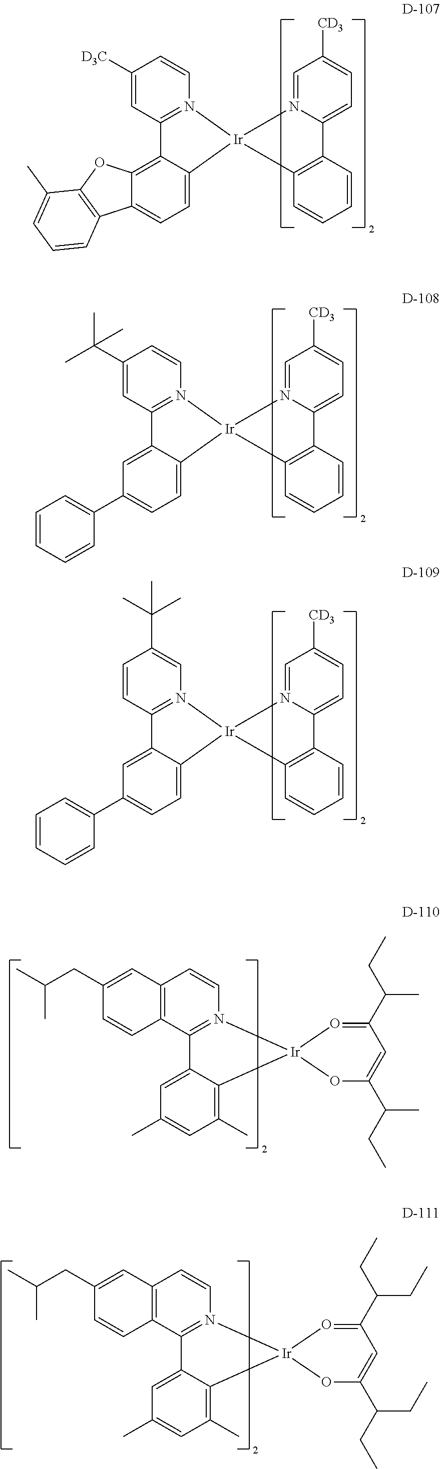

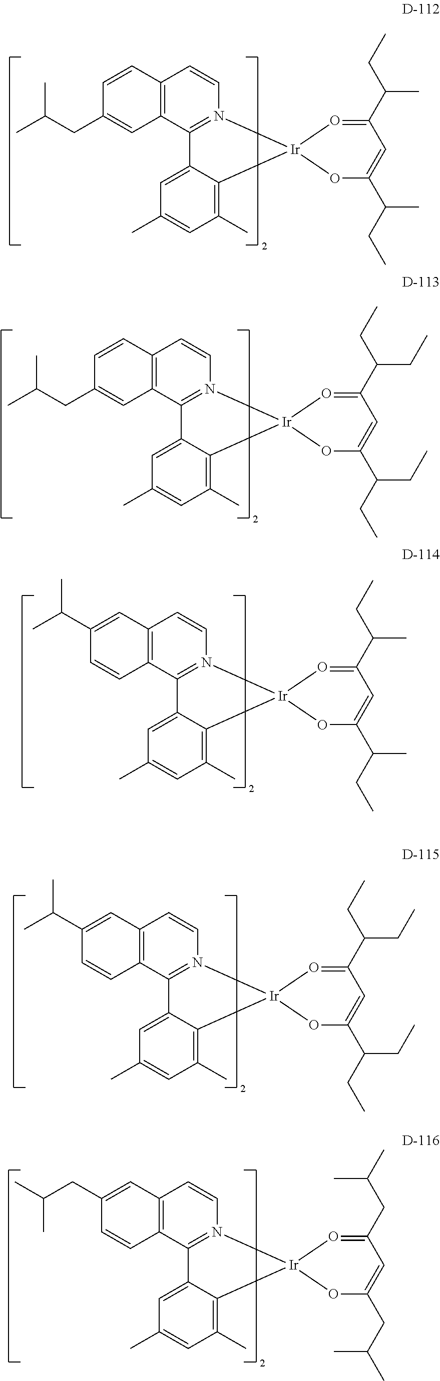

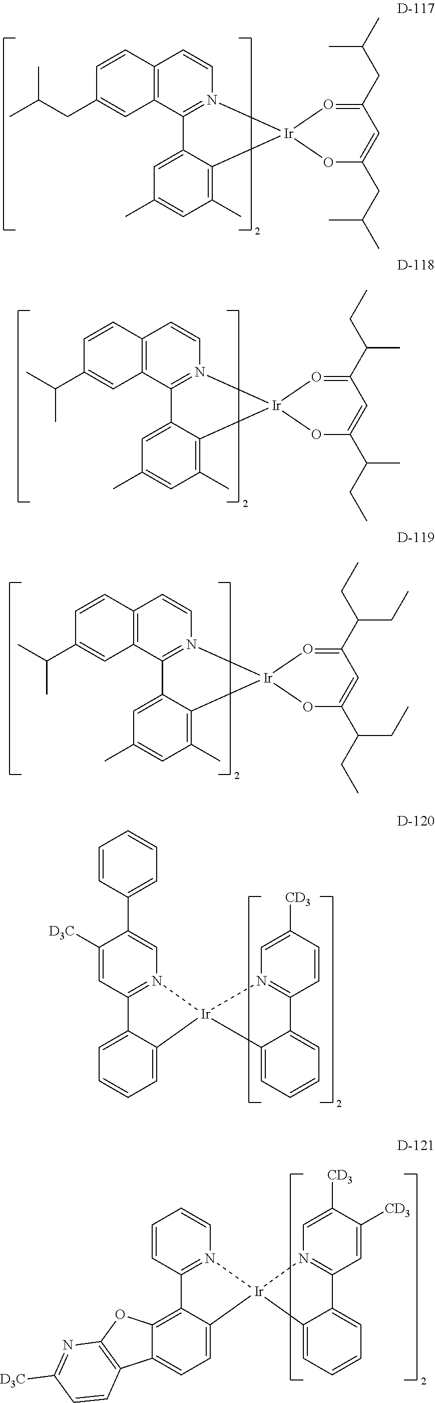

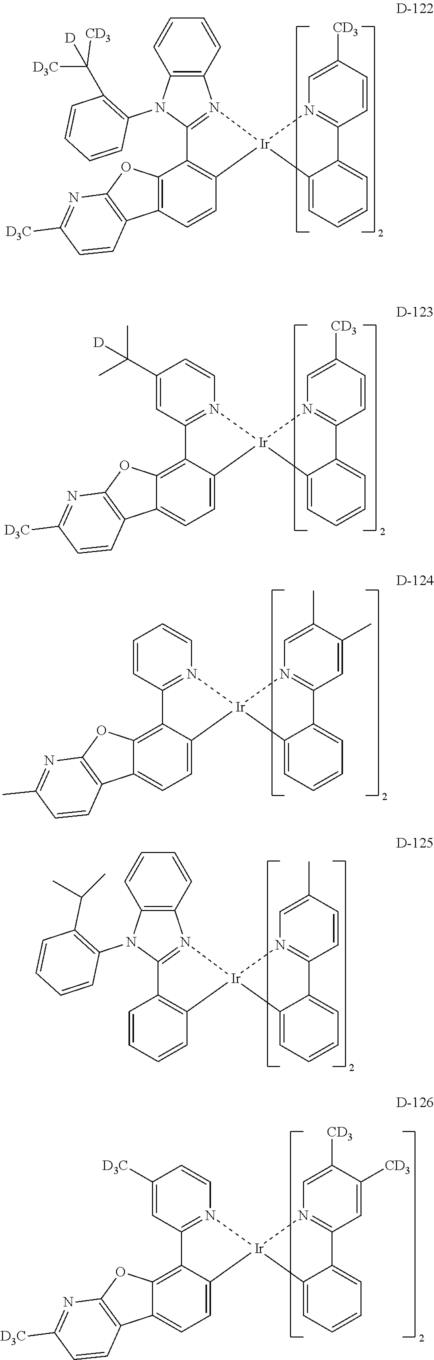

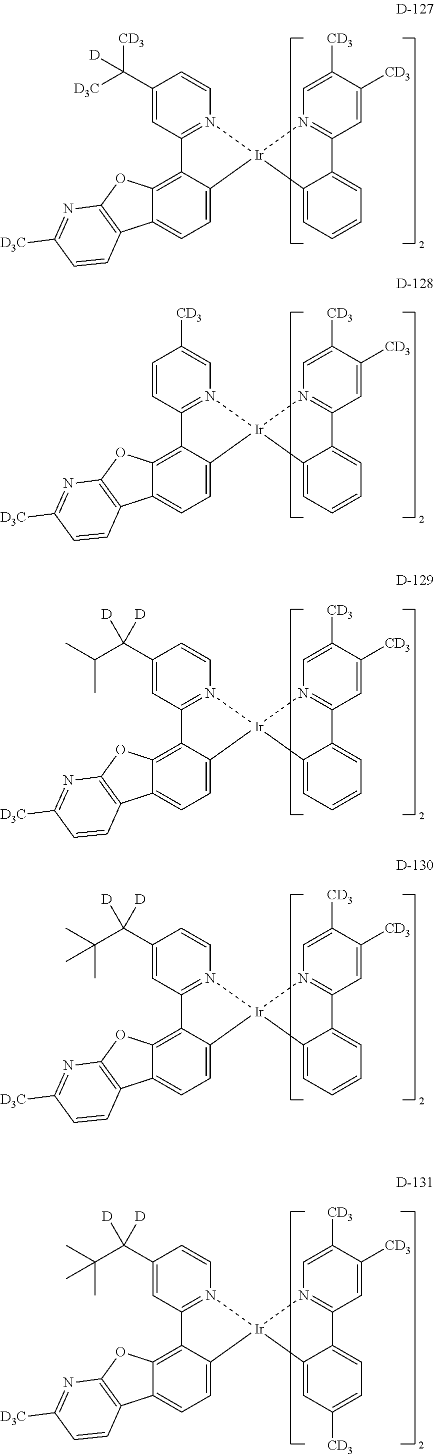

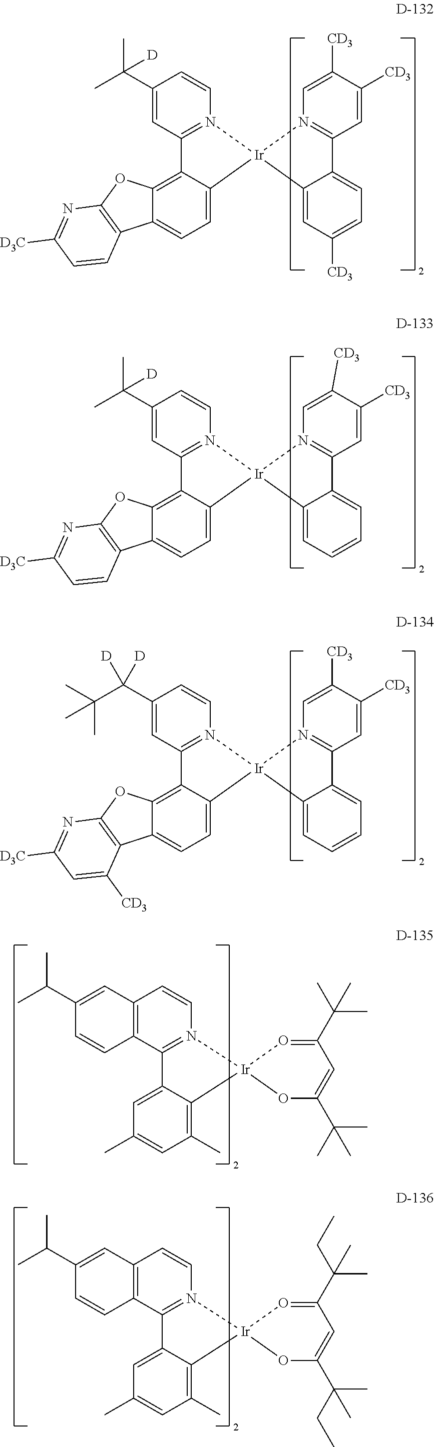

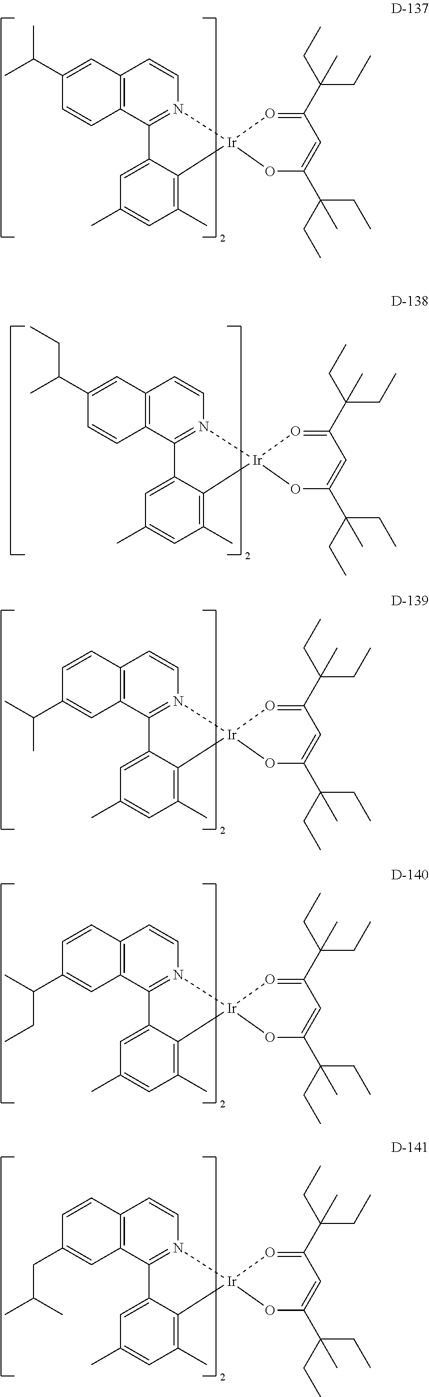

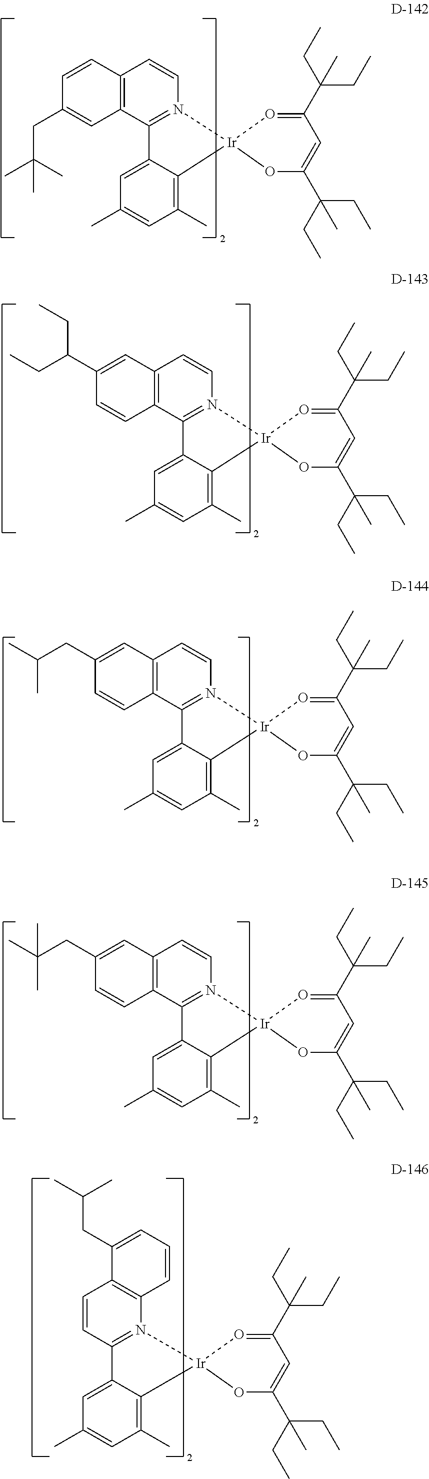

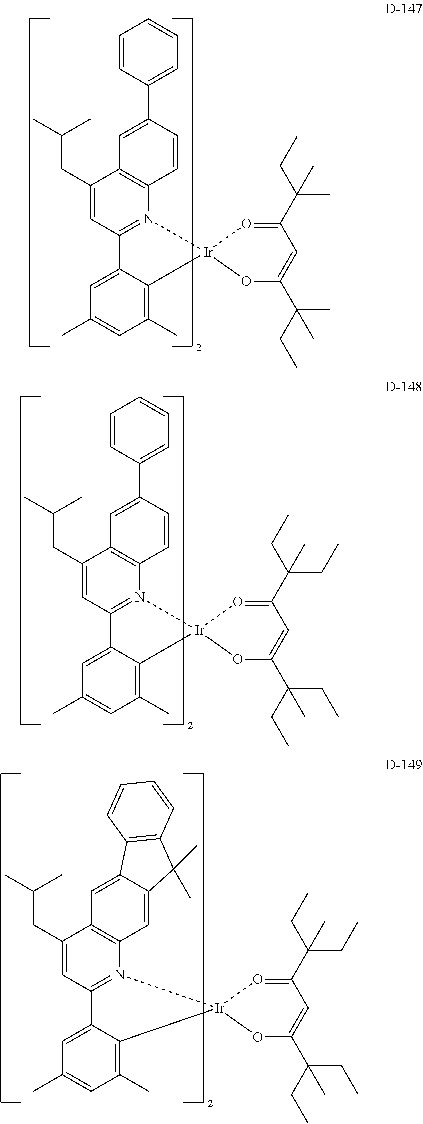

In another embodiment of the present disclosure, a composition for preparing an organic electroluminescent device is provided. The composition is preferably for preparing a light-emitting layer, a hole transport layer, a hole auxiliary layer, or a light-emitting auxiliary layer of an organic electroluminescent device and comprises the compound of the present disclosure. When there are two or more hole transport layers, the compound of the present disclosure may be comprised in the composition for preparing a hole transport layer (hole auxiliary layer) adjacent to the light-emitting layer.

An organic electroluminescent device according to the present disclosure comprises a cathode; an anode; and at least one organic layer between the cathode and the anode. The organic layer comprises a light-emitting layer, and may further comprise one or more layer selected from a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer. Each of the layers may further consist of multi-layers.

The cathode and the anode may each be formed with a transmissive conductive material, a transflective conductive material, or a reflective conductive material. The organic electroluminescent device may be a top emission type, a bottom emission type, or both-sides emission type according to the kinds of the material forming the cathode and the anode. In addition, the hole injection layer may be further doped with a p-dopant, and the electron injection layer may be further doped with an n-dopant.

The organic layer may further comprise at least one compound selected from the group consisting of arylamine-based compounds and styrylarylamine-based compounds.

In addition, in the organic electroluminescent device of the present disclosure, the organic layer may further comprise at least one metal selected from the group consisting of metals of Group 1, metals of Group 2, transition metals of period 4, transition metals of period 5, lanthanides and organic metals of d-transition elements of the Periodic Table, or at least one complex compound comprising said metal.

The organic electroluminescent device of the present disclosure may emit white light by further comprising at least one light-emitting layer comprising a blue, red or green light-emitting compound, which is known in the art. In addition, it may further comprise a yellow or orange light-emitting layer, if necessary.

In the organic electroluminescent device of the present disclosure, at least one layer selected from a chalcogenide layer, a metal halide layer and a metal oxide layer (hereinafter, “a surface layer”) may be preferably placed on an inner surface(s) of one or both electrodes. Specifically, a chalcogenide (including oxides) layer of silicon or aluminum is preferably placed on an anode surface of an electroluminescent medium layer, and a metal halide layer or a metal oxide layer is preferably placed on a cathode surface of an electroluminescent medium layer. Such a surface layer provides operation stability for the organic electroluminescent device. Preferably, the chalcogenide includes SiO_{X}(1≤X≤2), AlO_{X}(1≤X≤1.5), SiON, SiAlON, etc., the metal halide includes LiF, MgF₂, CaF₂, a rare earth metal fluoride, etc., and the metal oxide includes Cs₂O, Li₂O, MgO, SrO, BaO, CaO, etc.

A hole injection layer, a hole transport layer, an electron blocking layer, or a combination thereof can be used between the cathode and the light-emitting layer. The hole injection layer may be multi-layers in order to lower the hole injection barrier (or hole injection voltage) from the cathode to the hole transport layer or the electron blocking layer, wherein each of the multi-layers may use two compounds simultaneously. The hole transport layer or the electron blocking layer may also be multi-layers.

An electron buffer layer, a hole blocking layer, an electron transport layer, or an electron injection layer, or a combination thereof may be used between the light-emitting layer and the anode. The electron buffer layer may be multi-layers in order to control the injection of the electron and improve the interfacial properties between the light-emitting layer and the electron injection layer, wherein each of the multi-layers may use two compounds simultaneously. The hole blocking layer or the electron transport layer may also be multi-layers, wherein each of the multi-layers may use a plurality of compounds.

The light-emitting auxiliary layer may be placed between the cathode and the light-emitting layer, or between the anode and the light-emitting layer. When the light-emitting auxiliary layer is placed between the cathode and the light-emitting layer, it may be used for promoting the hole injection and/or hole transport, or for preventing the overflow of electrons. When the light-emitting auxiliary layer is placed between the anode and the light-emitting layer, it may be used for promoting the electron injection and/or electron transport, or for preventing the overflow of holes. Also, the hole auxiliary layer may be placed between the hole transport layer (or hole injection layer) and the light-emitting layer, and may be effective to promote or block the hole transport rate (or hole injection rate), thereby enabling the charge balance to be controlled. Further, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. When an organic electroluminescent device includes two or more hole transport layers, the hole transport layer, which is further included, may be used as a hole auxiliary layer or an electron blocking layer. The light-emitting auxiliary layer, the hole auxiliary layer or the electron blocking layer may have an effect of improving the efficiency and/or the lifetime of the organic electroluminescent device.

In addition, preferably, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to the light-emitting medium. Furthermore, the hole transport compound is oxidized to a cation, and thus it becomes easier to inject and transport holes from the mixed region to the light-emitting medium. Preferably, the oxidative dopant includes various Lewis acids and acceptor compounds; and the reductive dopant includes alkali metals, alkali metal compounds, alkaline earth metals, rare-earth metals, and mixtures thereof. The reductive dopant layer may be employed as a charge-generating layer to prepare an organic EL device having two or more light-emitting layers and emitting white light.

An organic electroluminescent material according to one embodiment of the present disclosure may be used as light-emitting materials for a white organic light-emitting device. The white organic light-emitting device has been suggested to have various structures such as a parallel side-by-side arrangement method, a stacking arrangement method, or CCM (color conversion material) method, etc., according to the arrangement of R (Red), G (Green) or YG (yellowish green), an B (blue) light-emitting units. In addition, the organic electroluminescent material according to one embodiment of the present disclosure may also be applied to the organic electroluminescent device comprising a QD (quantum dot).

In order to form each layer constituting the organic electroluminescent device of the present disclosure, dry film-forming methods such as vacuum deposition, sputtering, plasma, ion plating methods, etc., or wet film-forming methods such as ink jet printing, nozzle printing, slot coating, spin coating, dip coating, flow coating methods, etc., can be used.

When using a wet film-forming method, a thin film is formed by dissolving or dispersing the material constituting each layer in suitable solvents, such as ethanol, chloroform, tetrahydrofuran, dioxane, etc. The solvents are not specifically limited as long as the material constituting each layer is soluble or dispersible in the solvents, which do not cause any problems in the formation of a layer.

By using the organic electroluminescent device of the present disclosure, a display system, for example, for smartphones, tablets, notebooks, PCs, TVs, or vehicles, or a lighting system, for example, an indoor or outdoor lighting system, can be produced.

Hereinafter, the preparation method of the organic electroluminescent compounds of the present disclosure, the physical properties of the compounds, and the luminous properties of the organic electroluminescent device comprising the compounds will be explained in detail with reference to the representative compounds of the present disclosure. However, the present disclosure is not limited by the following examples.

### Example 1: Preparation of Compound C-30

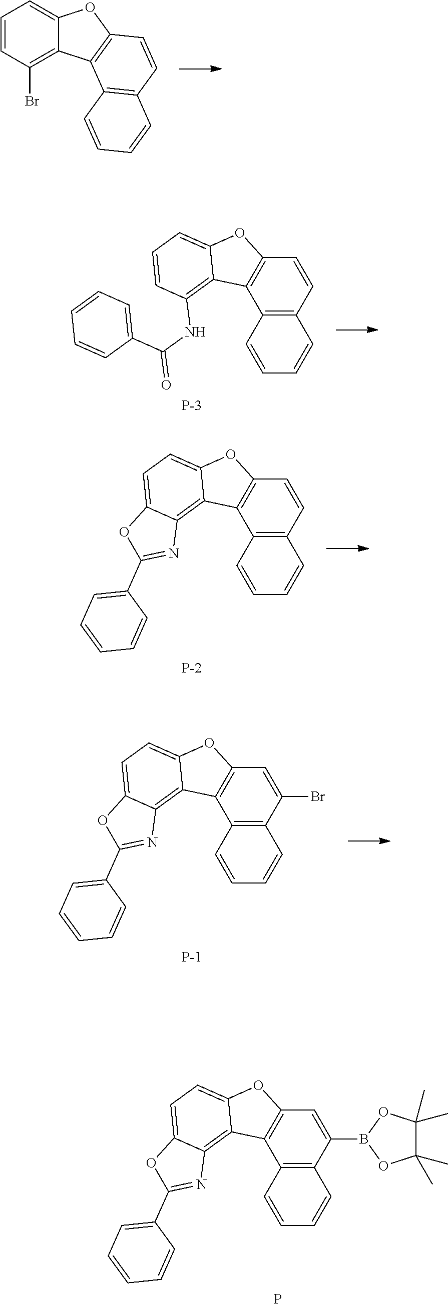

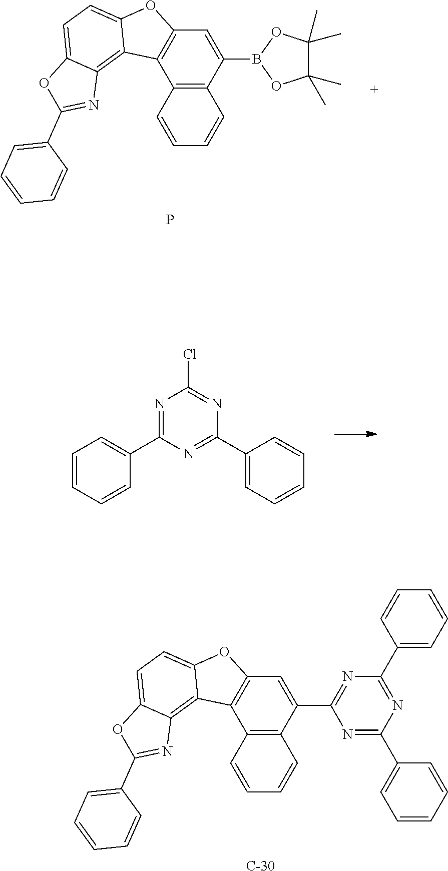

### Preparation of Compound P-3

11-bromonaphtho[2,1-b]benzofuran (33.5 g, 112.7 mmol), benzamide (13.6 g, 112.7 mmol), copper(I) iodide (32.2 g, 169 mmol), ethylenediamine (22.8 mL, 338.1 mmol) and tripotassium phosphate (47.8 g, 225.4 mmol) were added into a reaction vessel, and dissolved in 536 mL of o-xylene, and then stirred under refluxed for 24 hours. After cooling the mixture to room temperature and washing it with distilled water, an organic layer was extracted with ethyl acetate and dried with magnesium sulfate. Thereafter, the residue was separated by column chromatography to obtain Compound P-3 (23.5 g, yield: 61.8%).

### Preparation of Compound P-2

Compound P-3 (12 g, 35.5 mmol) and copper (II) triflate (2.57 g, 7.1 mmol) were added into a reaction vessel, and dissolved in 337 mL of dichlorobenzene, and then the mixture was stirred under refluxed for 48 hours. After removing the dichlorobenzene by distilled apparatus, the residue was separated by column chromatography to obtain Compound P-2 (4.7 g, yield: 39.4%).

### Preparation of Compound P-1

Compound P-2 (4.7 g, 14.0 mmol) and N-bromosuccinimide (2.62 g, 14.7 mmol) were added into a reaction vessel, and dissolved in 140 mL of dimethylformamide, and then stirred at room temperature for 24 hours. After completion of the reaction, an organic layer was extracted by introducing ethyl acetate and residual moisture was removed using magnesium sulfate. The residue was separated with column chromatography to obtain Compound P-1 (4.2 g, yield: 72%).

### Preparation of Compound P

Compound P-1 (4.1 g, 9.89 mmol), bis(pinacolato)diboron (3 g, 11.8 mmol), PdCl₂(PPh₃)₂ (0.347 g, 0.49 mmol) and potassium acetate (1.94 g, 19.7 mmol) were added into a reaction vessel, and dissolved in 66 mL of 1,4-dioxane, and then the mixture was refluxed at 130° C. for 5 hours. After completion of the reaction, the mixture was washed with distilled water and an organic layer was extracted by introducing ethyl acetate and residual moisture was removed using magnesium sulfate. The residue was separated with column chromatography to obtain Compound P (3.6 g, yield: 80%).

### Preparation of Compound C-30

Compound P (2.6 g, 5.63 mmol), 2-chloro-4,6-diphenyl-1,3,5-triazine (1.5 g, 5.6 mmol), Pd(PPh₃)₄ (0.323 g, 0.28 mmol) and potassium carbonate (2.3 g, 16.6 mmol) were added into a reaction vessel, and dissolved in a solvent mixture of 25.2 mL of toluene, 8.4 mL of ethanol and 8.4 mL of distilled water, and then the mixture was refluxed at 120° C. for 4 hours. After completion of the reaction, the mixture was washed with distilled water and an organic layer was extracted by introducing ethyl acetate and residual moisture was removed using magnesium sulfate. The residue was separated with column chromatography to obtain Compound C-30 (2.9 g, yield: 90%).

| Compound | MW | M.P. |
|---|---|---|
| C-30 | 566.62 | 327° C. |

### Example 2: Preparation of Compound C-171

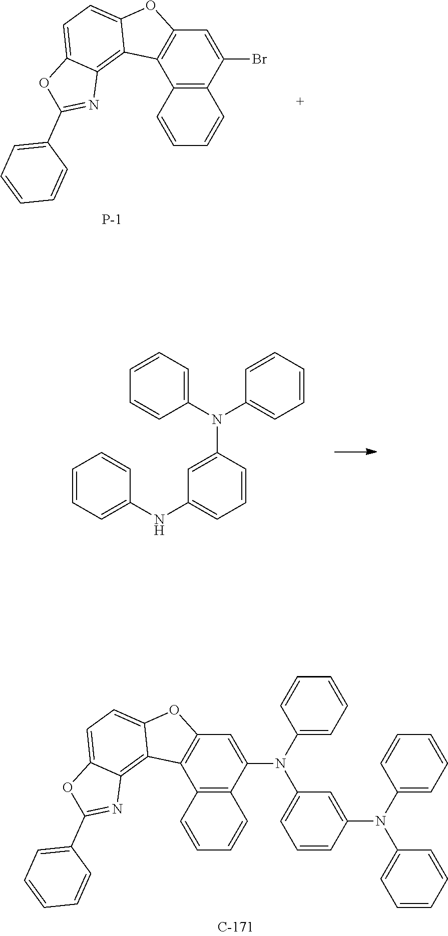

Compound P-1 (1.7 g, 4.1 mmol), N1,N1,N3-triphenylbenzene-1,3-diamine (1.25 g, 3.7 mmol), Pd₂(dba)₃ (0.169 g, 0.205 mmol), SPhos (0.152 g, 0.41 mmol) and sodium tert-butoxide (0.535 g, 6.15 mmol) were added into a reaction vessel, and dissolved in 20 mL of o-xylene, and then the mixture was refluxed at 180° C. for 1 hour. After completion of the reaction, the mixture was washed with distilled water and an organic layer was extracted by introducing ethyl acetate and residual moisture was removed using magnesium sulfate. The residue was separated with column chromatography to obtain Compound C-171 (2.6 g, yield: 96%).

| Compound | MW | M.P. |
|---|---|---|
| C-171 | 669.78 | 223.9° C. |

### Device Example 1: Producing an OLED Comprising the Organic Electroluminescent Compounds According to the Present Disclosure

An OLED of the present disclosure was produced as follows. First, a transparent electrode indium tin oxide (ITO) thin film (10 Ω/sq) on a glass substrate for an organic light-emitting diode (OLED) device (GEOMATEC CO., LTD., Japan) was subjected to an ultrasonic washing with acetone and isopropyl alcohol, sequentially, and was then stored in isopropyl alcohol. Next, the ITO substrate was mounted on a substrate holder of a vacuum vapor depositing apparatus. Compound HI-1 was introduced into a cell of said vacuum vapor depositing apparatus, and Compound HT-1 was introduced into another cell. The two materials were evaporated at different rates, and Compound HI-1 was deposited in a doping amount of 3 wt % based to the total amount of Compound HI-1 and Compound HT-1 to form a hole injection layer with a thickness of 10 nm. Subsequently, Compound HT-1 was deposited on the hole injection layer to form a first hole transport layer with a thickness of 80 nm. Next, Compound HT-2 was introduced into another cell of the vacuum vapor deposition apparatus and was evaporated by applying an electric current to the cell, thereby depositing a second hole transport layer with a thickness of 60 nm on the first hole transport layer. After forming the hole injection layer and the hole transport layers, a light-emitting layer was deposited thereon as follows:

The first host compound and the second host compound shown in Table 1 were introduced into one of two cells of the vacuum vapor depositing apparatus as hosts, and Compound D-39 was introduced into another cell as a dopant. The two host materials were evaporated at a rate of 1:1 and the dopant material was simultaneously evaporated at a different rate, and the dopant was doped in a doping amount of 3 wt % based on the total amount of the hosts and dopant to deposit a light-emitting layer with a thickness of 40 nm on the second hole transport layer. Next, Compound ETL-1 and Compound EIL-1 were deposited at a weight ratio of 50:50 to form an electron transport layer having a thickness of 35 nm on the light-emitting layer. After depositing Compound EIL-1 as an electron injection layer having a thickness of 2 nm on the electron transport layer, an Al cathode having a thickness of 80 nm was deposited on the electron injection layer by another vacuum vapor deposition apparatus. Thus, an OLED was produced. For each material, each compound used for producing the OLED was purified by vacuum sublimation at 10⁻⁶ torr.

### Comparative Example 1: Producing an OLED Comprising a Comparative Compound as a Host

An OLED was produced in the same manner as in Device Example 1, except that the second host compound shown in Table 1 was used as a second host of the light-emitting layer.

The driving voltage and light-emitting color at a luminance of 1,000 nit, and the time taken for luminance to reduce from 100% to 95% at a luminance of 10,000 nit (lifetime: T95) of the OLEDs produced in Example 1 and Comparative Example 1 are measured and provided in Table 1 below.

TABLE 1

| | | | Driving | Light- | Lifetime |
|---|---|---|---|---|---|
| | First | Second | Voltage | Emitting | T95 |
| | Host | Host | [V] | Color | [hr] |
| Device | H-195 | C-30 | 3.4 | Red | 117 |
| Example 1 | | | | | |
| Comparative | H-195 | A | 3.5 | Red | 61 |
| Example 1 | | | | | |

From Table 1 above, it can be confirmed that the OLED comprising the plurality of host materials according to the present disclosure (Device Example 1), exhibits significantly improved lifetime properties while having equivalent or higher driving voltages compared to the OLED not comprising the plurality of host materials according to the present disclosure (Comparative Example 1).

### Device Example 2 and Comparative Example 2: Producing an OLED Comprising the Organic Electroluminescent Compound According to the Present Disclosure or Comparative Compound as a Single Host

OLEDs were produced in the same manner as in Device Example 1, except that the host compound shown in Table 2 below was used alone as a host of the light-emitting layer.

The driving voltage, luminous efficiency, and light-emitting color at a luminance of 1,000 nit, and the time taken for luminance to reduce from 100% to 50% at 10,000 nit (lifetime: T50) of the OLEDs produced in Example 2 and the Comparative Example 2 are measured and provided in Table 2 below.

TABLE 2

| | | Driving | Luminous | Light- | Lifetime |
|---|---|---|---|---|---|
| | | Voltage | Efficiency | Emitting | T50 |
| | Host | [V] | [cd/A] | Color | [hr] |
| Device | C-30 | 4.3 | 21.4 | Red | 99 |
| Example 2 | | | | | |
| Comparative | A | 5.0 | 18.9 | Red | 58 |
| Example 2 | | | | | |

From Table 2 above, it can be confirmed that the OLED comprising the compound of the present disclosure as a host material (Device Example 2), exhibits significantly improved driving voltage, luminous efficiency and/or lifetime properties compared to the OLED comprising the comparative compound as a host material (Comparative Example 2).

The compounds used in the Device Examples and the Comparative Examples are shown in Table 3 below.

TABLE 3

| | |
|---|---|
| Hole Injection Layer/Hole Transport Layer | 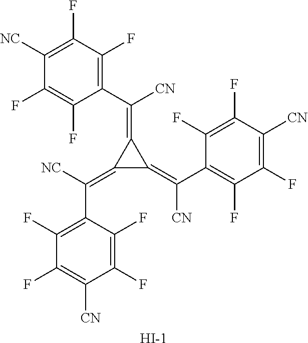 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 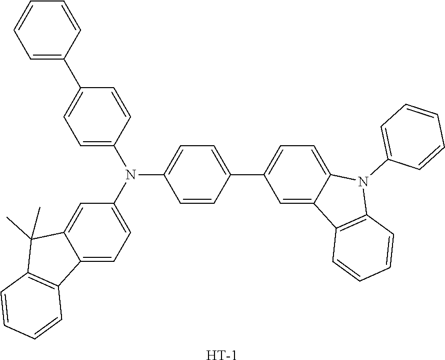 |
| | 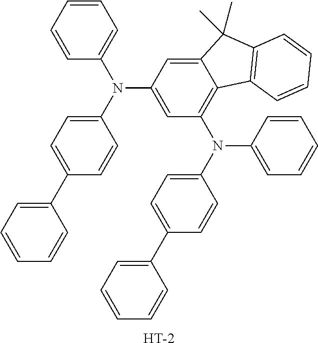 |
| Light- Emitting Layer | 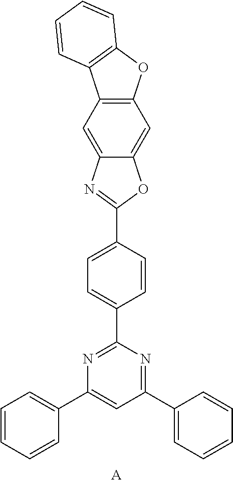 |
| | 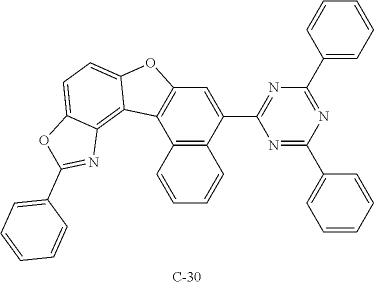 |
| | 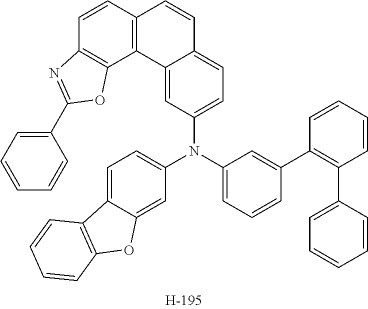 [SMILES: c1ccc(-c2nc3ccc4ccc5ccc(N(c6cccc(-c7ccccc7-c7ccccc7)c6)c6ccc7c(c6)oc6ccccc67)cc5c4c3o2)cc1] |
| | 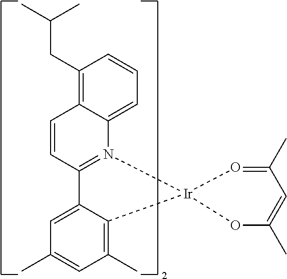 |
| | D-39 |
| Electron Transport Layer/ Electron Injection Layer | 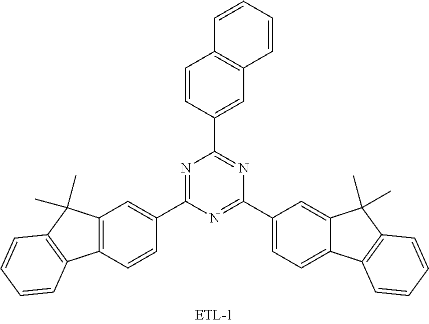 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 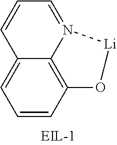 |

1. An organic electroluminescent compound represented by the following formula 1: 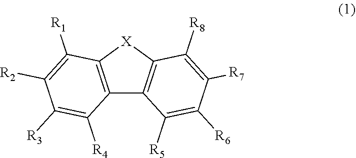
in Formula 1,
X represents O, S, or Se;
R₁ to R₈ each independently represent hydrogen, deuterium, a halogen, a cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, a substituted or unsubstituted (C1-C30)alkoxy, a substituted or unsubstituted amino, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30) arylamino, a substituted or unsubstituted (C2-C30)alkenyl(3- to 30-membered)heteroarylamino, or a substituted or unsubstituted (C6-C30)aryl(3- to 30-membered)heteroarylamino;
R₁ and R₂, R₂ and R₃, or R₃ and R₄ fuse with the following Formula 1-1 to form a ring(s), and R₅ and R₆, R₆ and R₇, or R₇ and R₈ fuse with the following formulas 1-2 or 1-3 to form a ring(s); 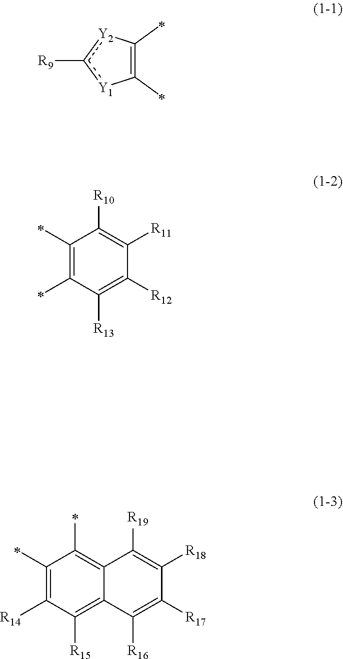
in formulas 1-1 to 1-3,
Y₁ and Y₂ each independently represent O, S, CR₂₀R₂₁, or NR₂₂;
R₉ to R₁₉ are each independently the same as the definition of R₁; and
* represents a site fused with Formula 1.

2. The organic electroluminescent compound according to claim 1, wherein Formula 1 is represented by any one of the following formulas 2-1 to 2-12: 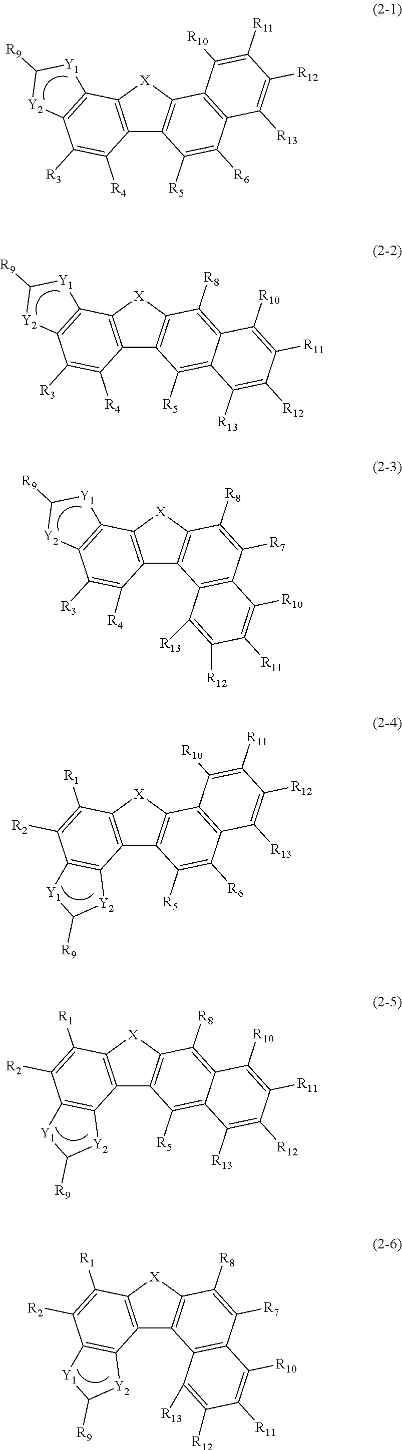 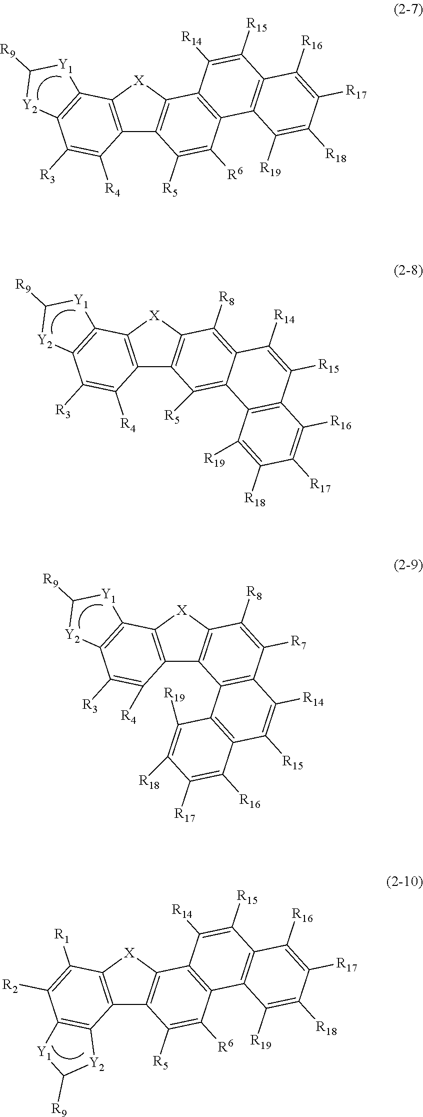 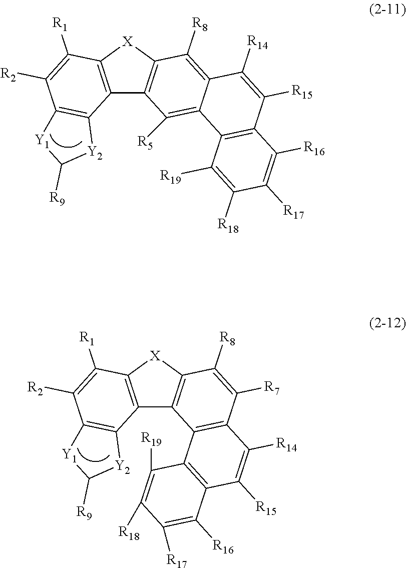
In formulas 2-1 to 2-11,
X, R₁ to R₁₉, Y₁, and Y₂ are as defined in claim 1.

3. The organic electroluminescent compound according to claim 1, wherein R₁ to R₁₉ each independently represent hydrogen, a substituted or unsubstituted phenyl, a substituted or unsubstituted naphthyl, a substituted or unsubstituted pyridyl, a substituted or unsubstituted pyrimidinyl, a substituted or unsubstituted triazinyl, a substituted or unsubstituted quinazolinyl, a substituted or unsubstituted quinoxalinyl, a substituted or unsubstituted (13-membered)heteroaryl, a substituted or unsubstituted benzoquinazolinyl, a substituted or unsubstituted benzoquinoxalinyl, a substituted or unsubstituted diphenylamino, a substituted or unsubstituted phenylnaphthylamino, a substituted or unsubstituted phenylbiphenylamino, a substituted or unsubstituted phenylterphenylamino, a substituted or unsubstituted phenylfluorenylamino, a substituted or unsubstituted phenyldibenzofuranylamino, a substituted or unsubstituted phenylcarbazolylamino, a substituted or unsubstituted naphthylbiphenylamino, a substituted or unsubstituted dibiphenylamino, a substituted or unsubstituted biphenyldibenzofuranylamino, or a substituted or unsubstituted biphenyldibenzothiophenylamino.

4. The organic electroluminescent compound according to claim 1, wherein the substituted alkyl, the substituted cycloalkyl, the substituted aryl, the substituted heteroaryl and the substituted alkoxy, each independently, are substituted with at least one selected from the group consisting of deuterium; a halogen; a cyano; a carboxyl; a nitro; a hydroxyl; a phosphine oxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl, a (C3-C30)cycloalkenyl; a (3- to 7-membered)heterocycloalkyl; a (C6-C30)aryloxy; a (C6-C30)arylthio; a (C6-C30)aryl unsubstituted or substituted with at least one of a (C1-C30) alkyl(s) and a di(C6-C30)arylamino(s); a (3- to 30-membered)heteroaryl unsubstituted or substituted with at least one of a (C6-C30)aryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; a fused ring group of a (C3-C30)aliphatic ring(s) and a (C6-C30)aromatic ring(s); an amino; a mono- or di-(C1-C30)alkylamino; a mono- or di-(C2-C30)alkenylamino; a mono- or di-(C6-C30)arylamino unsubstituted or substituted with a (C1-C30)alkyl(s); a mono- or di-(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a di(C6-C30)arylboronyl; a di(C1-C30) alkylboronyl; a (C1-C30)alkyl(C6-C30)arylboronyl; a (C6-C30)aryl(C1-C30)alkyl; and a (C1-C30)alkyl(C6-C30)aryl.

5. The organic electroluminescent compound according to claim 1, wherein the compound represented by Formula 1 is selected from the following compounds:

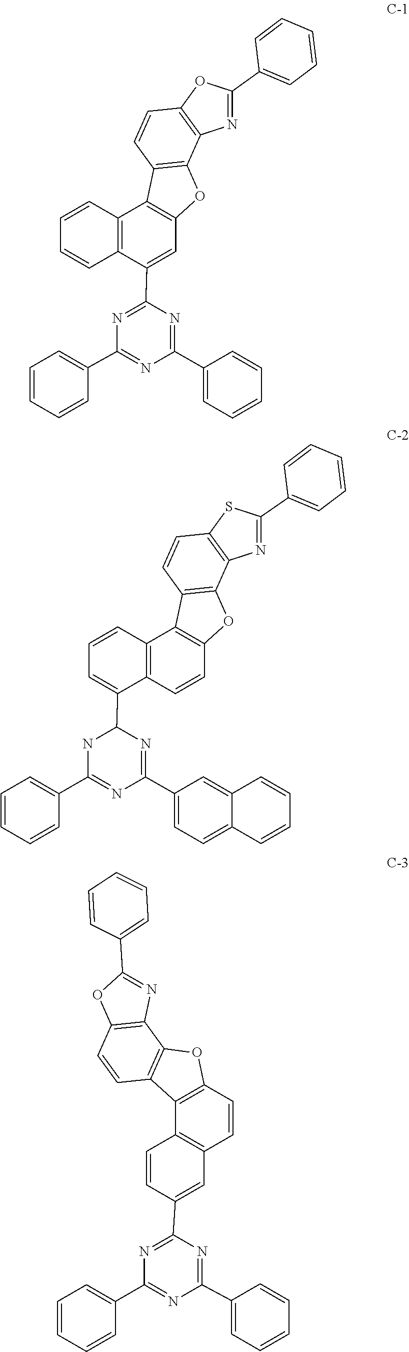

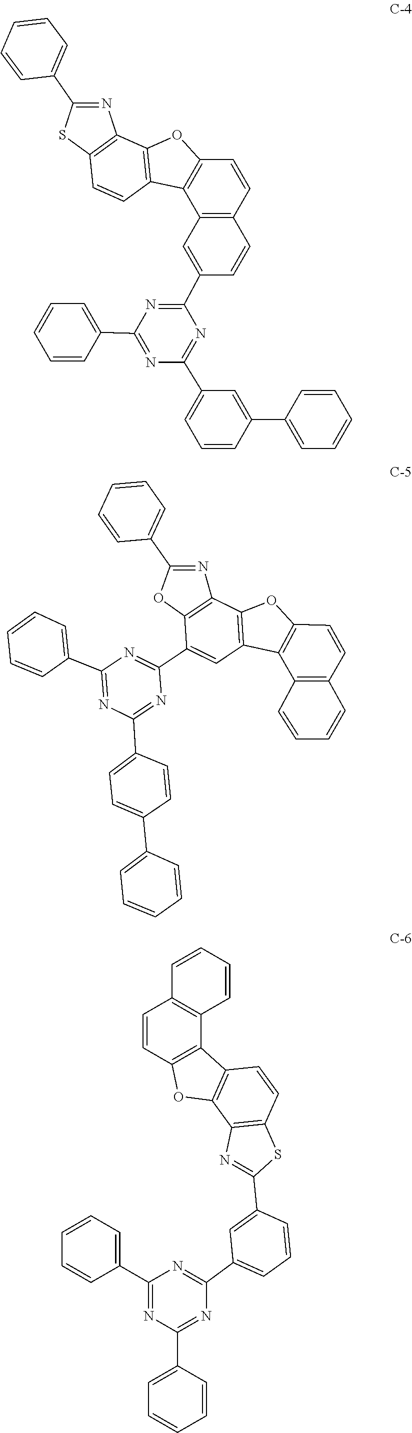 [SMILES: c1ccc(-c2cccc(-c3nc(-c4ccccc4)nc(-c4ccc5ccc6oc7c(ccc8sc(-c9ccccc9)nc87)c6c5c4)n3)c2)cc1 ; c1ccc(-c2ccc(-c3nc(-c4ccccc4)nc(-c4cc5c(oc6ccc7ccccc7c65)c5nc(-c6ccccc6)oc45)n3)cc2)cc1 ; c1ccc(-c2nc(-c3ccccc3)nc(-c3cccc(-c4nc5c(ccc6c5oc5ccc7ccccc7c56)s4)c3)n2)cc1]

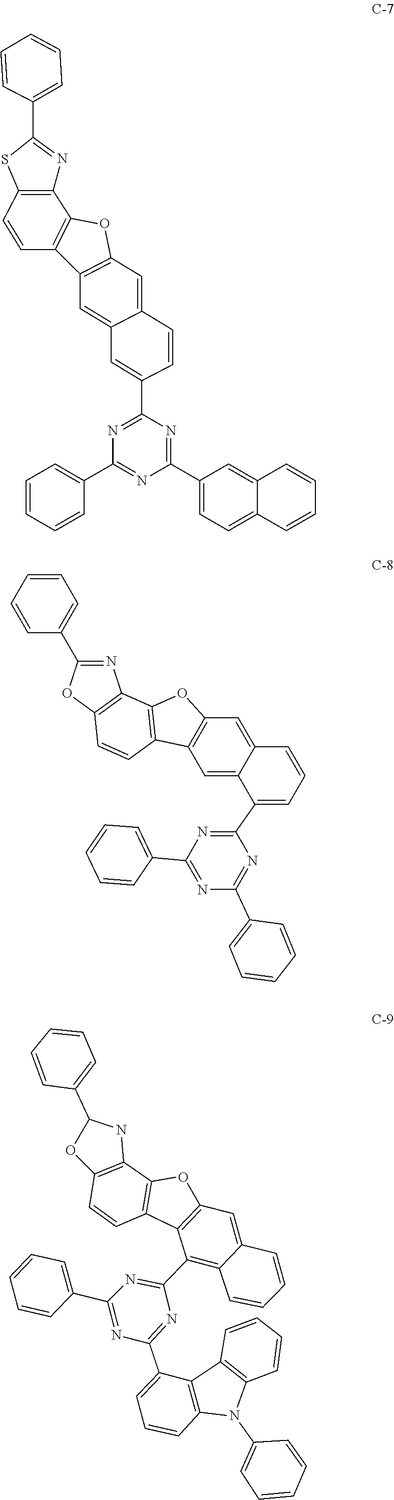

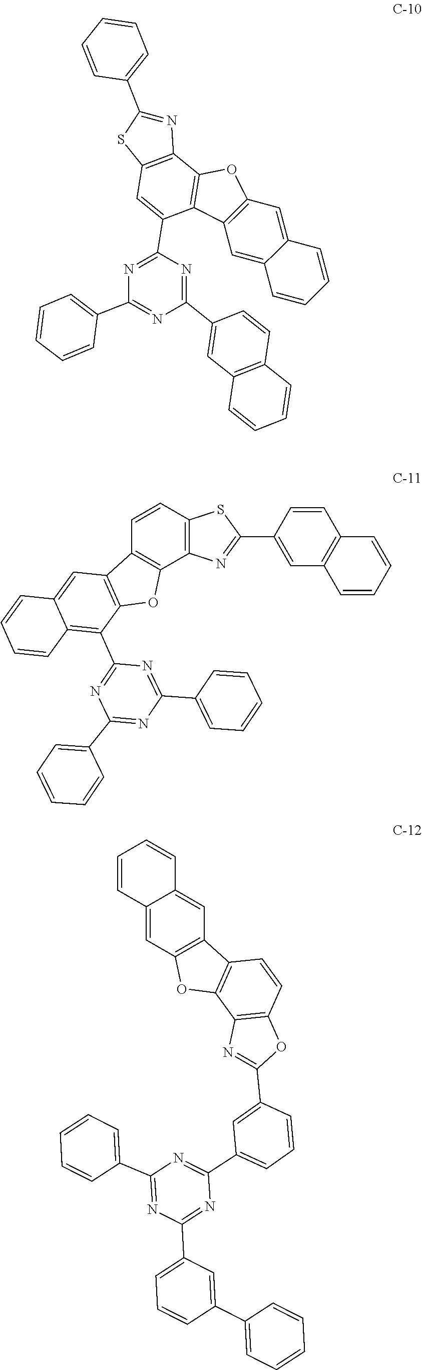 [SMILES: c1ccc(-c2nc(-c3ccc4ccccc4c3)nc(-c3cc4sc(-c5ccccc5)nc4c4oc5cc6ccccc6cc5c34)n2)cc1 ; c1ccc(-c2nc(-c3ccccc3)nc(-c3c4ccccc4cc4c3oc3c4ccc4sc(-c5ccc6ccccc6c5)nc43)n2)cc1 ; c1ccc(-c2cccc(-c3nc(-c4ccccc4)nc(-c4cccc(-c5nc6c(ccc7c8cc9ccccc9cc8oc76)o5)c4)n3)c2)cc1]

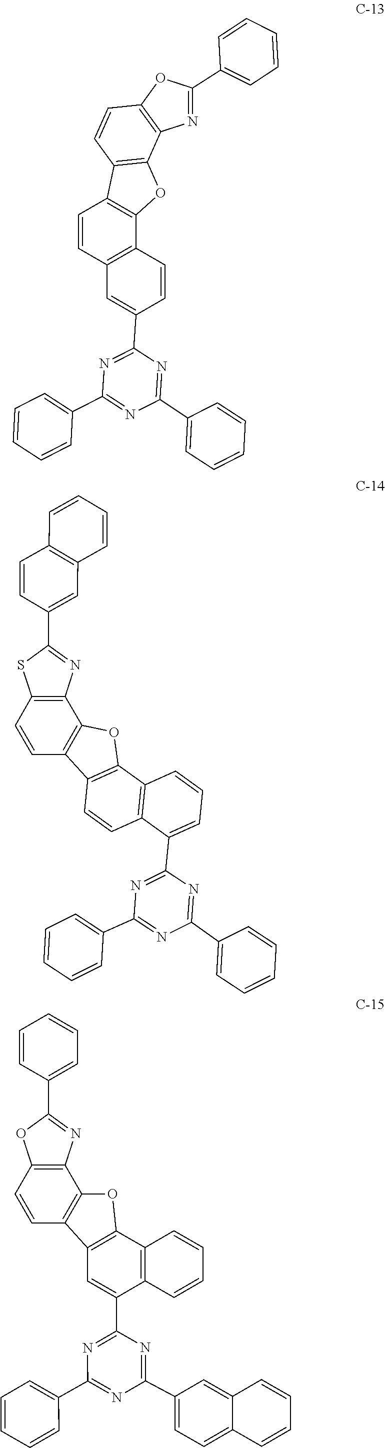

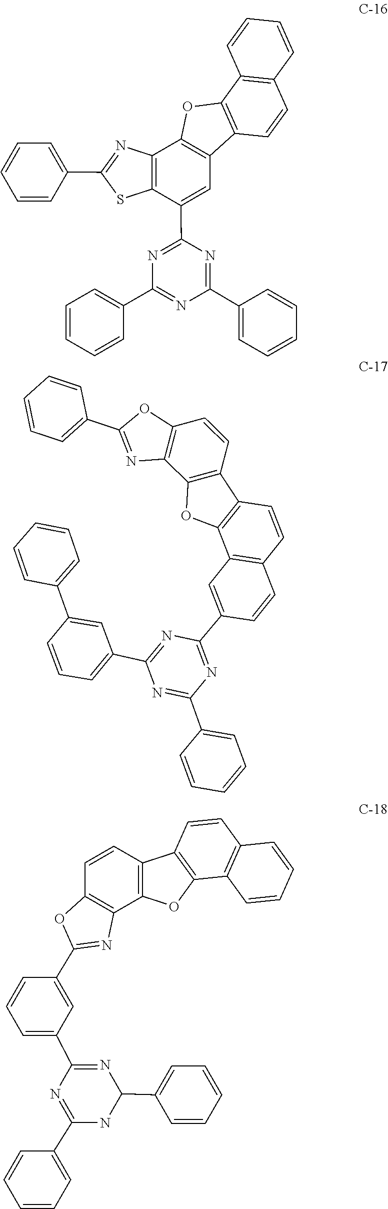

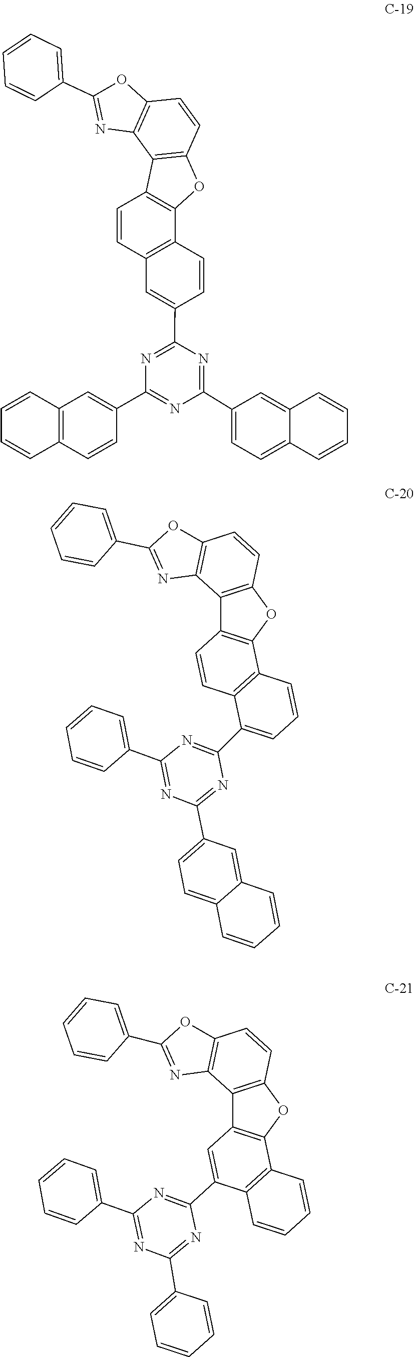 [SMILES: c1ccc(-c2nc3c(ccc4oc5c6ccc(-c7nc(-c8ccc9ccccc9c8)nc(-c8ccc9ccccc9c8)n7)cc6ccc5c43)o2)cc1 ; c1ccc(-c2nc(-c3ccc4ccccc4c3)nc(-c3cccc4c3ccc3c4oc4ccc5oc(-c6ccccc6)nc5c43)n2)cc1 ; c1ccc(-c2nc(-c3ccccc3)nc(-c3cc4c(oc5ccc6oc(-c7ccccc7)nc6c54)c4ccccc34)n2)cc1]

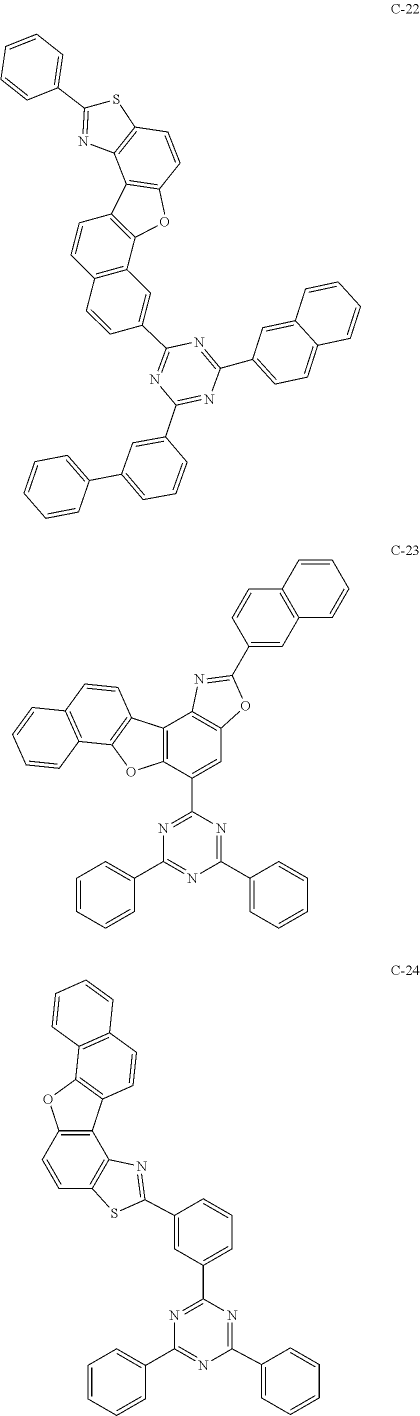 [SMILES: c1ccc(-c2cccc(-c3nc(-c4ccc5ccccc5c4)nc(-c4ccc5ccc6c(oc7ccc8sc(-c9ccccc9)nc8c76)c5c4)n3)c2)cc1 ; c1ccc(-c2nc(-c3ccccc3)nc(-c3cc4oc(-c5ccc6ccccc6c5)nc4c4c3oc3c5ccccc5ccc34)n2)cc1 ; c1ccc(-c2nc(-c3ccccc3)nc(-c3cccc(-c4nc5c(ccc6oc7c8ccccc8ccc7c65)s4)c3)n2)cc1]

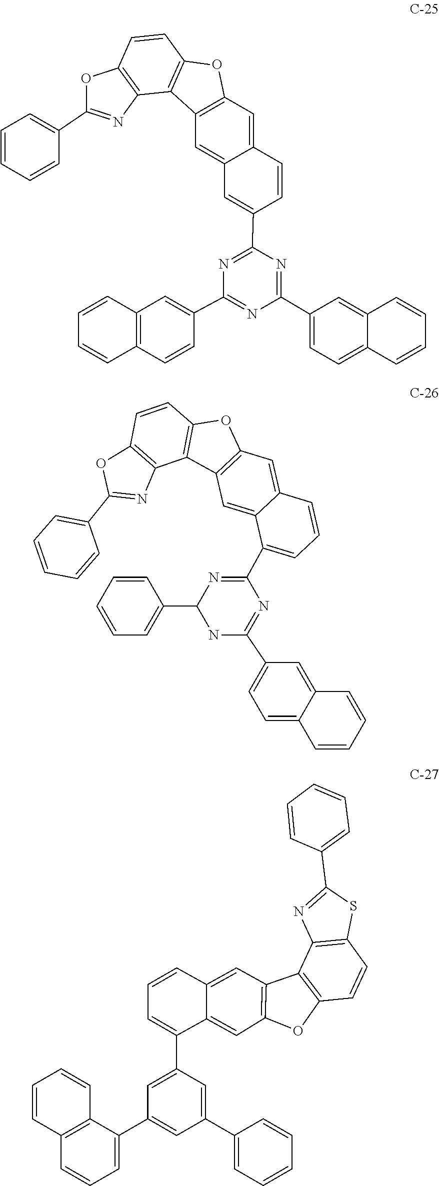

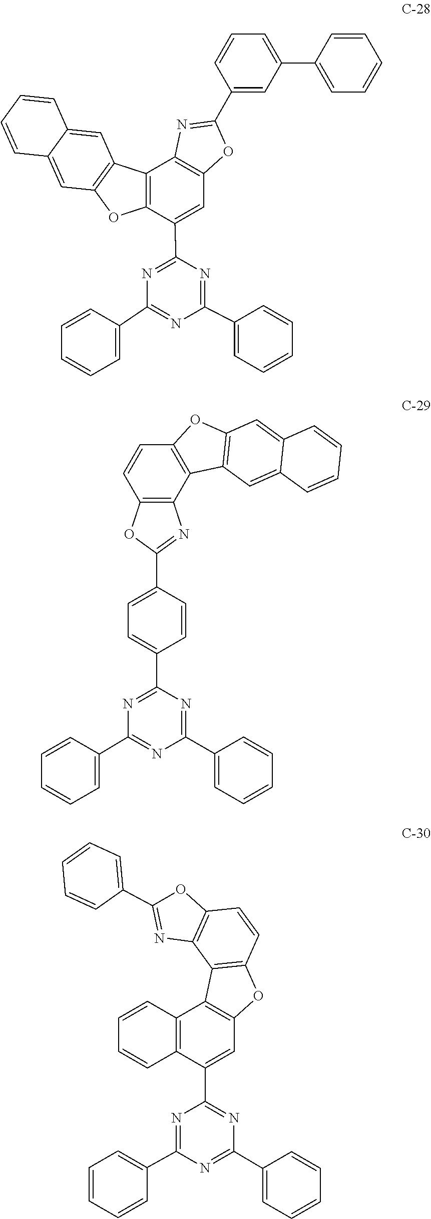

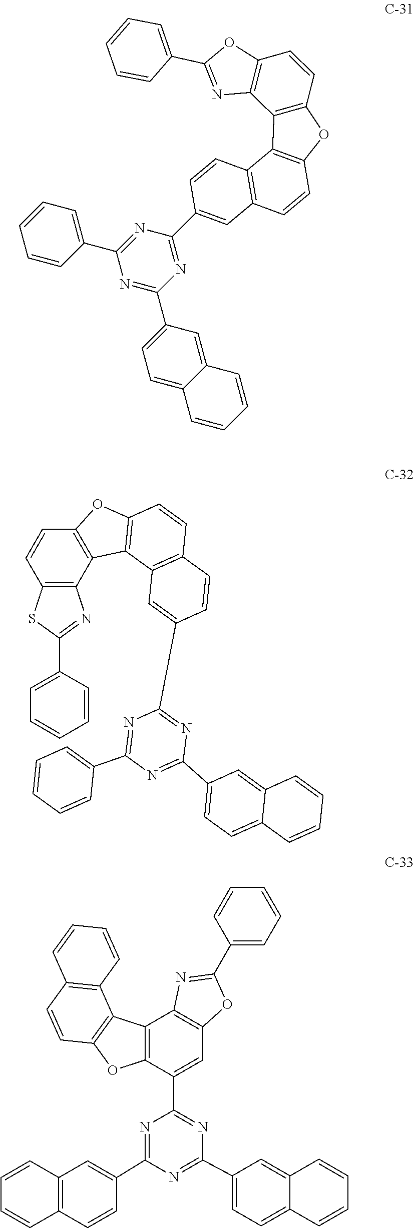 [SMILES: c1ccc(-c2nc(-c3ccc4ccccc4c3)nc(-c3ccc4c(ccc5oc6ccc7oc(-c8ccccc8)nc7c6c54)c3)n2)cc1 ; c1ccc(-c2nc(-c3ccc4ccccc4c3)nc(-c3ccc4ccc5oc6ccc7sc(-c8ccccc8)nc7c6c5c4c3)n2)cc1 ; c1ccc(-c2nc3c(cc(-c4nc(-c5ccc6ccccc6c5)nc(-c5ccc6ccccc6c5)n4)c4oc5ccc6ccccc6c5c43)o2)cc1]

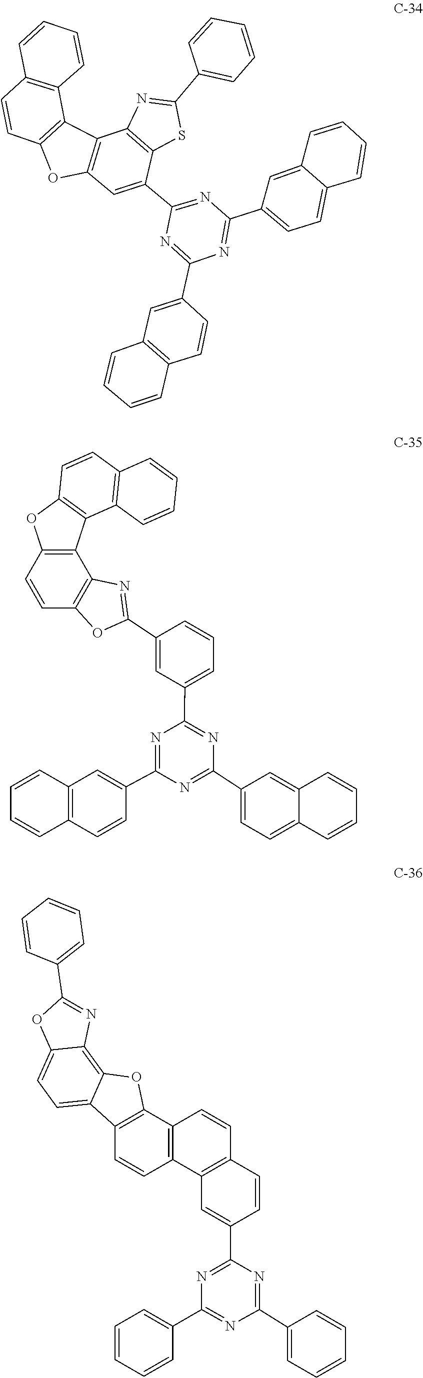 [SMILES: c1ccc(-c2nc3c(s2)c(-c2nc(-c4ccc5ccccc5c4)nc(-c4ccc5ccccc5c4)n2)cc2oc4ccc5ccccc5c4c23)cc1 ; c1cc(-c2nc(-c3ccc4ccccc4c3)nc(-c3ccc4ccccc4c3)n2)cc(-c2nc3c(ccc4oc5ccc6ccccc6c5c43)o2)c1 ; c1ccc(-c2nc(-c3ccccc3)nc(-c3ccc4ccc5c(ccc6c7ccc8oc(-c9ccccc9)nc8c7oc56)c4c3)n2)cc1]

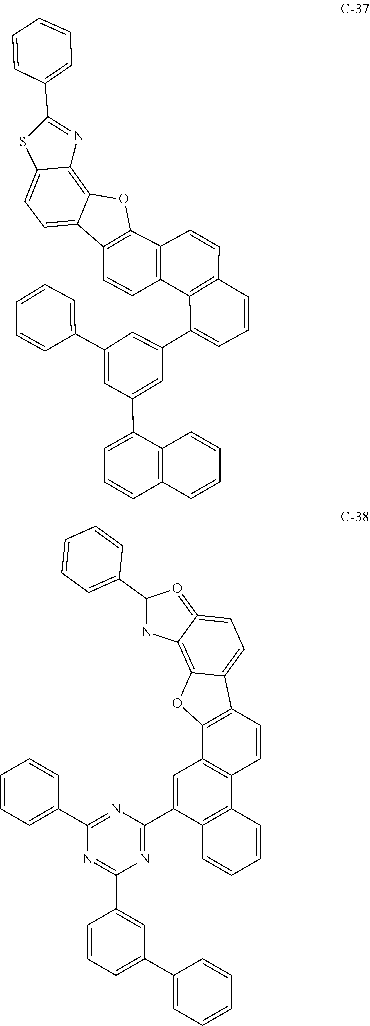

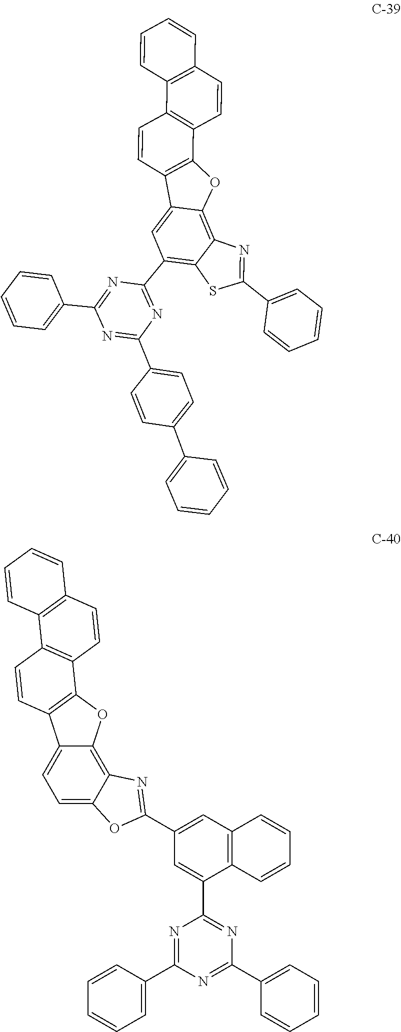 [SMILES: c1ccc(-c2ccc(-c3nc(-c4ccccc4)nc(-c4cc5c6ccc7c8ccccc8ccc7c6oc5c5nc(-c6ccccc6)sc45)n3)cc2)cc1 ; c1ccc(-c2nc(-c3ccccc3)nc(-c3cc(-c4nc5c(ccc6c7ccc8c9ccccc9ccc8c7oc65)o4)cc4ccccc34)n2)cc1]

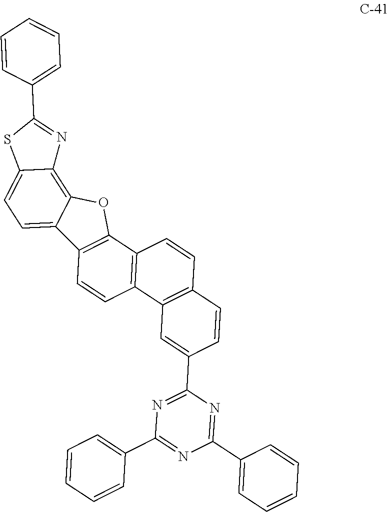 [SMILES: c1ccc(-c2nc(-c3ccccc3)nc(-c3ccc4ccc5c(ccc6c7ccc8sc(-c9ccccc9)nc8c7oc56)c4c3)n2)cc1]

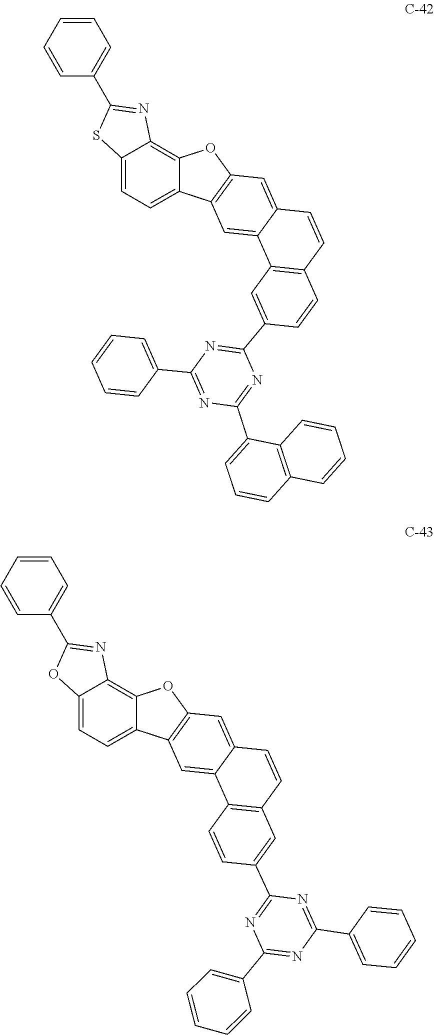 [SMILES: c1ccc(-c2nc(-c3ccc4ccc5cc6oc7c(ccc8sc(-c9ccccc9)nc87)c6cc5c4c3)nc(-c3cccc4ccccc34)n2)cc1 ; c1ccc(-c2nc(-c3ccccc3)nc(-c3ccc4c(ccc5cc6oc7c(ccc8oc(-c9ccccc9)nc87)c6cc54)c3)n2)cc1]

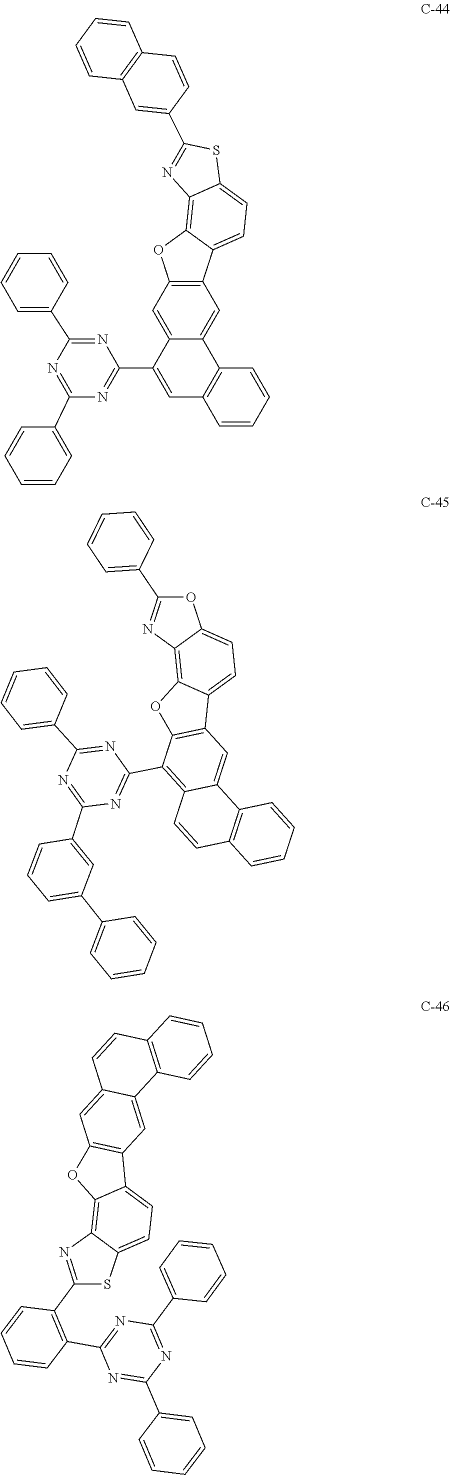 [SMILES: c1ccc(-c2nc(-c3ccccc3)nc(-c3cc4ccccc4c4cc5c(cc34)oc3c5ccc4sc(-c5ccc6ccccc6c5)nc43)n2)cc1 ; c1ccc(-c2cccc(-c3nc(-c4ccccc4)nc(-c4c5ccc6ccccc6c5cc5c4oc4c5ccc5oc(-c6ccccc6)nc54)n3)c2)cc1 ; c1ccc(-c2nc(-c3ccccc3)nc(-c3ccccc3-c3nc4c(ccc5c6cc7c(ccc8ccccc87)cc6oc54)s3)n2)cc1]

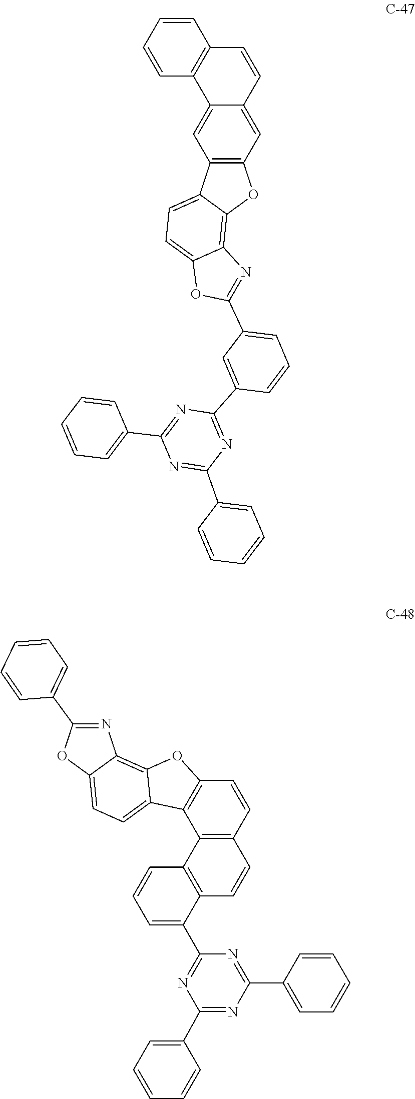 [SMILES: c1ccc(-c2nc(-c3ccccc3)nc(-c3cccc(-c4nc5c(ccc6c7cc8c(ccc9ccccc98)cc7oc65)o4)c3)n2)cc1 ; c1ccc(-c2nc(-c3ccccc3)nc(-c3cccc4c3ccc3ccc5oc6c(ccc7oc(-c8ccccc8)nc76)c5c34)n2)cc1]

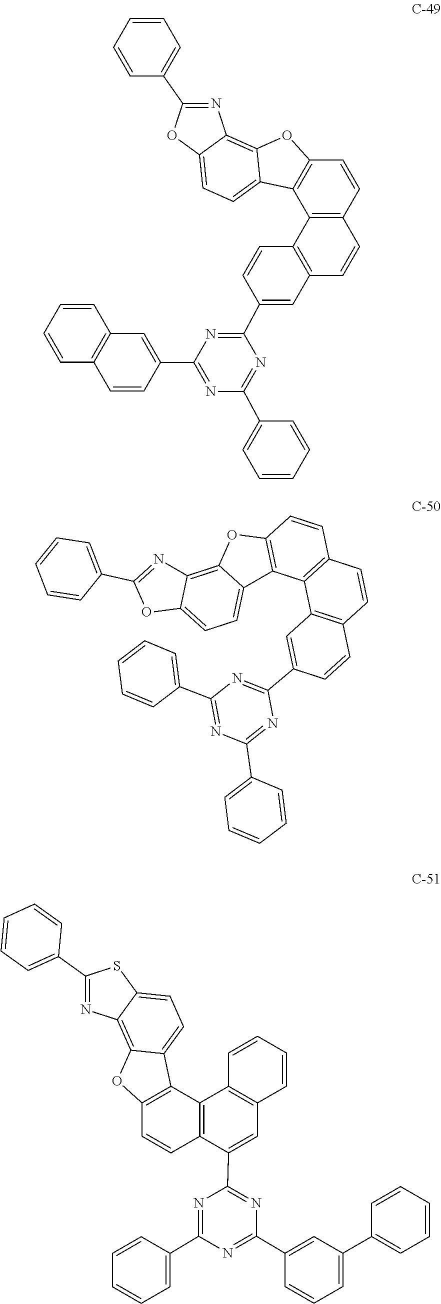

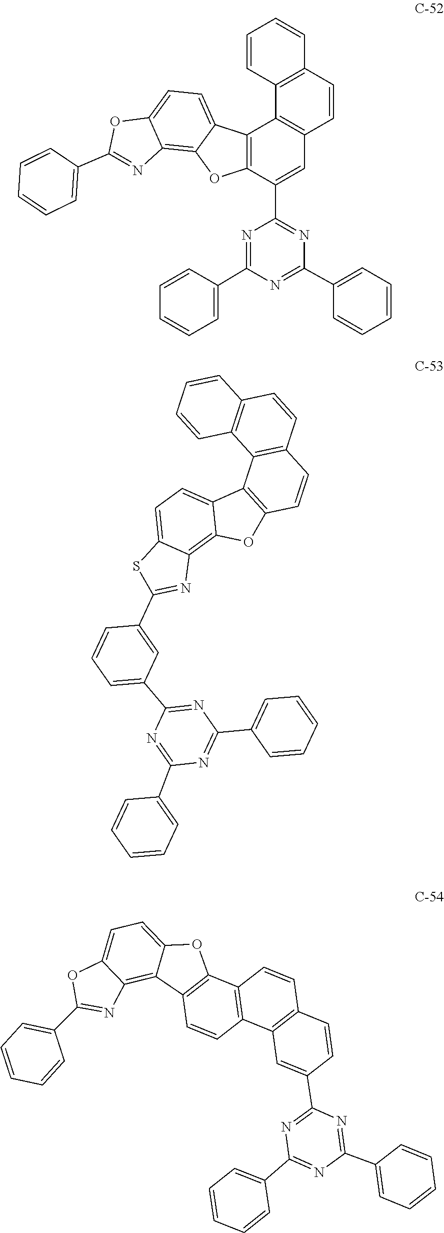 [SMILES: c1ccc(-c2nc(-c3ccccc3)nc(-c3cc4ccc5ccccc5c4c4c3oc3c4ccc4oc(-c5ccccc5)nc43)n2)cc1 ; c1ccc(-c2nc(-c3ccccc3)nc(-c3cccc(-c4nc5c(ccc6c5oc5ccc7ccc8ccccc8c7c56)s4)c3)n2)cc1 ; c1ccc(-c2nc(-c3ccccc3)nc(-c3ccc4ccc5c(ccc6c5oc5ccc7oc(-c8ccccc8)nc7c56)c4c3)n2)cc1]

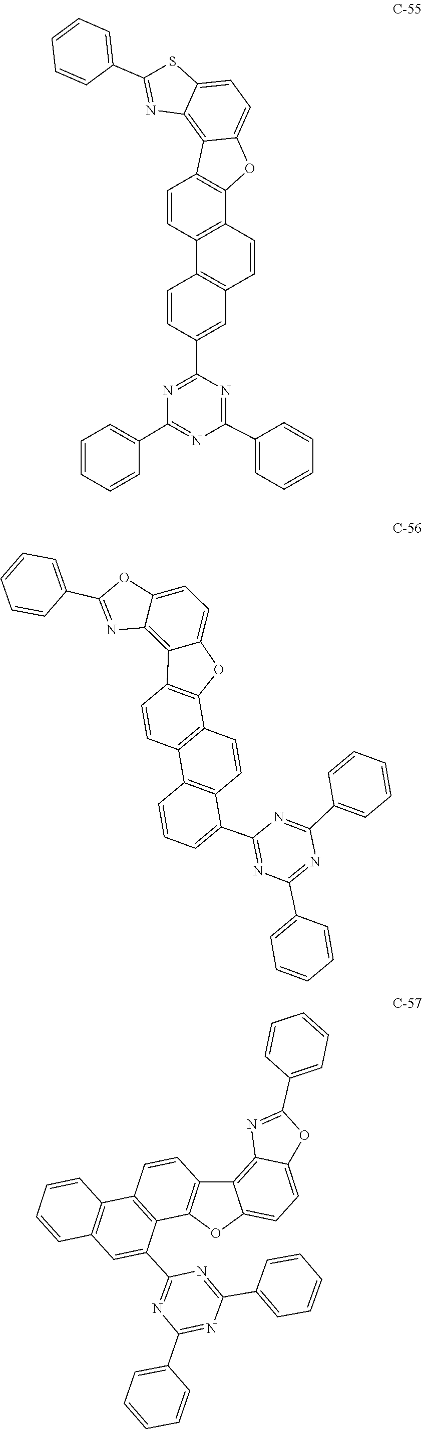

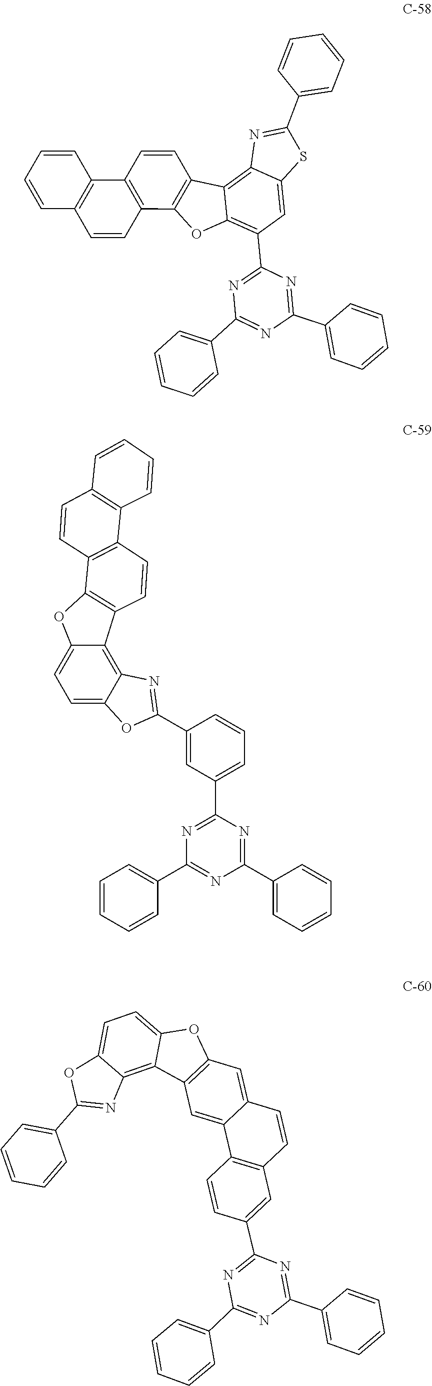 [SMILES: c1ccc(-c2nc(-c3ccccc3)nc(-c3cc4sc(-c5ccccc5)nc4c4c3oc3c5ccc6ccccc6c5ccc34)n2)cc1 ; c1ccc(-c2nc(-c3ccccc3)nc(-c3cccc(-c4nc5c(ccc6oc7c8ccc9ccccc9c8ccc7c65)o4)c3)n2)cc1 ; c1ccc(-c2nc(-c3ccccc3)nc(-c3ccc4c(ccc5cc6oc7ccc8oc(-c9ccccc9)nc8c7c6cc54)c3)n2)cc1]

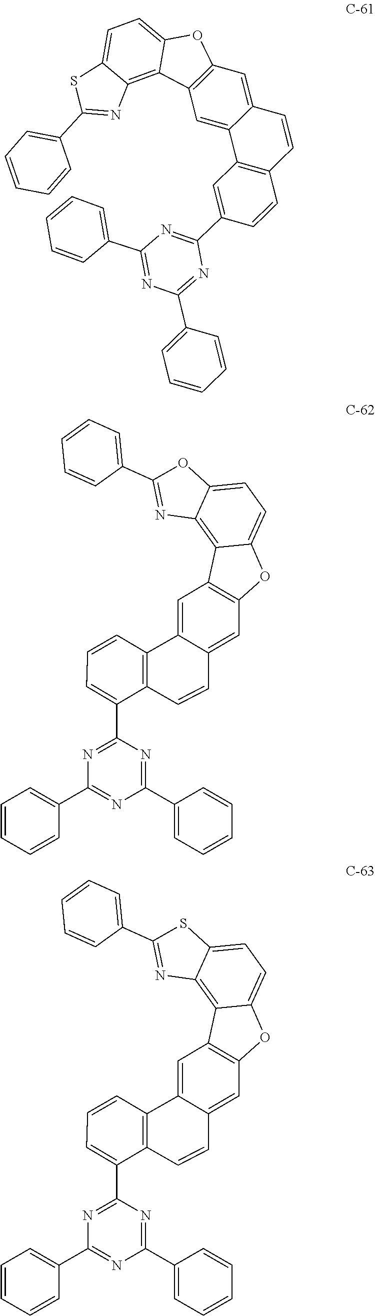 [SMILES: c1ccc(-c2nc(-c3ccccc3)nc(-c3ccc4ccc5cc6oc7ccc8sc(-c9ccccc9)nc8c7c6cc5c4c3)n2)cc1 ; c1ccc(-c2nc(-c3ccccc3)nc(-c3cccc4c3ccc3cc5oc6ccc7oc(-c8ccccc8)nc7c6c5cc34)n2)cc1 ; c1ccc(-c2nc(-c3ccccc3)nc(-c3cccc4c3ccc3cc5oc6ccc7sc(-c8ccccc8)nc7c6c5cc34)n2)cc1]

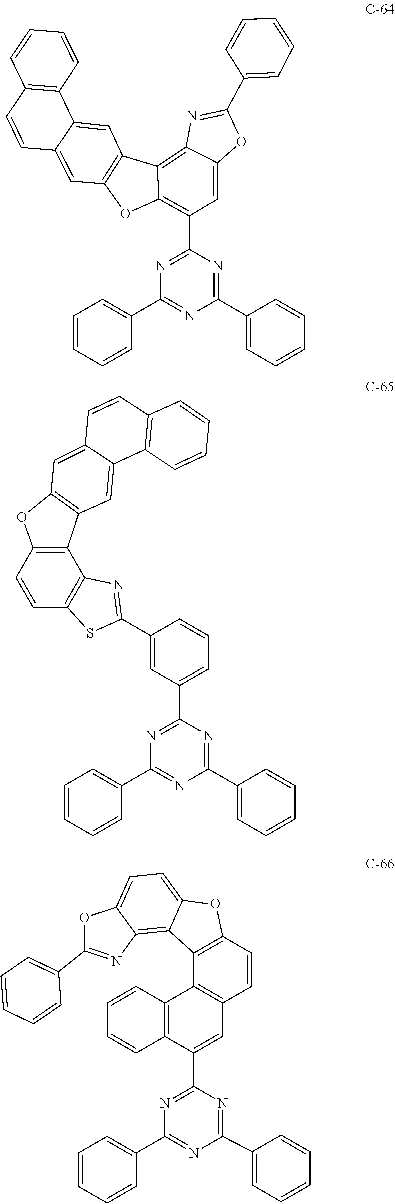

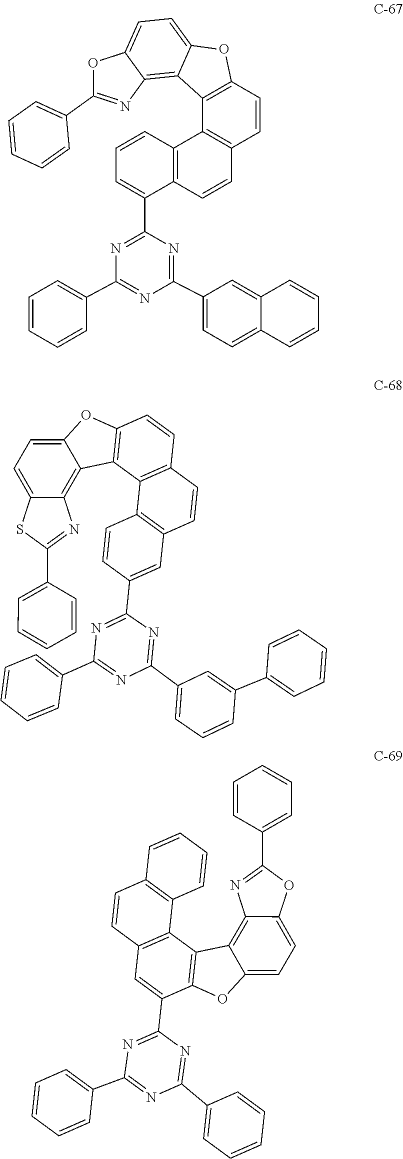 [SMILES: c1ccc(-c2nc(-c3ccc4ccccc4c3)nc(-c3cccc4c3ccc3ccc5oc6ccc7oc(-c8ccccc8)nc7c6c5c34)n2)cc1 ; c1ccc(-c2cccc(-c3nc(-c4ccccc4)nc(-c4ccc5c(ccc6ccc7oc8ccc9sc(-c%10ccccc%10)nc9c8c7c65)c4)n3)c2)cc1 ; c1ccc(-c2nc(-c3ccccc3)nc(-c3cc4ccc5ccccc5c4c4c3oc3ccc5oc(-c6ccccc6)nc5c34)n2)cc1]

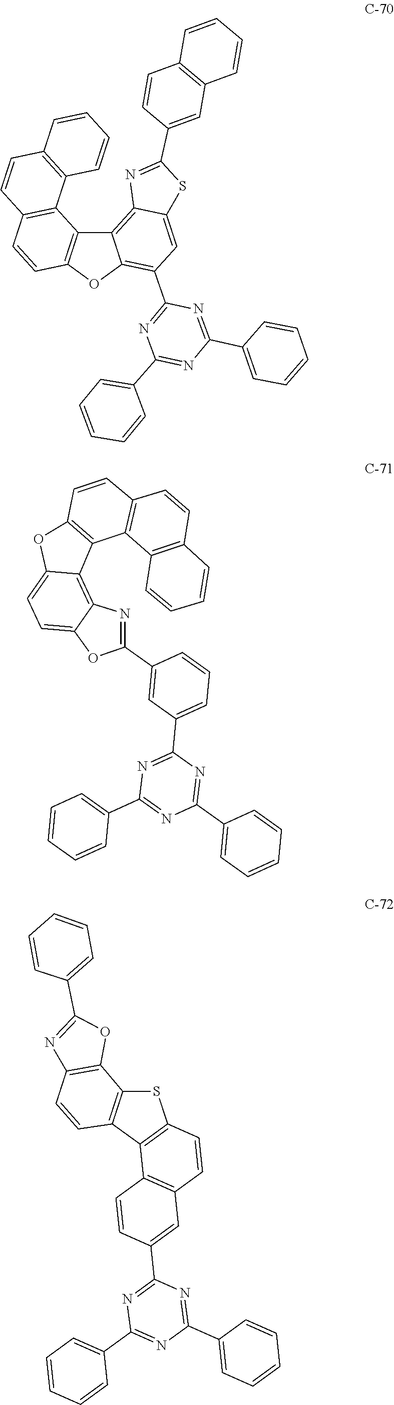

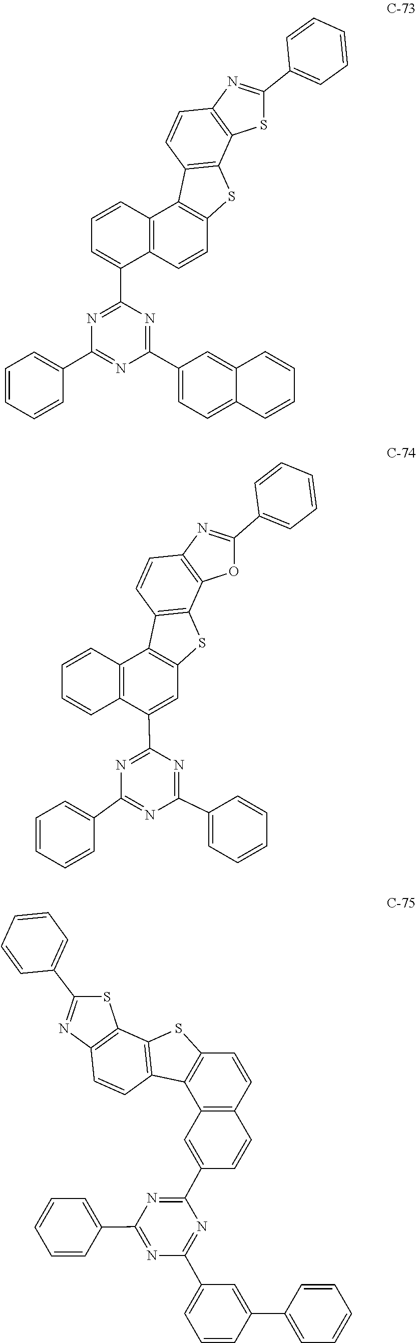 [SMILES: c1ccc(-c2nc(-c3ccc4ccccc4c3)nc(-c3cccc4c3ccc3sc5c(ccc6nc(-c7ccccc7)sc65)c34)n2)cc1 ; c1ccc(-c2nc(-c3ccccc3)nc(-c3cc4sc5c(ccc6nc(-c7ccccc7)oc65)c4c4ccccc34)n2)cc1 ; c1ccc(-c2cccc(-c3nc(-c4ccccc4)nc(-c4ccc5ccc6sc7c(ccc8nc(-c9ccccc9)sc87)c6c5c4)n3)c2)cc1]

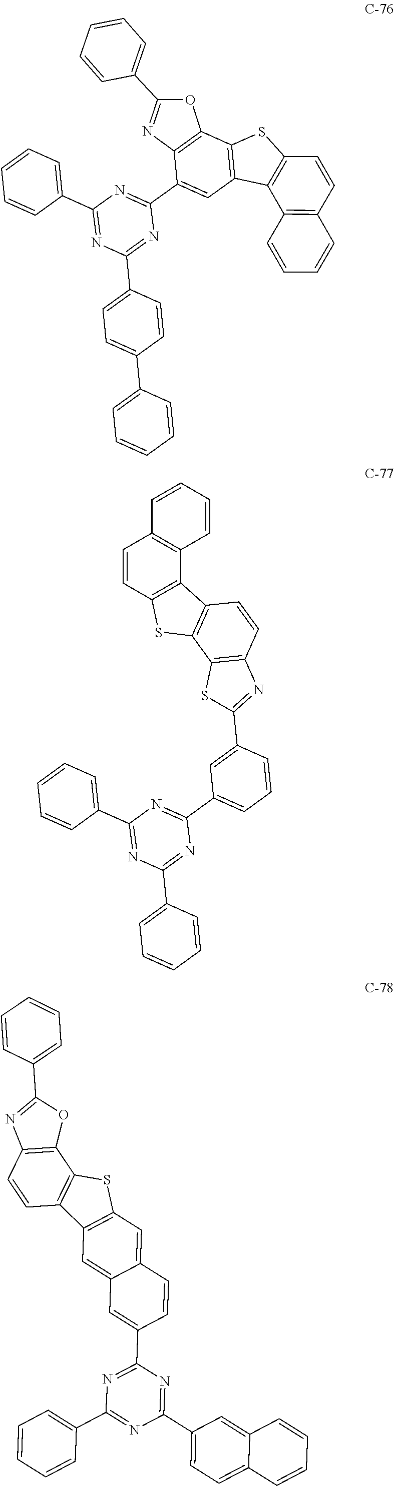

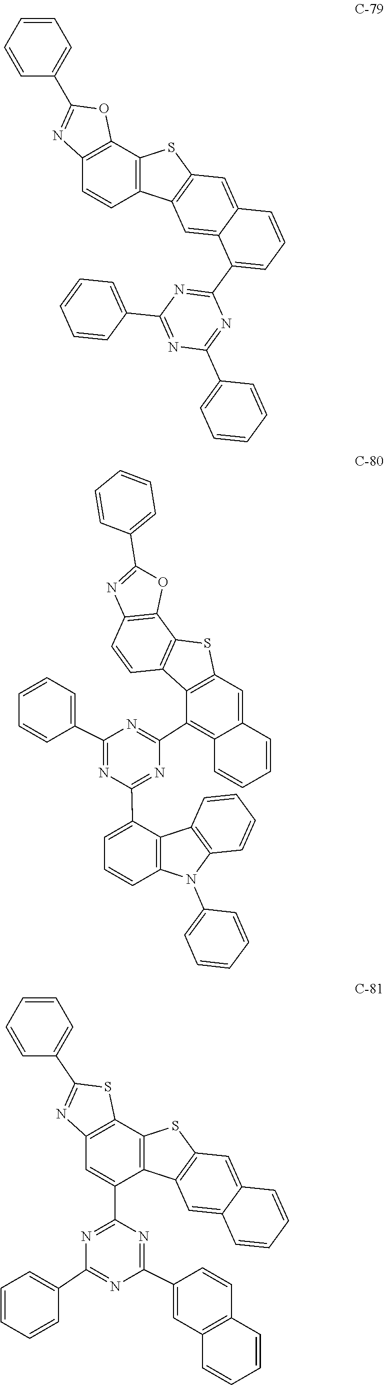 [SMILES: c1ccc(-c2nc(-c3ccccc3)nc(-c3cccc4cc5sc6c(ccc7nc(-c8ccccc8)oc76)c5cc34)n2)cc1 ; c1ccc(-c2nc(-c3c4ccccc4cc4sc5c(ccc6nc(-c7ccccc7)oc65)c34)nc(-c3cccc4c3c3ccccc3n4-c3ccccc3)n2)cc1 ; c1ccc(-c2nc(-c3ccc4ccccc4c3)nc(-c3cc4nc(-c5ccccc5)sc4c4sc5cc6ccccc6cc5c34)n2)cc1]

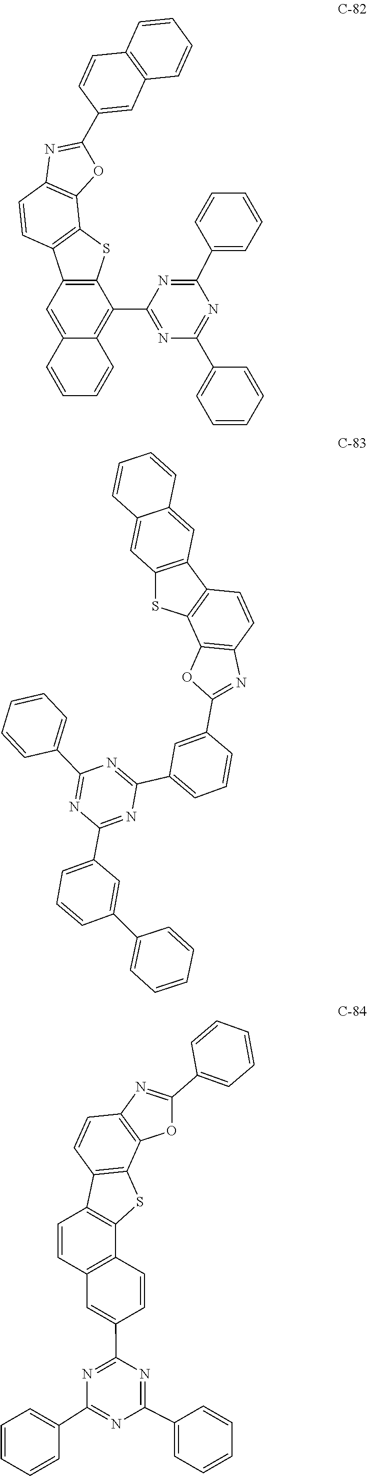 [SMILES: c1ccc(-c2nc(-c3ccccc3)nc(-c3c4ccccc4cc4c3sc3c4ccc4nc(-c5ccc6ccccc6c5)oc43)n2)cc1 ; c1ccc(-c2cccc(-c3nc(-c4ccccc4)nc(-c4cccc(-c5nc6ccc7c8cc9ccccc9cc8sc7c6o5)c4)n3)c2)cc1 ; c1ccc(-c2nc(-c3ccccc3)nc(-c3ccc4c(ccc5c6ccc7nc(-c8ccccc8)oc7c6sc45)c3)n2)cc1]

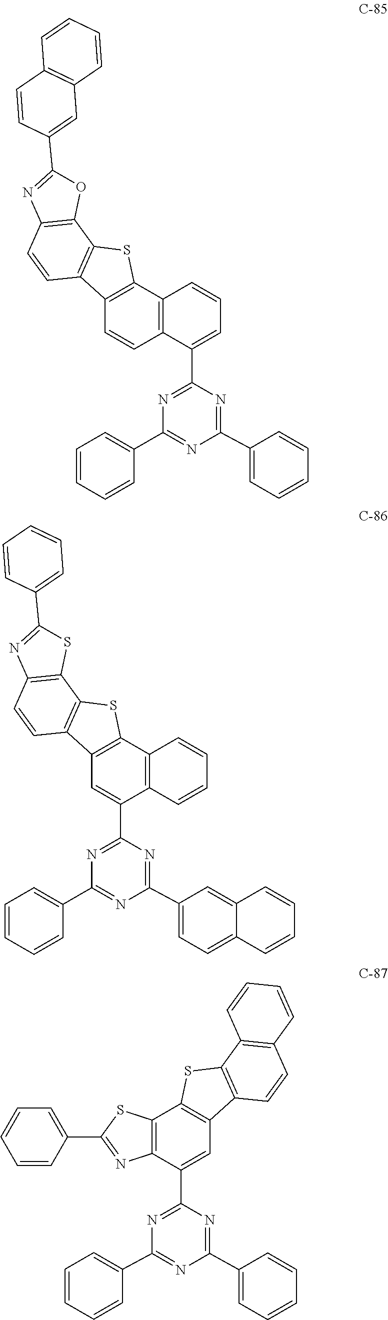

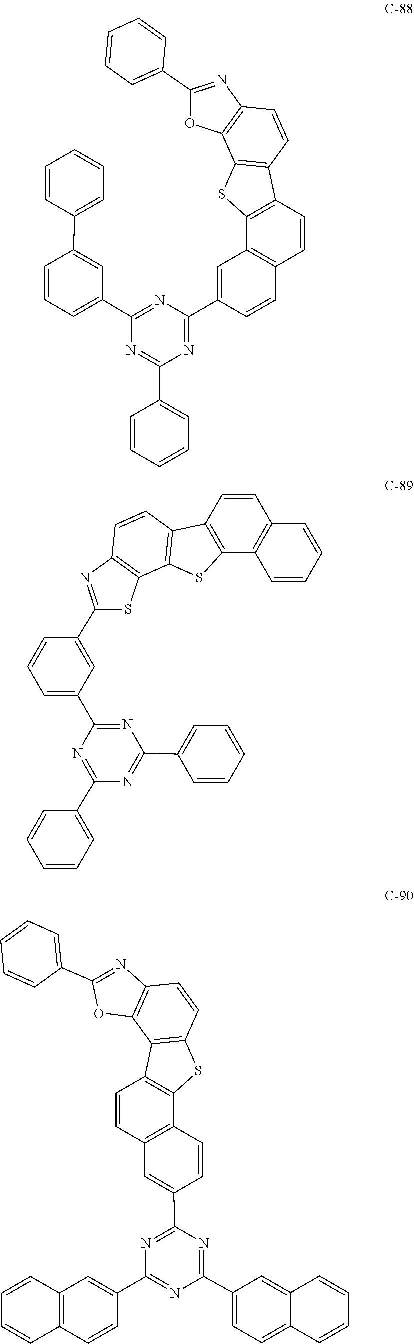 [SMILES: c1ccc(-c2cccc(-c3nc(-c4ccccc4)nc(-c4ccc5ccc6c7ccc8nc(-c9ccccc9)oc8c7sc6c5c4)n3)c2)cc1 ; c1ccc(-c2nc(-c3ccccc3)nc(-c3cccc(-c4nc5ccc6c7ccc8ccccc8c7sc6c5s4)c3)n2)cc1 ; c1ccc(-c2nc3ccc4sc5c6ccc(-c7nc(-c8ccc9ccccc9c8)nc(-c8ccc9ccccc9c8)n7)cc6ccc5c4c3o2)cc1]

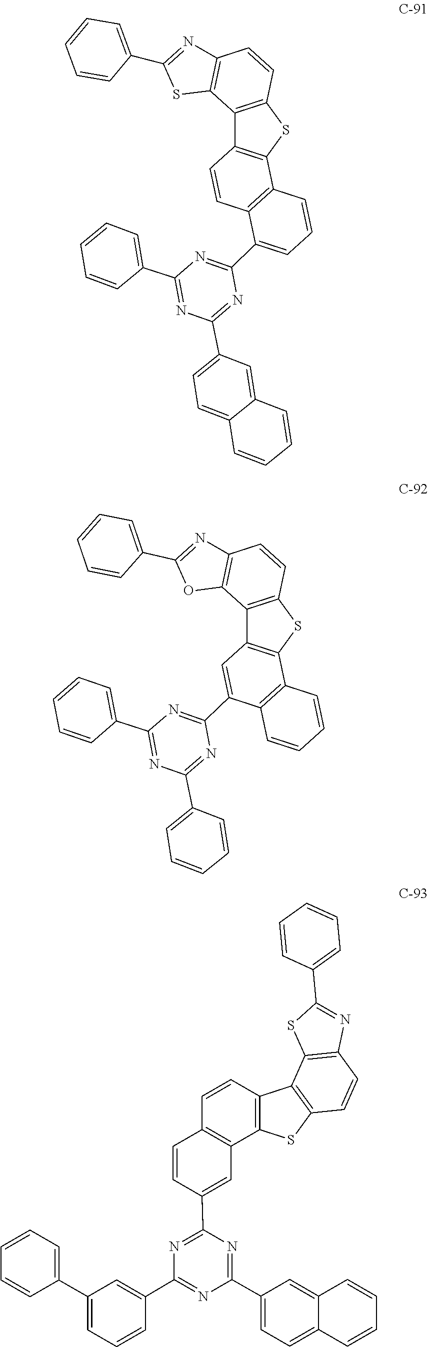 [SMILES: c1ccc(-c2nc(-c3ccc4ccccc4c3)nc(-c3cccc4c3ccc3c4sc4ccc5nc(-c6ccccc6)sc5c43)n2)cc1 ; c1ccc(-c2nc(-c3ccccc3)nc(-c3cc4c(sc5ccc6nc(-c7ccccc7)oc6c54)c4ccccc34)n2)cc1 ; c1ccc(-c2cccc(-c3nc(-c4ccc5ccccc5c4)nc(-c4ccc5ccc6c(sc7ccc8nc(-c9ccccc9)sc8c76)c5c4)n3)c2)cc1]

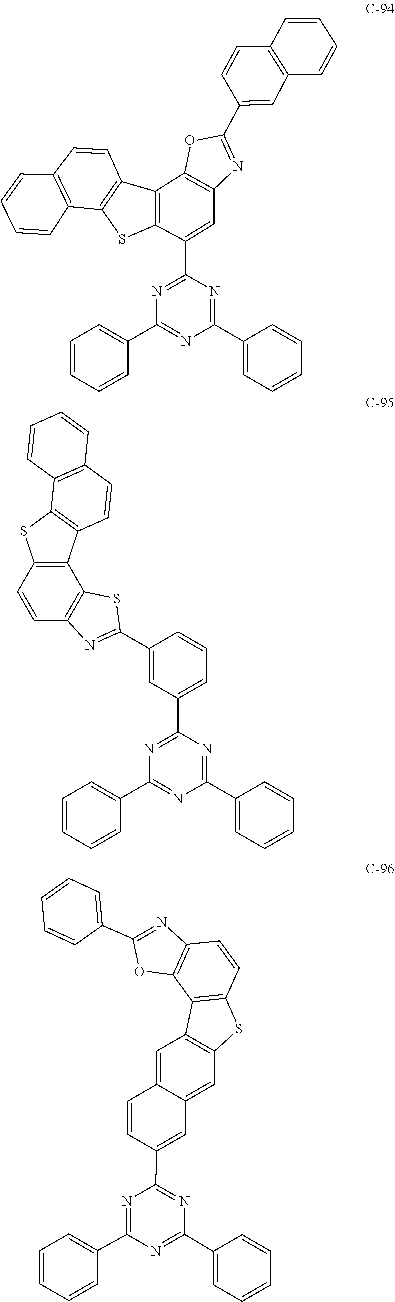

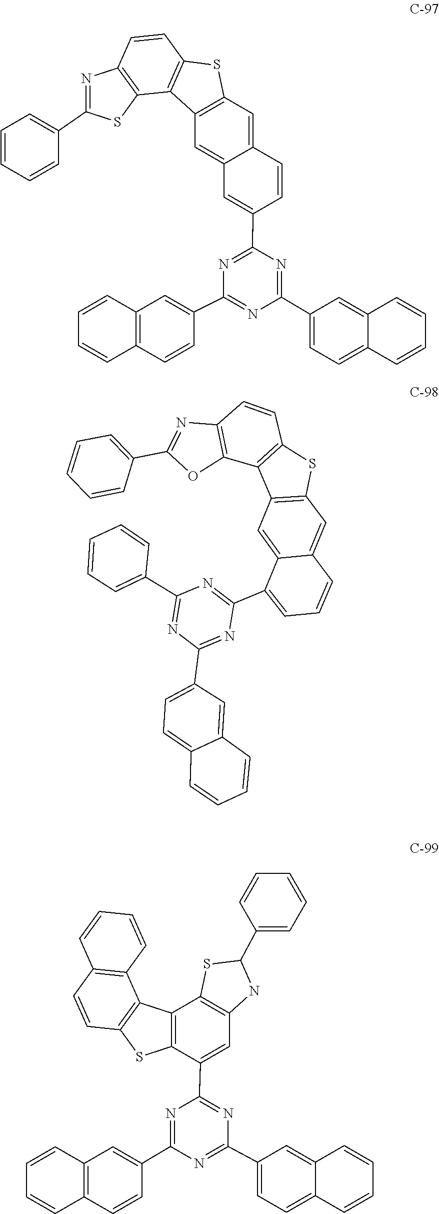

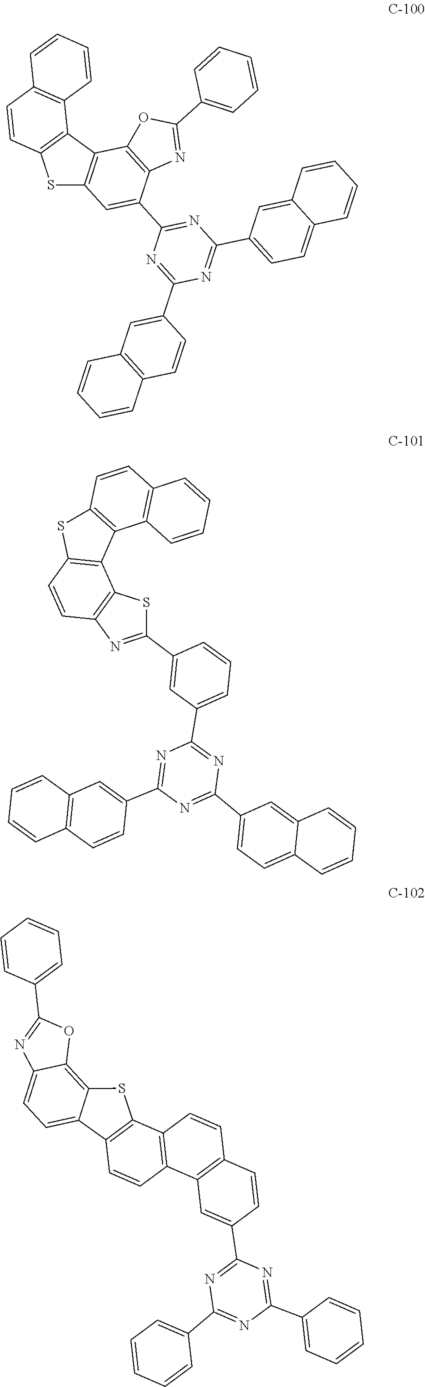

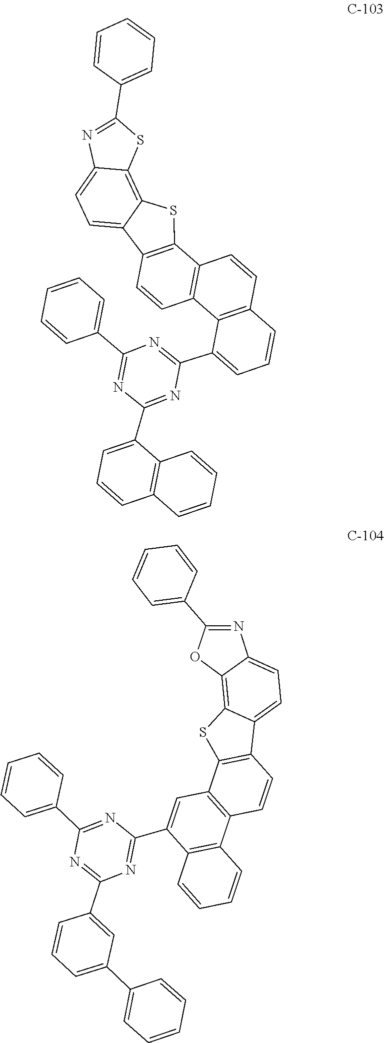

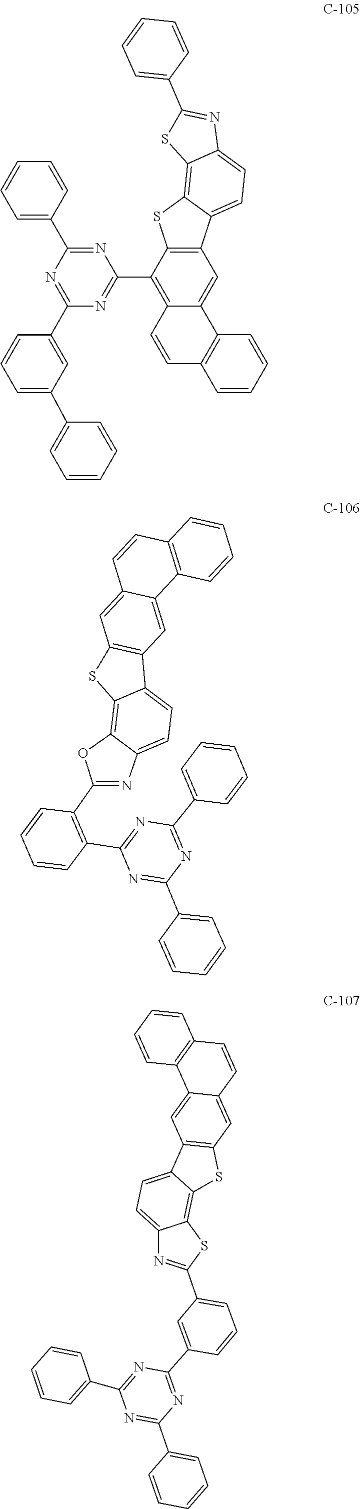

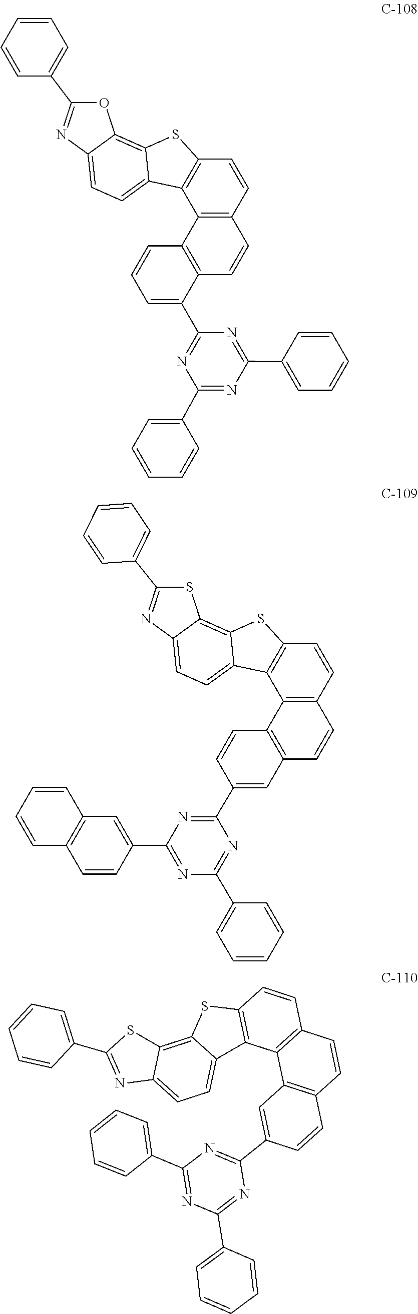

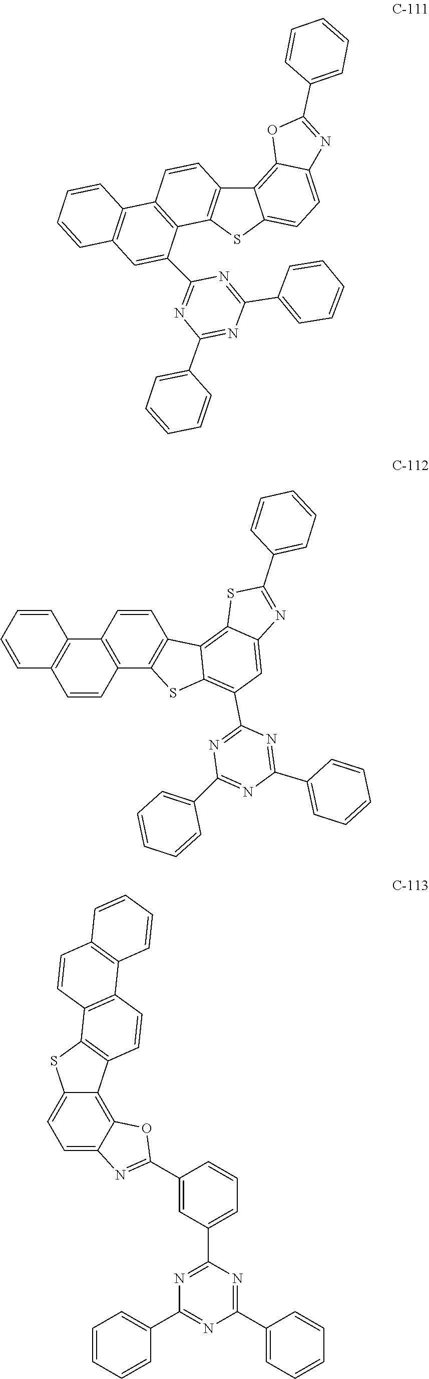 [SMILES: c1ccc(-c2nc(-c3ccccc3)nc(-c3cc4ccccc4c4ccc5c(sc6ccc7nc(-c8ccccc8)oc7c65)c34)n2)cc1 ; c1ccc(-c2nc(-c3ccccc3)nc(-c3cc4nc(-c5ccccc5)sc4c4c3sc3c5ccc6ccccc6c5ccc34)n2)cc1 ; c1ccc(-c2nc(-c3ccccc3)nc(-c3cccc(-c4nc5ccc6sc7c8ccc9ccccc9c8ccc7c6c5o4)c3)n2)cc1]

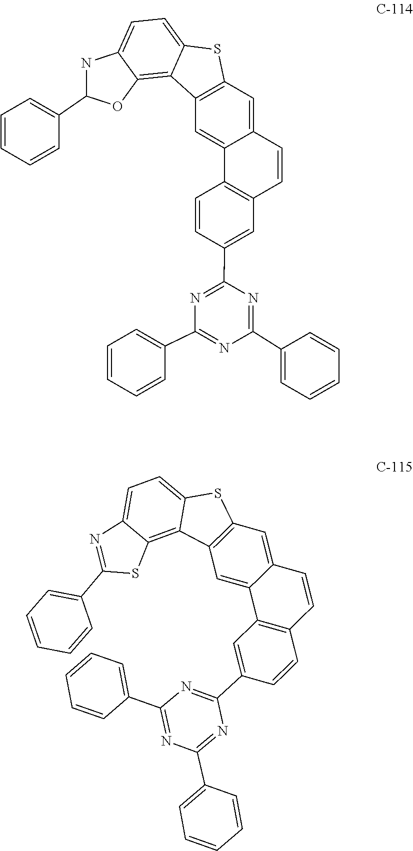

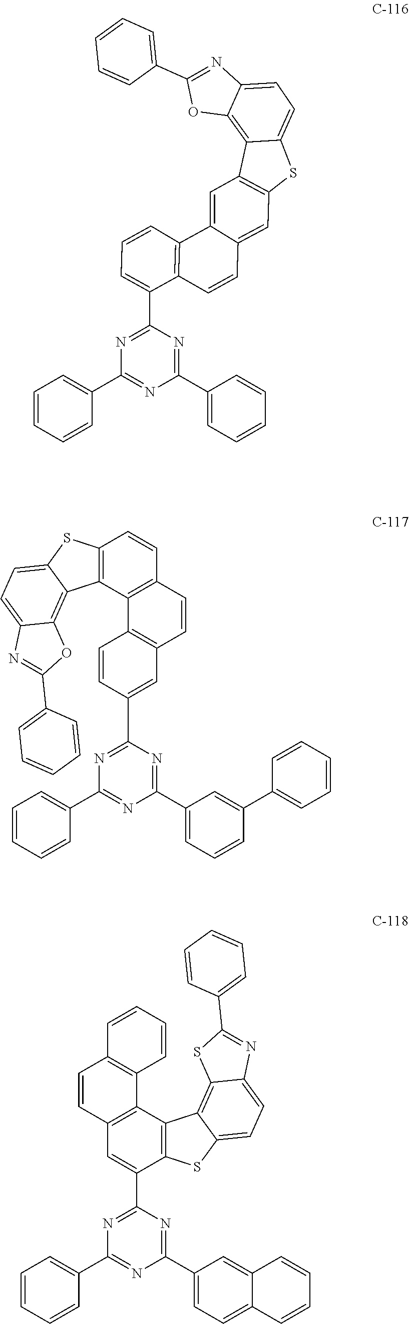 [SMILES: c1ccc(-c2nc(-c3ccccc3)nc(-c3cccc4c3ccc3cc5sc6ccc7nc(-c8ccccc8)oc7c6c5cc34)n2)cc1 ; c1ccc(-c2cccc(-c3nc(-c4ccccc4)nc(-c4ccc5c(ccc6ccc7sc8ccc9nc(-c%10ccccc%10)oc9c8c7c65)c4)n3)c2)cc1 ; c1ccc(-c2nc(-c3ccc4ccccc4c3)nc(-c3cc4ccc5ccccc5c4c4c3sc3ccc5nc(-c6ccccc6)sc5c34)n2)cc1]

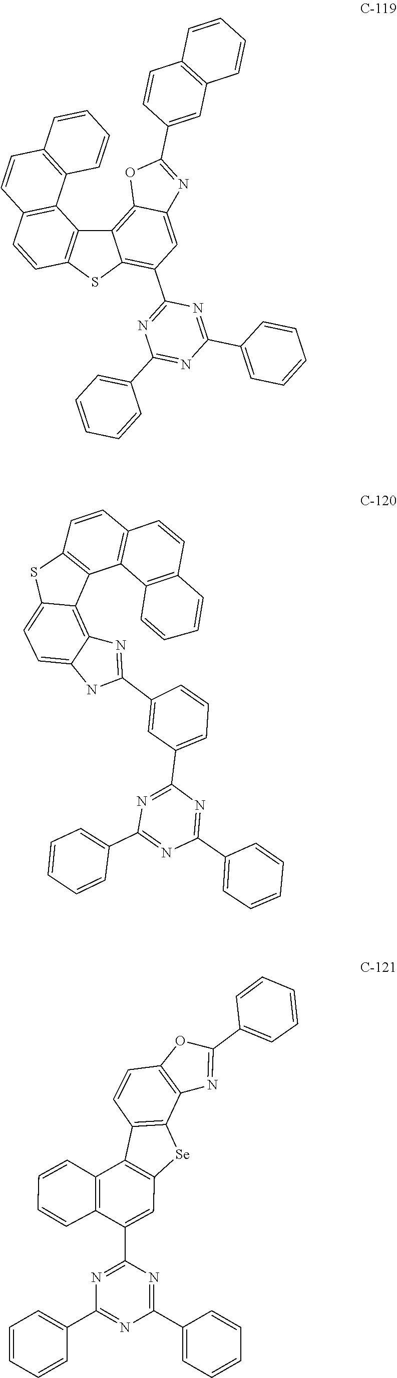 [SMILES: c1ccc(-c2nc(-c3ccccc3)nc(-c3cc4nc(-c5ccc6ccccc6c5)oc4c4c3sc3ccc5ccc6ccccc6c5c34)n2)cc1 ; c1ccc(-c2nc(-c3ccccc3)nc(-c3cccc(-c4nc5c(ccc6sc7ccc8ccc9ccccc9c8c7c65)[nH]4)c3)n2)cc1 ; c1ccc(-c2nc(-c3ccccc3)nc(-c3cc4[se]c5c(ccc6oc(-c7ccccc7)nc65)c4c4ccccc34)n2)cc1]

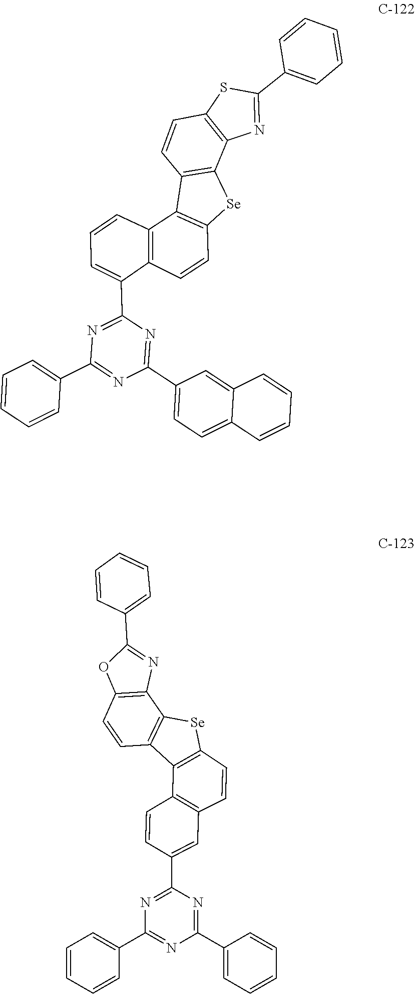 [SMILES: c1ccc(-c2nc(-c3ccc4ccccc4c3)nc(-c3cccc4c3ccc3[se]c5c(ccc6sc(-c7ccccc7)nc65)c34)n2)cc1 ; c1ccc(-c2nc(-c3ccccc3)nc(-c3ccc4c(ccc5[se]c6c(ccc7oc(-c8ccccc8)nc76)c54)c3)n2)cc1]

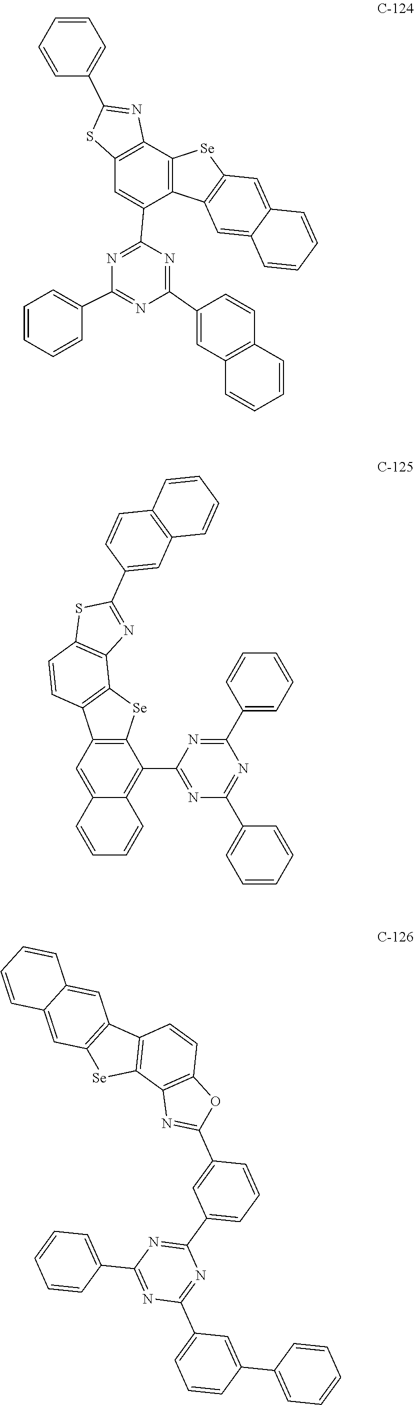 [SMILES: c1ccc(-c2nc(-c3ccc4ccccc4c3)nc(-c3cc4sc(-c5ccccc5)nc4c4[se]c5cc6ccccc6cc5c34)n2)cc1 ; c1ccc(-c2nc(-c3ccccc3)nc(-c3c4ccccc4cc4c3[se]c3c4ccc4sc(-c5ccc6ccccc6c5)nc43)n2)cc1 ; c1ccc(-c2cccc(-c3nc(-c4ccccc4)nc(-c4cccc(-c5nc6c(ccc7c8cc9ccccc9cc8[se]c76)o5)c4)n3)c2)cc1]

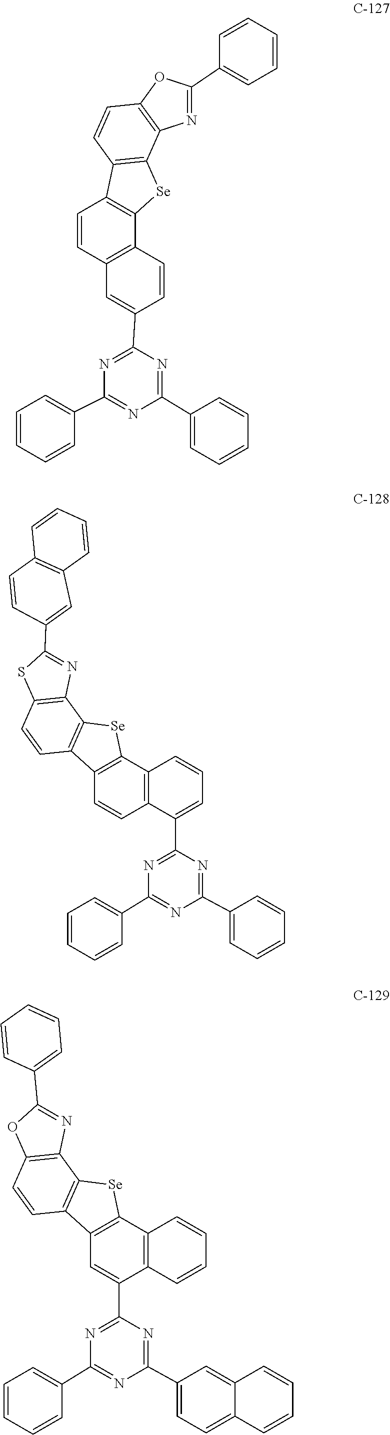 [SMILES: c1ccc(-c2nc(-c3ccccc3)nc(-c3ccc4c(ccc5c6ccc7oc(-c8ccccc8)nc7c6[se]c45)c3)n2)cc1 ; c1ccc(-c2nc(-c3ccccc3)nc(-c3cccc4c3ccc3c5ccc6sc(-c7ccc8ccccc8c7)nc6c5[se]c43)n2)cc1 ; c1ccc(-c2nc(-c3ccc4ccccc4c3)nc(-c3cc4c5ccc6oc(-c7ccccc7)nc6c5[se]c4c4ccccc34)n2)cc1]

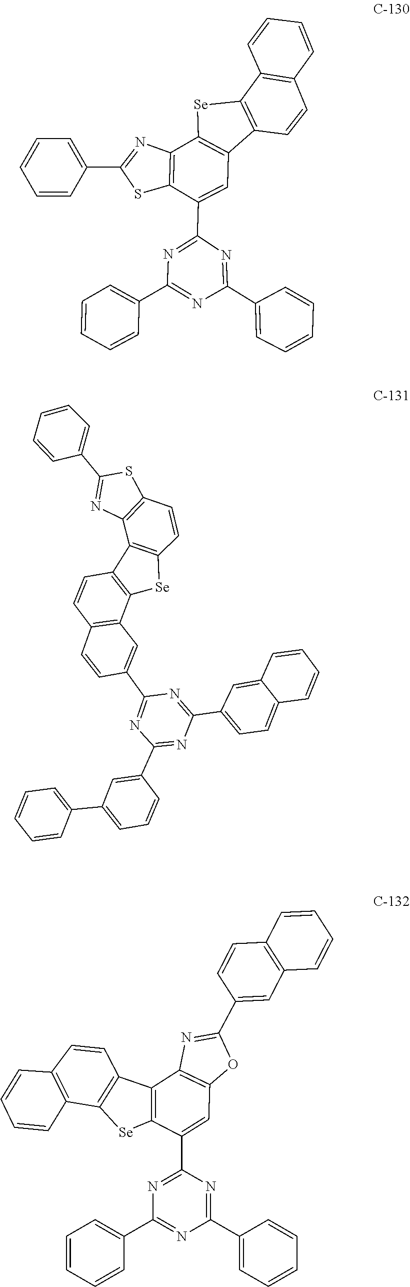 [SMILES: c1ccc(-c2nc(-c3ccccc3)nc(-c3cc4c5ccc6ccccc6c5[se]c4c4nc(-c5ccccc5)sc34)n2)cc1 ; c1ccc(-c2cccc(-c3nc(-c4ccc5ccccc5c4)nc(-c4ccc5ccc6c([se]c7ccc8sc(-c9ccccc9)nc8c76)c5c4)n3)c2)cc1 ; c1ccc(-c2nc(-c3ccccc3)nc(-c3cc4oc(-c5ccc6ccccc6c5)nc4c4c3[se]c3c5ccccc5ccc34)n2)cc1]

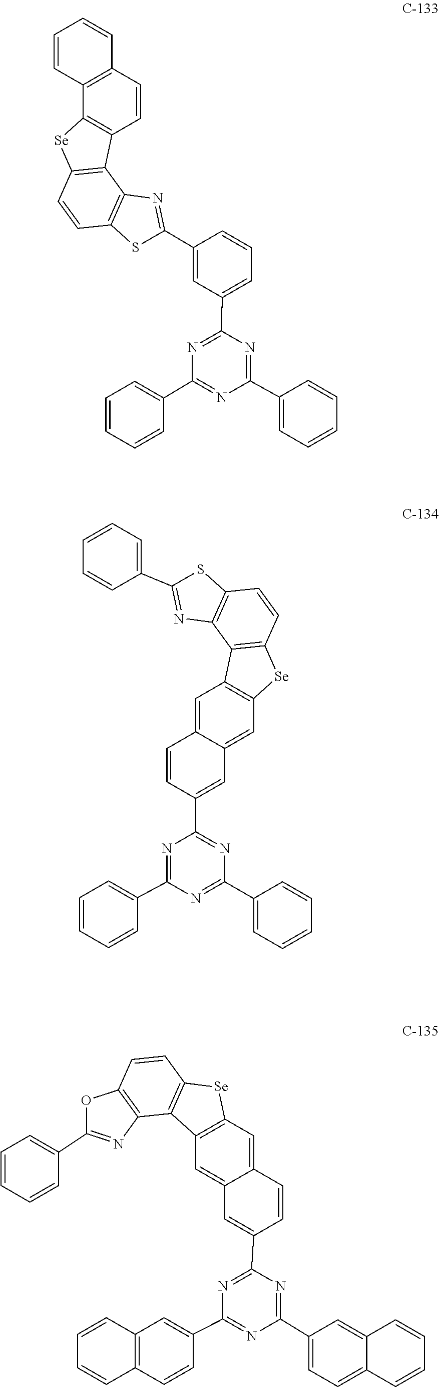 [SMILES: c1ccc(-c2nc(-c3ccccc3)nc(-c3cccc(-c4nc5c(ccc6[se]c7c8ccccc8ccc7c65)s4)c3)n2)cc1 ; c1ccc(-c2nc(-c3ccccc3)nc(-c3ccc4cc5c(cc4c3)[se]c3ccc4sc(-c6ccccc6)nc4c35)n2)cc1 ; c1ccc(-c2nc3c(ccc4[se]c5cc6ccc(-c7nc(-c8ccc9ccccc9c8)nc(-c8ccc9ccccc9c8)n7)cc6cc5c43)o2)cc1]

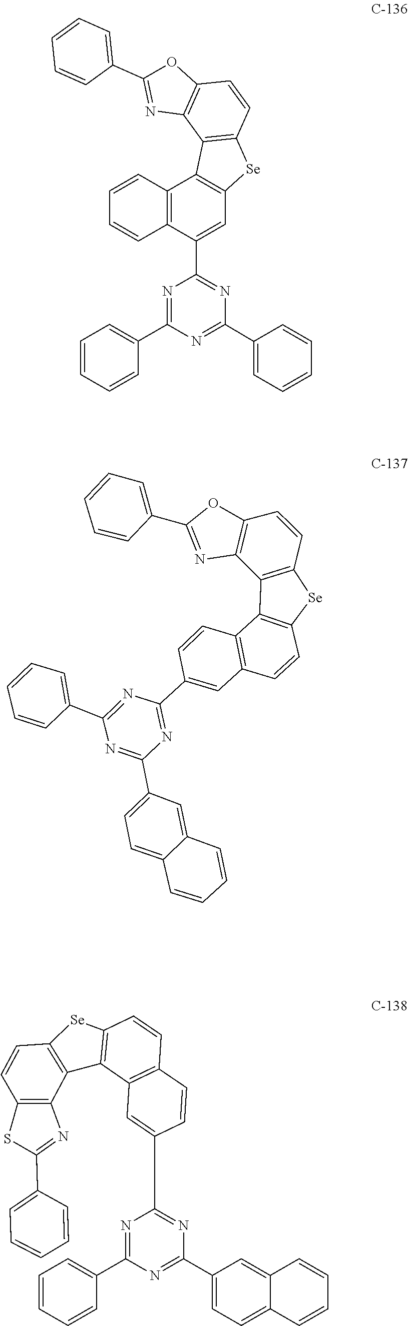 [SMILES: c1ccc(-c2nc(-c3ccccc3)nc(-c3cc4[se]c5ccc6oc(-c7ccccc7)nc6c5c4c4ccccc34)n2)cc1 ; c1ccc(-c2nc(-c3ccc4ccccc4c3)nc(-c3ccc4c(ccc5[se]c6ccc7oc(-c8ccccc8)nc7c6c54)c3)n2)cc1 ; c1ccc(-c2nc(-c3ccc4ccccc4c3)nc(-c3ccc4ccc5[se]c6ccc7sc(-c8ccccc8)nc7c6c5c4c3)n2)cc1]

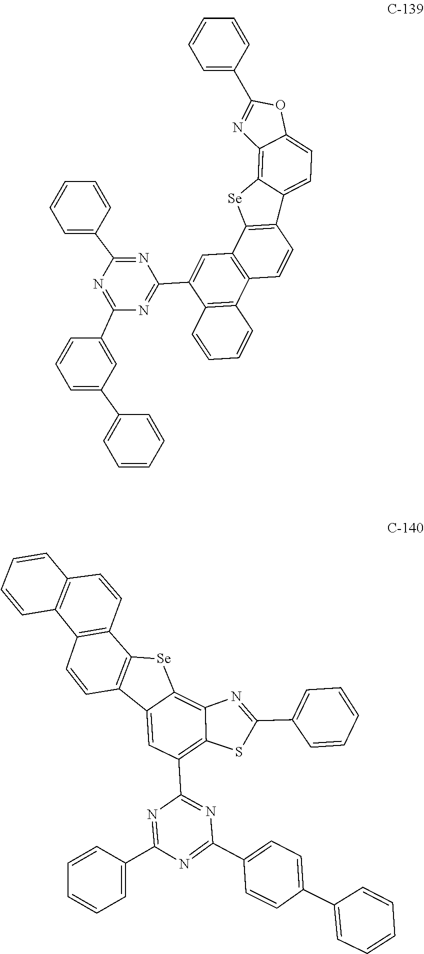 [SMILES: c1ccc(-c2cccc(-c3nc(-c4ccccc4)nc(-c4cc5c(ccc6c7ccc8oc(-c9ccccc9)nc8c7[se]c56)c5ccccc45)n3)c2)cc1 ; c1ccc(-c2ccc(-c3nc(-c4ccccc4)nc(-c4cc5c6ccc7c8ccccc8ccc7c6[se]c5c5nc(-c6ccccc6)sc45)n3)cc2)cc1]

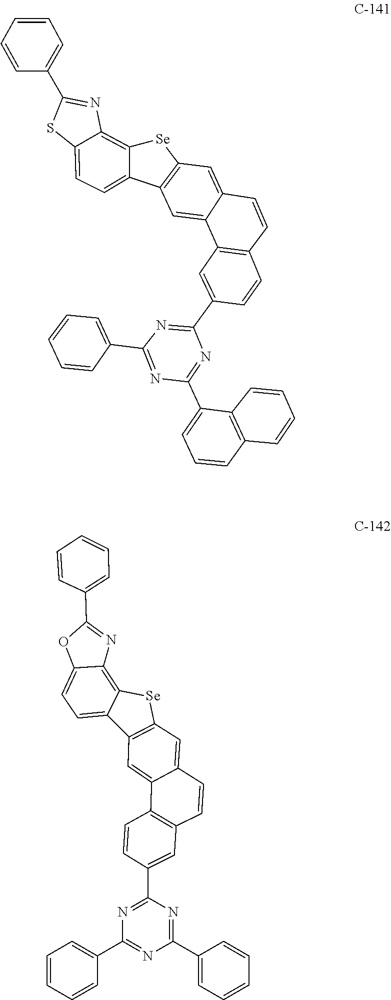 [SMILES: c1ccc(-c2nc(-c3ccc4ccc5cc6[se]c7c(ccc8sc(-c9ccccc9)nc87)c6cc5c4c3)nc(-c3cccc4ccccc34)n2)cc1 ; c1ccc(-c2nc(-c3ccccc3)nc(-c3ccc4c(ccc5cc6[se]c7c(ccc8oc(-c9ccccc9)nc87)c6cc54)c3)n2)cc1]

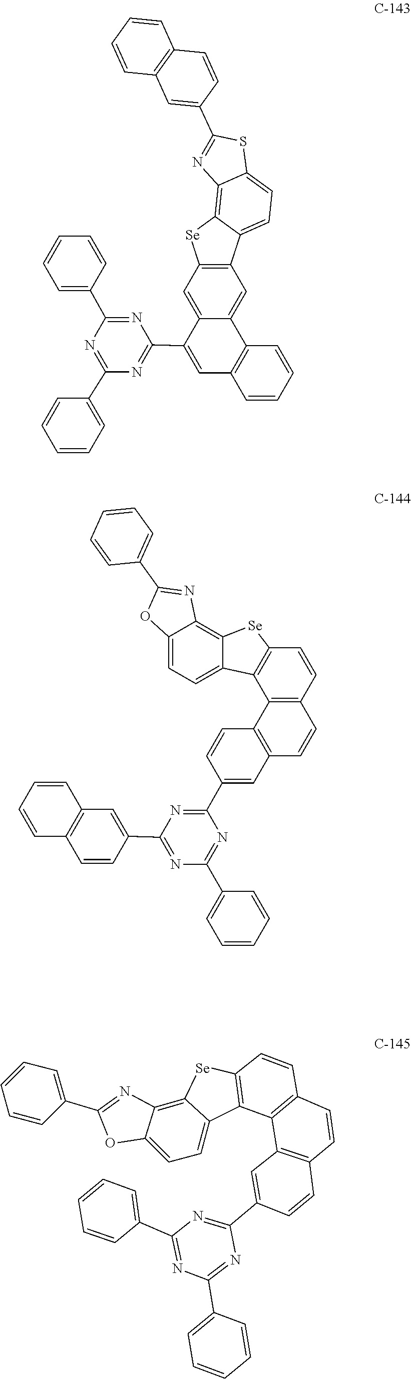

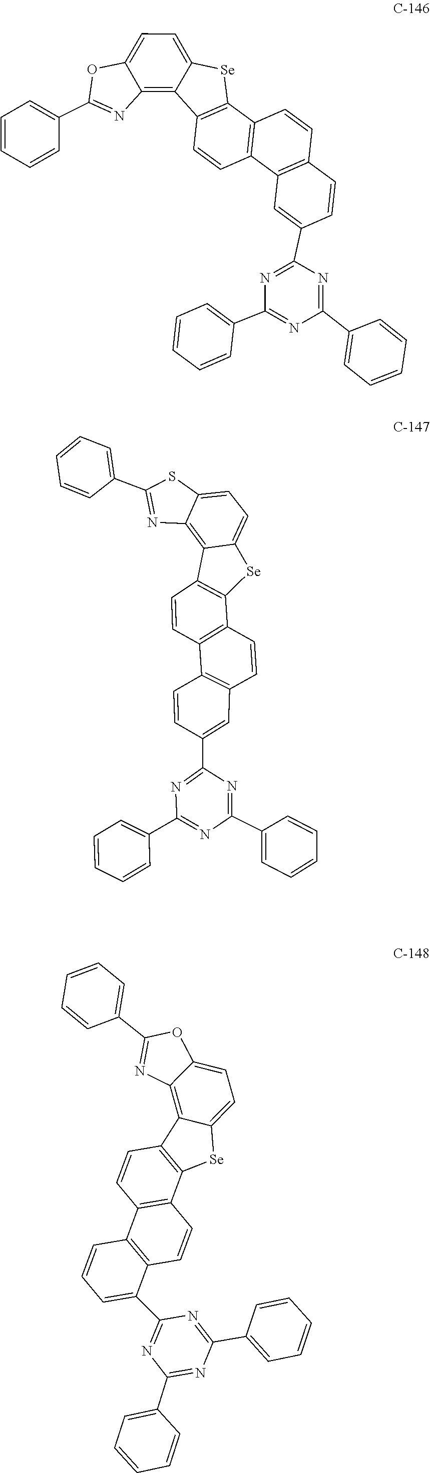 [SMILES: c1ccc(-c2nc(-c3ccccc3)nc(-c3ccc4ccc5c(ccc6c5[se]c5ccc7oc(-c8ccccc8)nc7c56)c4c3)n2)cc1 ; c1ccc(-c2nc(-c3ccccc3)nc(-c3ccc4c(ccc5c4ccc4c5[se]c5ccc6sc(-c7ccccc7)nc6c54)c3)n2)cc1 ; c1ccc(-c2nc(-c3ccccc3)nc(-c3cccc4c3ccc3c4ccc4c3[se]c3ccc5oc(-c6ccccc6)nc5c34)n2)cc1]

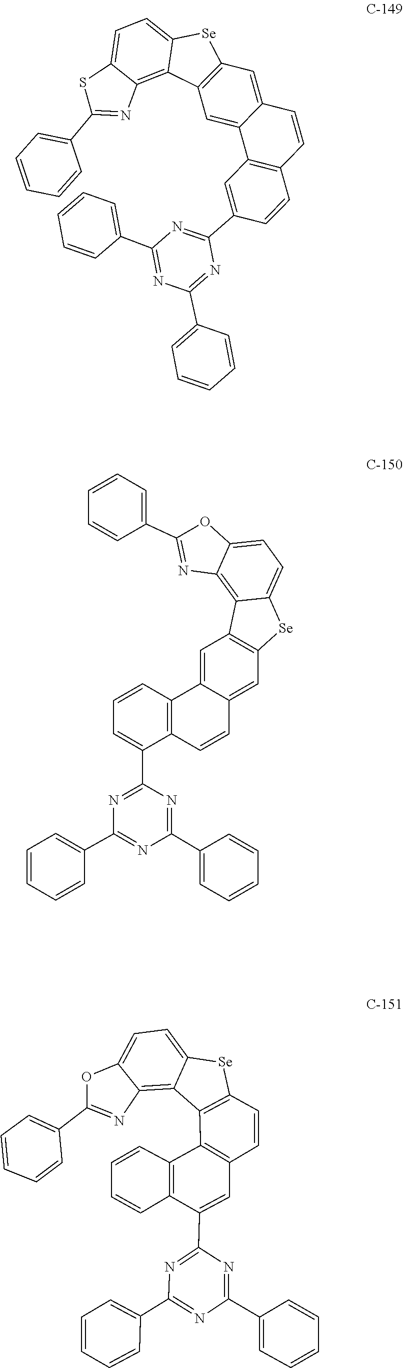 [SMILES: c1ccc(-c2nc(-c3ccccc3)nc(-c3ccc4ccc5cc6[se]c7ccc8sc(-c9ccccc9)nc8c7c6cc5c4c3)n2)cc1 ; c1ccc(-c2nc(-c3ccccc3)nc(-c3cccc4c3ccc3cc5[se]c6ccc7oc(-c8ccccc8)nc7c6c5cc34)n2)cc1 ; c1ccc(-c2nc(-c3ccccc3)nc(-c3cc4ccc5[se]c6ccc7oc(-c8ccccc8)nc7c6c5c4c4ccccc34)n2)cc1]

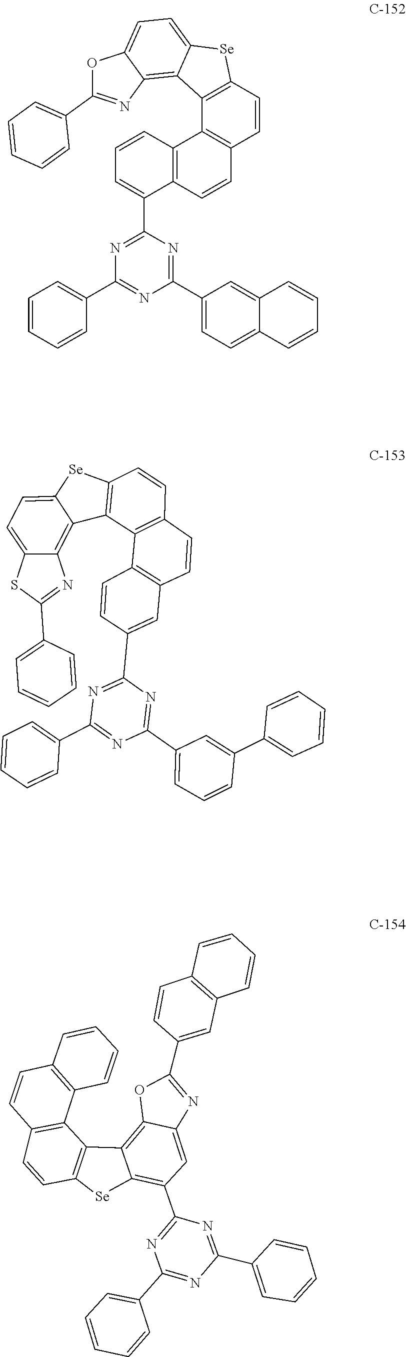 [SMILES: c1ccc(-c2nc(-c3ccc4ccccc4c3)nc(-c3cccc4c3ccc3ccc5[se]c6ccc7oc(-c8ccccc8)nc7c6c5c34)n2)cc1 ; c1ccc(-c2cccc(-c3nc(-c4ccccc4)nc(-c4ccc5c(ccc6ccc7[se]c8ccc9sc(-c%10ccccc%10)nc9c8c7c65)c4)n3)c2)cc1 ; c1ccc(-c2nc(-c3ccccc3)nc(-c3cc4nc(-c5ccc6ccccc6c5)oc4c4c3[se]c3ccc5ccc6ccccc6c5c34)n2)cc1]

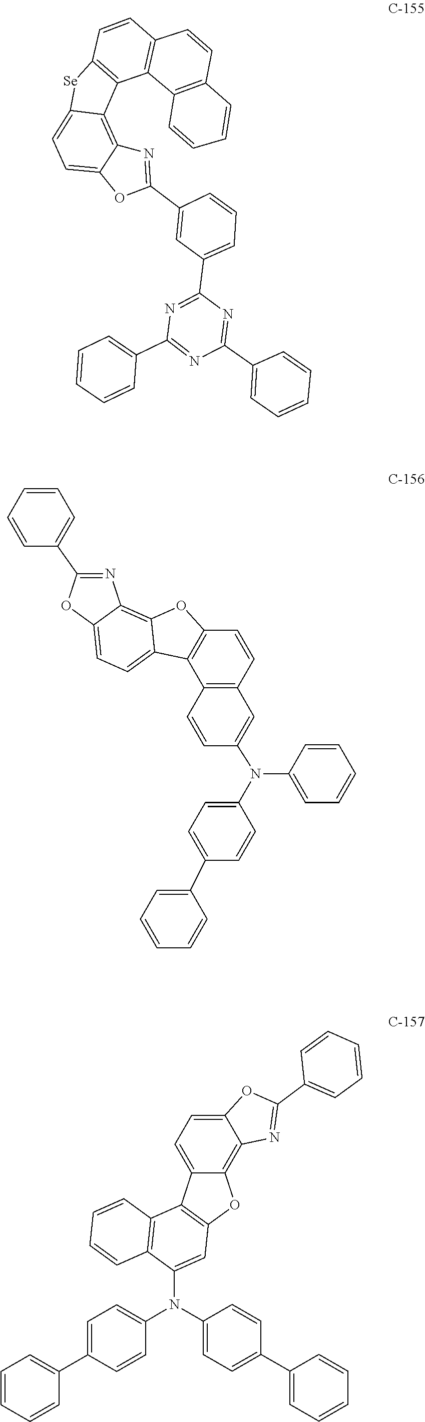 [SMILES: c1ccc(-c2nc(-c3ccccc3)nc(-c3cccc(-c4nc5c(ccc6[se]c7ccc8ccc9ccccc9c8c7c65)o4)c3)n2)cc1 ; c1ccc(-c2ccc(N(c3ccccc3)c3ccc4c(ccc5oc6c(ccc7oc(-c8ccccc8)nc76)c54)c3)cc2)cc1 ; c1ccc(-c2ccc(N(c3ccc(-c4ccccc4)cc3)c3cc4oc5c(ccc6oc(-c7ccccc7)nc65)c4c4ccccc34)cc2)cc1]

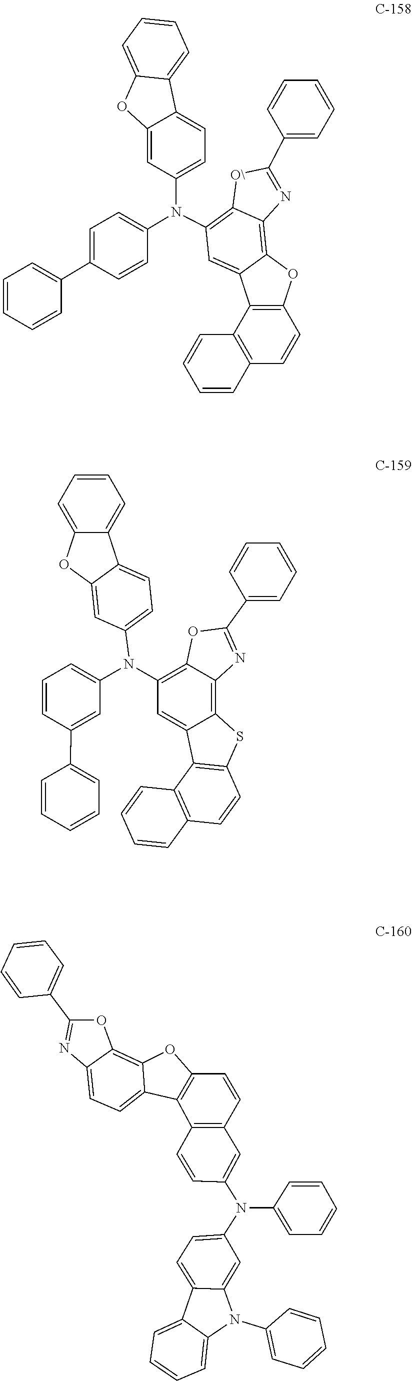

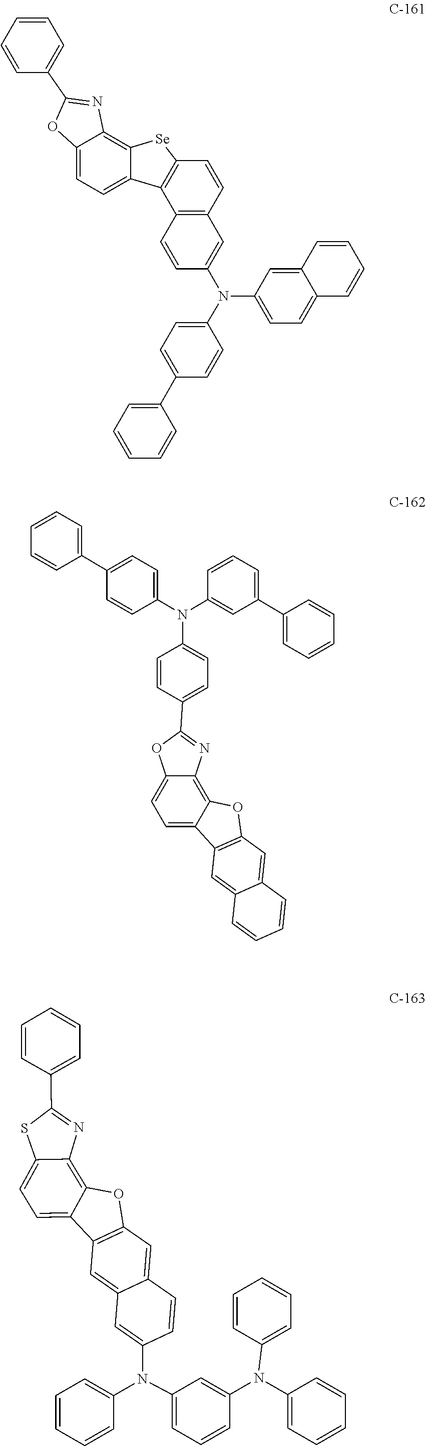

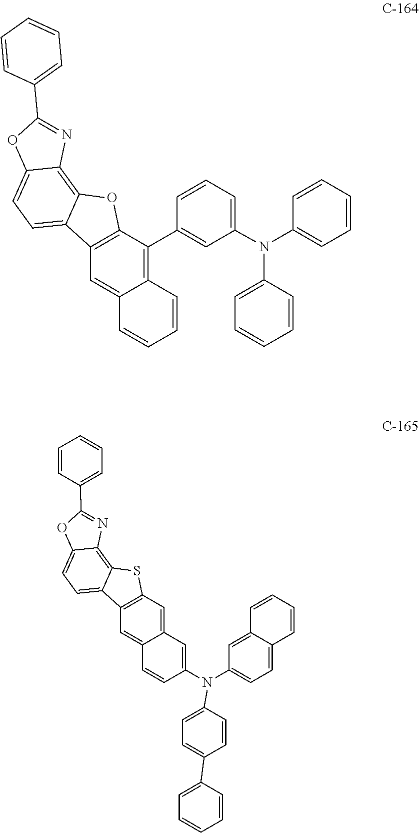 [SMILES: c1ccc(-c2nc3c(ccc4c5cc6ccccc6c(-c6cccc(N(c7ccccc7)c7ccccc7)c6)c5oc43)o2)cc1 ; c1ccc(-c2ccc(N(c3ccc4ccccc4c3)c3ccc4cc5c(cc4c3)sc3c5ccc4oc(-c5ccccc5)nc43)cc2)cc1]

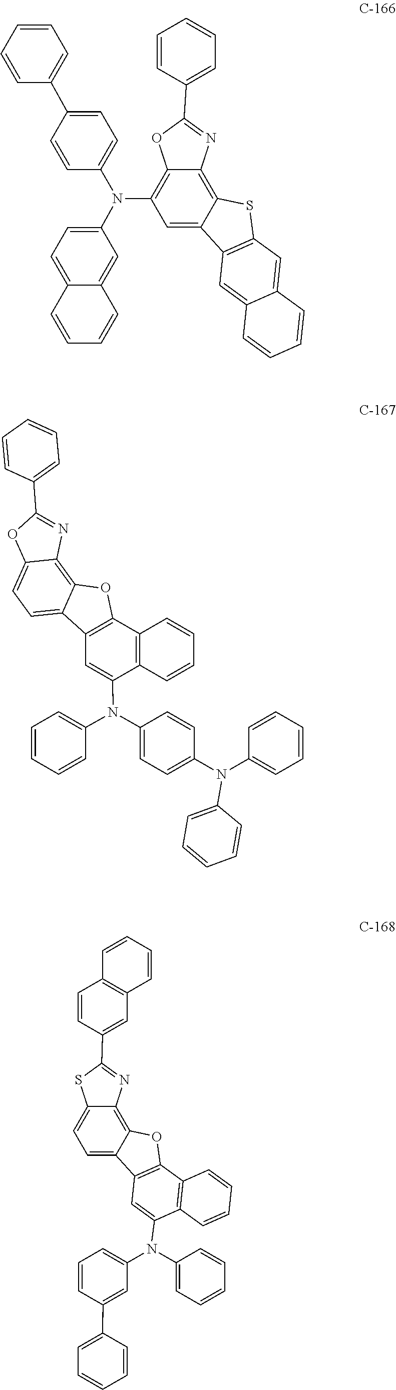 [SMILES: c1ccc(-c2ccc(N(c3ccc4ccccc4c3)c3cc4c5cc6ccccc6cc5sc4c4nc(-c5ccccc5)oc34)cc2)cc1 ; c1ccc(-c2nc3c(ccc4c5cc(N(c6ccccc6)c6ccc(N(c7ccccc7)c7ccccc7)cc6)c6ccccc6c5oc43)o2)cc1 ; c1ccc(-c2cccc(N(c3ccccc3)c3cc4c5ccc6sc(-c7ccc8ccccc8c7)nc6c5oc4c4ccccc34)c2)cc1]

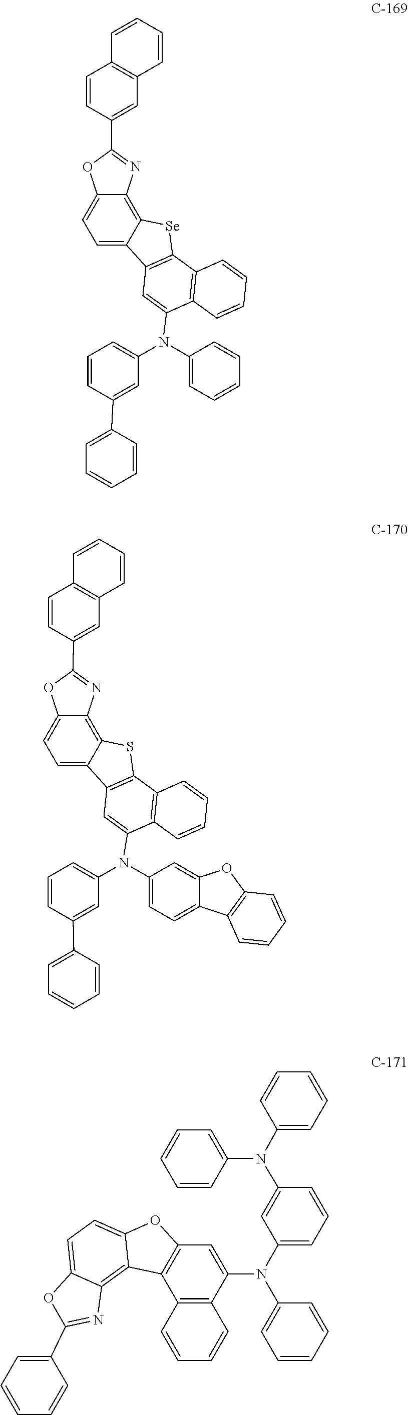 [SMILES: c1ccc(-c2cccc(N(c3ccccc3)c3cc4c5ccc6oc(-c7ccc8ccccc8c7)nc6c5[se]c4c4ccccc34)c2)cc1 ; c1ccc(-c2cccc(N(c3ccc4c(c3)oc3ccccc34)c3cc4c5ccc6oc(-c7ccc8ccccc8c7)nc6c5sc4c4ccccc34)c2)cc1 ; c1ccc(-c2nc3c(ccc4oc5cc(N(c6ccccc6)c6cccc(N(c7ccccc7)c7ccccc7)c6)c6ccccc6c5c43)o2)cc1]

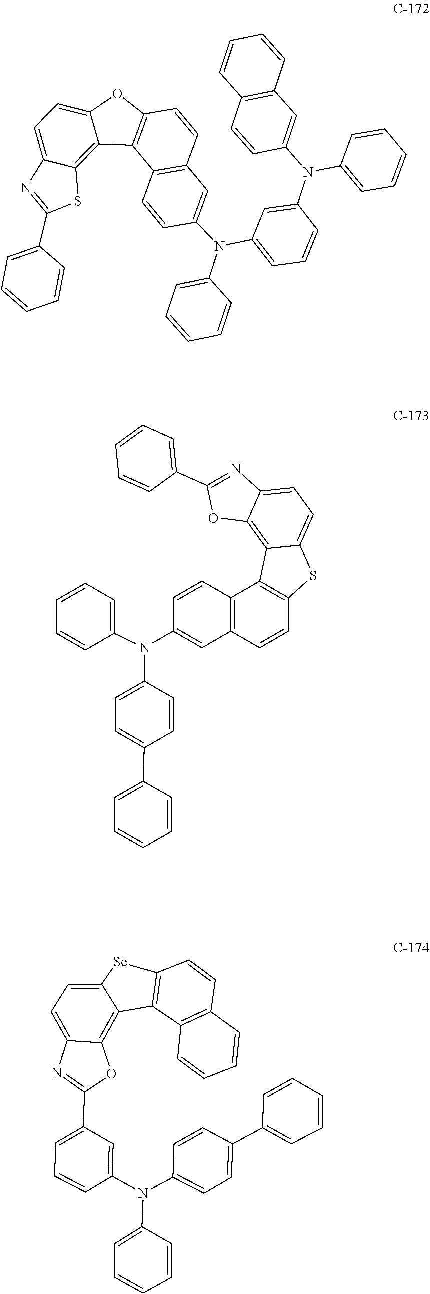 [SMILES: c1ccc(-c2nc3ccc4oc5ccc6cc(N(c7ccccc7)c7cccc(N(c8ccccc8)c8ccc9ccccc9c8)c7)ccc6c5c4c3s2)cc1 ; c1ccc(-c2ccc(N(c3ccccc3)c3ccc4c(ccc5sc6ccc7nc(-c8ccccc8)oc7c6c54)c3)cc2)cc1 ; c1ccc(-c2ccc(N(c3ccccc3)c3cccc(-c4nc5ccc6[se]c7ccc8ccccc8c7c6c5o4)c3)cc2)cc1]

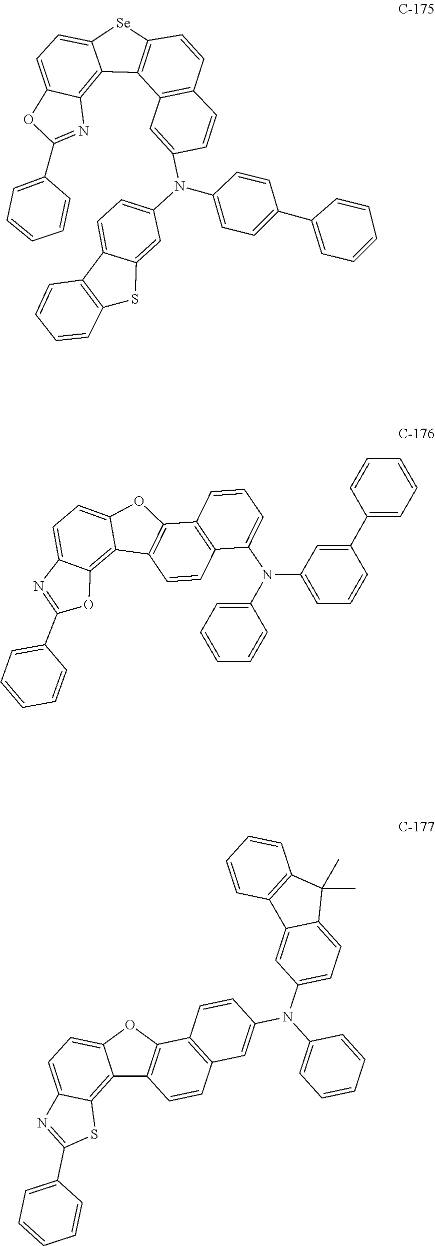 [SMILES: c1ccc(-c2ccc(N(c3ccc4c(c3)sc3ccccc34)c3ccc4ccc5[se]c6ccc7oc(-c8ccccc8)nc7c6c5c4c3)cc2)cc1 ; c1ccc(-c2cccc(N(c3ccccc3)c3cccc4c3ccc3c4oc4ccc5nc(-c6ccccc6)oc5c43)c2)cc1 ; CC1(C)c2ccccc2-c2cc(N(c3ccccc3)c3ccc4c(ccc5c4oc4ccc6nc(-c7ccccc7)sc6c45)c3)ccc21]

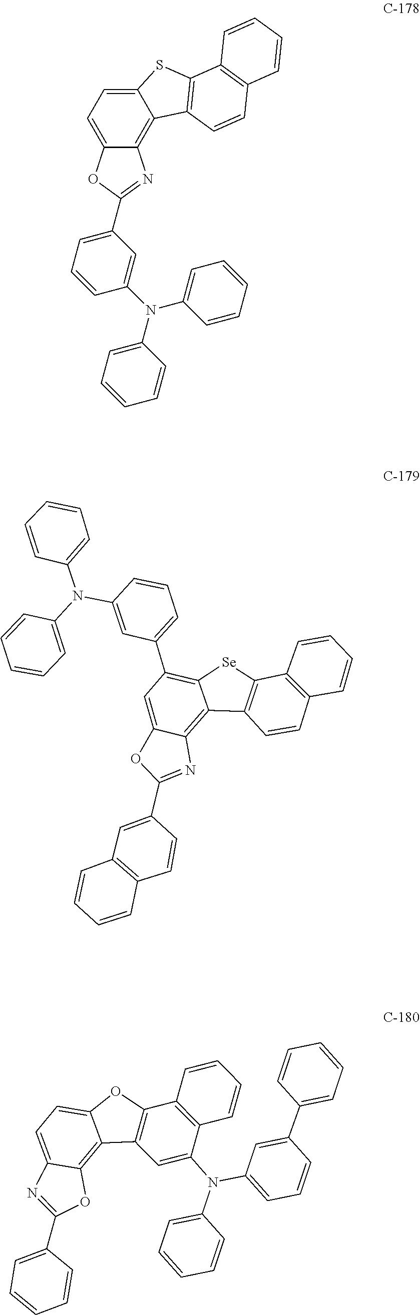 [SMILES: c1ccc(N(c2ccccc2)c2cccc(-c3nc4c(ccc5sc6c7ccccc7ccc6c54)o3)c2)cc1 ; c1ccc(N(c2ccccc2)c2cccc(-c3cc4oc(-c5ccc6ccccc6c5)nc4c4c3[se]c3c5ccccc5ccc34)c2)cc1 ; c1ccc(-c2cccc(N(c3ccccc3)c3cc4c(oc5ccc6nc(-c7ccccc7)oc6c54)c4ccccc34)c2)cc1]

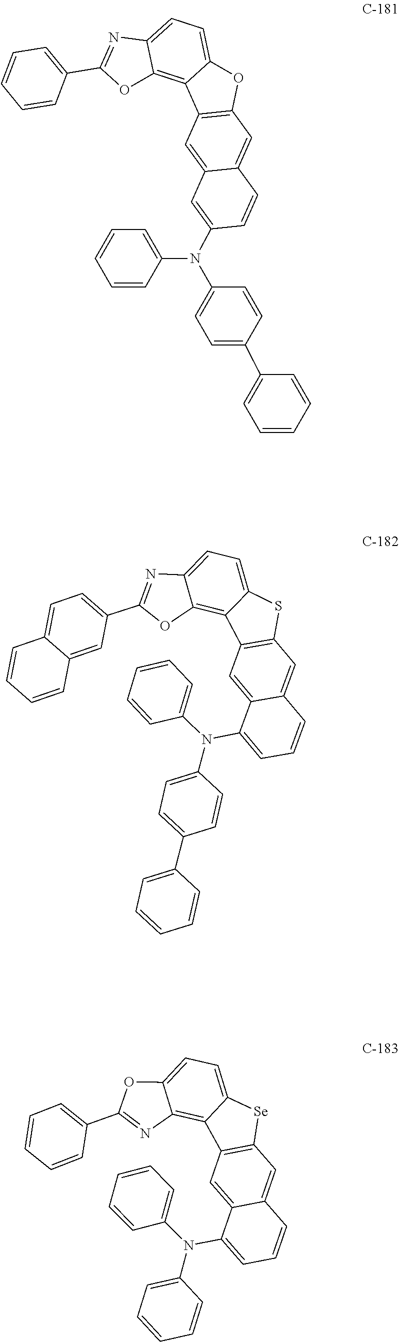 [SMILES: c1ccc(-c2ccc(N(c3ccccc3)c3ccc4cc5oc6ccc7nc(-c8ccccc8)oc7c6c5cc4c3)cc2)cc1 ; c1ccc(-c2ccc(N(c3ccccc3)c3cccc4cc5sc6ccc7nc(-c8ccc9ccccc9c8)oc7c6c5cc34)cc2)cc1 ; c1ccc(-c2nc3c(ccc4[se]c5cc6cccc(N(c7ccccc7)c7ccccc7)c6cc5c43)o2)cc1]

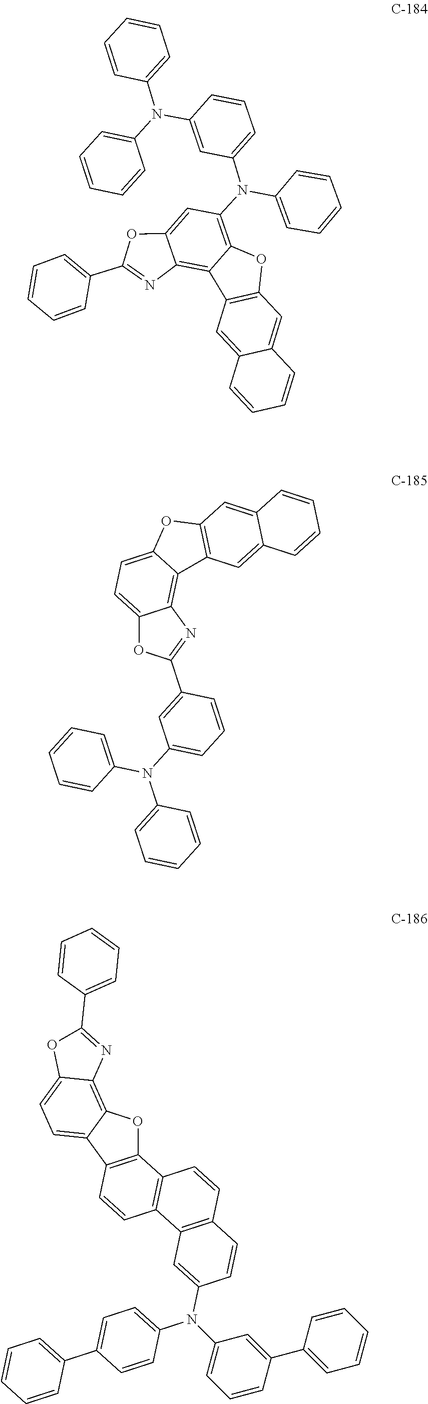 [SMILES: c1ccc(-c2nc3c(cc(N(c4ccccc4)c4cccc(N(c5ccccc5)c5ccccc5)c4)c4oc5cc6ccccc6cc5c43)o2)cc1 ; c1ccc(N(c2ccccc2)c2cccc(-c3nc4c(ccc5oc6cc7ccccc7cc6c54)o3)c2)cc1 ; c1ccc(-c2ccc(N(c3cccc(-c4ccccc4)c3)c3ccc4ccc5c(ccc6c7ccc8oc(-c9ccccc9)nc8c7oc56)c4c3)cc2)cc1]

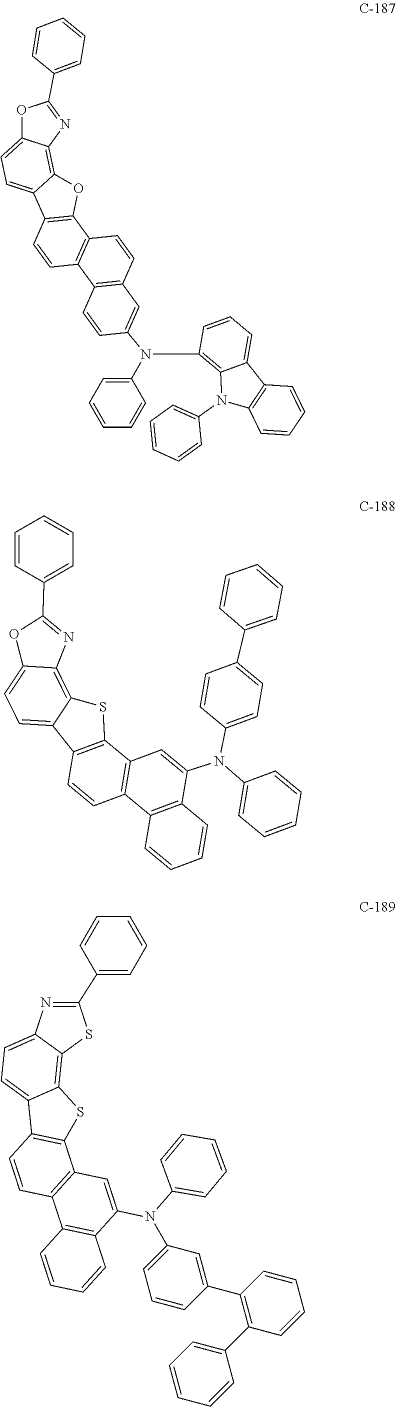 [SMILES: c1ccc(-c2nc3c(ccc4c5ccc6c7ccc(N(c8ccccc8)c8cccc9c%10ccccc%10n(-c%10ccccc%10)c89)cc7ccc6c5oc43)o2)cc1 ; c1ccc(-c2ccc(N(c3ccccc3)c3cc4c(ccc5c6ccc7oc(-c8ccccc8)nc7c6sc45)c4ccccc34)cc2)cc1 ; c1ccc(-c2nc3ccc4c5ccc6c7ccccc7c(N(c7ccccc7)c7cccc(-c8ccccc8-c8ccccc8)c7)cc6c5sc4c3s2)cc1]

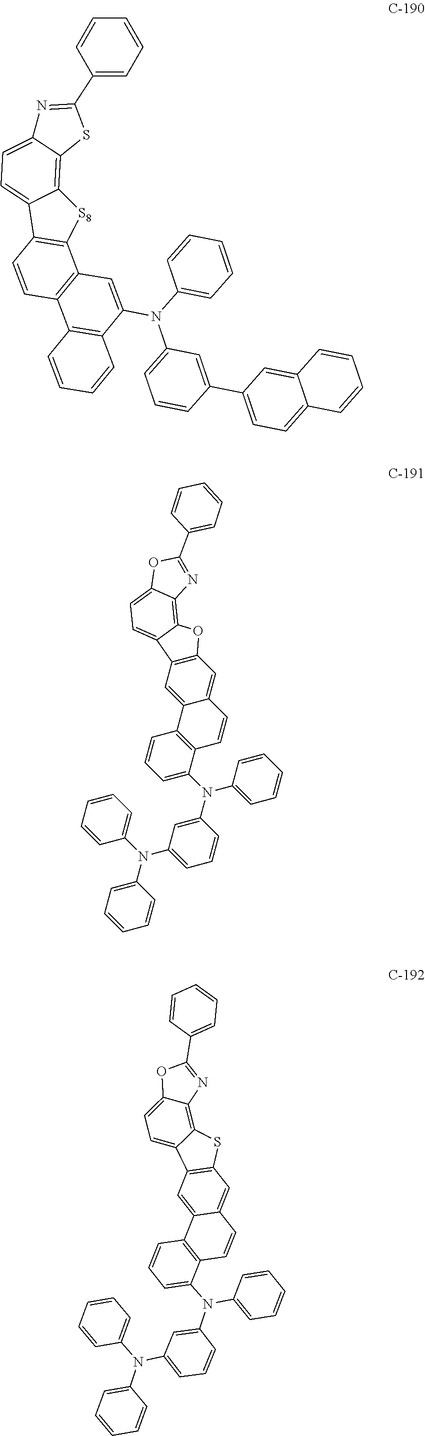

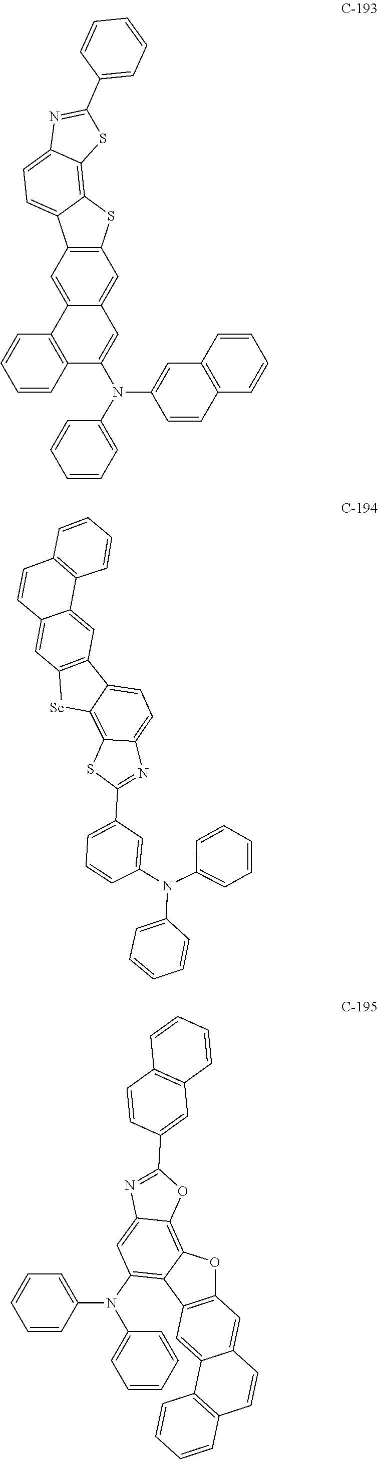

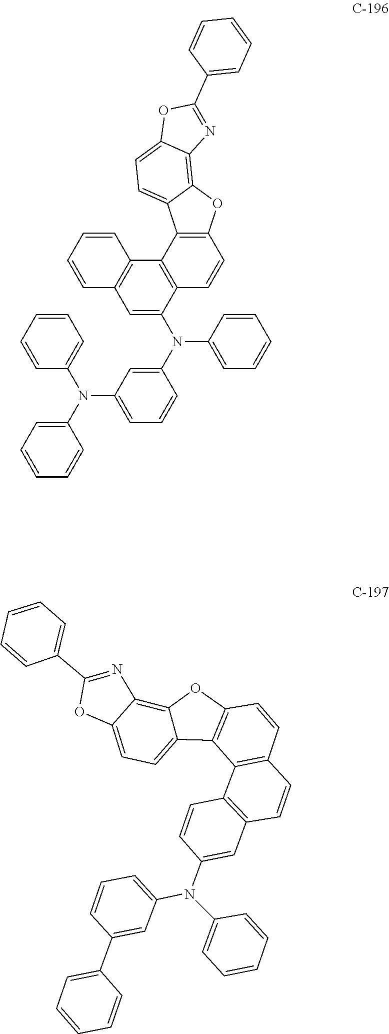 [SMILES: c1ccc(-c2nc3c(ccc4c3oc3ccc5c(N(c6ccccc6)c6cccc(N(c7ccccc7)c7ccccc7)c6)cc6ccccc6c5c34)o2)cc1 ; c1ccc(-c2cccc(N(c3ccccc3)c3ccc4c(ccc5ccc6oc7c(ccc8oc(-c9ccccc9)nc87)c6c54)c3)c2)cc1]

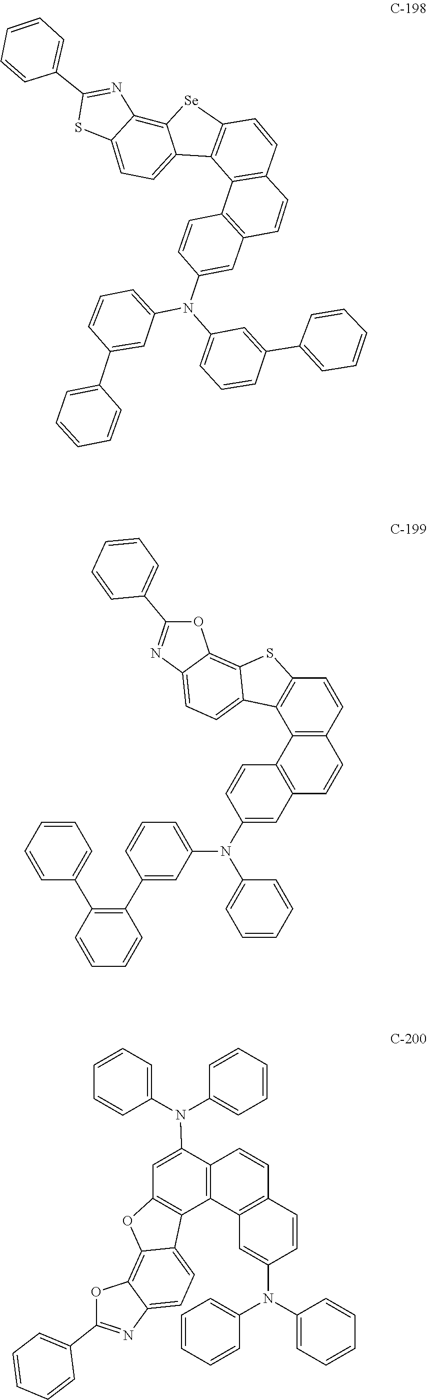

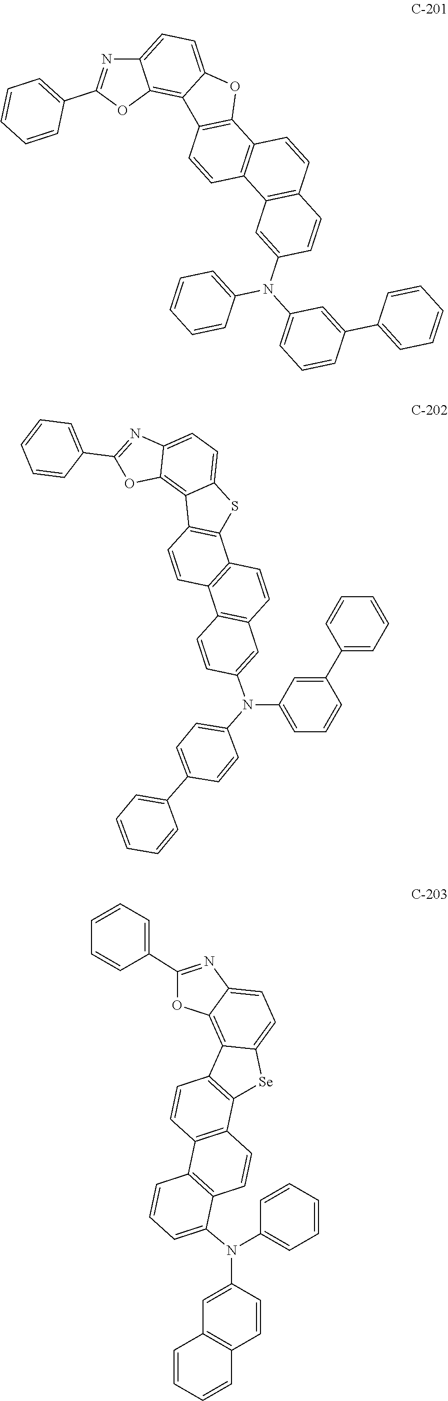 [SMILES: c1ccc(-c2cccc(N(c3ccccc3)c3ccc4ccc5c(ccc6c5oc5ccc7nc(-c8ccccc8)oc7c56)c4c3)c2)cc1 ; c1ccc(-c2ccc(N(c3cccc(-c4ccccc4)c3)c3ccc4c(ccc5c4ccc4c5sc5ccc6nc(-c7ccccc7)oc6c54)c3)cc2)cc1 ; c1ccc(-c2nc3ccc4[se]c5c6ccc7c(N(c8ccccc8)c8ccc9ccccc9c8)cccc7c6ccc5c4c3o2)cc1]

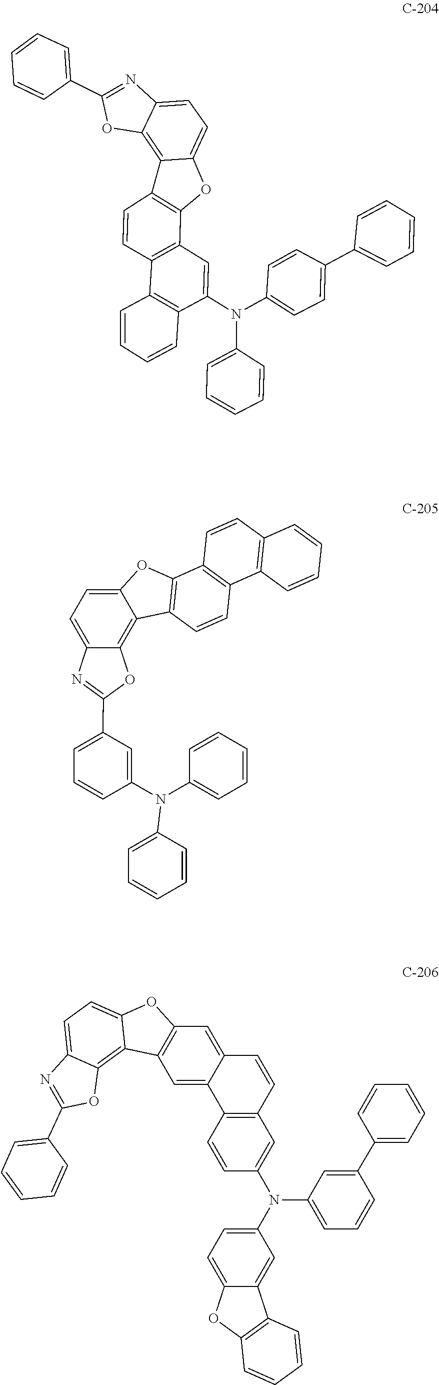 [SMILES: c1ccc(-c2ccc(N(c3ccccc3)c3cc4c(ccc5c4oc4ccc6nc(-c7ccccc7)oc6c45)c4ccccc34)cc2)cc1 ; c1ccc(N(c2ccccc2)c2cccc(-c3nc4ccc5oc6c7ccc8ccccc8c7ccc6c5c4o3)c2)cc1 ; c1ccc(-c2cccc(N(c3ccc4c(ccc5cc6oc7ccc8nc(-c9ccccc9)oc8c7c6cc54)c3)c3ccc4oc5ccccc5c4c3)c2)cc1]

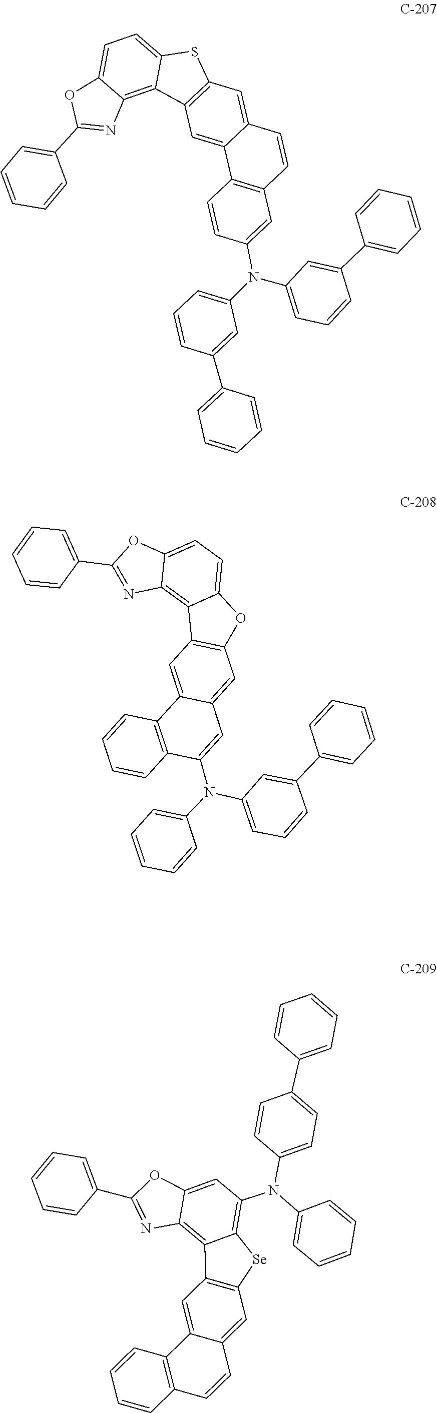 [SMILES: c1ccc(-c2cccc(N(c3cccc(-c4ccccc4)c3)c3ccc4c(ccc5cc6sc7ccc8oc(-c9ccccc9)nc8c7c6cc54)c3)c2)cc1 ; c1ccc(-c2cccc(N(c3ccccc3)c3cc4cc5oc6ccc7oc(-c8ccccc8)nc7c6c5cc4c4ccccc34)c2)cc1 ; c1ccc(-c2ccc(N(c3ccccc3)c3cc4oc(-c5ccccc5)nc4c4c3[se]c3cc5ccc6ccccc6c5cc34)cc2)cc1]

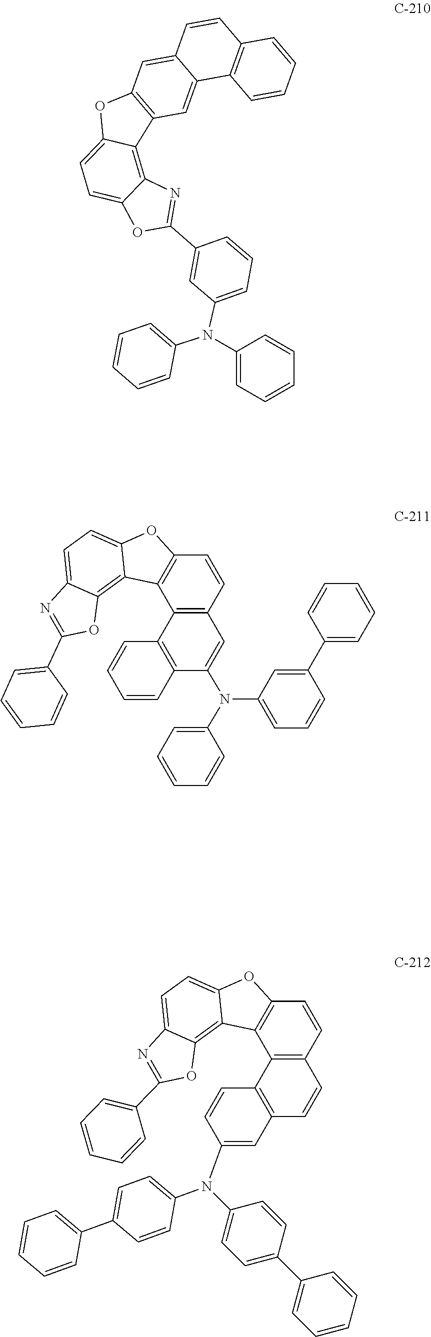 [SMILES: c1ccc(N(c2ccccc2)c2cccc(-c3nc4c(ccc5oc6cc7ccc8ccccc8c7cc6c54)o3)c2)cc1 ; c1ccc(-c2cccc(N(c3ccccc3)c3cc4ccc5oc6ccc7nc(-c8ccccc8)oc7c6c5c4c4ccccc34)c2)cc1 ; c1ccc(-c2ccc(N(c3ccc(-c4ccccc4)cc3)c3ccc4c(ccc5ccc6oc7ccc8nc(-c9ccccc9)oc8c7c6c54)c3)cc2)cc1]

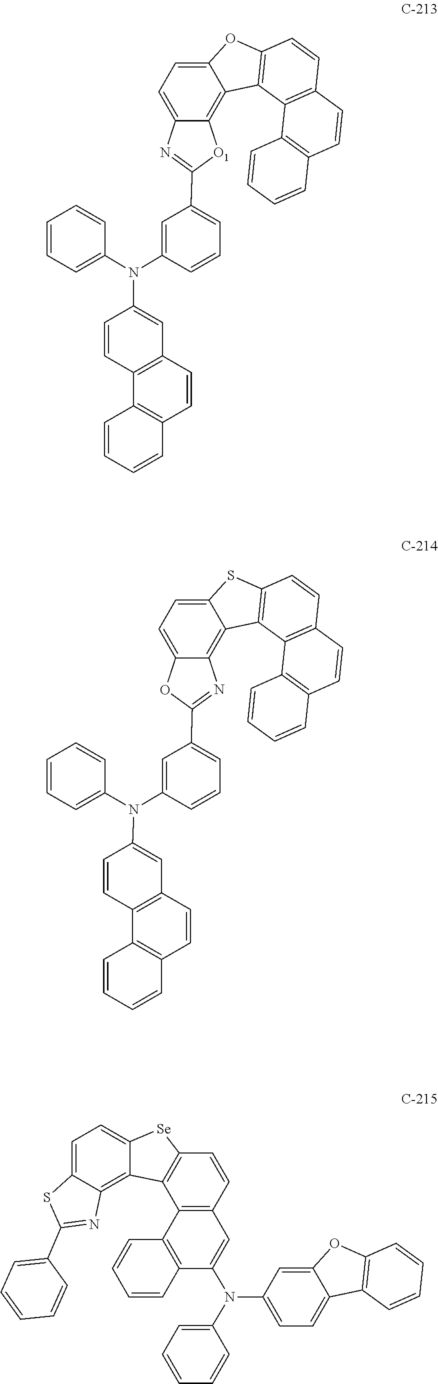

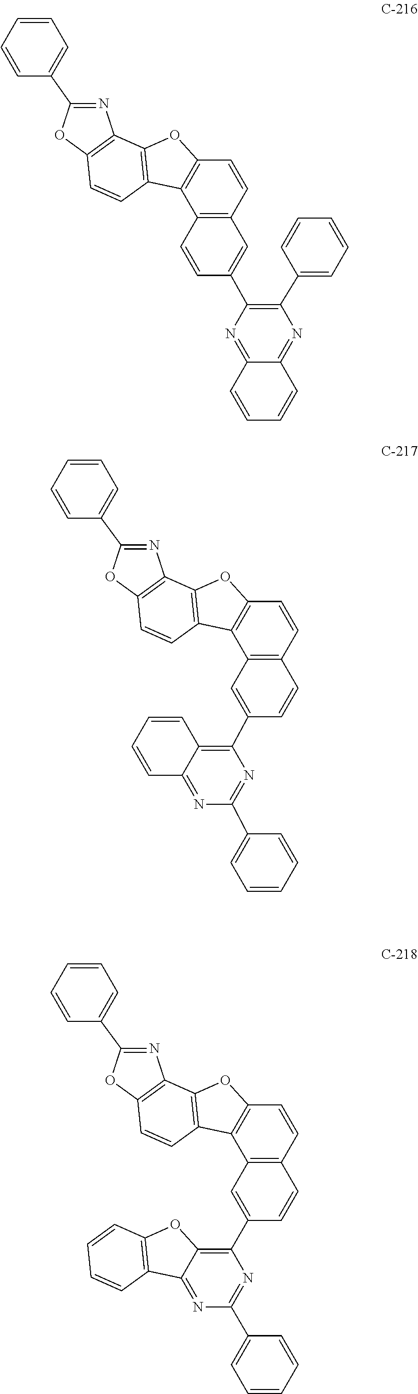 [SMILES: c1ccc(-c2nc3c(ccc4c3oc3ccc5cc(-c6nc7ccccc7nc6-c6ccccc6)ccc5c34)o2)cc1 ; c1ccc(-c2nc(-c3ccc4ccc5oc6c(ccc7oc(-c8ccccc8)nc76)c5c4c3)c3ccccc3n2)cc1 ; c1ccc(-c2nc(-c3ccc4ccc5oc6c(ccc7oc(-c8ccccc8)nc76)c5c4c3)c3oc4ccccc4c3n2)cc1]

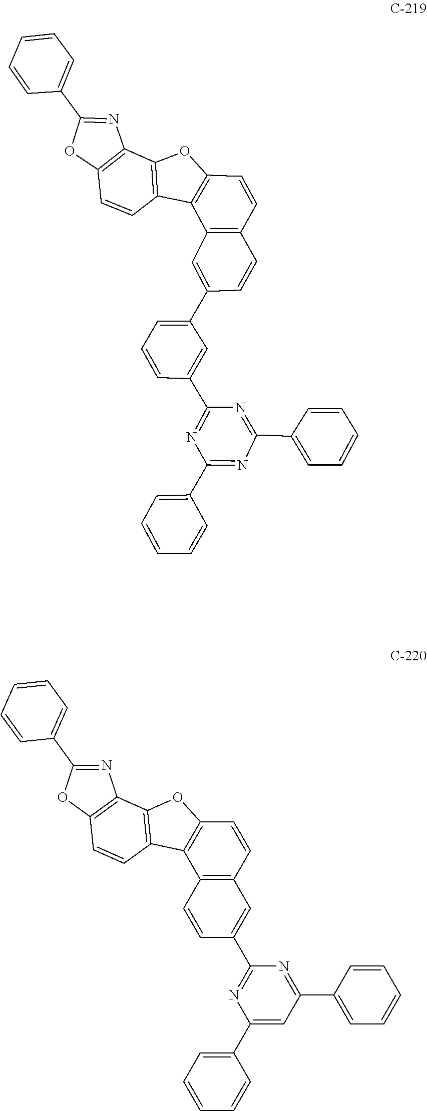 [SMILES: c1ccc(-c2nc(-c3ccccc3)nc(-c3cccc(-c4ccc5ccc6oc7c(ccc8oc(-c9ccccc9)nc87)c6c5c4)c3)n2)cc1 ; c1ccc(-c2cc(-c3ccccc3)nc(-c3ccc4c(ccc5oc6c(ccc7oc(-c8ccccc8)nc76)c54)c3)n2)cc1]

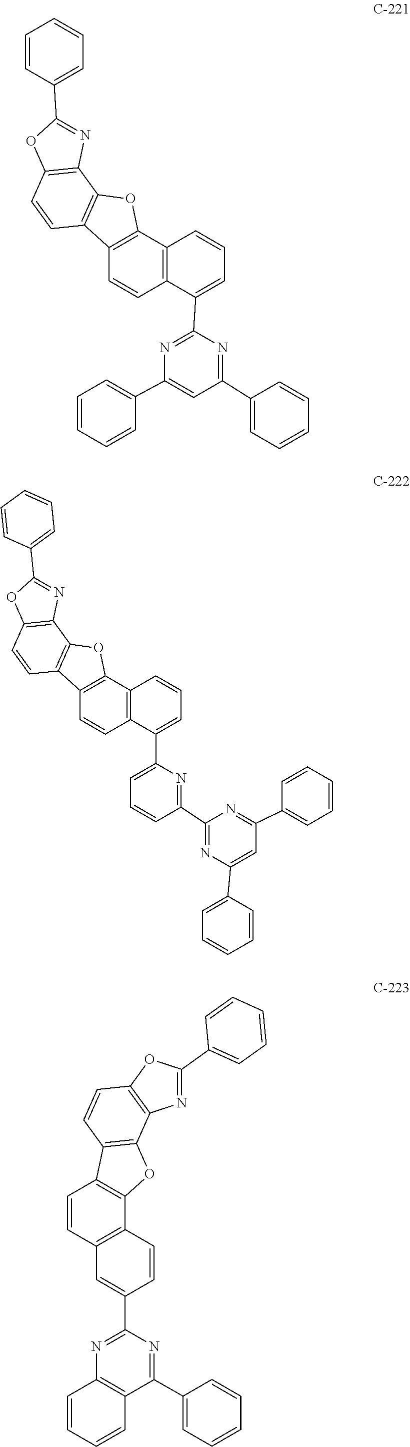

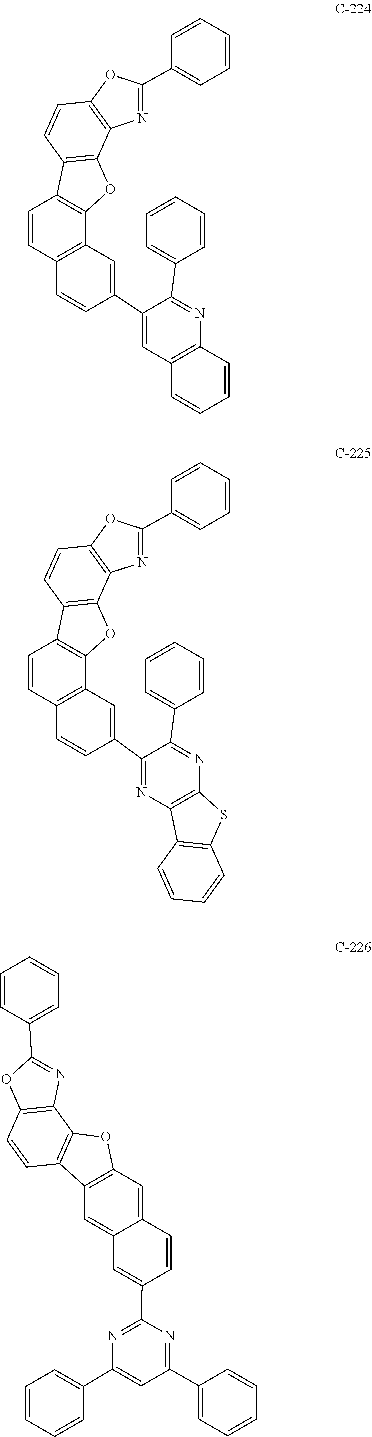 [SMILES: c1ccc(-c2nc3c(ccc4c5ccc6ccc(-c7cc8ccccc8nc7-c7ccccc7)cc6c5oc43)o2)cc1 ; c1ccc(-c2nc3c(ccc4c5ccc6ccc(-c7nc8c(nc7-c7ccccc7)sc7ccccc78)cc6c5oc43)o2)cc1 ; c1ccc(-c2cc(-c3ccccc3)nc(-c3ccc4cc5oc6c(ccc7oc(-c8ccccc8)nc76)c5cc4c3)n2)cc1]

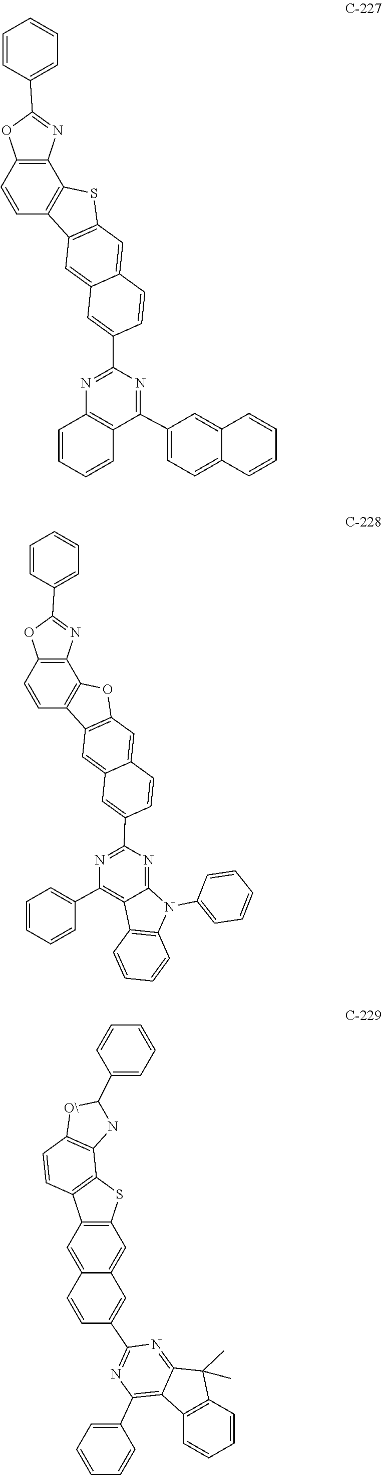

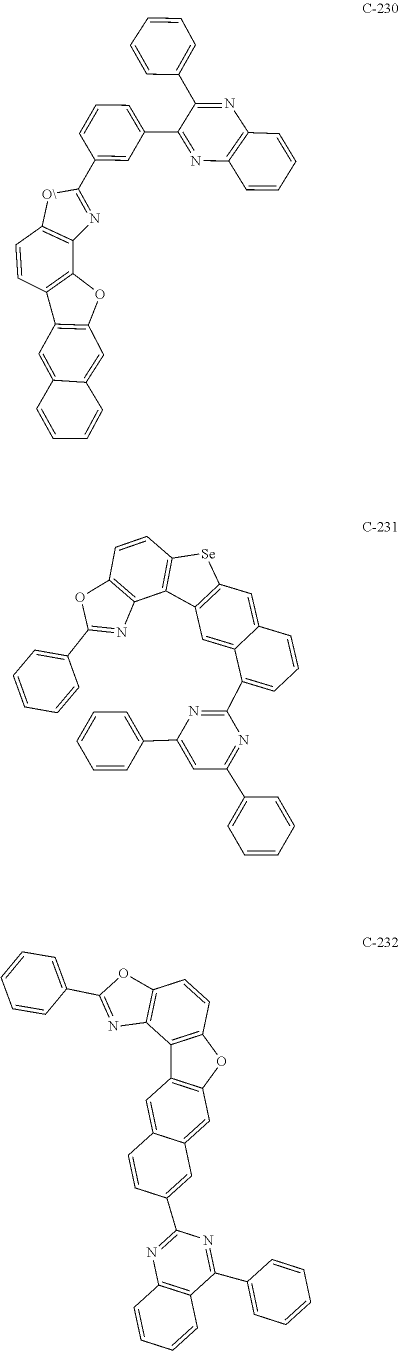

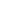

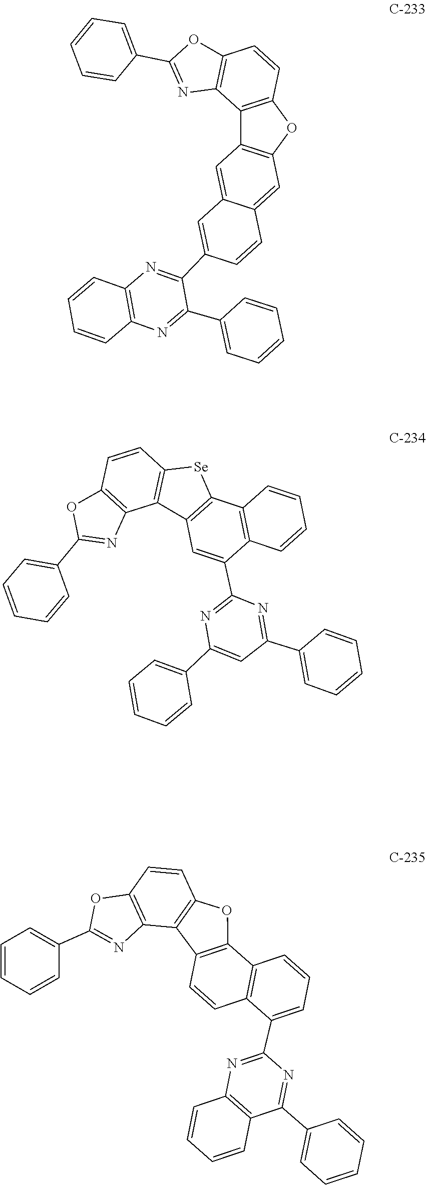 [SMILES: c1ccc(-c2nc3c(ccc4oc5cc6ccc(-c7nc8ccccc8nc7-c7ccccc7)cc6cc5c43)o2)cc1 ; c1ccc(-c2cc(-c3ccccc3)nc(-c3cc4c([se]c5ccc6oc(-c7ccccc7)nc6c54)c4ccccc34)n2)cc1 ; c1ccc(-c2nc3c(ccc4oc5c6cccc(-c7nc(-c8ccccc8)c8ccccc8n7)c6ccc5c43)o2)cc1]

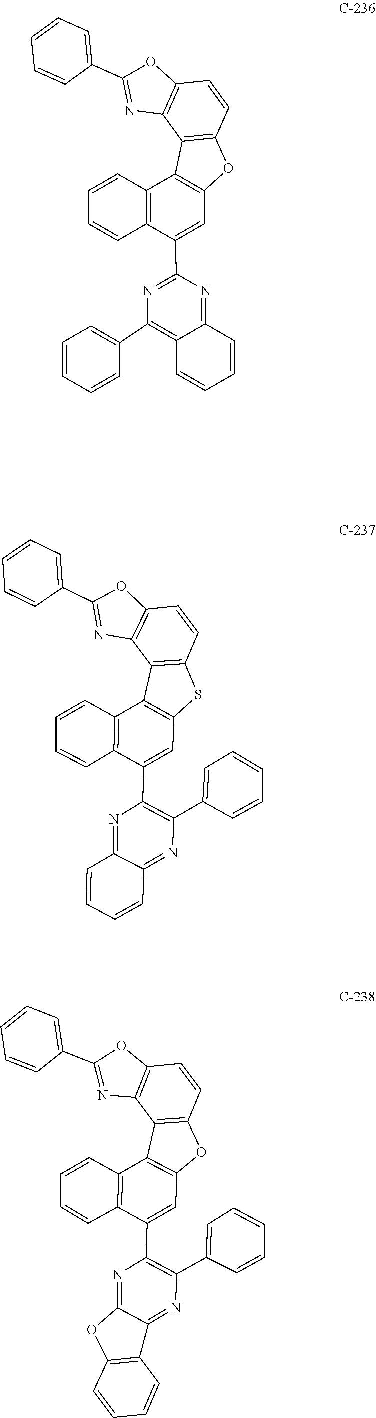 [SMILES: c1ccc(-c2nc3c(ccc4oc5cc(-c6nc(-c7ccccc7)c7ccccc7n6)c6ccccc6c5c43)o2)cc1 ; c1ccc(-c2nc3c(ccc4sc5cc(-c6nc7ccccc7nc6-c6ccccc6)c6ccccc6c5c43)o2)cc1 ; c1ccc(-c2nc3c(ccc4oc5cc(-c6nc7oc8ccccc8c7nc6-c6ccccc6)c6ccccc6c5c43)o2)cc1]

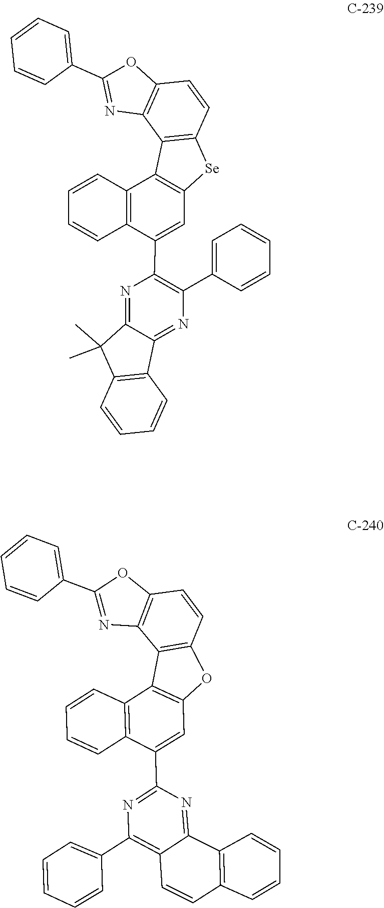 [SMILES: CC1(C)c2ccccc2-c2nc(-c3ccccc3)c(-c3cc4[se]c5ccc6oc(-c7ccccc7)nc6c5c4c4ccccc34)nc21 ; c1ccc(-c2nc3c(ccc4oc5cc(-c6nc(-c7ccccc7)c7ccc8ccccc8c7n6)c6ccccc6c5c43)o2)cc1]

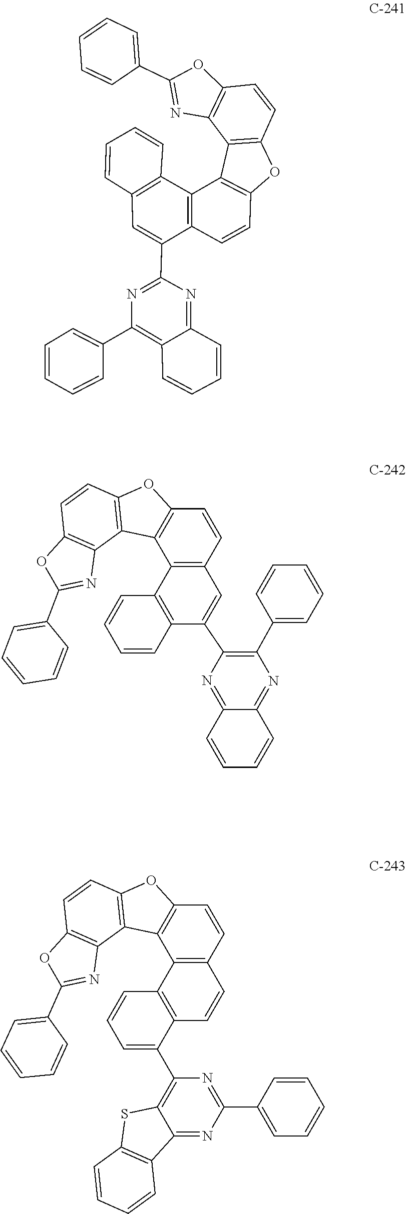 [SMILES: c1ccc(-c2nc3c(ccc4oc5ccc6c(-c7nc(-c8ccccc8)c8ccccc8n7)cc7ccccc7c6c5c43)o2)cc1 ; c1ccc(-c2nc3c(ccc4oc5ccc6cc(-c7nc8ccccc8nc7-c7ccccc7)c7ccccc7c6c5c43)o2)cc1 ; c1ccc(-c2nc(-c3cccc4c3ccc3ccc5oc6ccc7oc(-c8ccccc8)nc7c6c5c34)c3sc4ccccc4c3n2)cc1]

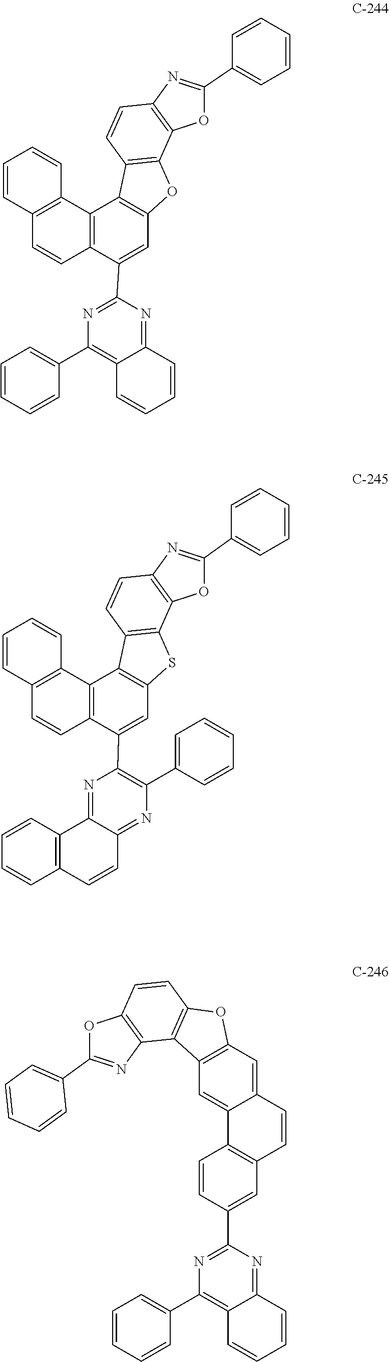 [SMILES: c1ccc(-c2nc3ccc4c(oc5cc(-c6nc(-c7ccccc7)c7ccccc7n6)c6ccc7ccccc7c6c54)c3o2)cc1 ; c1ccc(-c2nc3ccc4c(sc5cc(-c6nc7c(ccc8ccccc87)nc6-c6ccccc6)c6ccc7ccccc7c6c54)c3o2)cc1 ; c1ccc(-c2nc3c(ccc4oc5cc6ccc7cc(-c8nc(-c9ccccc9)c9ccccc9n8)ccc7c6cc5c43)o2)cc1]

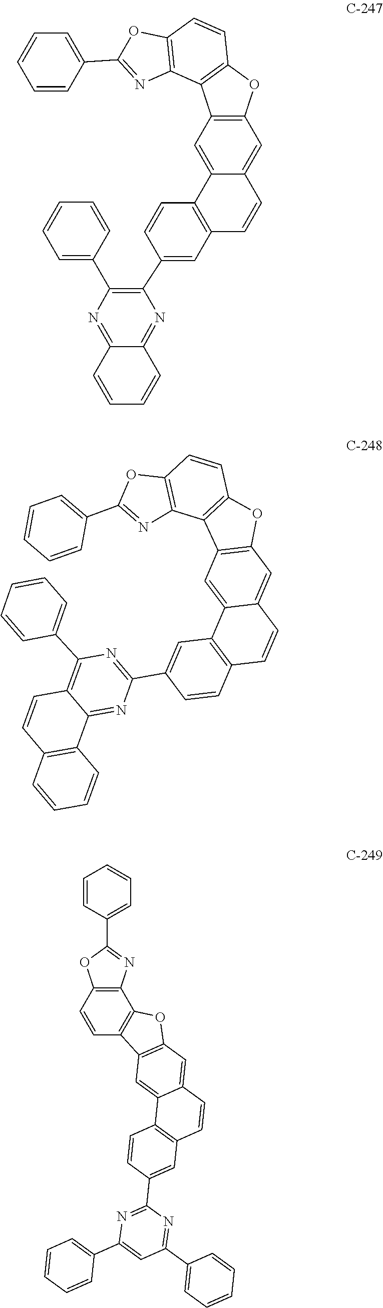

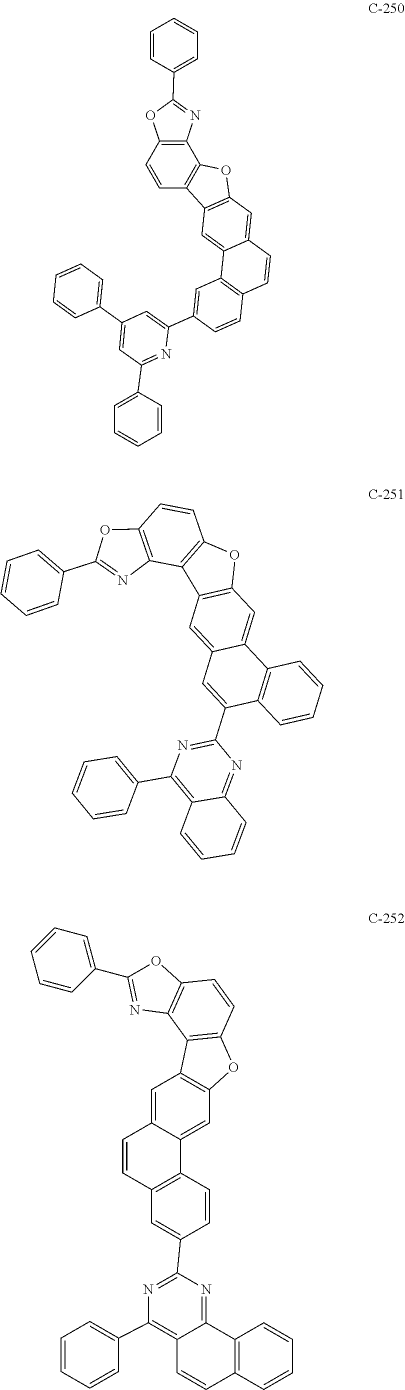 [SMILES: c1ccc(-c2cc(-c3ccccc3)nc(-c3ccc4ccc5cc6oc7c(ccc8oc(-c9ccccc9)nc87)c6cc5c4c3)c2)cc1 ; c1ccc(-c2nc3c(ccc4oc5cc6c(cc(-c7nc(-c8ccccc8)c8ccccc8n7)c7ccccc76)cc5c43)o2)cc1 ; c1ccc(-c2nc3c(ccc4oc5cc6c(ccc7cc(-c8nc(-c9ccccc9)c9ccc%10ccccc%10c9n8)ccc76)cc5c43)o2)cc1]

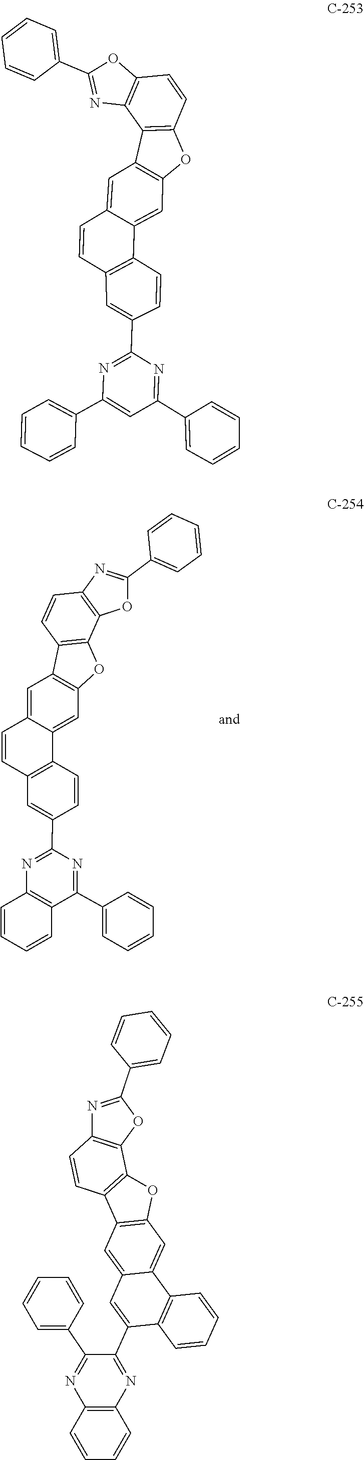 [SMILES: c1ccc(-c2cc(-c3ccccc3)nc(-c3ccc4c(ccc5cc6c(cc54)oc4ccc5oc(-c7ccccc7)nc5c46)c3)n2)cc1 ; c1ccc(-c2nc3ccc4c5cc6ccc7cc(-c8nc(-c9ccccc9)c9ccccc9n8)ccc7c6cc5oc4c3o2)cc1 ; c1ccc(-c2nc3ccc4c5cc6cc(-c7nc8ccccc8nc7-c7ccccc7)c7ccccc7c6cc5oc4c3o2)cc1]

6. An organic electroluminescent material comprising the organic electroluminescent compound according to claim 1.

7. An organic electroluminescent material comprising the organic electroluminescent compound according to claim 1 and any one of compound of following formulas 11 to 13. 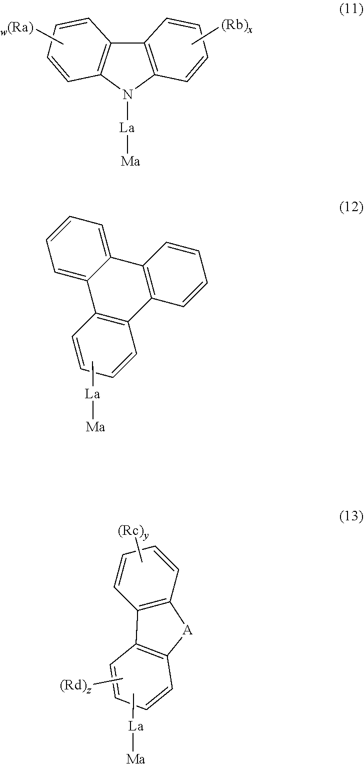
In formulas 11 to 13,
Ma represents a substituted or unsubstituted (C6-C30)aryl, a substituted or unsubstituted mono- or di-(C6-C30)arylamino, a substituted or unsubstituted (C6-C30)aryl(3- to 30-membered)heteroarylamino, or a substituted or unsubstituted (3- to 30-membered)heteroaryl;
La represents a single bond, a substituted or unsubstituted (C6-C30)arylene, or a substituted or unsubstituted (3- to 30-membered)heteroarylene;
A represents S, O, N(Re) or C(Rf)(Rg);
Ra to Rd, each independently, represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C60)aryl, a substituted or unsubstituted (3- to 30-membered)heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted (C1-C30)alkyl(C6-C30)arylamino, or a substituted or unsubstituted mono- or di-(C6-C30)arylamino; or may be linked to an adjacent substituent(s) to form a substituted or unsubstituted mono- or polycyclic (3- to 30-membered) alicyclic or aromatic ring, or the combination thereof, and the formed alicyclic or aromatic ring, or the combination thereof may contain at least one heteroatom selected from N, O, and S;
Re to Rg, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or Rf and Rg may be linked to each other to form a substituted or unsubstituted, mono- or polycyclic, (3- to 30-membered) alicyclic or aromatic ring, or the combination thereof, and the formed alicyclic or aromatic ring, or the combination thereof may contain at least one heteroatom selected from N, O, and S;
w to y, each independently, represent an integer of 1 to 4, and z represents an integer of 1 to 3; where if w to z are each an integer of 2 or more, each of Ra to each of Rd may be the same as or different from each other; and
the heteroaryl(ene) contains at least one heteroatom selected from B, N, O, S, Si, and P.

8. An organic electroluminescent device comprising the organic electroluminescent compound according to claim 1.

9. The organic electroluminescent device according to claim 8, wherein the organic electroluminescent compound is comprised in at least one of a light-emitting layer, a hole transport layer, and a hole auxiliary layer.

10. An organic electroluminescent material comprising the organic electroluminescent compound according to claim 1 and a compound represented by the following Formula 14. 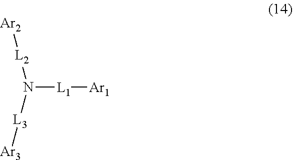
in Formula 14,
L₁ to L₃, each independently, represent a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;
Ar₁ to Ar₃, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or -L₄-N(Arₐ)(Ar_{b});
L₄ represents a single bond, a substituted or unsubstituted (C6-C30)arylene, or a substituted or unsubstituted (3- to 30-membered)heteroarylene;
Arₐ and Ar_{b}, each independently, represent hydrogen, a substituted or unsubstituted (C1-C30)alkyl, a substituted or unsubstituted (C2-C30)alkenyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl; and
with a proviso, the case where all of L₁ to L₃ are single bonds and all of Ar₁ to Ar₃ are hydrogen is excluded.

11. The organic electroluminescent material according to claim 10, wherein at least one of Ar₁ to Ar₃ in Formula 14 is represented by any one of the following formulas 15-1 to 15-5: 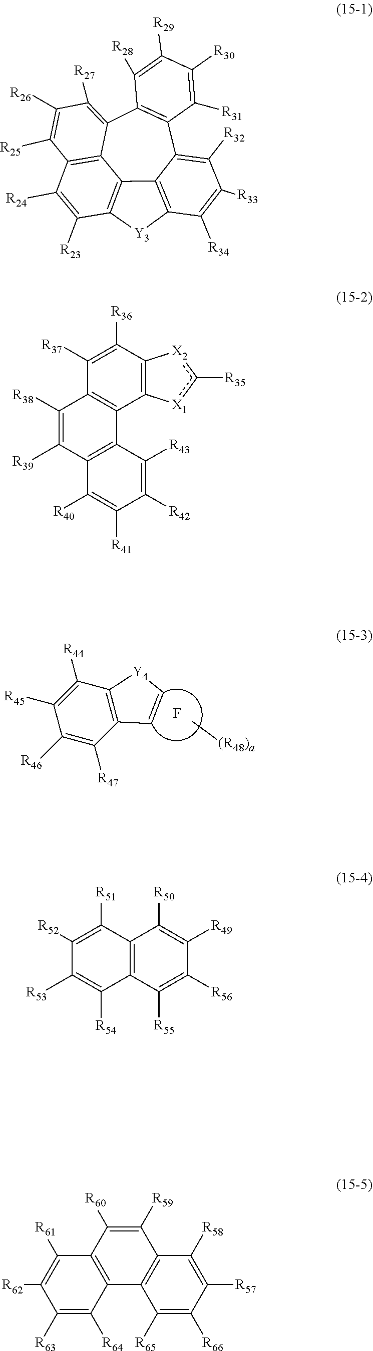
In formulas 15-1 to 15-5,
Y₃ represents O, S, N(R₆₇), or C(R₆₈)(R₆₉);
R₆₇ represents a site linked to any one of L₁ to L₃; or a substituted or unsubstituted (C6-C30)aryl, or a substituted or unsubstituted (3- to 30-membered)heteroaryl;
R₆₈ and R₆₉, each independently, represent a site linked to any one of L₁ to L₃, or a substituted or unsubstituted (C1-C30)alkyl, a substituted or unsubstituted (C6-C30)aryl, or a substituted or unsubstituted (3- to 30-membered)heteroaryl; or R₆₈ and R₆₉ may be linked to each other to form a ring(s);
R₂₃ to R₃₄, each independently, represent a site linked to any one of L₁ to L₃; hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring group of a (C3-C30) aliphatic ring(s) and a (C6-C30) aromatic ring(s); or may be linked to adjacent substituent(s) to form a ring(s);
R₃₅ represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring group of a (C3-C30) aliphatic ring(s) and a (C6-C30) aromatic ring(s);
R₃₆ to R₄₃, each independently, represent a site linked to any one of L₁ to L₃; hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring group of a (C3-C30) aliphatic ring(s) and a (C6-C30) aromatic ring(s); or may be linked to adjacent substituent(s) to form a ring(s);
X₁ and X₂, each independently, represent —O—, —S—, —Se— or —N═, and any one of X₁ and X₂ is —N═;
Y₄ represents O, S, N(R₇₀), or C(R₇₁)(R₇₂);
R₇₀ represents a site linked to any one of L₁ to L₃, or a substituted or unsubstituted (C6-C30)aryl;
R₇₁ and R₇₂, each independently, represent a site linked to any one of L₁ to L₃, or a substituted or unsubstituted (C1-C30)alkyl, a substituted or unsubstituted (C6-C30)aryl, or a substituted or unsubstituted (3- to 30-membered)heteroaryl; or R₇₁ and R₇₂ may be linked to each other to form a ring(s);
ring F is benzene, naphthalene or phenanthrene;
a represents an integer of 3 to 10;
R₄₄ to R₄₈, each independently, represent a site linked to any one of L₁ to L₃; hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring group of a (C3-C30) aliphatic ring(s) and a (C6-C30) aromatic ring(s); or may be linked to adjacent substituent(s) to form a ring(s);
R₄₉ to R₅₆, each independently, represent a site linked to any one of L₁ to L₃; hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring group of a (C3-C30) aliphatic ring(s) and a (C6-C30) aromatic ring(s); or may be linked to adjacent substituent(s) to form a ring(s);
R₅₇ to R₆₆, each independently, represent a site linked to any one of L₁ to L₃; hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring group of a (C3-C30) aliphatic ring(s) and a (C6-C30) aromatic ring(s); or may be linked to adjacent substituent(s) to form a ring(s).

12. The organic electroluminescent material according to claim 10, wherein the compound represented by Formula 14 is selected from the following compounds: 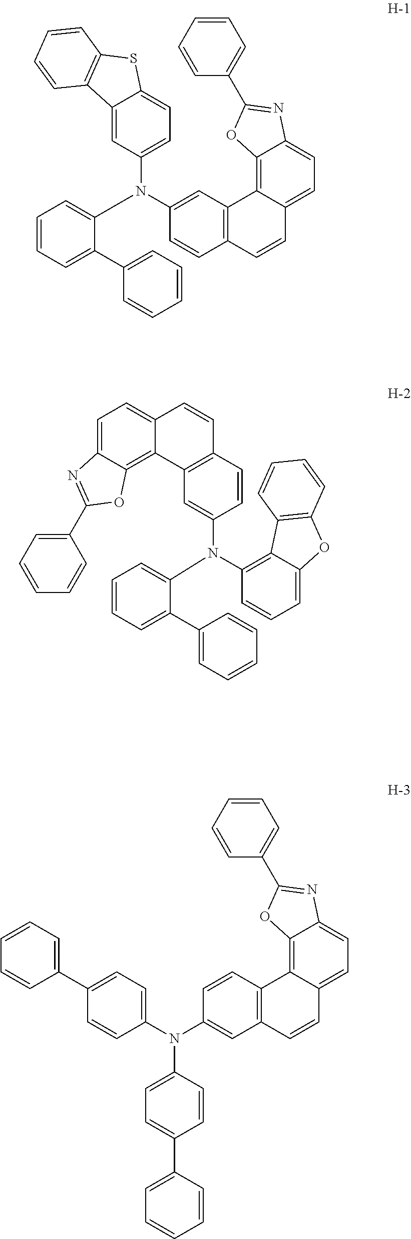 [SMILES: c1ccc(-c2nc3ccc4ccc5ccc(N(c6ccc7sc8ccccc8c7c6)c6ccccc6-c6ccccc6)cc5c4c3o2)cc1 ; c1ccc(-c2nc3ccc4ccc5ccc(N(c6ccccc6-c6ccccc6)c6cccc7oc8ccccc8c67)cc5c4c3o2)cc1 ; c1ccc(-c2ccc(N(c3ccc(-c4ccccc4)cc3)c3ccc4c(ccc5ccc6nc(-c7ccccc7)oc6c54)c3)cc2)cc1] 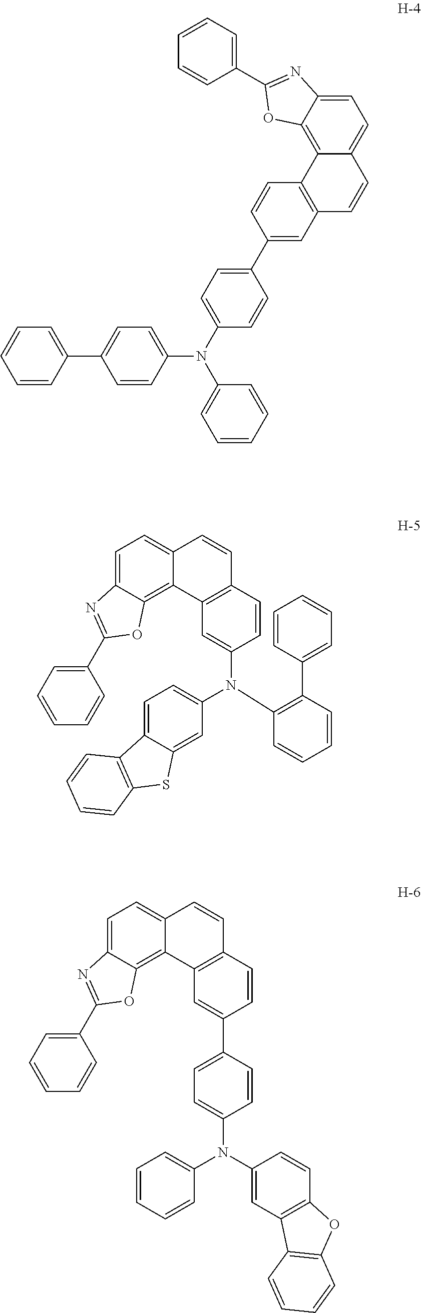 [SMILES: c1ccc(-c2ccc(N(c3ccccc3)c3ccc(-c4ccc5c(ccc6ccc7nc(-c8ccccc8)oc7c65)c4)cc3)cc2)cc1 ; c1ccc(-c2nc3ccc4ccc5ccc(N(c6ccc7c(c6)sc6ccccc67)c6ccccc6-c6ccccc6)cc5c4c3o2)cc1 ; c1ccc(-c2nc3ccc4ccc5ccc(-c6ccc(N(c7ccccc7)c7ccc8oc9ccccc9c8c7)cc6)cc5c4c3o2)cc1] 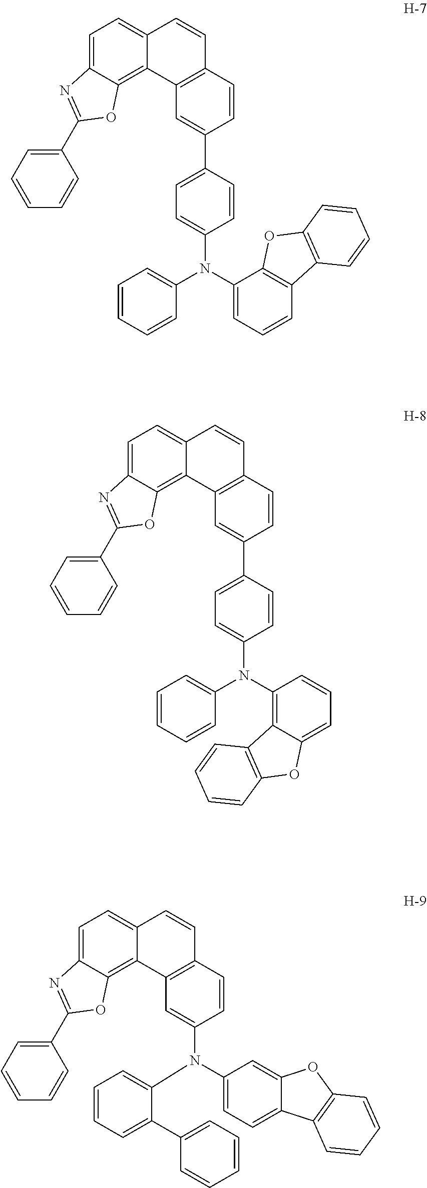 [SMILES: c1ccc(-c2nc3ccc4ccc5ccc(-c6ccc(N(c7ccccc7)c7cccc8c7oc7ccccc78)cc6)cc5c4c3o2)cc1 ; c1ccc(-c2nc3ccc4ccc5ccc(-c6ccc(N(c7ccccc7)c7cccc8oc9ccccc9c78)cc6)cc5c4c3o2)cc1 ; c1ccc(-c2nc3ccc4ccc5ccc(N(c6ccc7c(c6)oc6ccccc67)c6ccccc6-c6ccccc6)cc5c4c3o2)cc1] 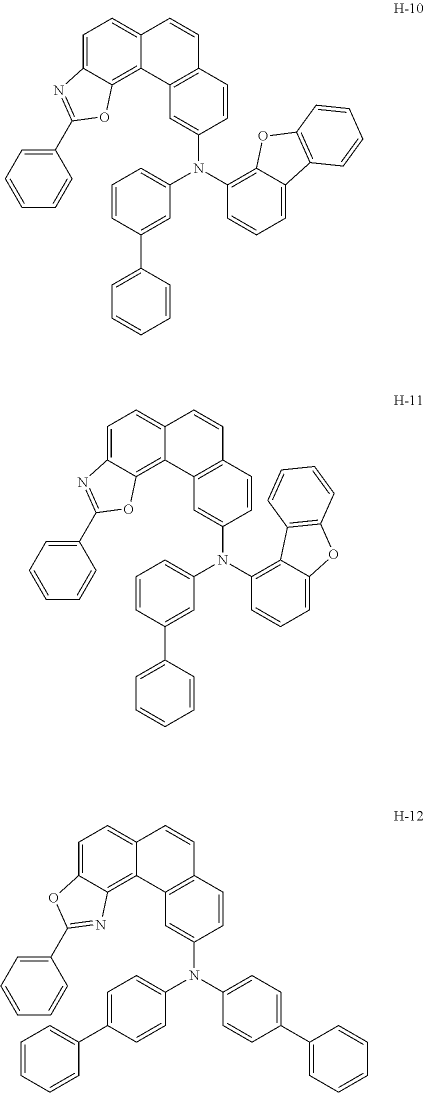 [SMILES: c1ccc(-c2cccc(N(c3ccc4ccc5ccc6nc(-c7ccccc7)oc6c5c4c3)c3cccc4c3oc3ccccc34)c2)cc1 ; c1ccc(-c2cccc(N(c3ccc4ccc5ccc6nc(-c7ccccc7)oc6c5c4c3)c3cccc4oc5ccccc5c34)c2)cc1 ; c1ccc(-c2ccc(N(c3ccc(-c4ccccc4)cc3)c3ccc4ccc5ccc6oc(-c7ccccc7)nc6c5c4c3)cc2)cc1] 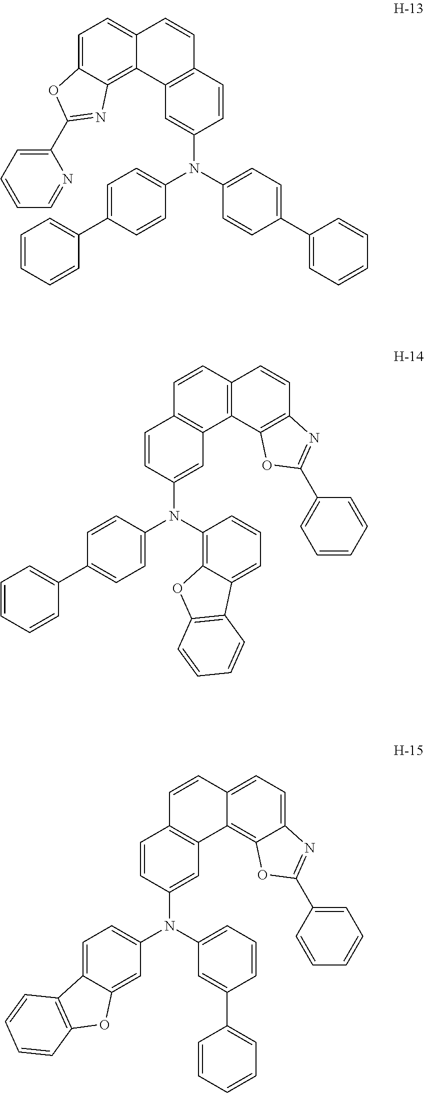 [SMILES: c1ccc(-c2ccc(N(c3ccc(-c4ccccc4)cc3)c3ccc4ccc5ccc6oc(-c7ccccn7)nc6c5c4c3)cc2)cc1 ; c1ccc(-c2ccc(N(c3ccc4ccc5ccc6nc(-c7ccccc7)oc6c5c4c3)c3cccc4c3oc3ccccc34)cc2)cc1 ; c1ccc(-c2cccc(N(c3ccc4c(c3)oc3ccccc34)c3ccc4ccc5ccc6nc(-c7ccccc7)oc6c5c4c3)c2)cc1] 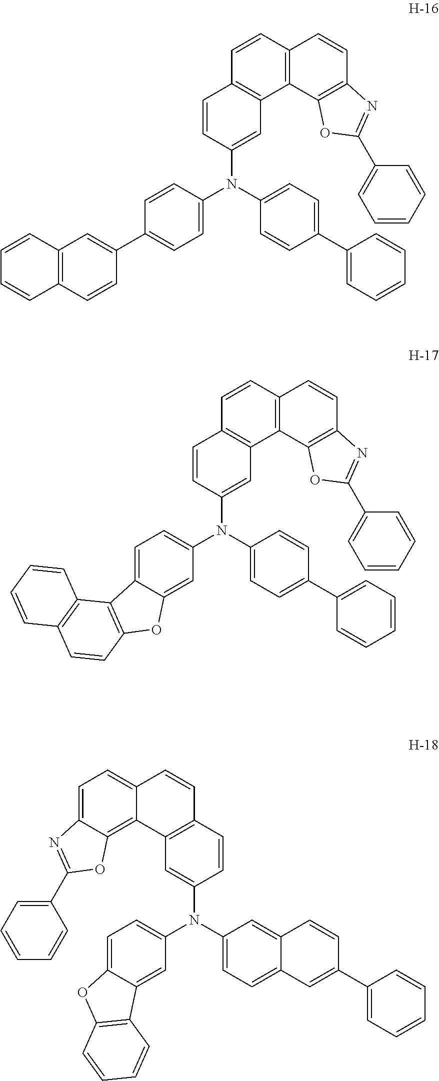 [SMILES: c1ccc(-c2ccc(N(c3ccc(-c4ccc5ccccc5c4)cc3)c3ccc4ccc5ccc6nc(-c7ccccc7)oc6c5c4c3)cc2)cc1 ; c1ccc(-c2ccc(N(c3ccc4c(c3)oc3ccc5ccccc5c34)c3ccc4ccc5ccc6nc(-c7ccccc7)oc6c5c4c3)cc2)cc1 ; c1ccc(-c2ccc3cc(N(c4ccc5oc6ccccc6c5c4)c4ccc5ccc6ccc7nc(-c8ccccc8)oc7c6c5c4)ccc3c2)cc1] 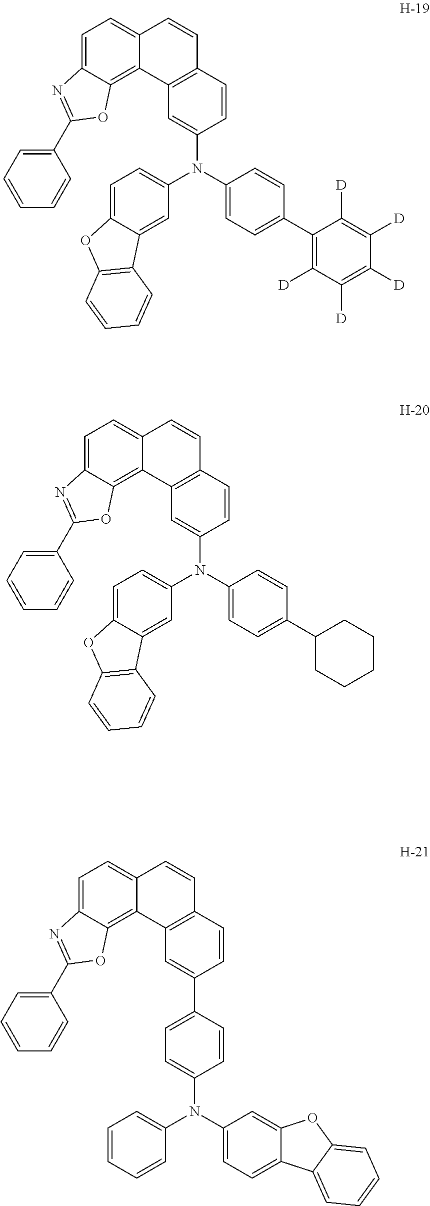 [SMILES: [2H]c1c([2H])c([2H])c(-c2ccc(N(c3ccc4oc5ccccc5c4c3)c3ccc4ccc5ccc6nc(-c7ccccc7)oc6c5c4c3)cc2)c([2H])c1[2H] ; c1ccc(-c2nc3ccc4ccc5ccc(N(c6ccc(C7CCCCC7)cc6)c6ccc7oc8ccccc8c7c6)cc5c4c3o2)cc1 ; c1ccc(-c2nc3ccc4ccc5ccc(-c6ccc(N(c7ccccc7)c7ccc8c(c7)oc7ccccc78)cc6)cc5c4c3o2)cc1] 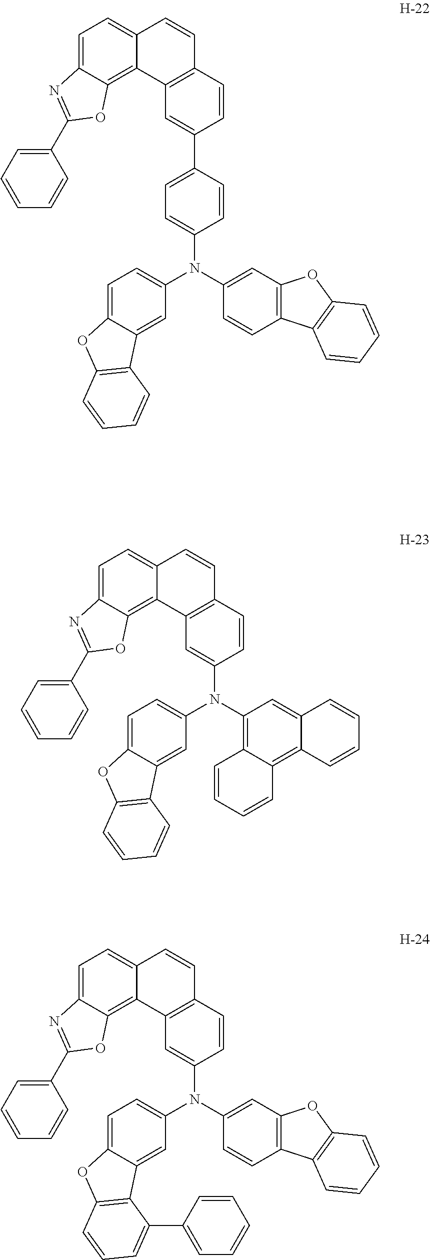 [SMILES: c1ccc(-c2nc3ccc4ccc5ccc(-c6ccc(N(c7ccc8c(c7)oc7ccccc78)c7ccc8oc9ccccc9c8c7)cc6)cc5c4c3o2)cc1 ; c1ccc(-c2nc3ccc4ccc5ccc(N(c6ccc7oc8ccccc8c7c6)c6cc7ccccc7c7ccccc67)cc5c4c3o2)cc1 ; c1ccc(-c2nc3ccc4ccc5ccc(N(c6ccc7c(c6)oc6ccccc67)c6ccc7oc8cccc(-c9ccccc9)c8c7c6)cc5c4c3o2)cc1] 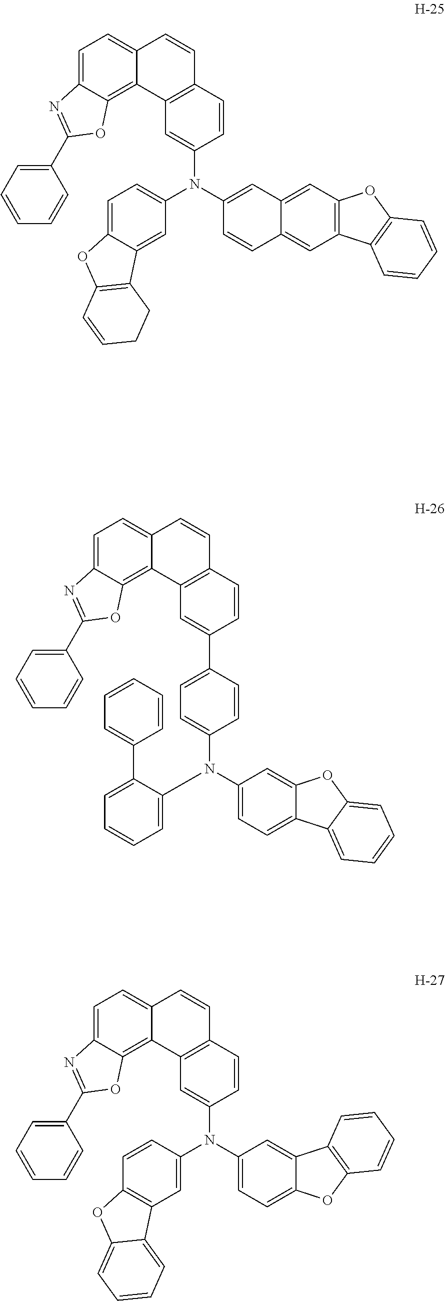 [SMILES: C1=Cc2oc3ccc(N(c4ccc5cc6c(cc5c4)oc4ccccc46)c4ccc5ccc6ccc7nc(-c8ccccc8)oc7c6c5c4)cc3c2CC1 ; c1ccc(-c2nc3ccc4ccc5ccc(-c6ccc(N(c7ccc8c(c7)oc7ccccc78)c7ccccc7-c7ccccc7)cc6)cc5c4c3o2)cc1 ; c1ccc(-c2nc3ccc4ccc5ccc(N(c6ccc7oc8ccccc8c7c6)c6ccc7oc8ccccc8c7c6)cc5c4c3o2)cc1] 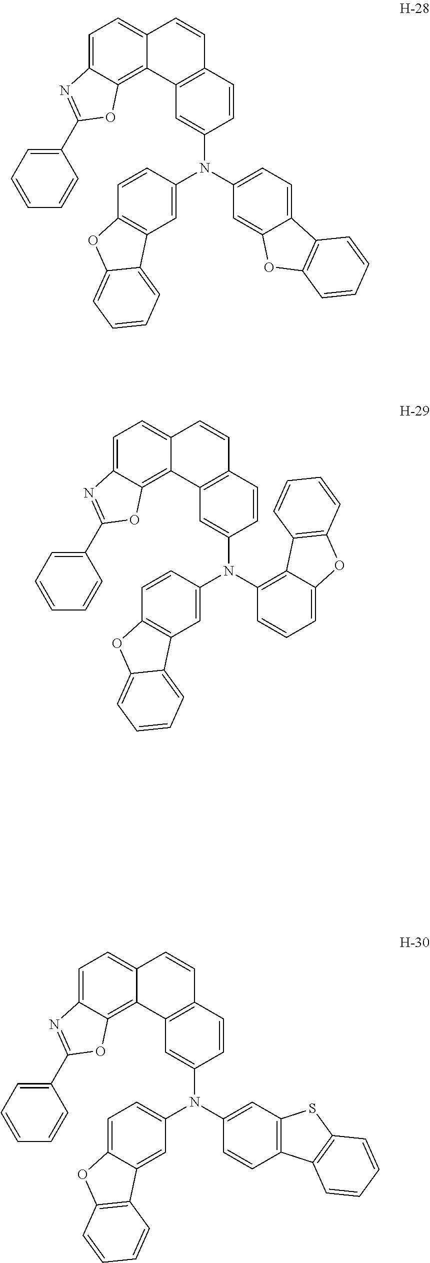 [SMILES: c1ccc(-c2nc3ccc4ccc5ccc(N(c6ccc7c(c6)oc6ccccc67)c6ccc7oc8ccccc8c7c6)cc5c4c3o2)cc1 ; c1ccc(-c2nc3ccc4ccc5ccc(N(c6ccc7oc8ccccc8c7c6)c6cccc7oc8ccccc8c67)cc5c4c3o2)cc1 ; c1ccc(-c2nc3ccc4ccc5ccc(N(c6ccc7c(c6)sc6ccccc67)c6ccc7oc8ccccc8c7c6)cc5c4c3o2)cc1] 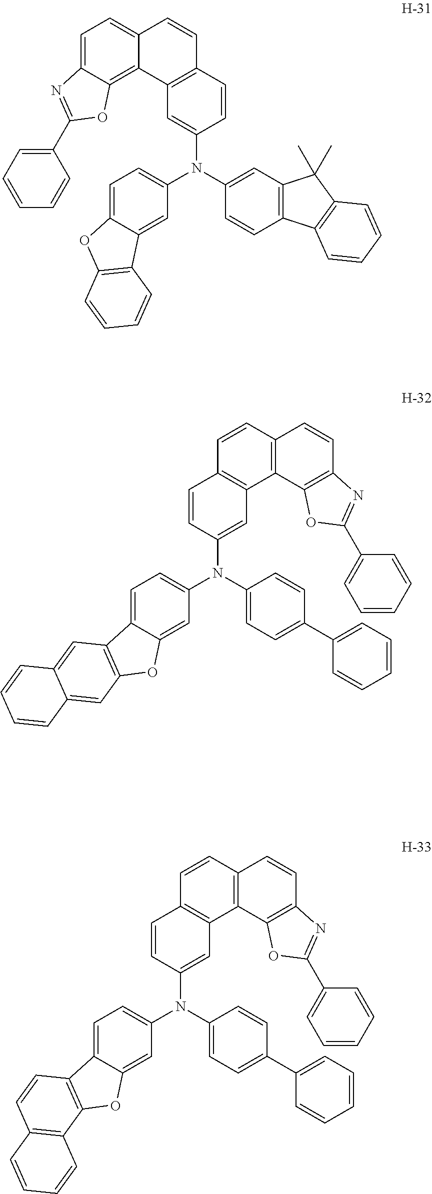 [SMILES: CC1(C)c2ccccc2-c2ccc(N(c3ccc4oc5ccccc5c4c3)c3ccc4ccc5ccc6nc(-c7ccccc7)oc6c5c4c3)cc21 ; c1ccc(-c2ccc(N(c3ccc4c(c3)oc3cc5ccccc5cc34)c3ccc4ccc5ccc6nc(-c7ccccc7)oc6c5c4c3)cc2)cc1 ; c1ccc(-c2ccc(N(c3ccc4c(c3)oc3c5ccccc5ccc43)c3ccc4ccc5ccc6nc(-c7ccccc7)oc6c5c4c3)cc2)cc1] 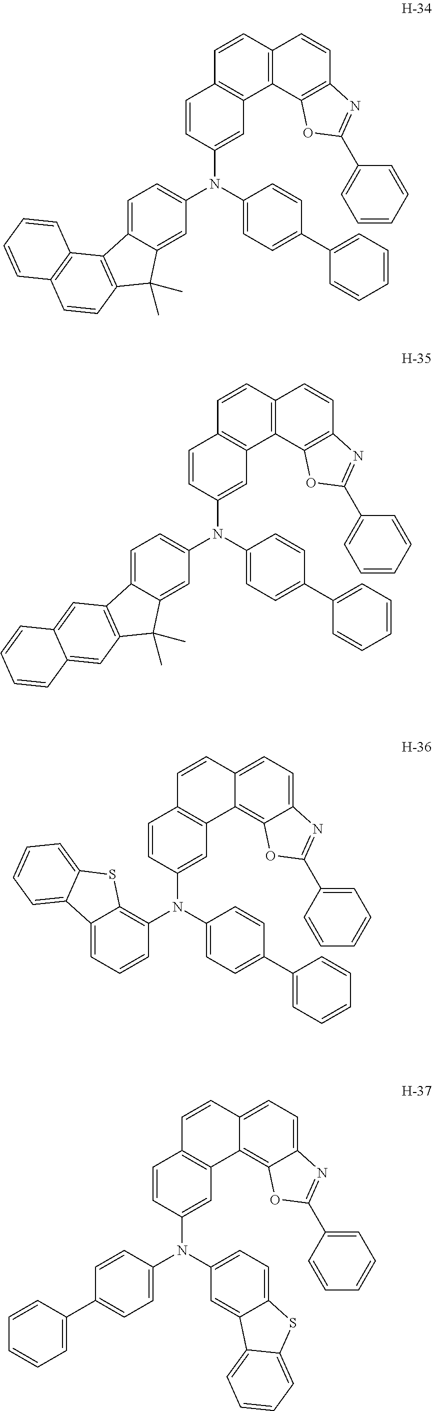 [SMILES: CC1(C)c2cc(N(c3ccc(-c4ccccc4)cc3)c3ccc4ccc5ccc6nc(-c7ccccc7)oc6c5c4c3)ccc2-c2c1ccc1ccccc21 ; CC1(C)c2cc(N(c3ccc(-c4ccccc4)cc3)c3ccc4ccc5ccc6nc(-c7ccccc7)oc6c5c4c3)ccc2-c2cc3ccccc3cc21 ; c1ccc(-c2ccc(N(c3ccc4ccc5ccc6nc(-c7ccccc7)oc6c5c4c3)c3cccc4c3sc3ccccc34)cc2)cc1 ; c1ccc(-c2ccc(N(c3ccc4sc5ccccc5c4c3)c3ccc4ccc5ccc6nc(-c7ccccc7)oc6c5c4c3)cc2)cc1] 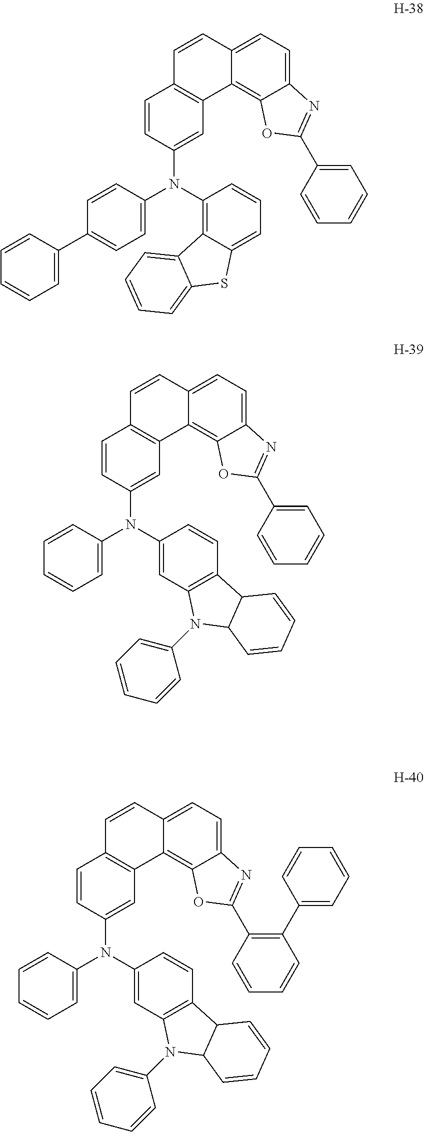 [SMILES: c1ccc(-c2ccc(N(c3ccc4ccc5ccc6nc(-c7ccccc7)oc6c5c4c3)c3cccc4sc5ccccc5c34)cc2)cc1 ; C1=CC2c3ccc(N(c4ccccc4)c4ccc5ccc6ccc7nc(-c8ccccc8)oc7c6c5c4)cc3N(c3ccccc3)C2C=C1 ; C1=CC2c3ccc(N(c4ccccc4)c4ccc5ccc6ccc7nc(-c8ccccc8-c8ccccc8)oc7c6c5c4)cc3N(c3ccccc3)C2C=C1] 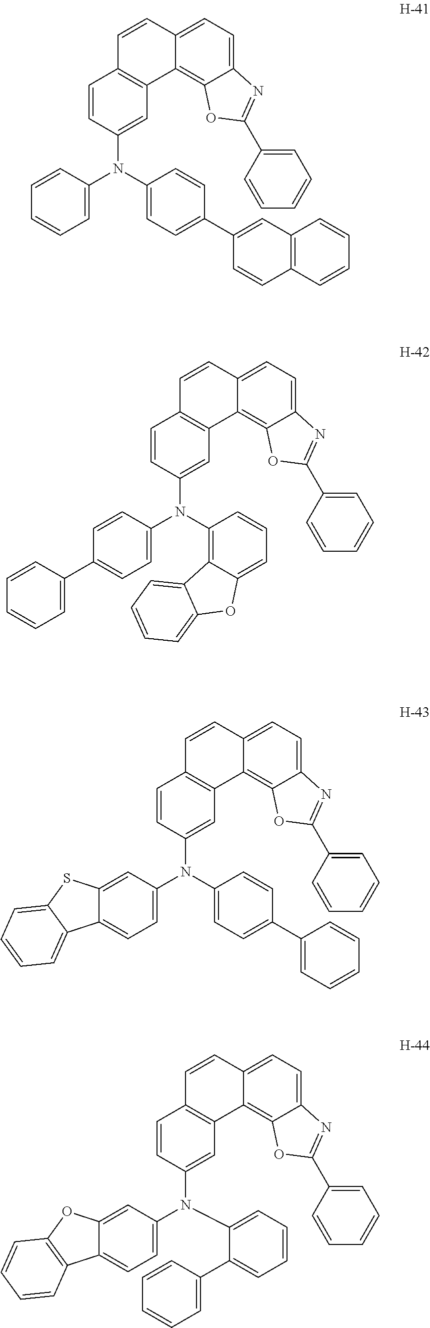 [SMILES: c1ccc(-c2nc3ccc4ccc5ccc(N(c6ccccc6)c6ccc(-c7ccc8ccccc8c7)cc6)cc5c4c3o2)cc1 ; c1ccc(-c2ccc(N(c3ccc4ccc5ccc6nc(-c7ccccc7)oc6c5c4c3)c3cccc4oc5ccccc5c34)cc2)cc1 ; c1ccc(-c2ccc(N(c3ccc4c(c3)sc3ccccc34)c3ccc4ccc5ccc6nc(-c7ccccc7)oc6c5c4c3)cc2)cc1 ; c1ccc(-c2nc3ccc4ccc5ccc(N(c6ccc7c(c6)oc6ccccc67)c6ccccc6-c6ccccc6)cc5c4c3o2)cc1] 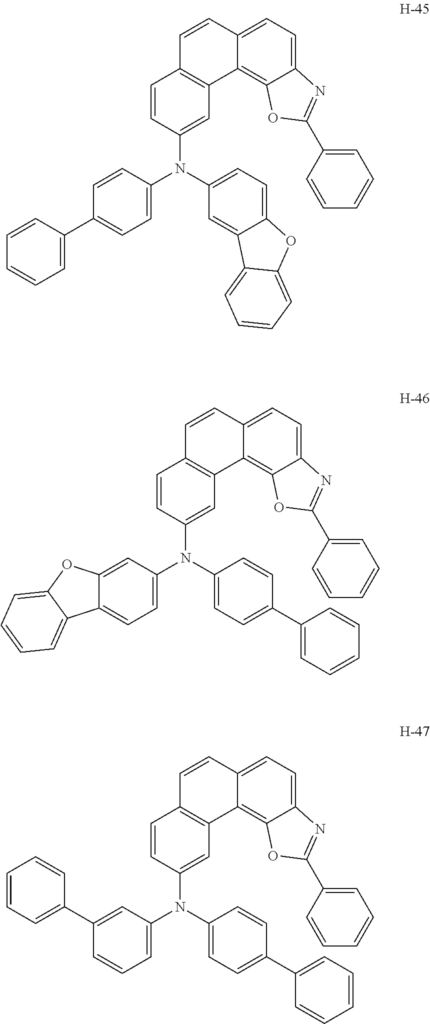 [SMILES: c1ccc(-c2ccc(N(c3ccc4oc5ccccc5c4c3)c3ccc4ccc5ccc6nc(-c7ccccc7)oc6c5c4c3)cc2)cc1 ; c1ccc(-c2ccc(N(c3ccc4c(c3)oc3ccccc34)c3ccc4ccc5ccc6nc(-c7ccccc7)oc6c5c4c3)cc2)cc1 ; c1ccc(-c2ccc(N(c3cccc(-c4ccccc4)c3)c3ccc4ccc5ccc6nc(-c7ccccc7)oc6c5c4c3)cc2)cc1] 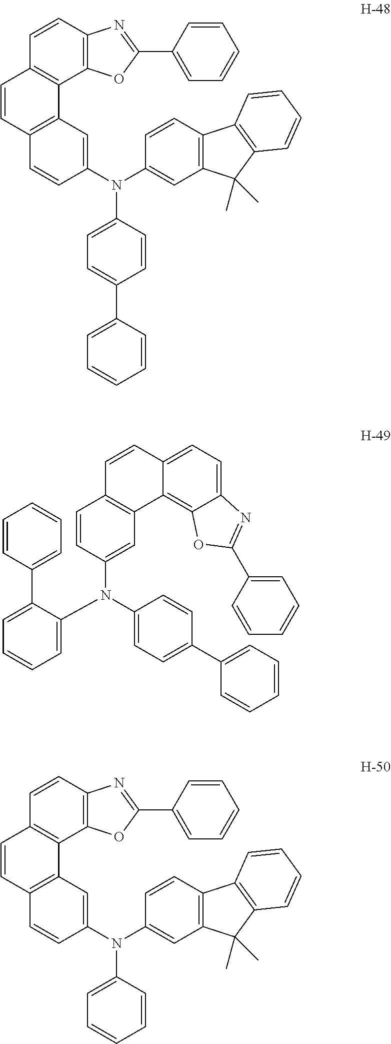 [SMILES: CC1(C)c2ccccc2-c2ccc(N(c3ccc(-c4ccccc4)cc3)c3ccc4ccc5ccc6nc(-c7ccccc7)oc6c5c4c3)cc21 ; c1ccc(-c2ccc(N(c3ccc4ccc5ccc6nc(-c7ccccc7)oc6c5c4c3)c3ccccc3-c3ccccc3)cc2)cc1 ; CC1(C)c2ccccc2-c2ccc(N(c3ccccc3)c3ccc4ccc5ccc6nc(-c7ccccc7)oc6c5c4c3)cc21] 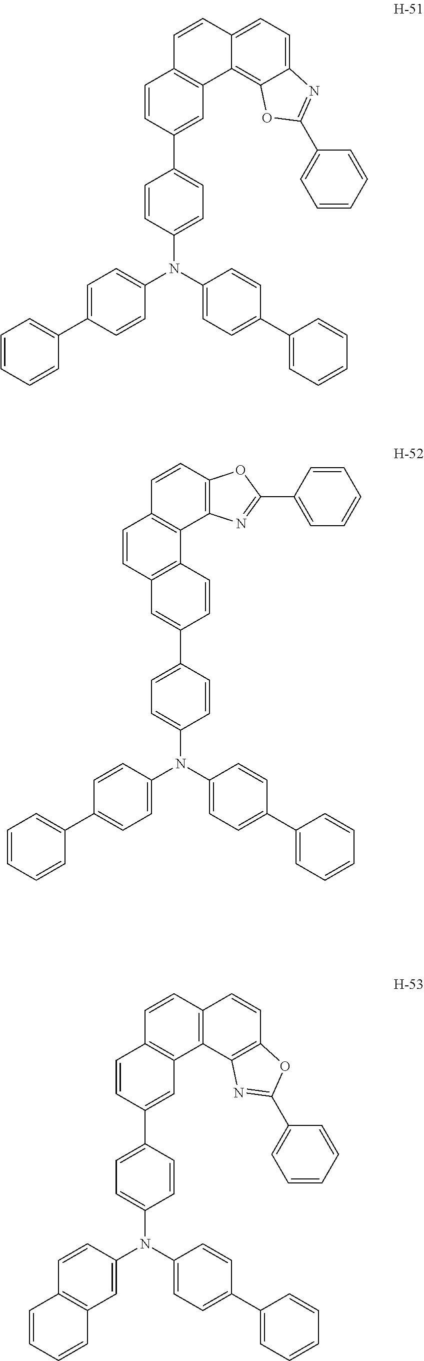 [SMILES: c1ccc(-c2ccc(N(c3ccc(-c4ccccc4)cc3)c3ccc(-c4ccc5ccc6ccc7nc(-c8ccccc8)oc7c6c5c4)cc3)cc2)cc1 ; c1ccc(-c2ccc(N(c3ccc(-c4ccccc4)cc3)c3ccc(-c4ccc5c(ccc6ccc7oc(-c8ccccc8)nc7c65)c4)cc3)cc2)cc1 ; c1ccc(-c2ccc(N(c3ccc(-c4ccc5ccc6ccc7oc(-c8ccccc8)nc7c6c5c4)cc3)c3ccc4ccccc4c3)cc2)cc1] 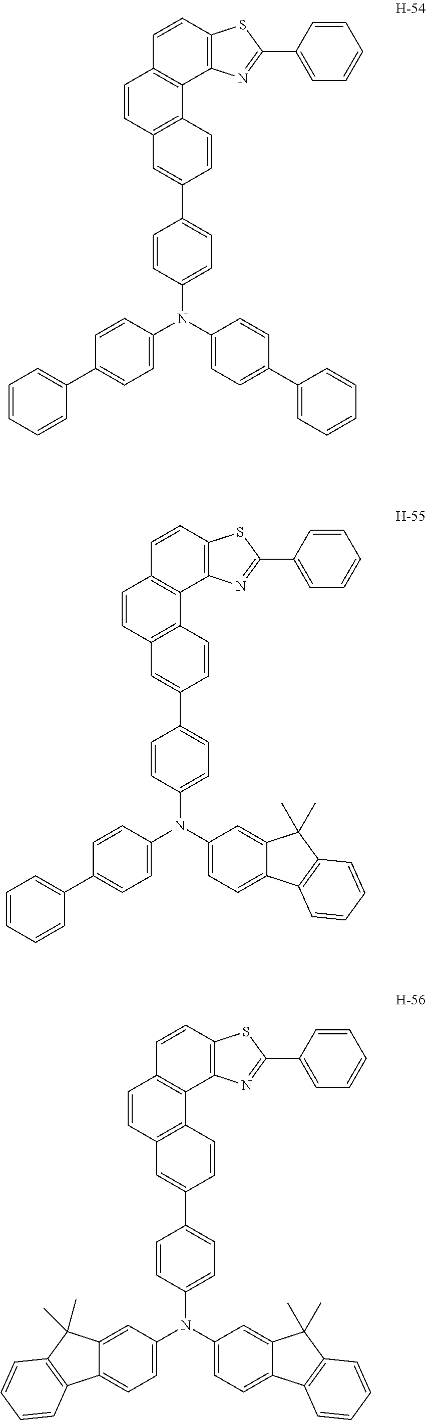 [SMILES: c1ccc(-c2ccc(N(c3ccc(-c4ccccc4)cc3)c3ccc(-c4ccc5c(ccc6ccc7sc(-c8ccccc8)nc7c65)c4)cc3)cc2)cc1 ; CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21 ; CC1(C)c2ccccc2-c2ccc(N(c3ccc(-c4ccc5c(ccc6ccc7sc(-c8ccccc8)nc7c65)c4)cc3)c3ccc4c(c3)C(C)(C)c3ccccc3-4)cc21] 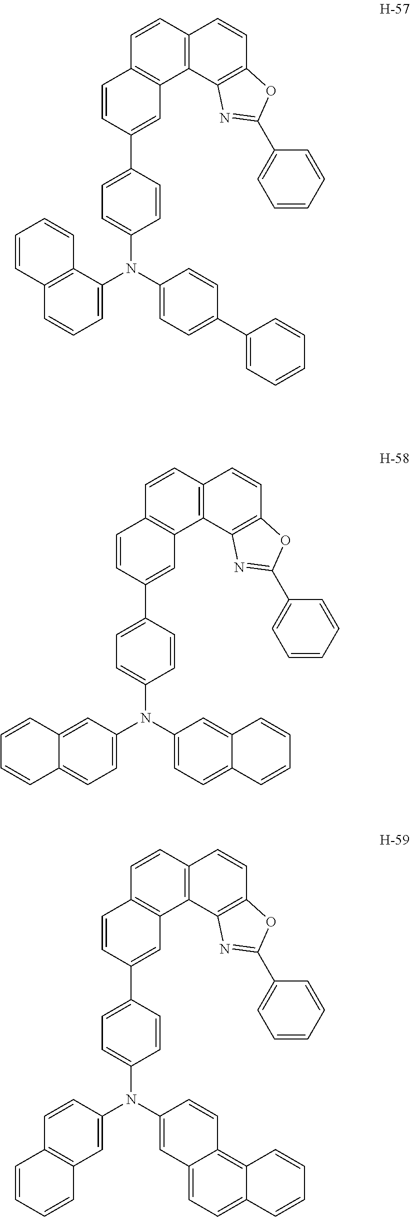 [SMILES: c1ccc(-c2ccc(N(c3ccc(-c4ccc5ccc6ccc7oc(-c8ccccc8)nc7c6c5c4)cc3)c3cccc4ccccc34)cc2)cc1 ; c1ccc(-c2nc3c(ccc4ccc5ccc(-c6ccc(N(c7ccc8ccccc8c7)c7ccc8ccccc8c7)cc6)cc5c43)o2)cc1 ; c1ccc(-c2nc3c(ccc4ccc5ccc(-c6ccc(N(c7ccc8ccccc8c7)c7ccc8c(ccc9ccccc98)c7)cc6)cc5c43)o2)cc1] 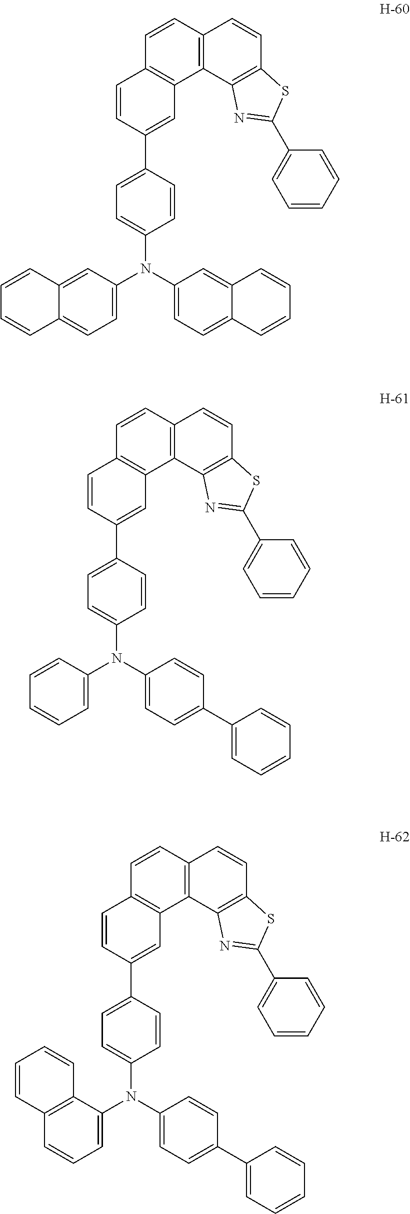 [SMILES: c1ccc(-c2nc3c(ccc4ccc5ccc(-c6ccc(N(c7ccc8ccccc8c7)c7ccc8ccccc8c7)cc6)cc5c43)s2)cc1 ; c1ccc(-c2ccc(N(c3ccccc3)c3ccc(-c4ccc5ccc6ccc7sc(-c8ccccc8)nc7c6c5c4)cc3)cc2)cc1 ; c1ccc(-c2ccc(N(c3ccc(-c4ccc5ccc6ccc7sc(-c8ccccc8)nc7c6c5c4)cc3)c3cccc4ccccc34)cc2)cc1] 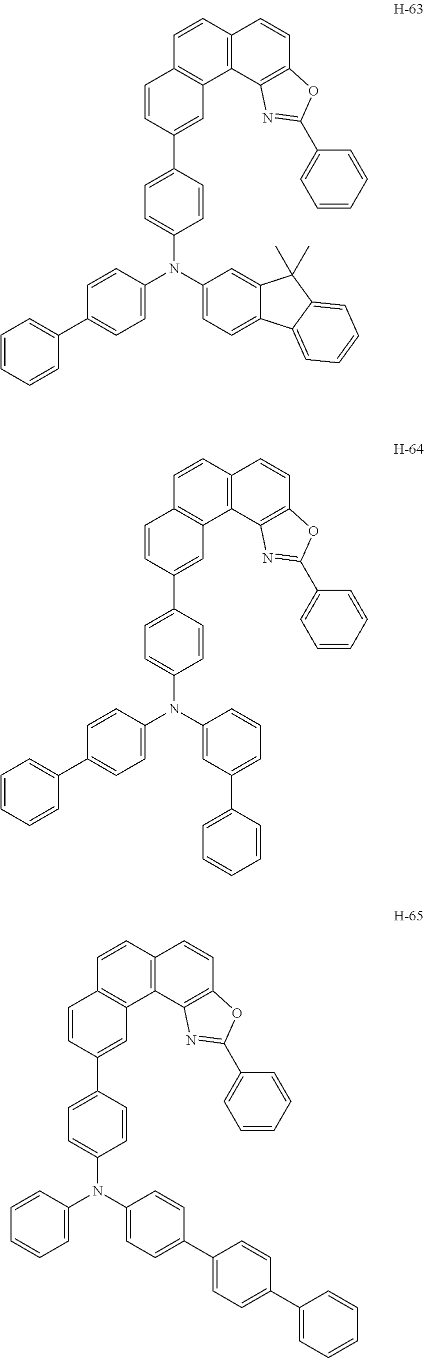 [SMILES: CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21 ; c1ccc(-c2ccc(N(c3ccc(-c4ccc5ccc6ccc7oc(-c8ccccc8)nc7c6c5c4)cc3)c3cccc(-c4ccccc4)c3)cc2)cc1 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1] 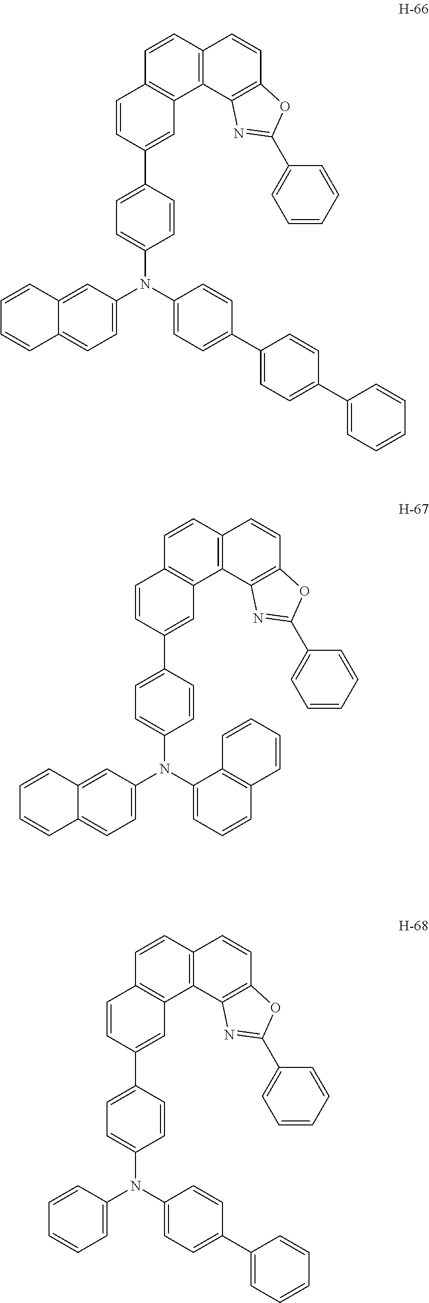 [SMILES: c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1 ; c1ccc(-c2nc3c(ccc4ccc5ccc(-c6ccc(N(c7ccc8ccccc8c7)c7cccc8ccccc78)cc6)cc5c43)o2)cc1 ; c1ccc(-c2ccc(N(c3ccccc3)c3ccc(-c4ccc5ccc6ccc7oc(-c8ccccc8)nc7c6c5c4)cc3)cc2)cc1] 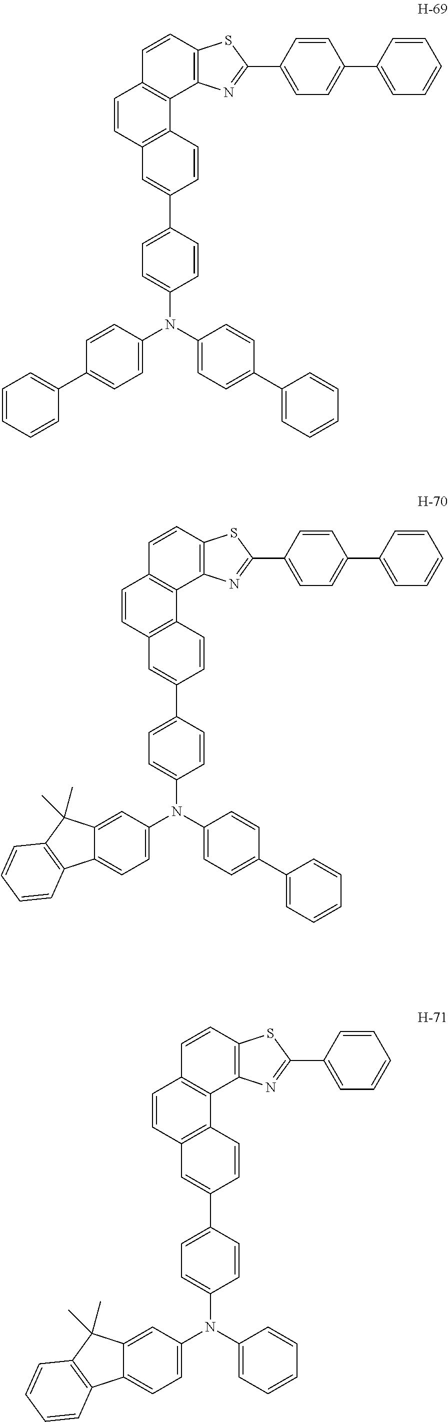 [SMILES: c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21 ; CC1(C)c2ccccc2-c2ccc(N(c3ccccc3)c3ccc(-c4ccc5c(ccc6ccc7sc(-c8ccccc8)nc7c65)c4)cc3)cc21] 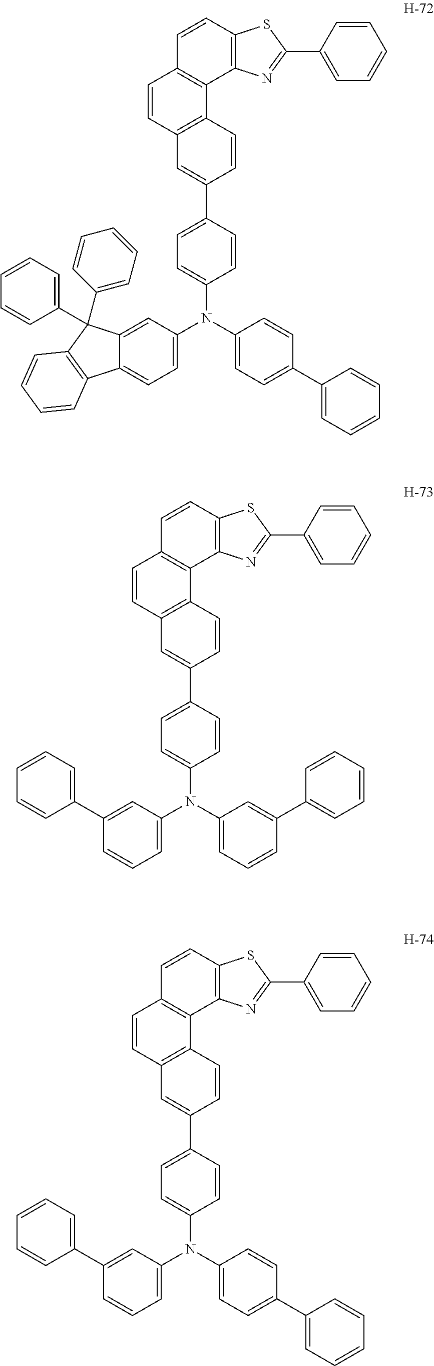 [SMILES: c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1 ; c1ccc(-c2cccc(N(c3ccc(-c4ccc5c(ccc6ccc7sc(-c8ccccc8)nc7c65)c4)cc3)c3cccc(-c4ccccc4)c3)c2)cc1 ; c1ccc(-c2ccc(N(c3ccc(-c4ccc5c(ccc6ccc7sc(-c8ccccc8)nc7c65)c4)cc3)c3cccc(-c4ccccc4)c3)cc2)cc1] 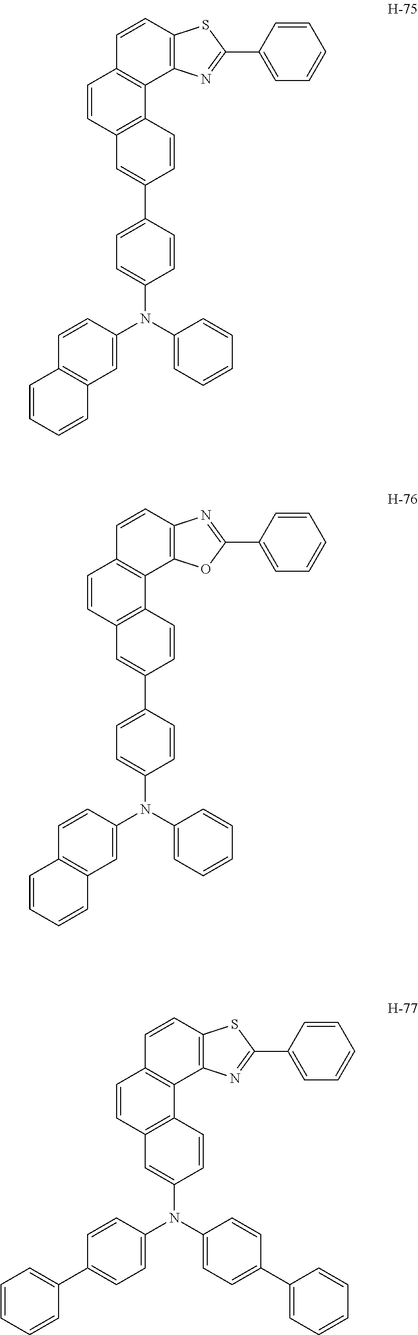 [SMILES: c1ccc(-c2nc3c(ccc4ccc5cc(-c6ccc(N(c7ccccc7)c7ccc8ccccc8c7)cc6)ccc5c43)s2)cc1 ; c1ccc(-c2nc3ccc4ccc5cc(-c6ccc(N(c7ccccc7)c7ccc8ccccc8c7)cc6)ccc5c4c3o2)cc1 ; c1ccc(-c2ccc(N(c3ccc(-c4ccccc4)cc3)c3ccc4c(ccc5ccc6sc(-c7ccccc7)nc6c54)c3)cc2)cc1] 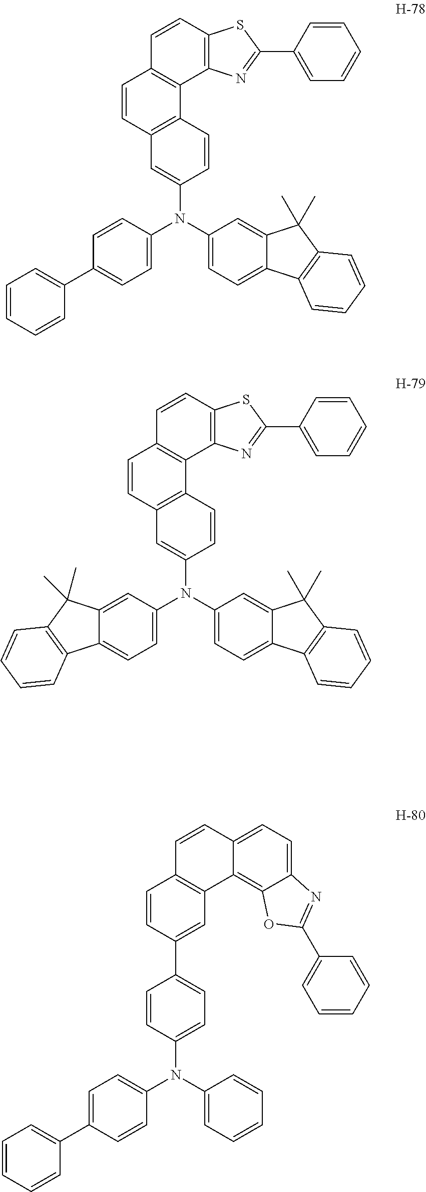 [SMILES: CC1(C)c2ccccc2-c2ccc(N(c3ccc(-c4ccccc4)cc3)c3ccc4c(ccc5ccc6sc(-c7ccccc7)nc6c54)c3)cc21 ; CC1(C)c2ccccc2-c2ccc(N(c3ccc4c(c3)C(C)(C)c3ccccc3-4)c3ccc4c(ccc5ccc6sc(-c7ccccc7)nc6c54)c3)cc21 ; c1ccc(-c2ccc(N(c3ccccc3)c3ccc(-c4ccc5ccc6ccc7nc(-c8ccccc8)oc7c6c5c4)cc3)cc2)cc1] 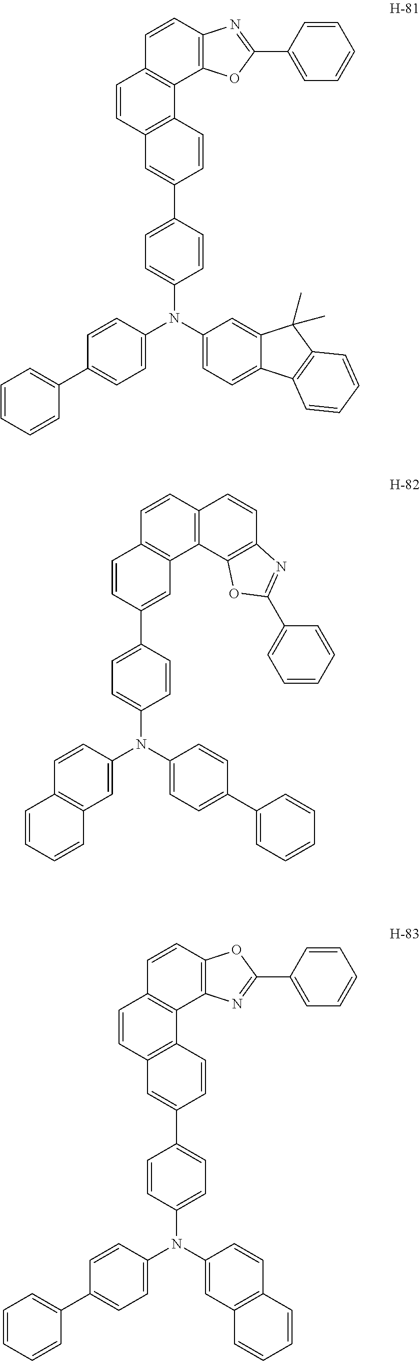 [SMILES: CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21 ; c1ccc(-c2ccc(N(c3ccc(-c4ccc5ccc6ccc7nc(-c8ccccc8)oc7c6c5c4)cc3)c3ccc4ccccc4c3)cc2)cc1 ; c1ccc(-c2ccc(N(c3ccc(-c4ccc5c(ccc6ccc7oc(-c8ccccc8)nc7c65)c4)cc3)c3ccc4ccccc4c3)cc2)cc1] 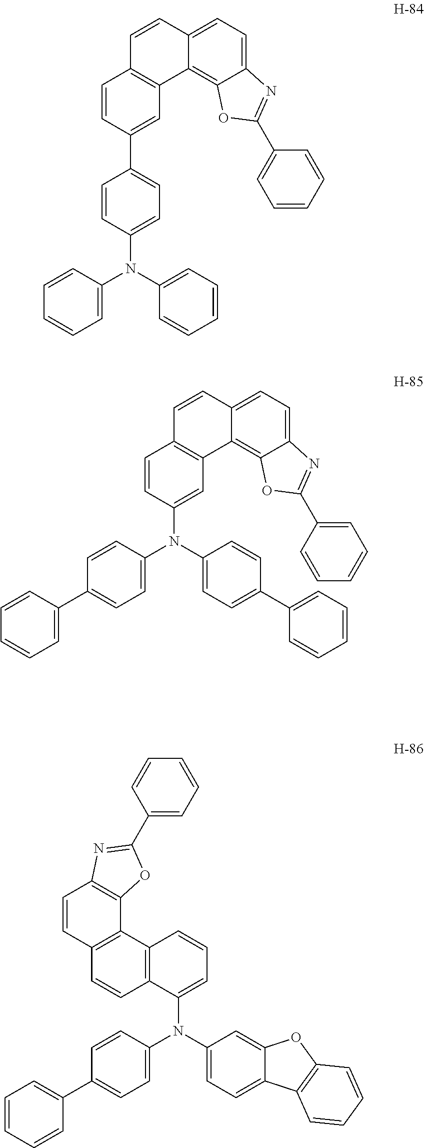 [SMILES: c1ccc(-c2nc3ccc4ccc5ccc(-c6ccc(N(c7ccccc7)c7ccccc7)cc6)cc5c4c3o2)cc1 ; c1ccc(-c2ccc(N(c3ccc(-c4ccccc4)cc3)c3ccc4ccc5ccc6nc(-c7ccccc7)oc6c5c4c3)cc2)cc1 ; c1ccc(-c2ccc(N(c3ccc4c(c3)oc3ccccc34)c3cccc4c3ccc3ccc5nc(-c6ccccc6)oc5c34)cc2)cc1] 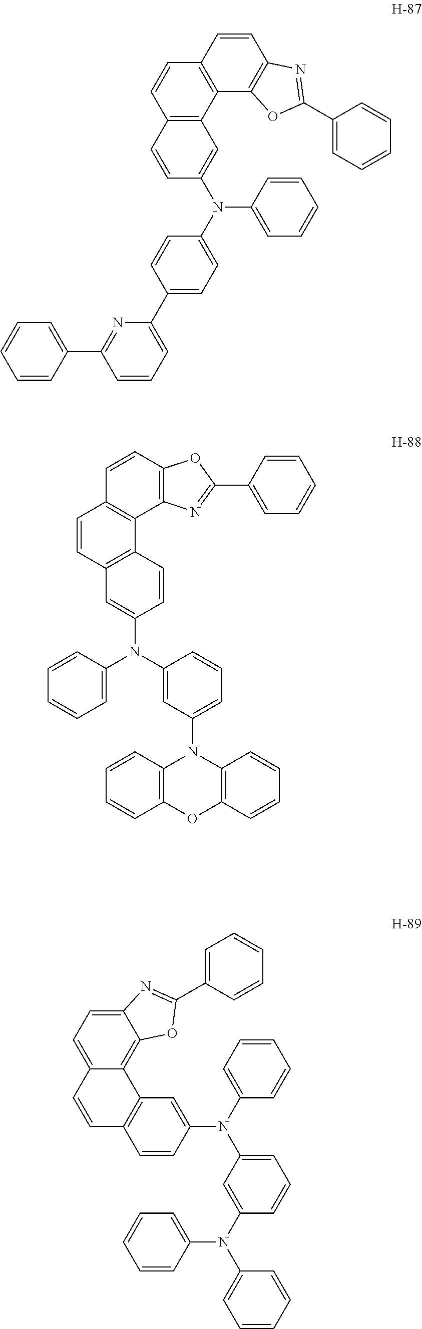 [SMILES: c1ccc(-c2cccc(-c3ccc(N(c4ccccc4)c4ccc5ccc6ccc7nc(-c8ccccc8)oc7c6c5c4)cc3)n2)cc1 ; c1ccc(-c2nc3c(ccc4ccc5cc(N(c6ccccc6)c6cccc(N7c8ccccc8Oc8ccccc87)c6)ccc5c43)o2)cc1 ; c1ccc(-c2nc3ccc4ccc5ccc(N(c6ccccc6)c6cccc(N(c7ccccc7)c7ccccc7)c6)cc5c4c3o2)cc1] 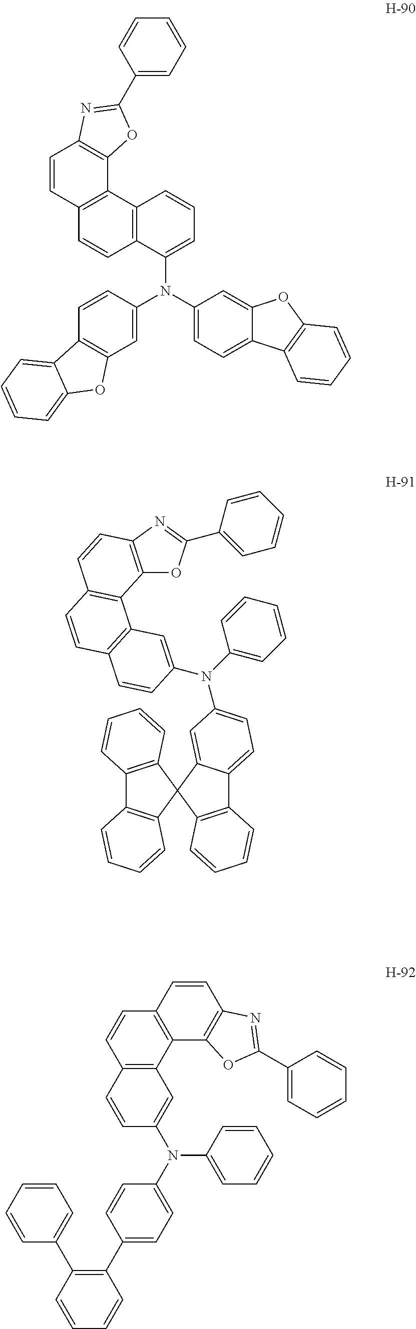 [SMILES: c1ccc(-c2nc3ccc4ccc5c(N(c6ccc7c(c6)oc6ccccc67)c6ccc7c(c6)oc6ccccc67)cccc5c4c3o2)cc1 ; c1ccc(-c2nc3ccc4ccc5ccc(N(c6ccccc6)c6ccc7c(c6)C6(c8ccccc8-c8ccccc86)c6ccccc6-7)cc5c4c3o2)cc1 ; c1ccc(-c2nc3ccc4ccc5ccc(N(c6ccccc6)c6ccc(-c7ccccc7-c7ccccc7)cc6)cc5c4c3o2)cc1] 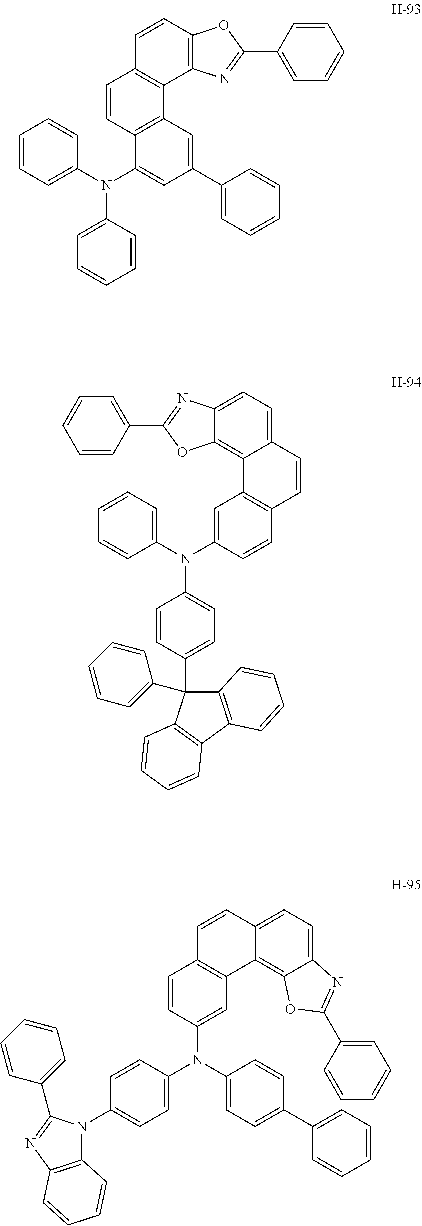 [SMILES: c1ccc(-c2cc(N(c3ccccc3)c3ccccc3)c3ccc4ccc5oc(-c6ccccc6)nc5c4c3c2)cc1 ; c1ccc(-c2nc3ccc4ccc5ccc(N(c6ccccc6)c6ccc(C7(c8ccccc8)c8ccccc8-c8ccccc87)cc6)cc5c4c3o2)cc1 ; c1ccc(-c2ccc(N(c3ccc(-n4c(-c5ccccc5)nc5ccccc54)cc3)c3ccc4ccc5ccc6nc(-c7ccccc7)oc6c5c4c3)cc2)cc1] 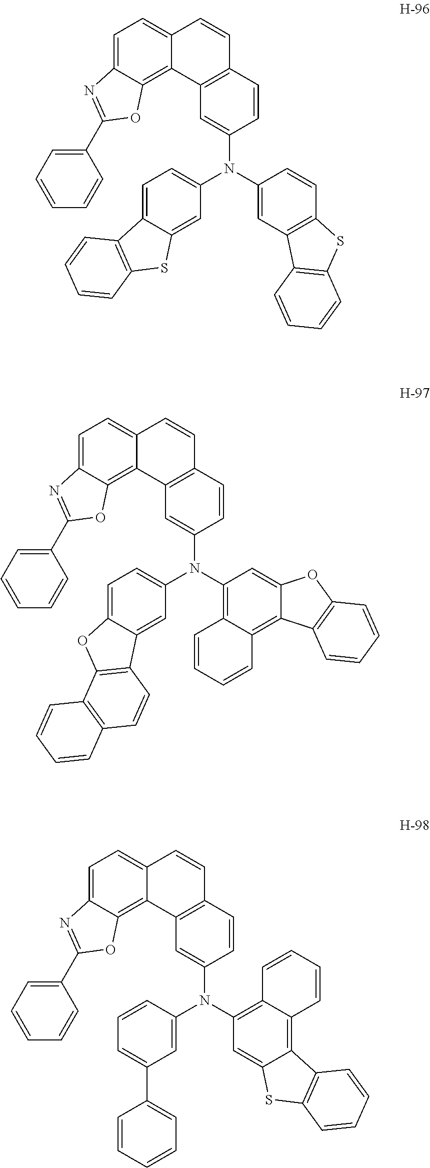 [SMILES: c1ccc(-c2nc3ccc4ccc5ccc(N(c6ccc7c(c6)sc6ccccc67)c6ccc7sc8ccccc8c7c6)cc5c4c3o2)cc1 ; c1ccc(-c2nc3ccc4ccc5ccc(N(c6ccc7oc8c9ccccc9ccc8c7c6)c6cc7oc8ccccc8c7c7ccccc67)cc5c4c3o2)cc1 ; c1ccc(-c2cccc(N(c3ccc4ccc5ccc6nc(-c7ccccc7)oc6c5c4c3)c3cc4sc5ccccc5c4c4ccccc34)c2)cc1] 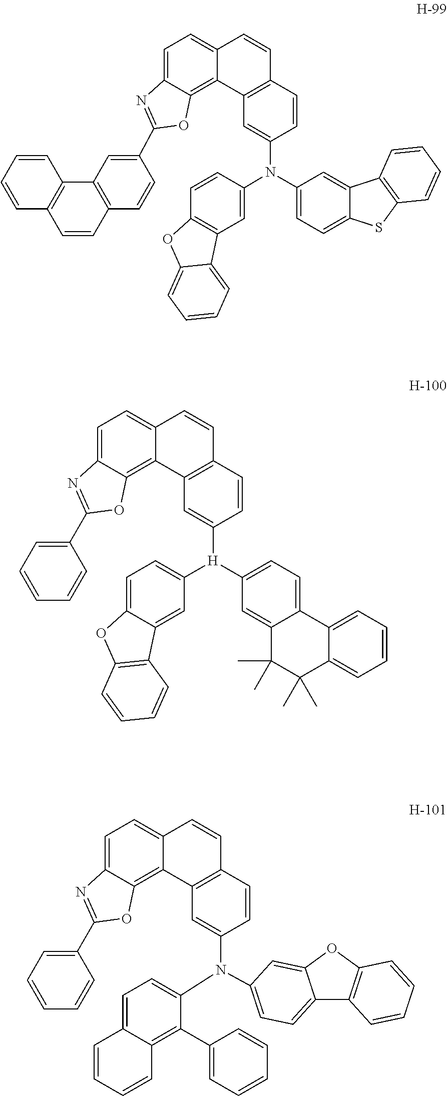 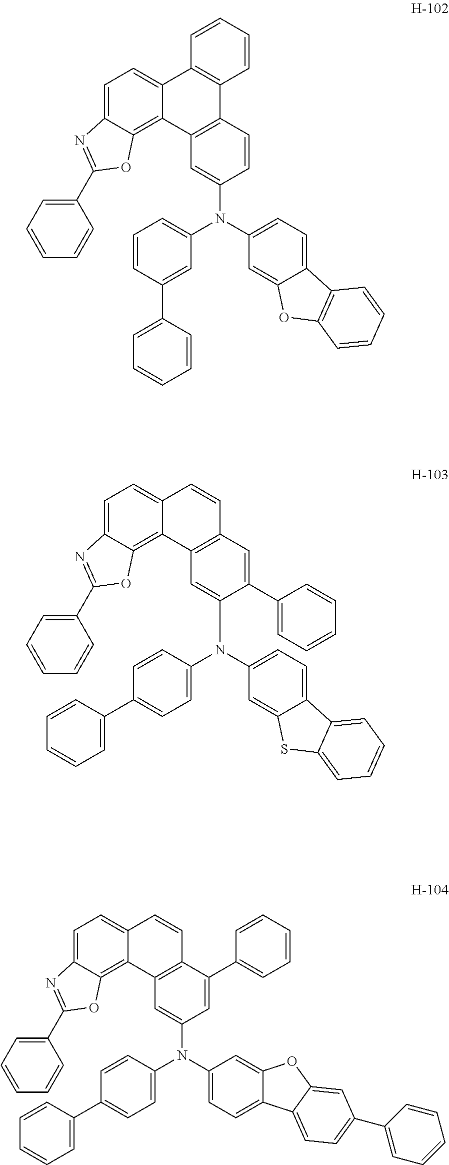 [SMILES: c1ccc(-c2cccc(N(c3ccc4c(c3)oc3ccccc34)c3ccc4c5ccccc5c5ccc6nc(-c7ccccc7)oc6c5c4c3)c2)cc1 ; c1ccc(-c2ccc(N(c3ccc4c(c3)sc3ccccc34)c3cc4c(ccc5ccc6nc(-c7ccccc7)oc6c54)cc3-c3ccccc3)cc2)cc1 ; c1ccc(-c2ccc(N(c3ccc4c(c3)oc3cc(-c5ccccc5)ccc34)c3cc(-c4ccccc4)c4ccc5ccc6nc(-c7ccccc7)oc6c5c4c3)cc2)cc1] 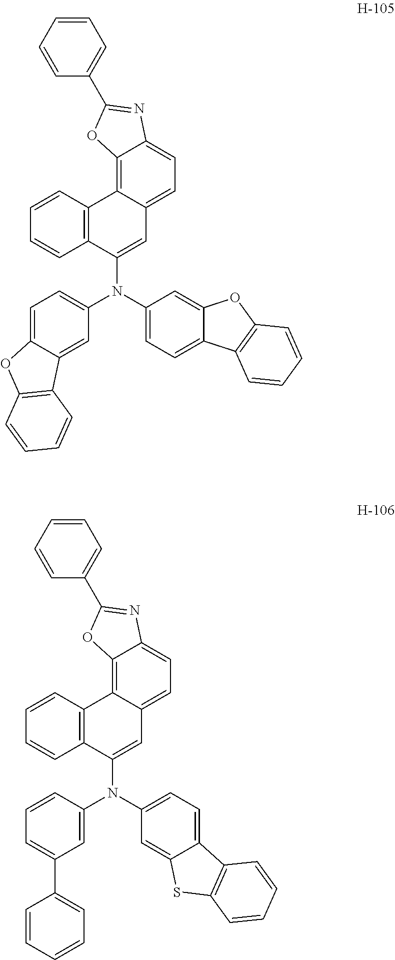 [SMILES: c1ccc(-c2nc3ccc4cc(N(c5ccc6c(c5)oc5ccccc56)c5ccc6oc7ccccc7c6c5)c5ccccc5c4c3o2)cc1 ; c1ccc(-c2cccc(N(c3ccc4c(c3)sc3ccccc34)c3cc4ccc5nc(-c6ccccc6)oc5c4c4ccccc34)c2)cc1] 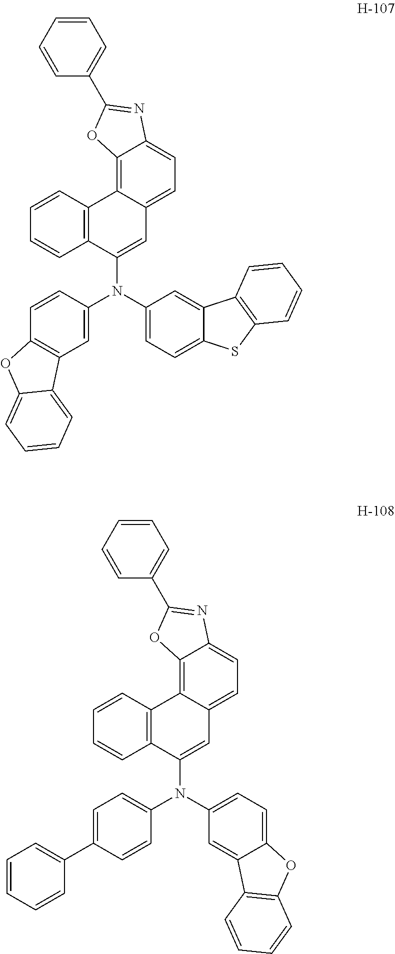 [SMILES: c1ccc(-c2nc3ccc4cc(N(c5ccc6oc7ccccc7c6c5)c5ccc6sc7ccccc7c6c5)c5ccccc5c4c3o2)cc1 ; c1ccc(-c2ccc(N(c3ccc4oc5ccccc5c4c3)c3cc4ccc5nc(-c6ccccc6)oc5c4c4ccccc34)cc2)cc1] 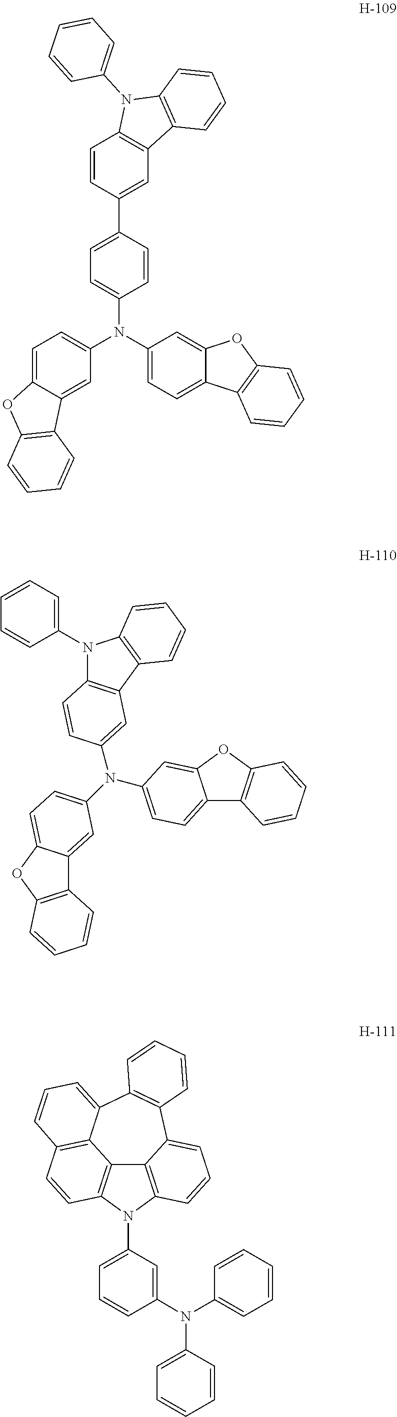 [SMILES: c1ccc(-n2c3ccccc3c3cc(-c4ccc(N(c5ccc6c(c5)oc5ccccc56)c5ccc6oc7ccccc7c6c5)cc4)ccc32)cc1 ; c1ccc(-n2c3ccccc3c3cc(N(c4ccc5c(c4)oc4ccccc45)c4ccc5oc6ccccc6c5c4)ccc32)cc1 ; c1ccc(N(c2ccccc2)c2cccc(-n3c4cccc5c4c4c6c(cccc6ccc43)-c3ccccc3-5)c2)cc1] 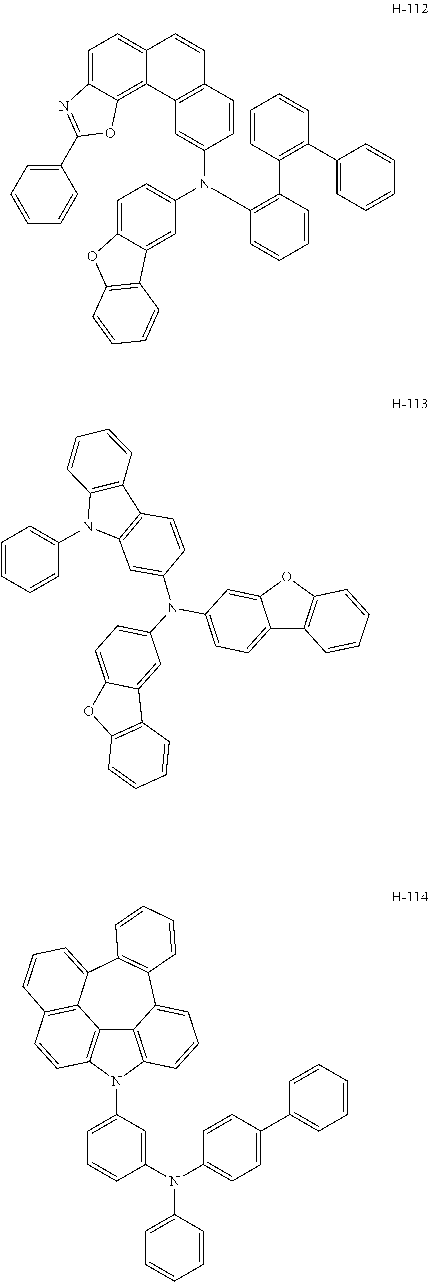 [SMILES: c1ccc(-c2nc3ccc4ccc5ccc(N(c6ccc7oc8ccccc8c7c6)c6ccccc6-c6ccccc6-c6ccccc6)cc5c4c3o2)cc1 ; c1ccc(-n2c3ccccc3c3ccc(N(c4ccc5c(c4)oc4ccccc45)c4ccc5oc6ccccc6c5c4)cc32)cc1 ; c1ccc(-c2ccc(N(c3ccccc3)c3cccc(-n4c5cccc6c5c5c7c(cccc7ccc54)-c4ccccc4-6)c3)cc2)cc1] 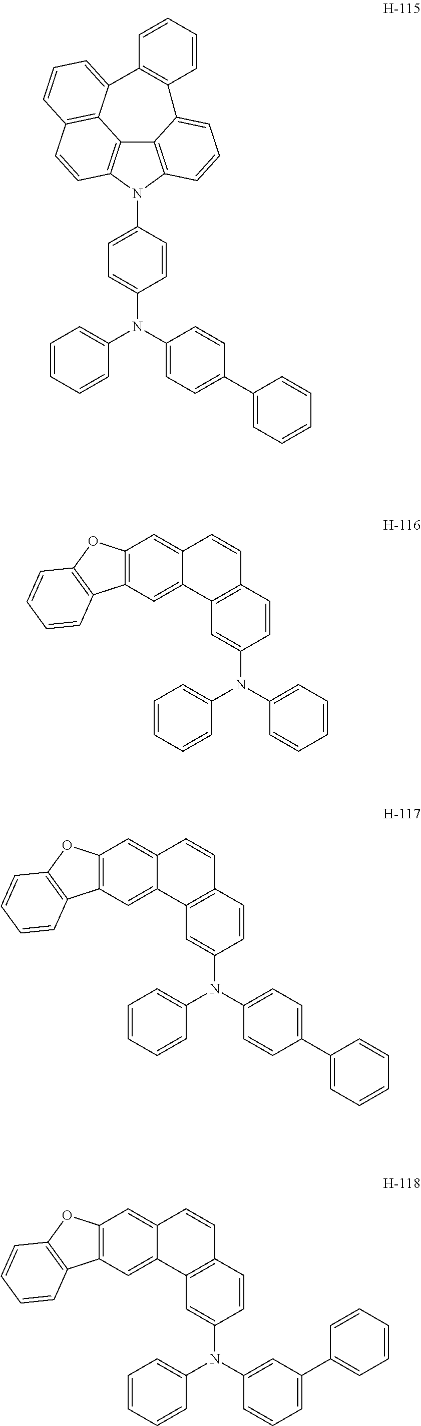 [SMILES: c1ccc(-c2ccc(N(c3ccccc3)c3ccc(-n4c5cccc6c5c5c7c(cccc7ccc54)-c4ccccc4-6)cc3)cc2)cc1 ; c1ccc(N(c2ccccc2)c2ccc3ccc4cc5oc6ccccc6c5cc4c3c2)cc1 ; c1ccc(-c2ccc(N(c3ccccc3)c3ccc4ccc5cc6oc7ccccc7c6cc5c4c3)cc2)cc1 ; c1ccc(-c2cccc(N(c3ccccc3)c3ccc4ccc5cc6oc7ccccc7c6cc5c4c3)c2)cc1] 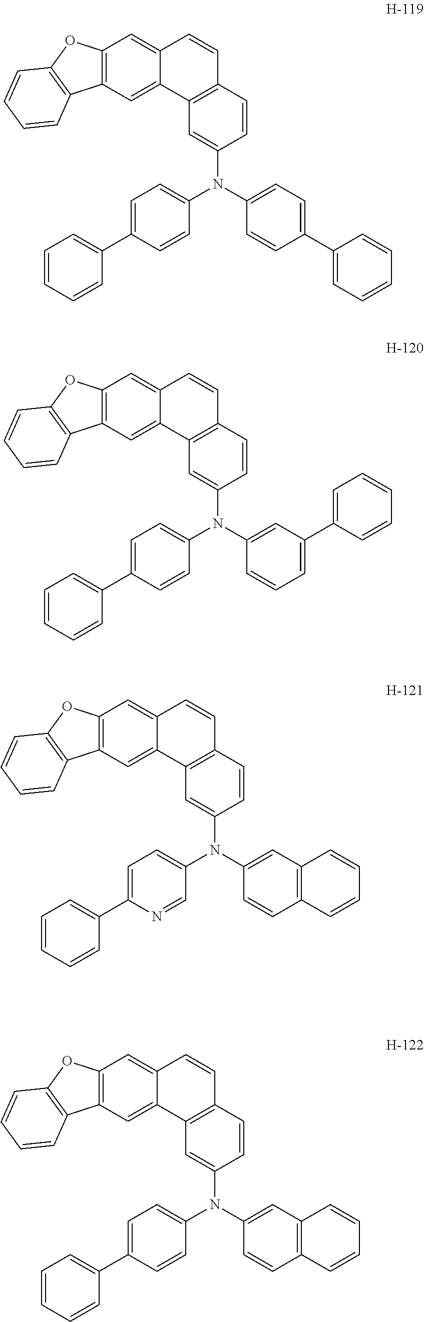 [SMILES: c1ccc(-c2ccc(N(c3ccc(-c4ccccc4)cc3)c3ccc4ccc5cc6oc7ccccc7c6cc5c4c3)cc2)cc1 ; c1ccc(-c2ccc(N(c3cccc(-c4ccccc4)c3)c3ccc4ccc5cc6oc7ccccc7c6cc5c4c3)cc2)cc1 ; c1ccc(-c2ccc(N(c3ccc4ccccc4c3)c3ccc4ccc5cc6oc7ccccc7c6cc5c4c3)cn2)cc1 ; c1ccc(-c2ccc(N(c3ccc4ccccc4c3)c3ccc4ccc5cc6oc7ccccc7c6cc5c4c3)cc2)cc1] 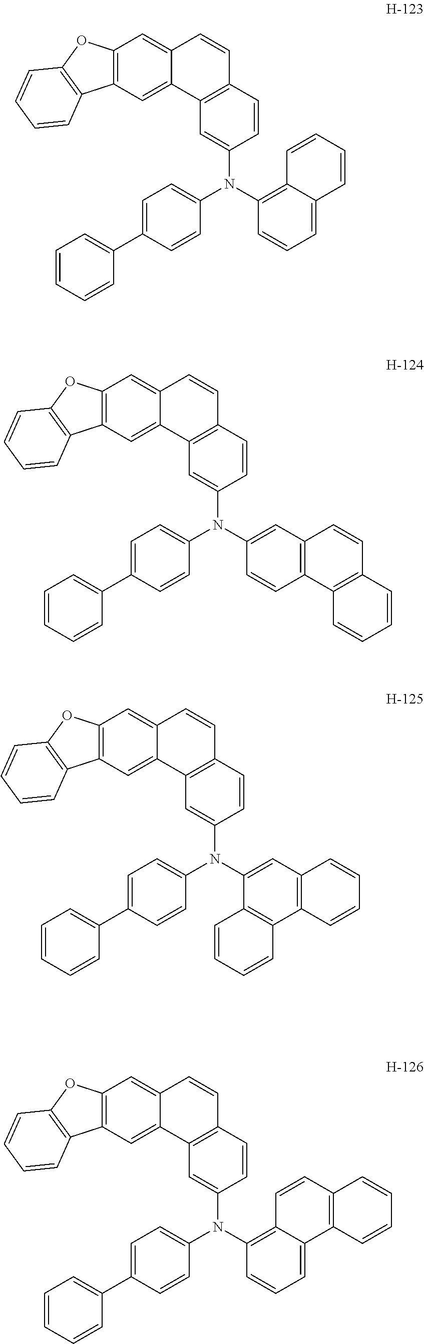 [SMILES: c1ccc(-c2ccc(N(c3ccc4ccc5cc6oc7ccccc7c6cc5c4c3)c3cccc4ccccc34)cc2)cc1 ; c1ccc(-c2ccc(N(c3ccc4c(ccc5ccccc54)c3)c3ccc4ccc5cc6oc7ccccc7c6cc5c4c3)cc2)cc1 ; c1ccc(-c2ccc(N(c3ccc4ccc5cc6oc7ccccc7c6cc5c4c3)c3cc4ccccc4c4ccccc34)cc2)cc1 ; c1ccc(-c2ccc(N(c3ccc4ccc5cc6oc7ccccc7c6cc5c4c3)c3cccc4c3ccc3ccccc34)cc2)cc1] 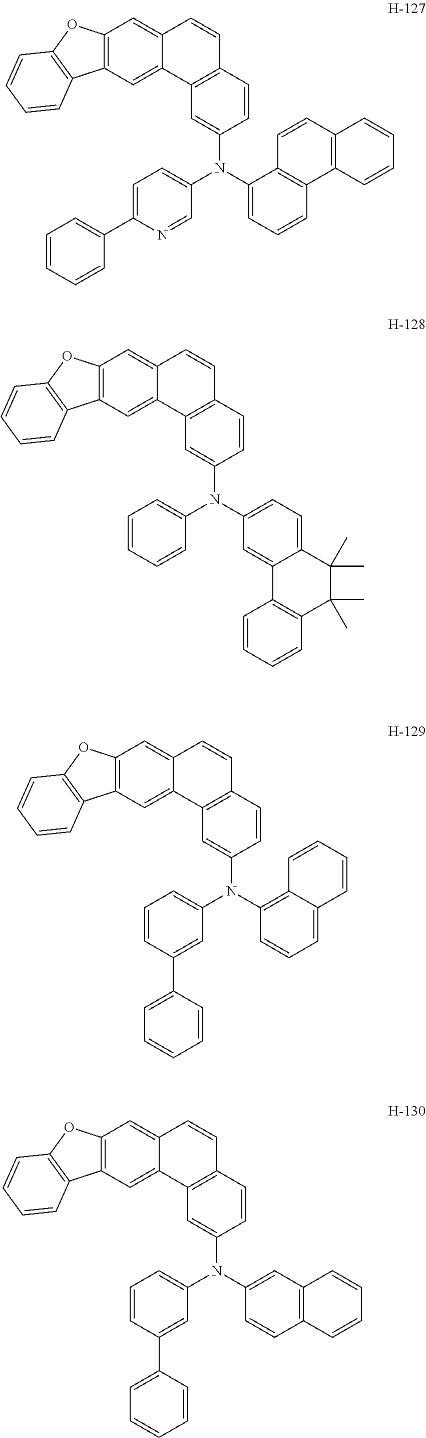 [SMILES: c1ccc(-c2ccc(N(c3ccc4ccc5cc6oc7ccccc7c6cc5c4c3)c3cccc4c3ccc3ccccc34)cn2)cc1 ; CC1(C)c2ccccc2-c2cc(N(c3ccccc3)c3ccc4ccc5cc6oc7ccccc7c6cc5c4c3)ccc2C1(C)C ; c1ccc(-c2cccc(N(c3ccc4ccc5cc6oc7ccccc7c6cc5c4c3)c3cccc4ccccc34)c2)cc1 ; c1ccc(-c2cccc(N(c3ccc4ccccc4c3)c3ccc4ccc5cc6oc7ccccc7c6cc5c4c3)c2)cc1] 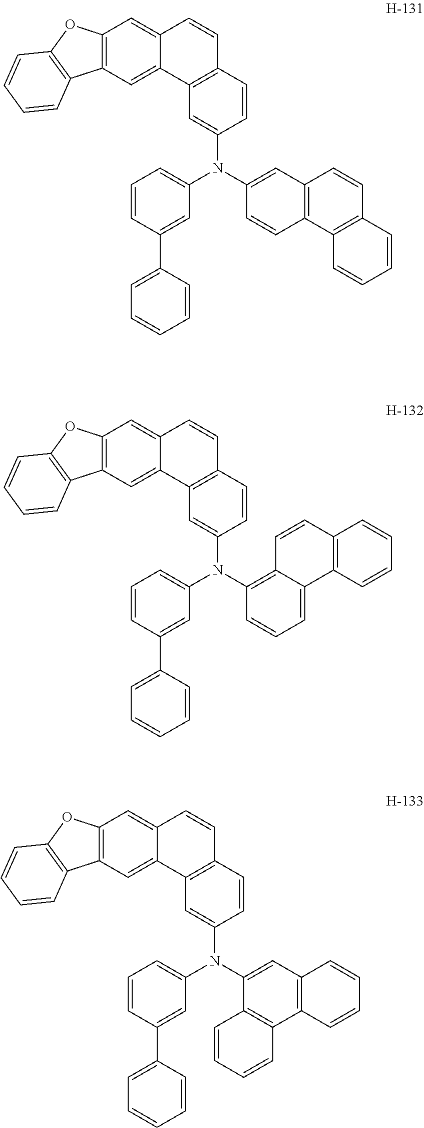 [SMILES: c1ccc(-c2cccc(N(c3ccc4c(ccc5ccccc54)c3)c3ccc4ccc5cc6oc7ccccc7c6cc5c4c3)c2)cc1 ; c1ccc(-c2cccc(N(c3ccc4ccc5cc6oc7ccccc7c6cc5c4c3)c3cccc4c3ccc3ccccc34)c2)cc1 ; c1ccc(-c2cccc(N(c3ccc4ccc5cc6oc7ccccc7c6cc5c4c3)c3cc4ccccc4c4ccccc34)c2)cc1] 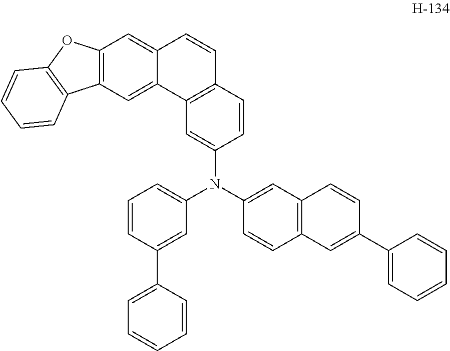 [SMILES: c1ccc(-c2cccc(N(c3ccc4cc(-c5ccccc5)ccc4c3)c3ccc4ccc5cc6oc7ccccc7c6cc5c4c3)c2)cc1] 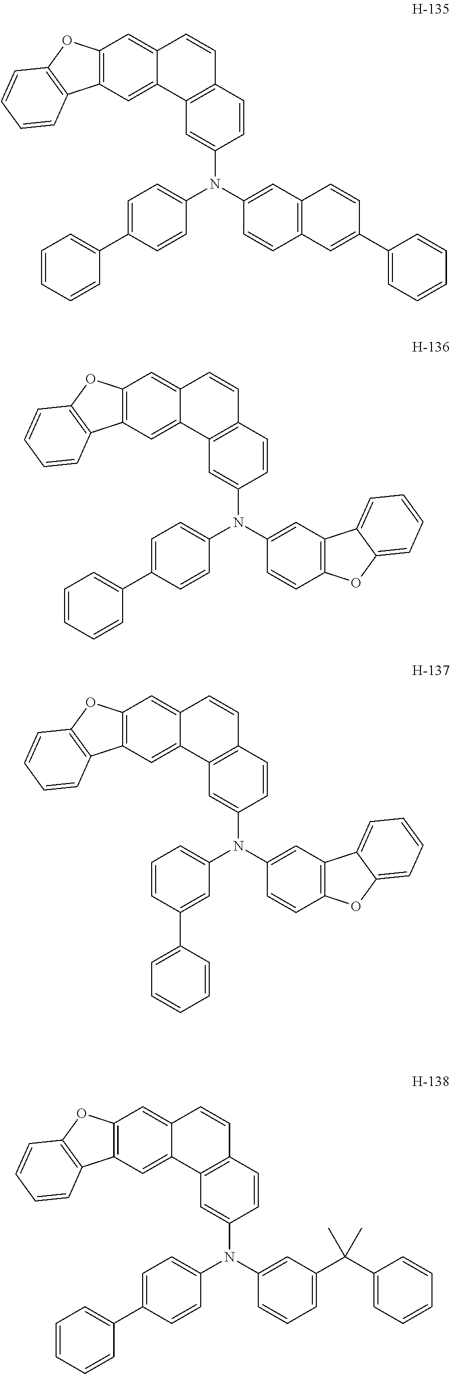 [SMILES: c1ccc(-c2ccc(N(c3ccc4cc(-c5ccccc5)ccc4c3)c3ccc4ccc5cc6oc7ccccc7c6cc5c4c3)cc2)cc1 ; c1ccc(-c2ccc(N(c3ccc4ccc5cc6oc7ccccc7c6cc5c4c3)c3ccc4oc5ccccc5c4c3)cc2)cc1 ; c1ccc(-c2cccc(N(c3ccc4ccc5cc6oc7ccccc7c6cc5c4c3)c3ccc4oc5ccccc5c4c3)c2)cc1 ; CC(C)(c1ccccc1)c1cccc(N(c2ccc(-c3ccccc3)cc2)c2ccc3ccc4cc5oc6ccccc6c5cc4c3c2)c1] 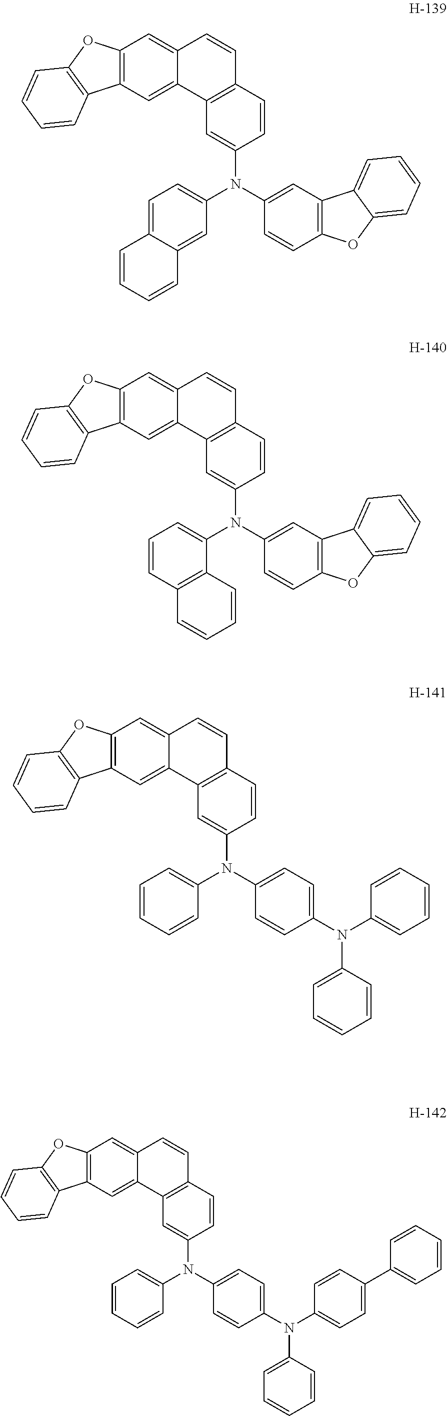 [SMILES: c1ccc2cc(N(c3ccc4ccc5cc6oc7ccccc7c6cc5c4c3)c3ccc4oc5ccccc5c4c3)ccc2c1 ; c1ccc2c(N(c3ccc4ccc5cc6oc7ccccc7c6cc5c4c3)c3ccc4oc5ccccc5c4c3)cccc2c1 ; c1ccc(N(c2ccccc2)c2ccc(N(c3ccccc3)c3ccc4ccc5cc6oc7ccccc7c6cc5c4c3)cc2)cc1 ; c1ccc(-c2ccc(N(c3ccccc3)c3ccc(N(c4ccccc4)c4ccc5ccc6cc7oc8ccccc8c7cc6c5c4)cc3)cc2)cc1] 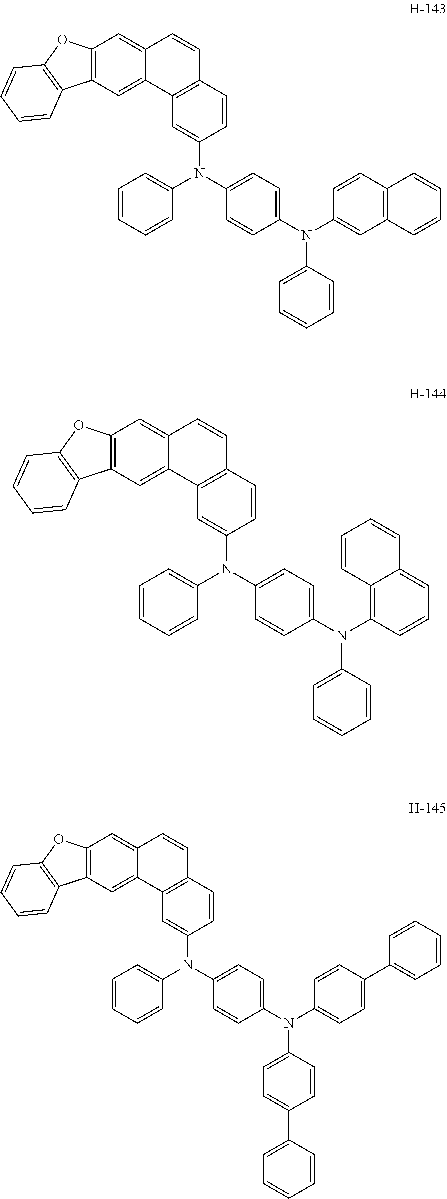 [SMILES: c1ccc(N(c2ccc(N(c3ccccc3)c3ccc4ccc5cc6oc7ccccc7c6cc5c4c3)cc2)c2ccc3ccccc3c2)cc1 ; c1ccc(N(c2ccc(N(c3ccccc3)c3cccc4ccccc34)cc2)c2ccc3ccc4cc5oc6ccccc6c5cc4c3c2)cc1 ; c1ccc(-c2ccc(N(c3ccc(-c4ccccc4)cc3)c3ccc(N(c4ccccc4)c4ccc5ccc6cc7oc8ccccc8c7cc6c5c4)cc3)cc2)cc1] 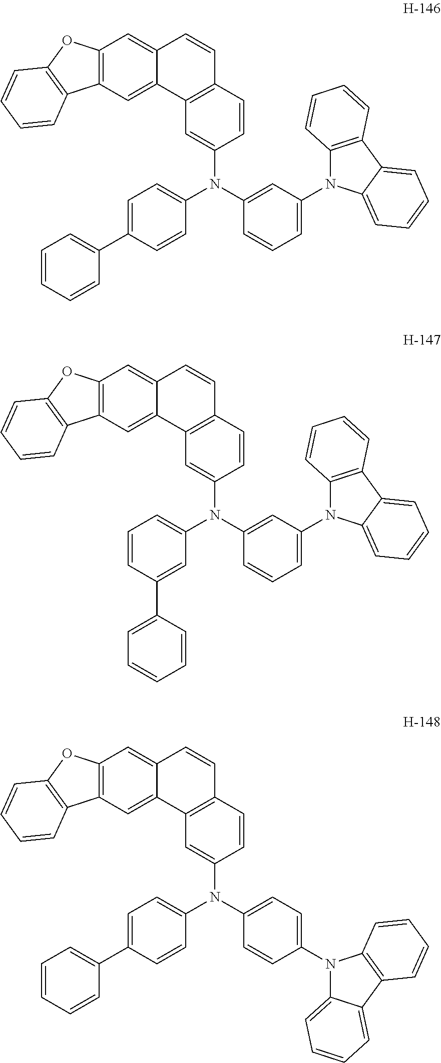 [SMILES: c1ccc(-c2ccc(N(c3cccc(-n4c5ccccc5c5ccccc54)c3)c3ccc4ccc5cc6oc7ccccc7c6cc5c4c3)cc2)cc1 ; c1ccc(-c2cccc(N(c3cccc(-n4c5ccccc5c5ccccc54)c3)c3ccc4ccc5cc6oc7ccccc7c6cc5c4c3)c2)cc1 ; c1ccc(-c2ccc(N(c3ccc(-n4c5ccccc5c5ccccc54)cc3)c3ccc4ccc5cc6oc7ccccc7c6cc5c4c3)cc2)cc1] 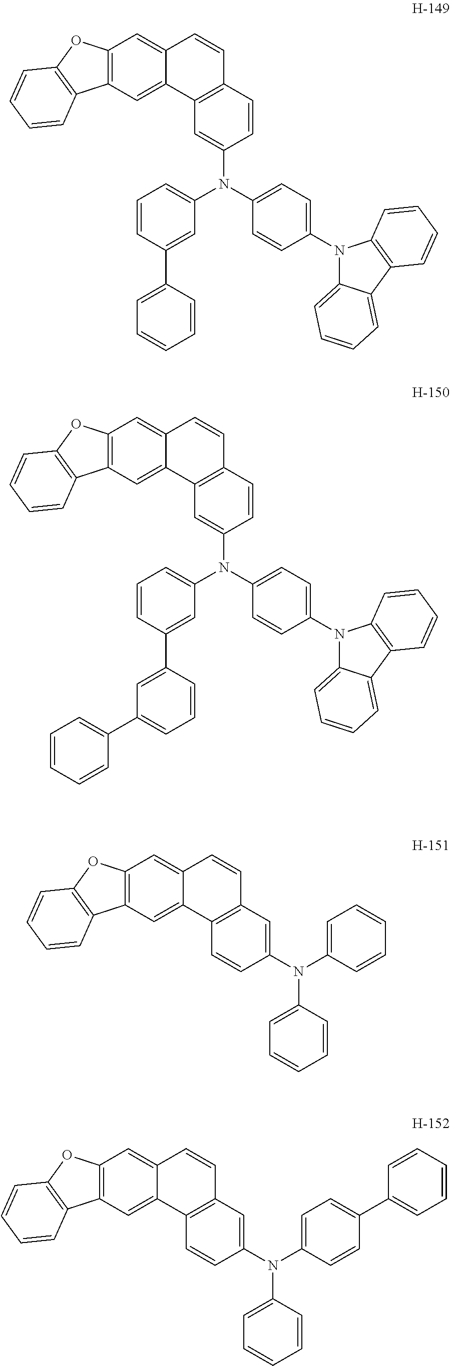 [SMILES: c1ccc(-c2cccc(N(c3ccc(-n4c5ccccc5c5ccccc54)cc3)c3ccc4ccc5cc6oc7ccccc7c6cc5c4c3)c2)cc1 ; c1ccc(-c2cccc(-c3cccc(N(c4ccc(-n5c6ccccc6c6ccccc65)cc4)c4ccc5ccc6cc7oc8ccccc8c7cc6c5c4)c3)c2)cc1 ; c1ccc(N(c2ccccc2)c2ccc3c(ccc4cc5oc6ccccc6c5cc43)c2)cc1 ; c1ccc(-c2ccc(N(c3ccccc3)c3ccc4c(ccc5cc6oc7ccccc7c6cc54)c3)cc2)cc1] 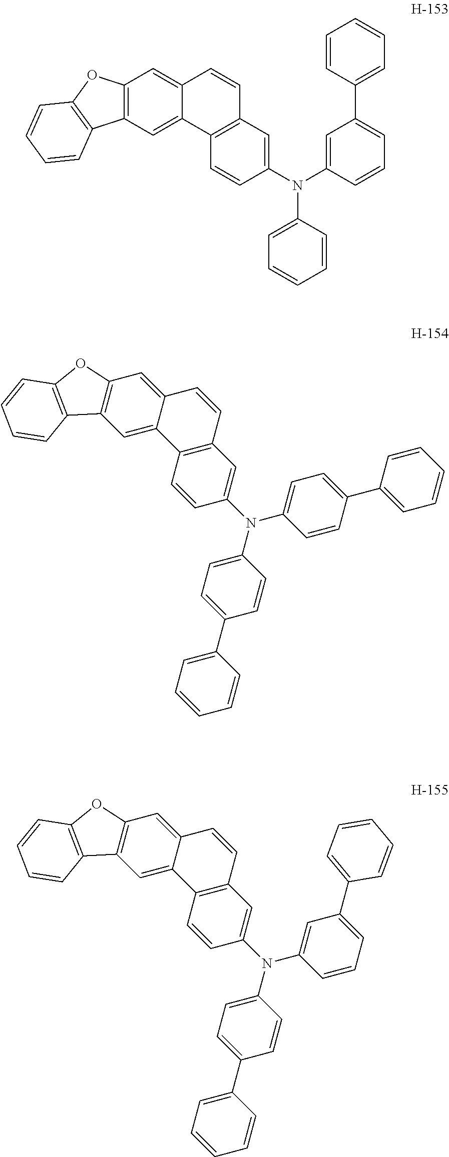 [SMILES: c1ccc(-c2cccc(N(c3ccccc3)c3ccc4c(ccc5cc6oc7ccccc7c6cc54)c3)c2)cc1 ; c1ccc(-c2ccc(N(c3ccc(-c4ccccc4)cc3)c3ccc4c(ccc5cc6oc7ccccc7c6cc54)c3)cc2)cc1 ; c1ccc(-c2ccc(N(c3cccc(-c4ccccc4)c3)c3ccc4c(ccc5cc6oc7ccccc7c6cc54)c3)cc2)cc1] 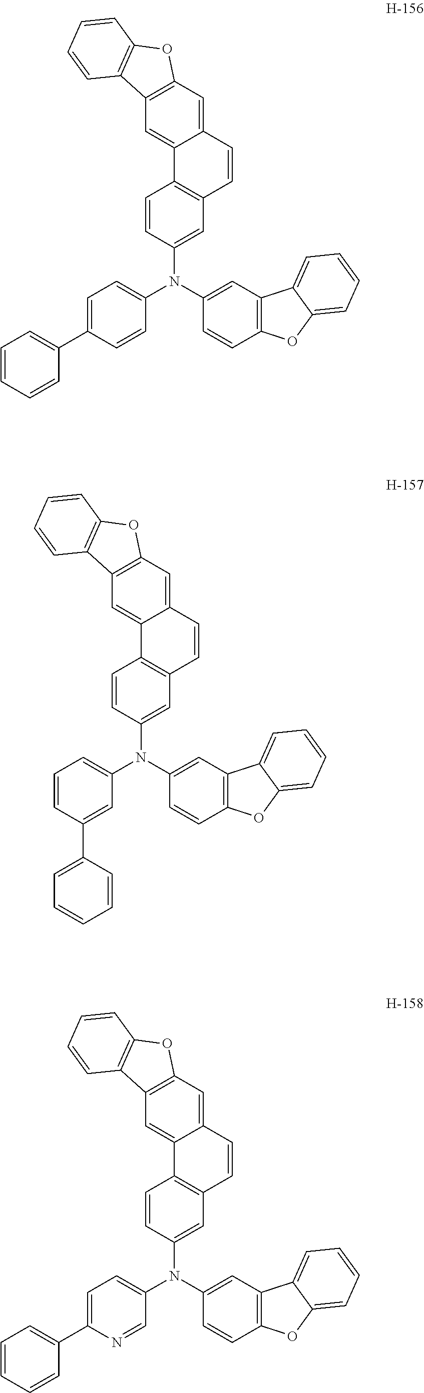 [SMILES: c1ccc(-c2ccc(N(c3ccc4c(ccc5cc6oc7ccccc7c6cc54)c3)c3ccc4oc5ccccc5c4c3)cc2)cc1 ; c1ccc(-c2cccc(N(c3ccc4c(ccc5cc6oc7ccccc7c6cc54)c3)c3ccc4oc5ccccc5c4c3)c2)cc1 ; c1ccc(-c2ccc(N(c3ccc4c(ccc5cc6oc7ccccc7c6cc54)c3)c3ccc4oc5ccccc5c4c3)cn2)cc1] 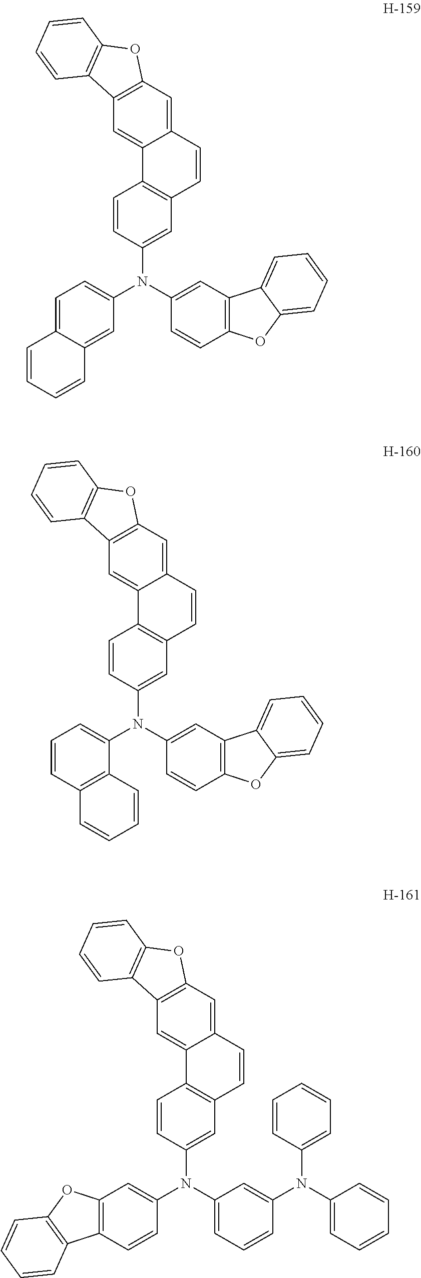 [SMILES: c1ccc2cc(N(c3ccc4c(ccc5cc6oc7ccccc7c6cc54)c3)c3ccc4oc5ccccc5c4c3)ccc2c1 ; c1ccc2c(N(c3ccc4c(ccc5cc6oc7ccccc7c6cc54)c3)c3ccc4oc5ccccc5c4c3)cccc2c1 ; c1ccc(N(c2ccccc2)c2cccc(N(c3ccc4c(ccc5cc6oc7ccccc7c6cc54)c3)c3ccc4c(c3)oc3ccccc34)c2)cc1] 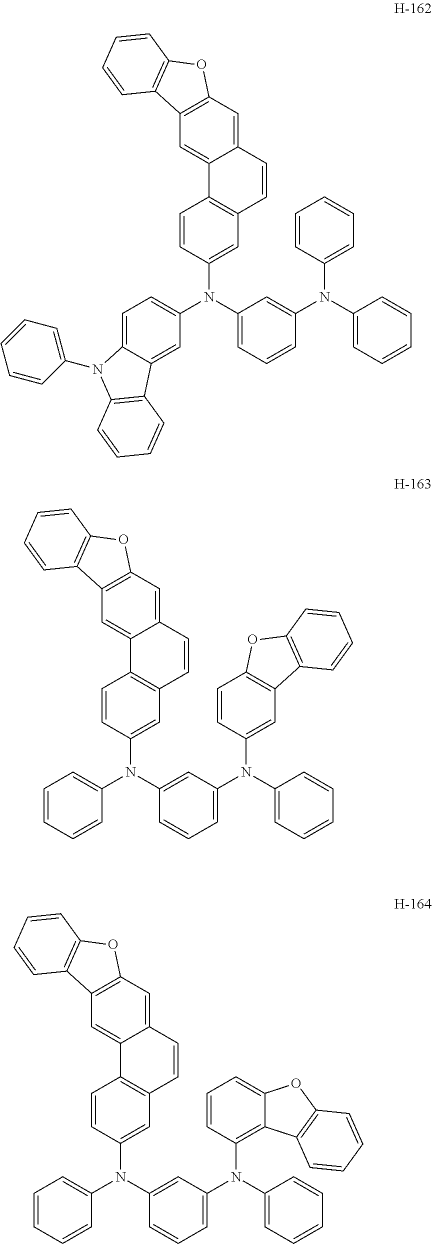 [SMILES: c1ccc(N(c2ccccc2)c2cccc(N(c3ccc4c(ccc5cc6oc7ccccc7c6cc54)c3)c3ccc4c(c3)c3ccccc3n4-c3ccccc3)c2)cc1 ; c1ccc(N(c2cccc(N(c3ccccc3)c3ccc4oc5ccccc5c4c3)c2)c2ccc3c(ccc4cc5oc6ccccc6c5cc43)c2)cc1 ; c1ccc(N(c2cccc(N(c3ccccc3)c3cccc4oc5ccccc5c34)c2)c2ccc3c(ccc4cc5oc6ccccc6c5cc43)c2)cc1] 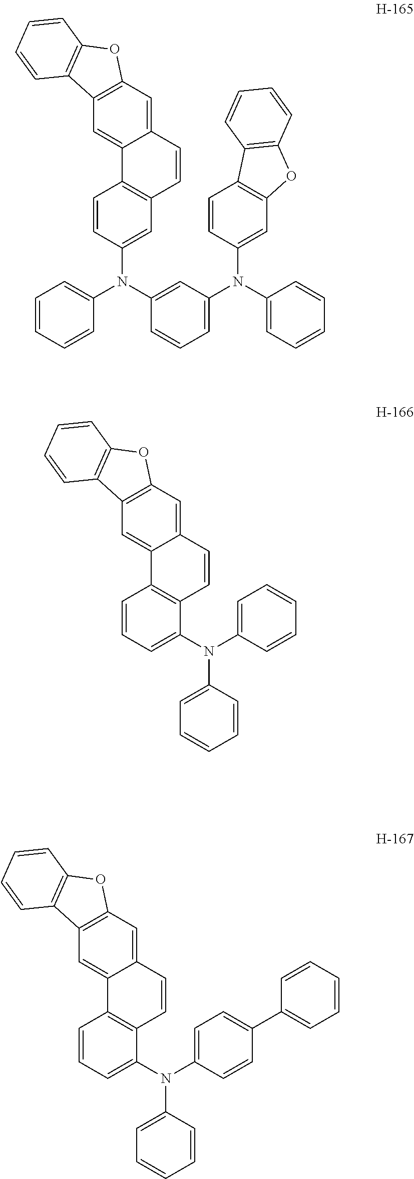 [SMILES: c1ccc(N(c2cccc(N(c3ccccc3)c3ccc4c(c3)oc3ccccc34)c2)c2ccc3c(ccc4cc5oc6ccccc6c5cc43)c2)cc1 ; c1ccc(N(c2ccccc2)c2cccc3c2ccc2cc4oc5ccccc5c4cc23)cc1 ; c1ccc(-c2ccc(N(c3ccccc3)c3cccc4c3ccc3cc5oc6ccccc6c5cc34)cc2)cc1] 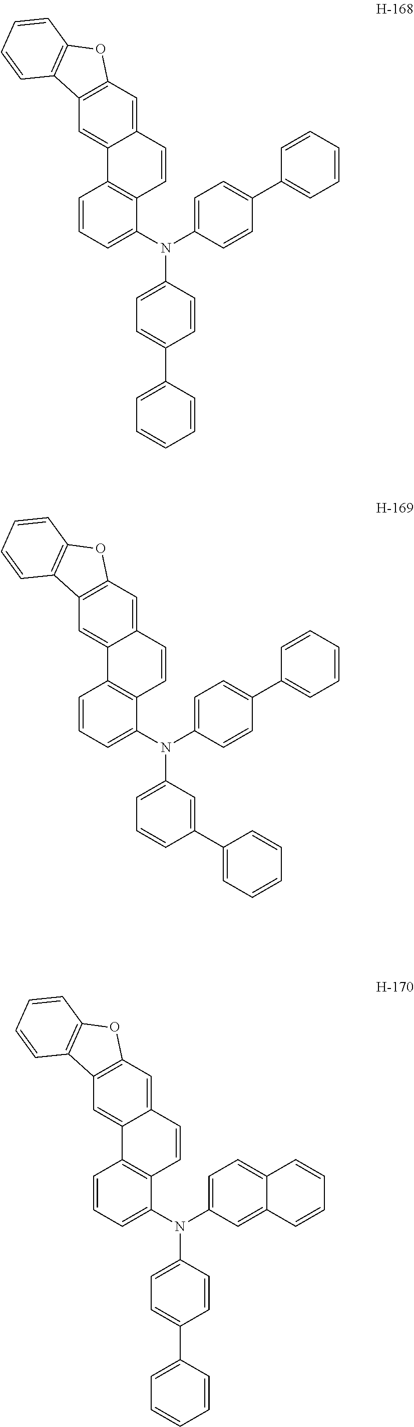 [SMILES: c1ccc(-c2ccc(N(c3ccc(-c4ccccc4)cc3)c3cccc4c3ccc3cc5oc6ccccc6c5cc34)cc2)cc1 ; c1ccc(-c2ccc(N(c3cccc(-c4ccccc4)c3)c3cccc4c3ccc3cc5oc6ccccc6c5cc34)cc2)cc1 ; c1ccc(-c2ccc(N(c3ccc4ccccc4c3)c3cccc4c3ccc3cc5oc6ccccc6c5cc34)cc2)cc1] 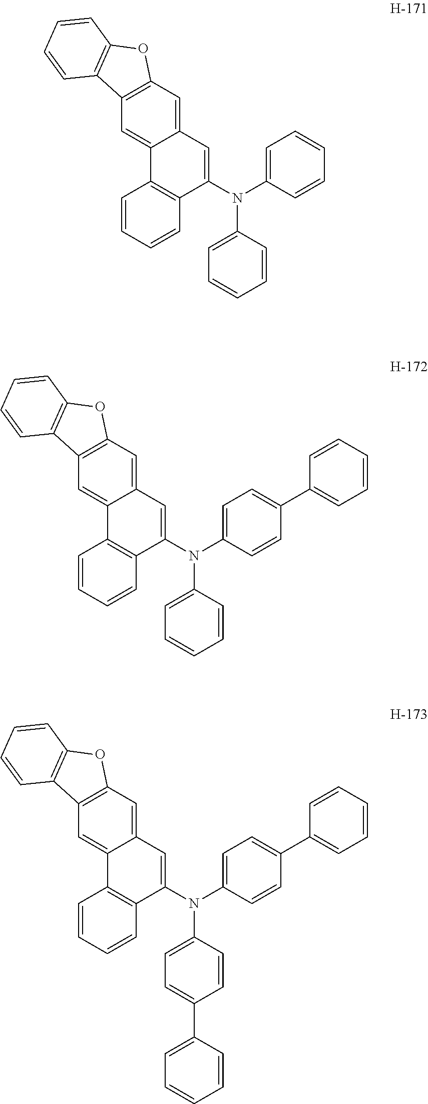 [SMILES: c1ccc(N(c2ccccc2)c2cc3cc4oc5ccccc5c4cc3c3ccccc23)cc1 ; c1ccc(-c2ccc(N(c3ccccc3)c3cc4cc5oc6ccccc6c5cc4c4ccccc34)cc2)cc1 ; c1ccc(-c2ccc(N(c3ccc(-c4ccccc4)cc3)c3cc4cc5oc6ccccc6c5cc4c4ccccc34)cc2)cc1] 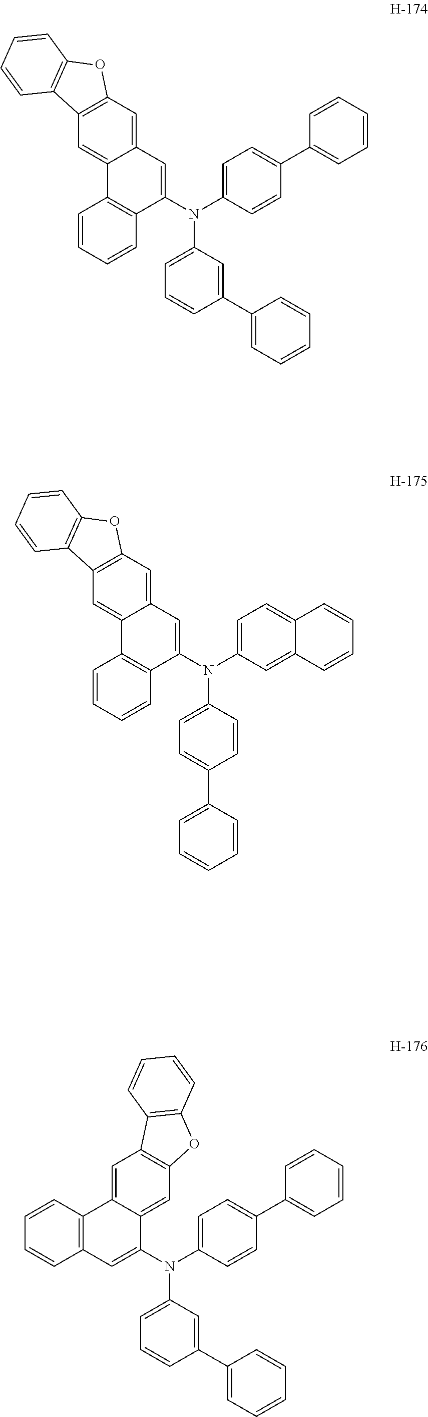 [SMILES: c1ccc(-c2ccc(N(c3cccc(-c4ccccc4)c3)c3cc4cc5oc6ccccc6c5cc4c4ccccc34)cc2)cc1 ; c1ccc(-c2ccc(N(c3ccc4ccccc4c3)c3cc4cc5oc6ccccc6c5cc4c4ccccc34)cc2)cc1 ; c1ccc(-c2ccc(N(c3cccc(-c4ccccc4)c3)c3cc4ccccc4c4cc5c(cc34)oc3ccccc35)cc2)cc1] 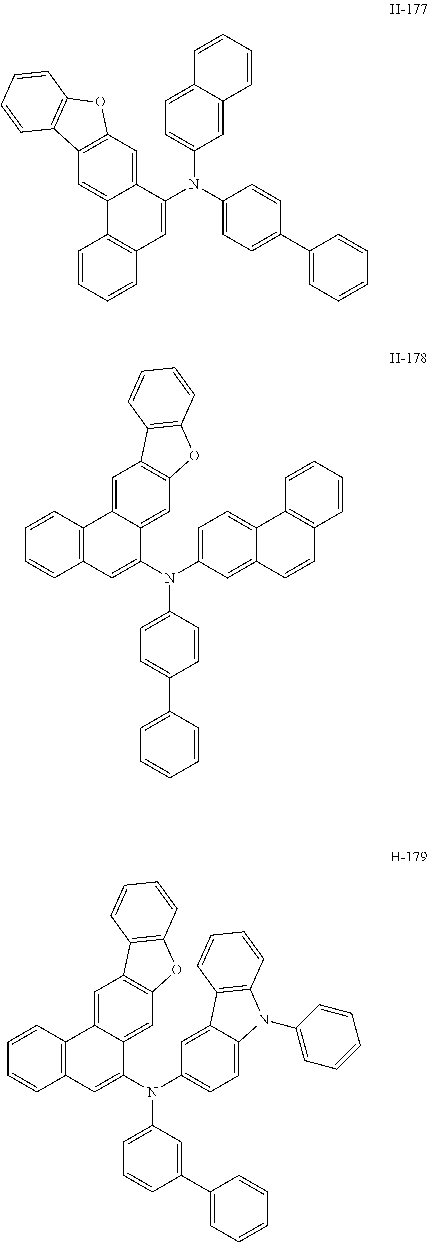 [SMILES: c1ccc(-c2ccc(N(c3ccc4ccccc4c3)c3cc4ccccc4c4cc5c(cc34)oc3ccccc35)cc2)cc1 ; c1ccc(-c2ccc(N(c3ccc4c(ccc5ccccc54)c3)c3cc4ccccc4c4cc5c(cc34)oc3ccccc35)cc2)cc1 ; c1ccc(-c2cccc(N(c3ccc4c(c3)c3ccccc3n4-c3ccccc3)c3cc4ccccc4c4cc5c(cc34)oc3ccccc35)c2)cc1] 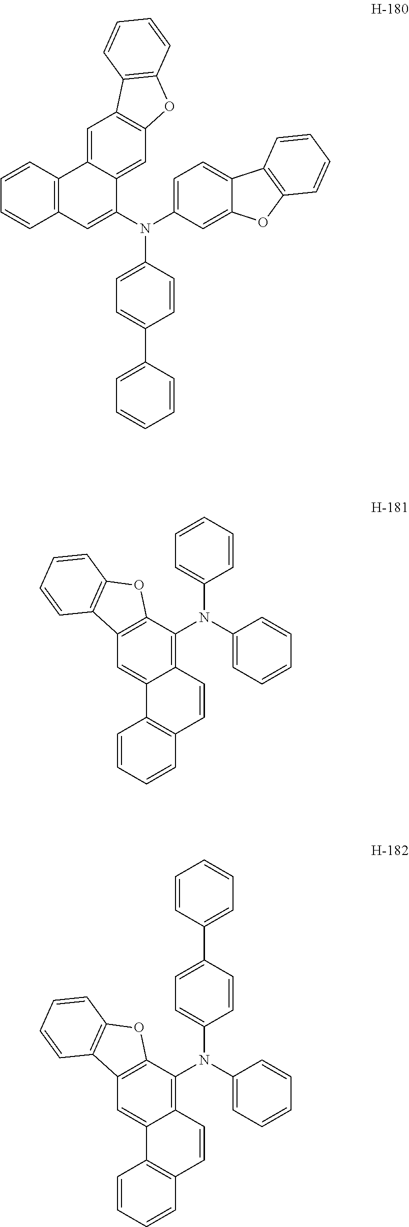 [SMILES: c1ccc(-c2ccc(N(c3ccc4c(c3)oc3ccccc34)c3cc4ccccc4c4cc5c(cc34)oc3ccccc35)cc2)cc1 ; c1ccc(N(c2ccccc2)c2c3ccc4ccccc4c3cc3c2oc2ccccc23)cc1 ; c1ccc(-c2ccc(N(c3ccccc3)c3c4ccc5ccccc5c4cc4c3oc3ccccc34)cc2)cc1] 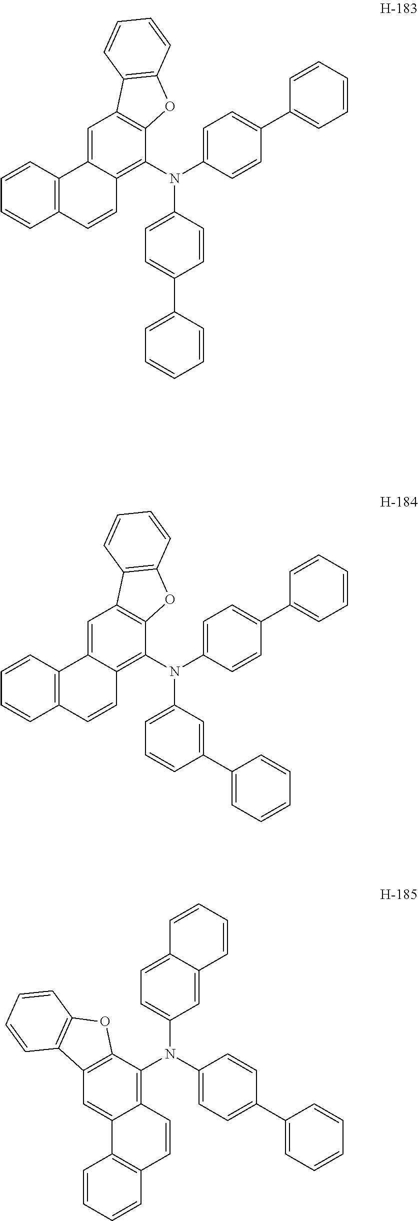 [SMILES: c1ccc(-c2ccc(N(c3ccc(-c4ccccc4)cc3)c3c4ccc5ccccc5c4cc4c3oc3ccccc34)cc2)cc1 ; c1ccc(-c2ccc(N(c3cccc(-c4ccccc4)c3)c3c4ccc5ccccc5c4cc4c3oc3ccccc34)cc2)cc1 ; c1ccc(-c2ccc(N(c3ccc4ccccc4c3)c3c4ccc5ccccc5c4cc4c3oc3ccccc34)cc2)cc1] 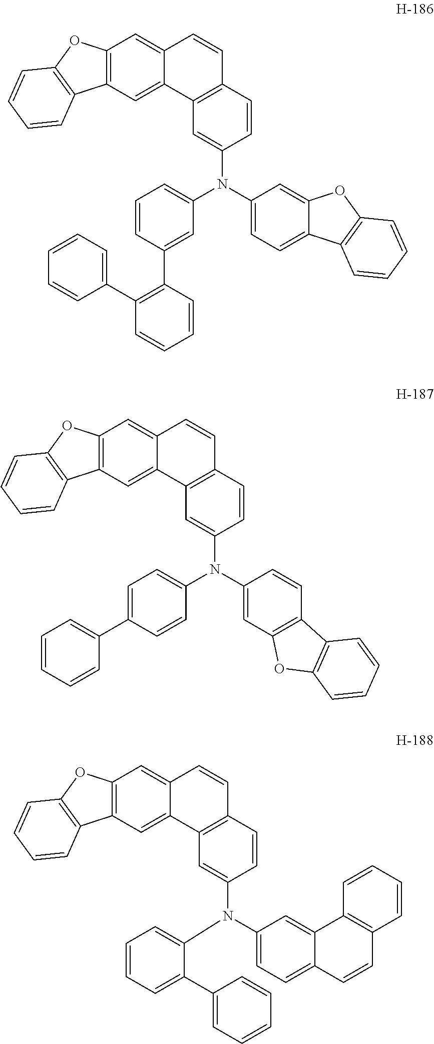 [SMILES: c1ccc(-c2ccccc2-c2cccc(N(c3ccc4c(c3)oc3ccccc34)c3ccc4ccc5cc6oc7ccccc7c6cc5c4c3)c2)cc1 ; c1ccc(-c2ccc(N(c3ccc4c(c3)oc3ccccc34)c3ccc4ccc5cc6oc7ccccc7c6cc5c4c3)cc2)cc1 ; c1ccc(-c2ccccc2N(c2ccc3ccc4ccccc4c3c2)c2ccc3ccc4cc5oc6ccccc6c5cc4c3c2)cc1] 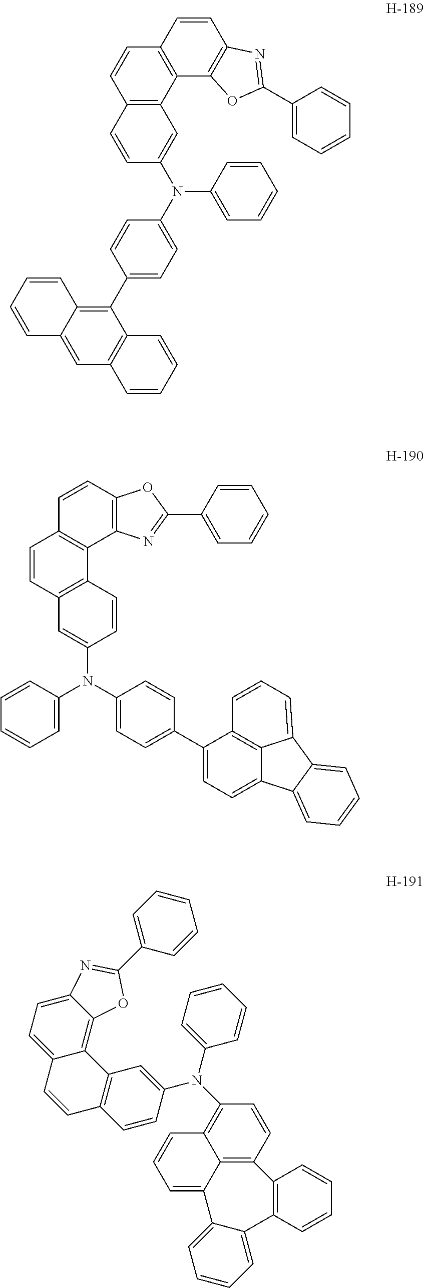 [SMILES: c1ccc(-c2nc3ccc4ccc5ccc(N(c6ccccc6)c6ccc(-c7c8ccccc8cc8ccccc78)cc6)cc5c4c3o2)cc1 ; c1ccc(-c2nc3c(ccc4ccc5cc(N(c6ccccc6)c6ccc(-c7ccc8c9c(cccc79)-c7ccccc7-8)cc6)ccc5c43)o2)cc1 ; c1ccc(-c2nc3ccc4ccc5ccc(N(c6ccccc6)c6ccc7c8c(cccc68)-c6ccccc6-c6ccccc6-7)cc5c4c3o2)cc1] 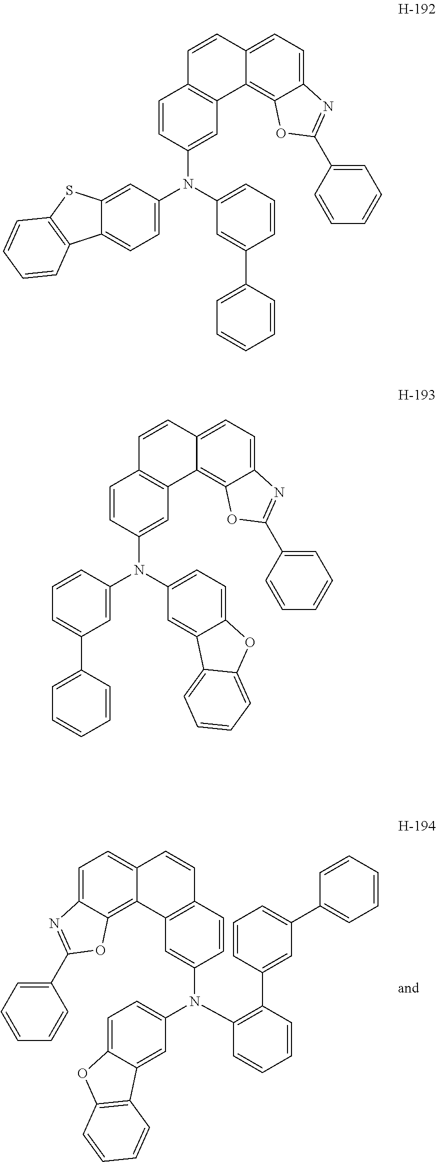 [SMILES: c1ccc(-c2cccc(N(c3ccc4c(c3)sc3ccccc34)c3ccc4ccc5ccc6nc(-c7ccccc7)oc6c5c4c3)c2)cc1 ; c1ccc(-c2cccc(N(c3ccc4oc5ccccc5c4c3)c3ccc4ccc5ccc6nc(-c7ccccc7)oc6c5c4c3)c2)cc1 ; c1ccc(-c2cccc(-c3ccccc3N(c3ccc4oc5ccccc5c4c3)c3ccc4ccc5ccc6nc(-c7ccccc7)oc6c5c4c3)c2)cc1] 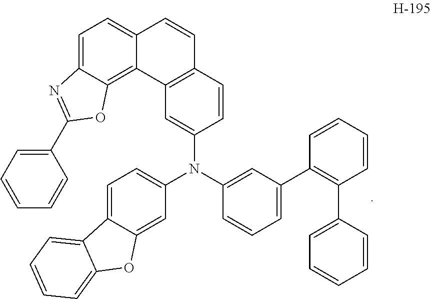 [SMILES: c1ccc(-c2nc3ccc4ccc5ccc(N(c6cccc(-c7ccccc7-c7ccccc7)c6)c6ccc7c(c6)oc6ccccc67)cc5c4c3o2)cc1]
